(19) United States
(12) Patent Application Publication
(10) Pub. No.: US 2026/0297087 A1
(43) Pub. Date: Oct. 1, 2026

(54) Pyridoimidazolone Compounds, Preparation Thereof and Therapeutic Uses Thereof

(71) Applicant: Sanofi, Paris (FR)

(72) Inventors: Franck Slowinski, Paris (FR); Jidong Zhang, Paris (FR); Amélie Dommergue, Paris (FR); Thomas Maier, Frankfurt am Main (DE); Elisabeth Speckmeier, Frankfurt am Main (DE)

(21) Appl. No.: 19/477,873

(22) PCT Filed: Apr. 26, 2024
(86) PCT No.: PCT/EP2024/061677

§ 371 Date: Oct. 23, 2025

(30) Foreign Application Priority Data
Apr. 28, 2023 (EP) 23315138.0

(51) Int. Cl. C07D 471/04; A61K 31/4545; A61K 31/5377; A61P 35/00

(52) U.S. Cl. CPC C07D 471/04; A61K 31/4545; A61K 31/5377; A61P 35/00

(57) ABSTRACT

Compounds are provided which can inhibit ERK5. Also provided are pharmaceutical compositions and medical uses of the same, including the use in treating or preventing conditions such as cancers.

Compounds are provided which can inhibit ERK5. Also provided are pharmaceutical compositions and medical uses of the same, including the use in treating or preventing conditions such as cancers.

### SUMMARY

The mitogen-activated protein kinase (MAPK) cascade is a highly-conserved cellular pathway which transmits signals from the cell surface to the nucleus. The pathway plays an important role in cell proliferation, differentiation, and migration and it is well known to be involved in the development of cancer. Proteins in the pathway include the extracellular signal-regulated kinase (ERK) proteins; among those, ERK5 (expressed from the MAPK7 gene) plays an important role in cell proliferation, as well as epithelial development and neural differentiation (see, e.g., Nishimoto et al., EMBO Reports (2006) 7(8):782-786). ERK5 is unique among the ERK proteins, having a large C-terminal domain which contains a transcriptional activation domain (TAD) as well as a nuclear localization signal and two proline-rich regions (see, e.g., Guo et al., Exp Ther Med. (2020) 19:1997-2007). Autophosphorylation of the TAD is required for transcriptional activation (see, e.g., Morimoto et al., J Biol Chem. (2007) 282(49):35449-35456).

ERK5 plays an important role in controlling cell proliferation and cell cycle progression, for example via direct or indirect phosphorylation of MEF2C, cMYC, SGK1, RSK, FOS, and FRA1 among others (see, e.g., Paudel et al., Int J Mol Sci. (2021) 22:7594-7614; Terasawa et al., Genes to Cells (2003) 8(3):263-273). The involvement of ERK5 in numerous biological pathways means that its activity is associated with many aspects of cancer progression, including tumour angiogenesis, metastasis, inflammation, sustained proliferation, and evasion of growth suppression. It therefore presents an attractive target for modulating disease pathology and treatment in a wide range of conditions. In previous studies, ERK5 inhibition or down-regulation has been shown to block tumorigenesis in murine leukaemia cells, reduce growth of chronic myeloid leukaemia cells, inhibit growth of breast cancer and multiple myeloma cells, suppress colon cancer cell proliferation, and have to have an impact on renal cell carcinoma, mesothelioma, adenocarcinoma, neuroblastoma and hepatocellular carcinoma cell growth or survival, among others (see, e.g., Stecca et al., Int J Mol Sci. (2019) 20:1426-1446).

ERK5 inhibition thus represents a promising approach to tackle a broad range of cancers. Several ERK5 inhibitors have been developed and some are under clinical review. For example, WO 2019/170543 (Bayer AG and Bayer Pharma AG) discloses compounds which are said to be active as ERK5 inhibitors in the μM to nM concentration range.

Despite recent progress in cancer treatment with the development of targeted therapies and immunotherapies, not all cancer patients can be offered an efficient therapeutic solution. There is therefore a need to identify and develop new drugs. The present disclosure seeks to address this need by providing novel compounds for use as ERK5 inhibitors and for the treatment of ERK5 related diseases and conditions.

Accordingly, a first aspect provides a compound, being of Formula (N-I)

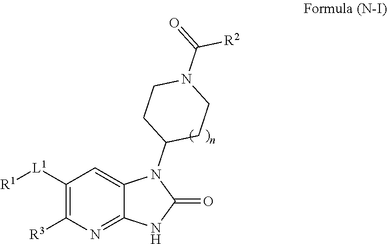

or a pharmaceutically acceptable salt thereof, wherein:
R¹ is selected from halo, —(C₁-C₆)alkyl, —(C₃-C₇)cycloalkyl, 4- to 10-membered heterocycloalkyl, and 5- to 10-membered heteroaryl, wherein R¹ is optionally substituted with one or more occurrences of R^{A},
   wherein each R^{A} is independently selected from halo, —OH, oxo, ═N—OH, —(C₁-C₃)alkyl, and —O(C₁-C₃)alkyl, wherein each occurrence of —(C₁-C₃)alkyl is optionally substituted by one or more groups independently selected from halo and —OH,
   and/or wherein two occurrences of R^{A} may be taken together with the atom(s) to which they are attached to form a (C₃-C₆)cycloalkyl group or a 3- to 6-membered heterocyclic group, wherein the cycloalkyl or heterocyclic group is optionally substituted by one or more halo;
L¹ is selected from a direct bond, —O—, and —CH₂—;
R² is —(C₆-C₁₀)aryl, wherein R² is substituted with one, or two occurrences of R^{B},
   wherein each R^{B} is independently selected
from —OH, —NH₂, —SF₅, —NHC(O)OC(CH₃)₃, and —O(C₁-C₃)alkyl, wherein each occurrence of —O(C₁-C₃)alkyl is optionally substituted by one or more halo;
R³ is selected from —H, —OH, and —(C₁-C₃)alkyl; and
n is 0, or 1.

In embodiments, R¹ is selected from:

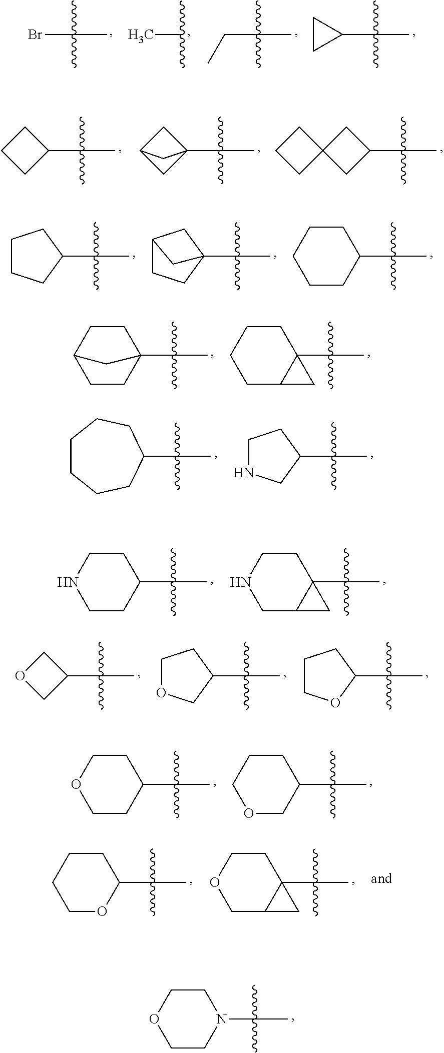

wherein R¹ is optionally substituted by one or two occurrences of R^{A} as defined hereinbefore. In embodiments, each R^{A} is independently selected from —F, —OH, oxo, ═N—OH, —OCH₃, and —(C₁-C₂)alkyl optionally substituted by one or more substituents selected from —F and —OH.

In embodiments, L¹ is —O— or —CH₂—.

In embodiments, R² is selected from:

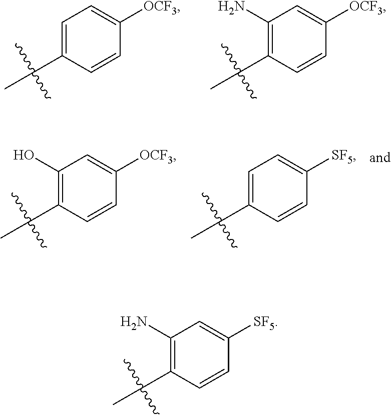

A further aspect provides a compound, being of Formula (N-II)

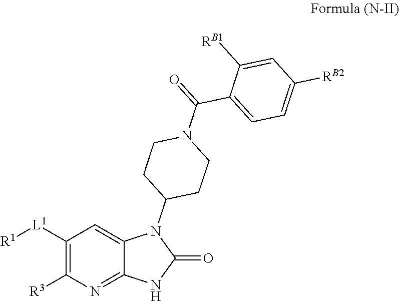

or a pharmaceutically acceptable salt thereof, wherein:
R¹ is selected from halo (e.g., —Br), —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo (e.g., —F), —OH, oxo, —O(C₁-C₃)alkyl, and —(C₁-C₃)alkyl optionally substituted by one or more groups independently selected from halo (e.g., —F) and —OH,
   and/or wherein two occurrences of R^{A} may be taken together with the atom(s) to which they are attached to form a (C₃-C₆)cycloalkyl or 3- to 6-membered heterocycloalkyl group, wherein the cycloalkyl group is optionally substituted by —F;
L¹ is selected from a direct bond, —O—, and —CH₂—;
R^{B1} is selected from —H, —OH, —NH₂, and —NHC(O)OC(CH₃)₃;
R^{B2} is selected from —SF₅, and —OCF₃; and
R³ is selected from —H, and —(C₁-C₃)alkyl (e.g., —CH₃).

In embodiments R is selected from:

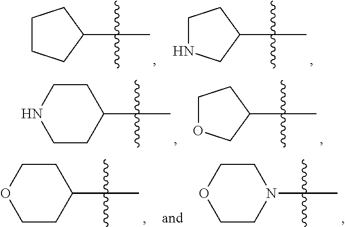

and wherein R¹ is optionally substituted by one occurrence of R^{A} as defined hereinbefore.

A further aspect provides a compound, being of Formula (III) or Formula (IV)

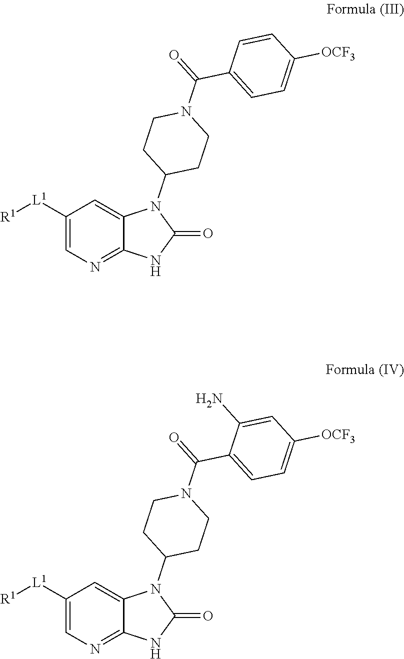

or a pharmaceutically acceptable salt thereof, wherein R¹ and L¹ are as defined hereinbefore.

A further aspect provides a compound, being of Formula (V), Formula (VI), or Formula (VII)

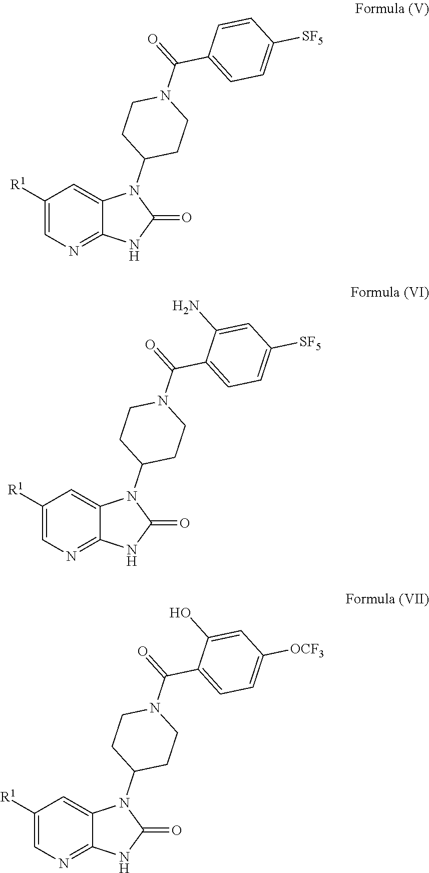

or a pharmaceutically acceptable salt thereof, wherein R¹ is selected from —(C₃-C₇)cycloalkyl, and 4- to 10-membered heterocycloalkyl, wherein R¹ is optionally substituted with one or more occurrences of R^{A}, and wherein each R^{A} is independently —(C₁-C₃)alkyl optionally substituted by —OH.

A further aspect provides a compound selected from the group consisting of:
1-[1-[2-amino-4-(trifluoromethoxy)benzoyl]-4-piperidyl]-6-tetrahydropyran-4-yloxy-3H-imidazo[4,5-b]pyridin-2-one,
1-[1-[2-amino-4-(trifluoromethoxy)benzoyl]-4-piperidyl]-6-tetrahydropyran-4-yl-3H-imidazo[4,5-b]pyridin-2-one,
1-[1-[2-amino-4-(trifluoromethoxy)benzoyl]-4-piperidyl]-6-N-morpholino-3H-imidazo[4,5-b]pyridin-2-one,
6-tetrahydropyran-4-yloxy-1-[1-[4-(trifluoromethoxy)benzoyl]-4-piperidyl]-3H-imidazo[4,5-b]pyridin-2-one,
6-[(1-methyl-4-piperidyl)oxy]-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-(1-methylpyrrolidin-3-yl)oxy-3H-imidazo[4,5-b]pyridin-2-one,
1-[1-[2-amino-4-(trifluoromethoxy)benzoyl]-4-piperidyl]-6-[(1-methyl-4-piperidyl)oxy]-3H-imidazo[4,5-b]pyridin-2-one,
6-N-morpholino-1-[1-[4-(trifluoromethoxy)benzoyl]-4-piperidyl]-3H-imidazo[4,5-b]pyridin-2-one,
6-(1-methylpyrrolidin-3-yl)oxy-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[2-(hydroxymethyl)morpholin-4-yl]-3H-imidazo[4,5-b]pyridin-2-one,
6-[2-(hydroxymethyl)morpholin-4-yl]-1-[1-[4-(trifluoromethoxy)benzoyl]-4-piperidyl]-3H-imidazo[4,5-b]pyridin-2-one,
1-[1-[2-amino-4-(pentafluoro-λ⁶-sulfanyl)benzoyl]-4-piperidyl]-6-tetrahydropyran-4-yl-3H-imidazo[4,5-b]pyridin-2-one,
6-tetrahydrofuran-3-yl-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-tetrahydrofuran-3-yl-3H-imidazo[4,5-b]pyridin-2-one,
6-cyclopentyl-1-[1-[4-(trifluoromethoxy)benzoyl]-4-piperidyl]-3H-imidazo[4,5-b]pyridin-2-one,
1-[1-[2-hydroxy-4-(trifluoromethoxy)benzoyl]-4-piperidyl]-6-tetrahydropyran-4-yl-3H-imidazo[4,5-b]pyridin-2-one,
1-[1-[4-(pentafluoro-λ⁶-sulfanyl)benzoyl]-4-piperidyl]-6-tetrahydropyran-4-yl-3H-imidazo[4,5-b]pyridin-2-one,
1-[1-[2-amino-4-(trifluoromethoxy)benzoyl]-4-piperidyl]-5-methyl-6-tetrahydropyran-4-yl-3H-imidazo[4,5-b]pyridin-2-one,
1-[1-[2-amino-4-(trifluoromethoxy)benzoyl]-4-piperidyl]-6-(2-fluorospiro[3.3]heptan-6-yl)-3H-imidazo[4,5-b]pyridin-2-one,
1-[1-[2-amino-4-(trifluoromethoxy)benzoyl]-4-piperidyl]-6-(2-oxo-4-piperidyl)-3H-imidazo[4,5-b]pyridin-2-one,
1-[1-[2-amino-4-(trifluoromethoxy)benzoyl]-4-piperidyl]-6-(tetrahydrofuran-3-ylmethyl)-3H-imidazo[4,5-b]pyridin-2-one,
1-[1-[2-amino-4-(trifluoromethoxy)benzoyl]-4-piperidyl]-6-(2-methoxyethyl)-3H-imidazo[4,5-b]pyridin-2-one,
1-[1-[2-amino-4-(trifluoromethoxy)benzoyl]-4-piperidyl]-6-tetrahydrofuran-2-yl-3H-imidazo[4,5-b]pyridin-2-one,
trans-6-(4-methoxycyclohexyl)-1-[1-[4-(trifluoromethoxy)benzoyl]-4-piperidyl]-3H-imidazo[4,5-b]pyridin-2-one,
cis-6-(4-methoxycyclohexyl)-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-(3-azabicyclo[4.1.0]heptan-6-yl)-3H-imidazo[4,5-b]pyridin-2-one,
1-[1-[2-amino-4-(trifluoromethoxy)benzoyl]-4-piperidyl]-6-cyclobutyl-3H-imidazo[4,5-b]pyridin-2-one,
1-[1-[2-amino-4-(trifluoromethoxy)benzoyl]-4-piperidyl]-6-[3-(trifluoromethyl)-1-bicyclo[1.1.1]pentanyl]-3H-imidazo[4,5-b]pyridin-2-one,
1-[1-[2-amino-4-(trifluoromethoxy)benzoyl]-4-piperidyl]-6-(3-oxabicyclo[4.1.0]heptan-6-yl)-3H-imidazo[4,5-b]pyridin-2-one,
1-[1-[2-amino-4-(trifluoromethoxy)benzoyl]-4-piperidyl]-6-(1-bicyclo[2.1.1]hexanyl)-3H-imidazo[4,5-b]pyridin-2-one,
1-[1-[2-amino-4-(trifluoromethoxy)benzoyl]-4-piperidyl]-6-[3-(difluoromethyl)-1-bicyclo[1.1.1]pentanyl]-3H-imidazo[4,5-b]pyridin-2-one,
1-[1-[2-amino-4-(trifluoromethoxy)benzoyl]-4-piperidyl]-6-norcaran-1-yl-3H-imidazo[4,5-b]pyridin-2-one,
1-[1-[2-amino-4-(trifluoromethoxy)benzoyl]-4-piperidyl]-6-(4,4-difluorocyclohexyl)-3H-imidazo[4,5-b]pyridin-2-one,
1-[1-[2-amino-4-(trifluoromethoxy)benzoyl]-4-piperidyl]-6-cyclohexyl-3H-imidazo[4,5-b]pyridin-2-one,
1-[1-[2-amino-4-(trifluoromethoxy)benzoyl]-4-piperidyl]-6-norbornan-2-yl-3H-imidazo[4,5-b]pyridin-2-one,
1-[1-[2-amino-4-(trifluoromethoxy)benzoyl]-4-piperidyl]-6-tetrahydropyran-2-yl-3H-imidazo[4,5-b]pyridin-2-one,
1-[1-[2-amino-4-(trifluoromethoxy)benzoyl]-4-piperidyl]-6-(tetrahydropyran-4-ylmethyl)-3H-imidazo[4,5-b]pyridin-2-one,
1-[1-[2-amino-4-(trifluoromethoxy)benzoyl]-4-piperidyl]-6-tetrahydropyran-3-yl-3H-imidazo[4,5-b]pyridin-2-one,
6-tetrahydropyran-4-yl-1-[1-[4-(trifluoromethoxy)benzoyl]-4-piperidyl]-3H-imidazo[4,5-b]pyridin-2-one,
6-(oxetan-3-ylmethyl)-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[tetrahydrofuran-3-yl]oxy-3H-imidazo[4,5-b]pyridin-2-one,
trans-1-[1-[2-amino-4-(trifluoromethoxy)benzoyl]-4-piperidyl]-6-(4-methoxycyclohexyl)-3H-imidazo[4,5-b]pyridin-2-one,
cis-1-[1-[2-amino-4-(trifluoromethoxy)benzoyl]-4-piperidyl]-6-(4-methoxycyclohexyl)-3H-imidazo[4,5-b]pyridin-2-one,
1-[1-[2-amino-4-(trifluoromethoxy)benzoyl]-4-piperidyl]-6-[1-methylpyrrolidin-3-yl]oxy-3H-imidazo[4,5-b]pyridin-2-one,
1-[1-[2-amino-4-(pentafluoro-λ⁶-sulfanyl)benzoyl]pyrrolidin-3-yl]-6-tetrahydropyran-4-yl-3H-imidazo[4,5-b]pyridin-2-one,
1-[1-[4-(pentafluoro-λ⁶-sulfanyl)benzoyl]pyrrolidin-3-yl]-6-tetrahydropyran-4-yl-3H-imidazo[4,5-b]pyridin-2-one,
trans-1-[1-[2-amino-4-(trifluoromethoxy)benzoyl]-4-piperidyl]-6-(4-hydroxycyclohexyl)-3H-imidazo[4,5-b]pyridin-2-one,
cis-1-[1-[2-amino-4-(trifluoromethoxy)benzoyl]-4-piperidyl]-6-(4-hydroxycyclohexyl)-3H-imidazo[4,5-b]pyridin-2-one,
1-[1-[2-amino-4-(trifluoromethoxy)benzoyl]-4-piperidyl]-6-[1-(2,2,2-trifluoroethyl)-4-piperidyl]-3H-imidazo[4,5-b]pyridin-2-one,
1-[1-[2-amino-4-(trifluoromethoxy)benzoyl]-4-piperidyl]-6-(1-methyl-4-piperidyl)-3H-imidazo[4,5-b]pyridin-2-one,
1-[1-[2-amino-4-(trifluoromethoxy)benzoyl]-4-piperidyl]-6-tetrahydrofuran-3-yloxy-3H-imidazo[4,5-b]pyridin-2-one,
6-tetrahydrofuran-3-yloxy-1-[1-[4-(trifluoromethoxy)benzoyl]-4-piperidyl]-3H-imidazo[4,5-b]pyridin-2-one,
trans-6-(4-hydroxycyclohexyl)-1-[1-[4-(trifluoromethoxy)benzoyl]-4-piperidyl]-3H-imidazo[4,5-b]pyridin-2-one,
cis-6-(4-hydroxycyclohexyl)-1-[1-[4-(trifluoromethoxy)benzoyl]-4-piperidyl]-3H-imidazo[4,5-b]pyridin-2-one,
tert-butyl N-[2-[4-(6-bromo-2-oxo-3H-imidazo[4,5-b]pyridin-1-yl)piperidine-1-carbonyl]-5-(trifluoromethoxy)phenyl]carbamate,
6-bromo-1-[1-[4-(trifluoromethoxy)benzoyl]-4-piperidyl]-3H-imidazo[4,5-b]pyridin-2-one,
trans-6-(4-hydroxycycloheptyl)-1-[1-[4-(trifluoromethoxy)benzoyl]-4-piperidyl]-3H-imidazo[4,5-b]pyridin-2-one,
cis-6-(4-hydroxycycloheptyl)-1-[1-[4-(trifluoromethoxy)benzoyl]-4-piperidyl]-3H-imidazo[4,5-b]pyridin-2-one,
6-(4-oxocycloheptyl)-1-[1-[4-(trifluoromethoxy)benzoyl]-4-piperidyl]-3H-imidazo[4,5-b]pyridin-2-one,
6-[4-hydroxyiminocycloheptyl]-1-[1-[4-(trifluoromethoxy)benzoyl]-4-piperidyl]-3H-imidazo[4,5-b]pyridin-2-one,
trans-6-(3-hydroxycycloheptyl)-1-[1-[4-(trifluoromethoxy)benzoyl]-4-piperidyl]-3H-imidazo[4,5-b]pyridin-2-one,
cis-6-(3-hydroxycycloheptyl)-1-[1-[4-(trifluoromethoxy)benzoyl]-4-piperidyl]-3H-imidazo[4,5-b]pyridin-2-one,
   and the pharmaceutically acceptable salts thereof.

A further aspect provides a pharmaceutical composition comprising the compound defined hereinbefore and at least one pharmaceutically acceptable excipient or carrier.

A further aspect provides a compound, or a pharmaceutical composition, as defined hereinbefore for use in therapy.

A further aspect provides a compound, or a pharmaceutical composition, as defined hereinbefore for use in the treatment or prevention of cancer.

In embodiments, the cancer is characterized by increased MAPK7 expression and/or increased ERK5 activity.

In embodiments, the cancer is selected from leukaemia (e.g., chronic myeloid leukaemia), breast cancer, multiple myeloma, colon cancer, colorectal cancer, lung cancer, pancreatic cancer, renal cell carcinoma, mesothelioma, adenocarcinoma, neuroblastoma, melanoma, and hepatocellular carcinoma.

### DETAILED DESCRIPTION

Although specific embodiments of the present disclosure will now be described with reference to the description and examples, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, exemplary methods, devices, and materials are now described. All technical and patent publications cited herein are incorporated herein by reference in their entirety.

The practice of the present disclosure will employ, unless otherwise indicated, conventional techniques of chemical synthesis, tissue culture, immunology, molecular biology, microbiology, cell biology, recombinant DNA, etc., which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, molecular weight, etc., including ranges, are approximations which are varied (+) or (−) by increments of, e.g., 0.1 or 1.0, where appropriate. It is to be understood, although not always explicitly stated, that all numerical designations are preceded by the term “about”, which is used to denote a conventional level of variability. For example, a numerical designation which is “about” a given value may vary by ±10% of said value; alternatively, the variation may be ±5%, ±2%, or ±1% of the value. It also is to be understood, although not always explicitly stated, that the reagents described herein are merely exemplary and that equivalents of such are known in the art.

As used in the specification and claims, the singular forms “a”, “an”, and “the” include plural references unless the context clearly dictates otherwise. For example, the term “a cell” includes a plurality of cells, including mixtures thereof. Unless specifically stated or obvious from context, as used herein, the term “or” is understood to be inclusive. The term “including” is used herein to mean, and is used interchangeably with, the phrase “including but not limited to”.

As used herein, the term “comprising” or “comprises” is intended to mean that the compositions and methods include the recited elements, without excluding other elements. “Consisting essentially of” when used to define compositions and methods, shall mean excluding other elements of any essential significance for the stated purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure. Use of the term “comprising” herein is intended to encompass, and to disclose, the corresponding statements in which the term “comprising” is replaced by “consisting essentially of” or “consisting of”.

A “subject,” “individual”, or “patient” is used interchangeably herein, and refers to a vertebrate, such as a mammal. Mammals include, but are not limited to, rodents, farm animals, sport animals, pets, and primates; for example murines, rats, rabbit, simians, bovines, ovines, porcines, canines, felines, equines, and humans. In a particular embodiment, the mammal is a human.

“Administering” is defined herein as a means of providing an agent or a composition containing the agent to a subject in a manner that results in the agent being contacted with (e.g., being inside) the subject's body. Such an administration can be by any route including, without limitation, oral, transdermal (e.g., by the vagina, rectum, or oral mucosa), by injection (e.g., subcutaneous, intravenous, parenteral, intraperitoneal, or into the central nervous system), or by inhalation (e.g., oral or nasal). Administration may also involve providing a substance or composition to a part of the surface of the subject's body, for example by topical administration to the skin. Pharmaceutical preparations are, of course, given by forms suitable for each administration route.

“Treating” or “treatment” of a disease includes: (1) preventing the disease, i.e. causing the clinical symptoms of the disease not to develop in a patient that may be predisposed to the disease but does not yet experience or display symptoms of the disease; (2) inhibiting the disease, i.e. arresting or reducing the development of the disease or its clinical symptoms; and/or (3) relieving the disease, i.e. causing regression of the disease or its clinical symptoms.

The term “suffering” as it relates to the term “treatment” refers to a patient or individual who has been diagnosed with or is predisposed to the disease. A patient may also be referred to being “at risk of suffering” from a disease because of a history of disease in their family lineage or because of the presence of genetic mutations associated with the disease. A patient at risk of a disease has not yet developed all or some of the characteristic pathologies of the disease.

An “effective amount” or “therapeutically effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including, for example, the activity of the specific compound employed, the age, body weight, general health, sex, and diet of the subject, the time of administration, the rate of excretion, the drug combination, the severity of the particular disorder being treated and the form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to treat (e.g., improve) one or more symptoms associated with the condition. The total daily dose may be administered in single or divided doses and may, at the physician's discretion, fall outside of the typical range given herein.

As used herein, the terms “increased” and “elevated” are used interchangeably and encompass any measurable increase in a biological function and/or a biological activity and/or a concentration. For example, an increase can be by at least about 10%, e.g. at least about 15%, 20%, 25%, 30%, 35%, 40%, 45%, 50%, 55%, 60%, 65%, 70%, 75%, 80%, 85%, 90%, or 95%, such as at least about 95%, 96%, 97%, 98%, 99%, or 100%. Thus, an increase can be by at least about 2-fold, 3-fold, 4-fold, 5-fold, 6-fold, 7-fold, 8-fold, 9-fold, or 10-fold, such as at least about 20-fold, 25-fold, 50-fold, 100-fold, or higher, relative to a control or baseline amount or function, or activity, or concentration.

As used herein, the terms “increased expression” and/or “increased activity” of a substance, such as ERK5, in a sample or cancer or patient, typically refers to an increase in the amount of the substance (e.g., of the MAPK7 gene product or ERK5 protein), although it may also denote an increase in the biological activity of the substance (e.g., constitutive activation of phosphorylation and/or reduced discrimination of phosphorylation sites of ERK5). For example, an increase can be by an amount of about 5%, e.g., about 10%, 15%, 20%, 25%, 30%, 35%, 40%, 45%, 50%, 55%, 60%, 65%, 70%, 75%, 80%, 85%, 90%, or 95%, such as about 96%, 97%, 98%, 99%, or 100%. Thus, the increase can be about 2-fold, 3-fold, 4-fold, 5-fold, 6-fold, 7-fold, 8-fold, 9-fold, or 10-fold, such as about 20-fold, 25-fold, 50-fold, 100-fold, or higher, relative to the amount (or activity) of the substance, such as ERK5, in a control sample or control samples, such as an individual or group of individuals who are not suffering from the disease or disorder (e.g. cancer) or an internal control, as determined by techniques known in the art. A subject can also be determined to have an “increased expression” or “increased activity” of ERK5 if the expression and/or activity of ERK5 is increased by one standard deviation, two standard deviations, three standard deviations, four standard deviations, five standard deviations, or more, relative to the mean (average) or median amount of ERK5 in a control group of samples or a baseline group of samples or a retrospective analysis of patient samples. As practiced in the art, such control or baseline expression levels can be previously determined, or measured prior to the measurement in the sample or cancer or subject, or can be obtained from a database of such control samples.

As used herein, the term “pharmaceutically acceptable excipient” encompasses any of the standard pharmaceutical excipients, for example as described in Remington's Pharmaceutical Sciences (20th ed., Mack Publishing Co. 2000). Such excipients include carriers such as a phosphate buffered saline solution, water, and emulsions, such as an oil/water or water/oil emulsion, and various types of wetting agents. Pharmaceutical compositions also can include stabilizers, preservatives, adjuvants, fillers, binders, lubricants, and the like.

As used herein, the term “alkyl” means a saturated linear or branched free radical consisting essentially of carbon atoms and a corresponding number of hydrogen atoms. Exemplary alkyl groups include methyl, ethyl, n-propyl, isopropyl, n-butyl, isobutyl, etc. Other alkyl groups will be readily apparent to those of skill in the art given the benefit of the present disclosure.

The terms “(C₁-C₃)alkyl”, “(C₁-C₆)alkyl”, etc., have equivalent meanings, i.e., a saturated linear or branched free radical consisting essentially of 1 to 3 (or 1 to 6) carbon atoms and a corresponding number of hydrogen atoms. The definition of “alkyl” also applies in the context of other groups which comprise alkyl groups, such as “—O(C₁-C₃)alkyl”. The term “haloalkyl” means an alkyl group which is substituted by one or more halogens. Exemplary haloalkyl groups include trifluoromethyl, trifluoroethyl, difluoroethyl, pentafluoroethyl, chloromethyl, etc. One or more carbon atoms in the backbone of an alkyl group may be substituted by (or bonded to) a heteroatom by a multiple bond (e.g., a double bond); for example, a carbon atom of the alkyl group may be bonded to oxygen via a double bond (i.e., substituted by oxo to provide a carbonyl function). The presence of such a substituent does not prevent the carbon backbone of the free radical being considered as an alkyl group.

As used herein, the term “cyclic group” means a saturated, partially or fully unsaturated, or aromatic group having at least 3 to 10 atoms (i.e., ring atoms) that form a ring. Where a cyclic group is defined as having a certain number of members, the term “members”, “membered” and the like is used to denote the number of ring atoms in said cyclic group. For example, a 5-membered cyclic group (e.g., a 5-membered heterocyclic group) contains 5 ring atoms. It will be appreciated that a cyclic group may be part of a larger cyclic system; for example, bicyclo[4.3.0]nonane comprises two carbocyclic groups, namely a cyclohexane group and a cyclopentane group, which are fused to form the carbocyclic system which makes up the molecule. The term “cyclic group” is intended to encompass both carbocyclic groups as well as heterocyclic groups. The term “carbocyclic” refers to a group having at least 3 to 9 carbon atoms that form a ring. The term “heterocyclic” refers to a group having at least 3 to 10 atoms that form a ring, wherein at least 1 to 9 of said ring atoms are carbon and the remaining at least 1 to 9 ring atom(s) (i.e., hetero ring atom(s)) are selected independently from the group consisting of nitrogen, sulphur, and oxygen.

The term “spiro” or “spirocyclic” as used herein in relation to cyclic groups denotes that a first cyclic group within a multicyclic system is attached to a second cyclic group within said multicyclic system, wherein the ring atoms of said first cyclic group and the ring atoms of said second cyclic group have only one atom in common, i.e., said first and second cyclic groups share only one common ring atom. For example, the spiro[5.5]undecanyl group comprises two cyclohexane rings which have a single carbon ring atom in common.

The term “fused” as used herein in relation to cyclic groups denotes that a first cyclic group within a multicyclic system is attached to a second cyclic group within said multicyclic system, wherein the ring atoms of said first cyclic group and the ring atoms of said second cyclic group have two adjacent atoms in common, i.e., said first and second cyclic groups share two common ring atoms. For example, the bicyclo[4.4.0]decanyl group comprises two cyclohexane rings which have two adjacent carbon ring atoms in common.

The term “bridged” as used herein in relation to cyclic groups denotes that a first cyclic group within a multicyclic system is attached to a second cyclic group within said multicyclic system, wherein the ring atoms of said first cyclic group and the ring atoms of said second cyclic group have more than two adjacent atoms in common, i.e., said first and second cyclic groups share three or more common ring atoms. For example, the bicyclo[3.3.1]nonanyl group comprises two cyclohexane rings which have three adjacent carbon ring atoms in common.

Within the structural formulae described herein, any ring system (including any spiro, fused, or bridged ring system) may be connected to other parts of a molecule through any atom having suitable valency. For example, a bicyclic ring may be connected to another part of the molecule through a ring atom (e.g., a secondary carbon atom or heteroatom such as N), or a bridgehead (e.g., a tertiary carbon atom). Spiro, fused, and bridged rings may be fully unsaturated, partially unsaturated, or fully saturated, and may have aromatic character in one or more of their constituent rings.

As used herein, the term “cycloalkyl” means a saturated free radical having at least 3 to 9 carbon atoms (i.e., ring atoms) that form a ring. Exemplary cycloalkyl groups include cyclopropyl, cyclobutyl, cyclopentyl and cyclohexyl. It will be appreciated that the cycloalkyl group may be monocyclic or multicyclic (e.g., fused, bridged, or spirocyclic). In the case of multicyclic cycloalkyl groups, there are further rings, e.g. 1 or more further rings, all of which contain from 3 to 7 carbon atoms (i.e., ring atoms). Exemplary cycloalkyl groups having such further rings include bicyclo[1.1.1]pentanyl. The term “(C₃-C₇)cycloalkyl” denotes that the cycloalkyl group contains from 3 to 7 carbon atoms in the ring portion of the group, which may be monocyclic or multicyclic (e.g., fused, bridged, or spirocyclic), for example cyclopropanyl (having 3 ring carbon atoms) or bicyclo[1.1.1]pentanyl (having 5 ring carbon atoms). One or more ring atoms of a cycloalkyl group may be substituted by (i.e., bonded to) a heteroatom by a double bond (e.g., cycloalkyl substituted by oxo). The presence of such a substituent does not prevent the carbon backbone of the free radical being considered as a cycloalkyl group.

As used herein, the term “aryl” means an aromatic free radical having at least 6 carbon atoms (i.e., ring atoms) that form a ring. It will be appreciated that the aryl group may be monocyclic or multicyclic (e.g., fused). In the case of multicyclic aryl groups, there are further rings, e.g. 1 or more further rings, all of which contain at least 3 carbon atoms (i.e., ring atoms). The further rings may also contain one or more heteroatoms and they may be saturated, unsaturated, or aromatic. A multicyclic aryl group is typically attached to the rest of the molecule via an aromatic ring, and typically not via a ring containing a heteroatom. In embodiments, the multicyclic aryl group does not contain any ring heteroatoms. Examples of aryl groups include phenyl and naphthalenyl, as well as indenyl and indanyl groups. Other aryl groups include, for example, tetrahydroisoquinolinyl bonded to the rest of the molecule via its phenyl ring. The term “(C₆-C₁₀)aryl” denotes that the aryl group contains from 6 to 10 carbon atoms in the ring portion of the group, which may be monocyclic or multicyclic (e.g., fused), for example phenyl (having 6 ring carbon atoms) or indanyl (having 9 ring carbon atoms). In embodiments, (C₆-C₁₀)aryl is phenyl.

As used herein, the term “heterocycloalkyl” means a saturated free radical having at least 3 to 10 atoms (i.e., ring atoms) that form a ring, wherein at least 1 to 9 of said ring atoms are carbon and the remaining at least 1 to 9 ring atom(s) (i.e., hetero ring atom(s)) are selected independently from the group consisting of nitrogen, sulphur, and oxygen. In embodiments, the hetero ring atom(s) are selected independently from the group consisting of nitrogen and oxygen. For example, the term “4- to 10-membered heterocycloalkyl” means a saturated free radical containing from 4 to 10 ring atoms, of which one or more is a hetero ring atom. Heterocycloalkyl rings may have oxo substituents, typically adjacent to a heteroatom (e.g., 2-oxopyrrolidinyl), but the oxygen atom does not form part of the ring and is excluded from the number of ring atoms. The presence of such a substituent does not prevent the ring (or rings) of the free radical being considered as a heterocycloalkyl group. Exemplary heterocycloalkyl groups include tetrahydrofuranyl, piperidinyl, morpholinyl and piperazinyl. Any ring sulphur atom may optionally carry one or more pendant (i.e., non-ring) oxygen atoms, as found in, e.g., a sulfolanyl group. In the case of multicyclic heterocyclic groups, there are further rings, e.g. 1 or more further rings, all of which contain from 3 to 7 ring atoms selected from carbon, nitrogen, sulphur, and oxygen. The further rings may be saturated, or partially or fully unsaturated (e.g., having aromatic character). Multicyclic heterocyclic groups include fused, bridged and spirocyclic ring systems. Where a multicyclic heterocycloalkyl group contains an unsaturated fused ring, the group is typically not bonded to the rest of the molecule via that fused ring. Exemplary multicyclic heterocyclic groups include 2-oxa-6-azaspiro[3.3]heptane, 3,6-diazabicyclo[3.2.0]heptane, 2,6-diazabicyclo[3.2.0]heptane, and octahydropyrrolo[1,2-a]pyrazine. Where a heterocycloalkyl group is described as being “X- to Y-membered” (where X and Y are integers), this means that the heterocycloalkyl group contains a total number of ring atoms from X to Y. Thus, for example, a “4- to 7-membered heterocycloalkyl group” contains a total of 4, 5, 6, or 7 ring atoms, for example tetrahydropyranyl (6 ring atoms).

As used herein, the term “heteroaryl” means an aromatic (i.e., having aromatic character) free radical typically containing from 5 to 10 ring atoms, wherein 1 to 9 of said ring atoms are carbon and the remaining 1 to 9 ring atom(s) (i.e., hetero ring atom(s)) are selected independently from the group consisting of nitrogen, sulphur, and oxygen. In embodiments, the hetero ring atoms are selected from nitrogen and sulphur. It will be appreciated that the heteroaryl group may be monocyclic or multicyclic (e.g., fused). In the case of multicyclic heteroaryl groups, there are further rings, e.g. 1 or more further rings, all of which contain at least 3 atoms (i.e., ring atoms), which further rings may optionally be aromatic. Examples of heteroaryl groups include monocyclic groups such as pyridyl, and 2-oxopyridinyl, as well as multicyclic groups such as indolyl. Where a heteroaryl group is described as being “X- to Y-membered”, this means that the heteroaryl group contains a total number of ring atoms from X to Y. Thus, for example, a “5- to 10-membered heteroaryl group” contains a total of 5, 6, 7, 8, 9, or 10 ring atoms, for example indolyl (9 ring atoms). In embodiments, 5- to 10-membered heteroaryl is 4,5,6,7-tetrahydrothieno[3,2-c]pyridine.

As used herein, the terms “halo” and “halogen” mean fluorine, chlorine, bromine, or iodine. These terms are used interchangeably and may refer to a halogen free radical group or to a halogen atom as such. Those of skill in the art will readily be able to ascertain the identification of which in view of the context in which this term is used in the present disclosure. In embodiments, the halogen is selected from fluorine and bromine, e.g. fluorine.

As used herein, the term “oxo” means a free radical wherein an oxygen atom is connected to the atom bearing this radical via a double bond. For example, where a carbon atom carries an oxo radical it forms a carbon-oxygen double bond. It will be appreciated that not all atoms within a given structure can be substituted by oxo, and that this will depend on the free valency of the atom to be substituted.

The compounds of the present disclosure are described, inter alia, by way of structural formulae. It will be appreciated that these formulae typically show only one form (e.g., resonance form, tautomeric form, etc.) of the compound, whereas certain compounds may exist in more than one such form. This will be readily apparent to the skilled reader. The present disclosure includes all possible tautomers of the compounds characterised by the structural formulae hereinbefore and below, including as single tautomers, or as any mixture of tautomers in any ratio. For example, a pyridoimidazolone moiety (as shown, e.g., in Formula (I), and which may be referred to as 3H-imidazo[4,5-b]pyridin-2-one or 3H-imidazo[4,5-b]pyridin-2-ol), may be illustrated by any of the below tautomeric forms, which are used interchangeably throughout this description:

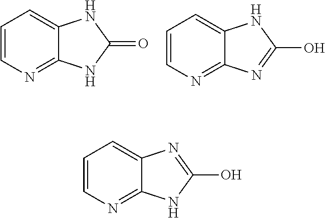

It will also be appreciated that certain of the present compounds may exist in one or more isomeric (e.g., stereoisomeric) forms. The present disclosure includes all possible stereoisomers, enantiomers, diastereomers, etc. of the compounds described hereinbefore and below, as well as cis- and trans-forms and conformers of the same. The purification and the separation of isomers may be accomplished by methods described hereinafter, as well as by techniques known in the art. For example, optical isomers of the compounds can be obtained by resolution of the racemic mixture of diastereoisomeric salts thereof (e.g., using an optically active acid or base, or by the formation of covalent diastereomers). A different process for separation of optical isomers involves the use of chiral chromatography (e.g., HPLC columns using a chiral phase), with or without conventional derivatization. Enzymatic separation, with or without derivatisation, may also be useful, and optically active compounds of the present disclosure can likewise be obtained by chiral syntheses utilizing optically active starting materials. The present disclosure includes all possible stereoisomers of the compounds described herein as single stereoisomers, or as any mixture of said stereoisomers, e.g. (R)- or (S)-isomers, in any ratio.

The compounds of the disclosure may exist in the form of free acids or bases, or may exist as addition salts with suitable acids or bases. For example, basic compounds of Formula (I) may be provided as pharmaceutically acceptable acid addition salts with an acid such as HCl, TFA, or formic acid (e.g., TFA). Methods for forming salts are described below and are also known in the art (see, e.g., Berge et al., J Pharm Sci. (1977) 66:1-19).

As used herein, the term “pharmaceutically acceptable” when used in connection with salts means a salt of a currently disclosed compound that may be administered without any resultant substantial undesirable biological effect(s) or any resultant deleterious interaction(s) with any other component of a pharmaceutical composition in which it may be contained.

A group defined as “optionally substituted” may be either unsubstituted, or substituted with one or more substituents, e.g. 1, 2, 3, 4, 5, 6, or more substituents. In embodiments, a substituted group has 1 to 4 substituents, e.g. 1, 2, or 3 substituents. In embodiments, a substituted group has 1 or 2 substituents. In embodiments, a substituted group has 3 substituents.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Compositions and methods provided herein may be combined with one or more of any of the other compositions and methods provided herein.

The following abbreviations and empirical formulae are used herein:
ABC ammonium bicarbonate
Ac Acetyl
ACN acetonitrile (MeCN)
ATP adenosine triphosphate
Boc tert-butyloxy carbonyl
CDI 1,1′-carbonyldiimidazole
DAD diode-array detection
DCM dichloromethane
DHP dihydropyran
DIPEA diisopropylethylamine
DMF N,N-dimethylformamide
DMSO dimethyl sulfoxide
4EBP1 eukaryotic translation initiation factor 4E-binding protein 1
ERK extracellular signal-regulated kinase
EtOAc/AcOEt ethyl acetate (also EA)
FRET Forster resonance energy transfer
HATU hexafluorophosphate azabenzotriazole tetramethyl uronium
HPLC high performance liquid chromatography
Ir[dF(CF₃)ppy]₂(dtbpy))PF₆ [4,4′-Bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate
LC/MS liquid chromatography/mass spectrometry
LED light-emitting diode
LiHMDS lithium bis(trimethylsilyl)amide
min minute(s)
MS mass spectrometry
OBD optimum bed density
Pd/C palladium on carbon
PdG2SPhos chloro(2-dicyclohexylphosphino-2′,6′-dimethoxy-1,1′-biphenyl)[2-(2′-amino-1,1′-biphenyl)]palladium(II) (also SPhosPdG2)
Pd(OAc)₂ palladium(II) diacetate
Pin pinacol (e.g. B₂Pin₂=bis(pinacolato)diboron)
PPTS pyridinium p-toluenesulfonate
rac racemic mixture
RPMI Roswell Park Memorial Institute medium
RuPhos 2-dicyclohexylphosphino-2′,6′-diisopropoxybiphenyl
TAD transcriptional activation domain
TATU O-(7-azabenzotriazole-1-yl)-N,N,N′,N′-tetramethyluronium
tetrafluoroborate
TBAF tetrabutylammonium fluoride
TBTU 2-(1H-benzotriazole-1-yl)-1,1,3,3-tetramethylaminium
tetrafluoroborate
TEA triethylamine
TFA trifluoroacetic acid
THF tetrahydrofuran
UPLC ultra-high performance liquid chromatography
UV ultraviolet
XPhos 2-dicyclohexylphosphino-2′,4′,6′-triisopropylbiphenyl
XPhosPdG4 methanesulfonato(2-dicyclohexylphosphino-2′,4′,6′-tri-isopropyl-1,1′-biphenyl)(2′-methylamino-1,1′-biphenyl-2-yl)palladium(II); CAS Registry No. 1599466-81-5

### Compounds

In a first aspect the present disclosure provides a compound being of Formula (N-I)

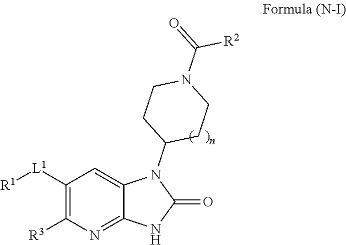

or a pharmaceutically acceptable salt thereof, wherein:
R¹ is selected from halo, —(C₁-C₆)alkyl, —(C₃-C₇)cycloalkyl, 4- to 10-membered heterocycloalkyl, and 5- to 10-membered heteroaryl, wherein R¹ is optionally substituted with one or more occurrences of R^{A},
   wherein each R^{A} is independently selected from halo, —OH, oxo, ═N—OH, —(C₁-C₃)alkyl, and —O(C₁-C₃)alkyl, wherein each occurrence of —(C₁-C₃)alkyl is optionally substituted by one or more groups independently selected from halo and —OH,
   and/or wherein two occurrences of R^{A} may be taken together with the atom(s) to which they are attached to form a (C₃-C₆)cycloalkyl group or a 3- to 6-membered heterocyclic group, wherein the cycloalkyl or heterocyclic group is optionally substituted by one or more halo;
L¹ is selected from a direct bond, —O—, and —CH₂—;
R² is —(C₆-C₁₀)aryl, wherein R² is substituted with one, or two occurrences of R^{B},
   wherein each R^{B} is independently selected
from —OH, —NH₂, —SF₅, —NHC(O)OC(CH₃)₃, and —O(C₁-C₃)alkyl, wherein each occurrence of —O(C₁-C₃)alkyl is optionally substituted by one or more halo;
R³ is selected from —H, —OH, and —(C₁-C₃)alkyl; and
n is 0, or 1.

In embodiments, the compound is a compound of Formula (N-I) or a pharmaceutically acceptable salt thereof, wherein:
R¹ is selected from halo, —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo, —OH, oxo, ═N—OH, —(C₁-C₃)alkyl, and —O(C₁-C₃)alkyl, wherein each occurrence of —(C₁-C₃)alkyl is optionally substituted by one or more groups independently selected from halo and —OH,
   and/or wherein two occurrences of R^{A} may be taken together with the atom(s) to which they are attached to form a (C₃-C₆)cycloalkyl group or a 3- to 6-membered heterocyclic group, wherein the cycloalkyl or heterocyclic group is optionally substituted by one or more halo;
L¹ is selected from a direct bond, —O—, and —CH₂—;
R² is —(C₆-C₁₀)aryl, wherein R² is substituted with one, or two occurrences of R^{B},
   wherein each R^{B} is independently selected
from —OH, —NH₂, —SF₅, —NHC(O)OC(CH₃)₃, and —O(C₁-C₃)alkyl, wherein each occurrence of —O(C₁-C₃)alkyl is optionally substituted by one or more halo;
R³ is selected from —H, and —(C₁-C₃)alkyl; and
n is 0, or 1.

In another aspect, the disclosure provides a compound being of Formula (I)

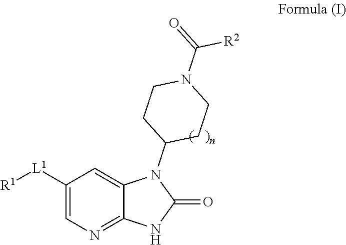

or a pharmaceutically acceptable salt thereof, wherein:
R¹ is selected from —(C₁-C₆)alkyl, —(C₃-C₇)cycloalkyl, 4- to 10-membered heterocycloalkyl, and 5- to 10-membered heteroaryl, wherein R¹ is optionally substituted with one or more occurrences of R^{A},
   wherein each R^{A} is independently selected from halo, —OH, —(C₁-C₃)alkyl, and —O(C₁-C₃)alkyl, wherein each occurrence of —(C₁-C₃)alkyl is optionally substituted by one or more groups independently selected from halo and —OH,
   and/or wherein two occurrences of R^{A} may be taken together with the atom(s) to which they are attached to form a (C₃-C₆)cycloalkyl group or a 3- to 6-membered heterocyclic group;
L¹ is selected from a direct bond and —O—;
R² is —(C₆-C₁₀)aryl, wherein R² is substituted with one, or two occurrences of R^{B},
   wherein each R^{B} is independently selected from —OH, —NH₂, —SF₅, and —O(C₁-C₃)alkyl, wherein each occurrence of —O(C₁-C₃)alkyl is optionally substituted by one or more halo; and
n is 0, or 1.

In embodiments, the compound is a compound of Formula (I) or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo, —OH, —(C₁-C₃)alkyl, and —O(C₁-C₃)alkyl, wherein each occurrence of —(C₁-C₃)alkyl is optionally substituted by one or more groups independently selected from halo and —OH,
   and/or wherein two occurrences of R^{A} may be taken together with the atom(s) to which they are attached to form a (C₃-C₆)cycloalkyl group or a 3- to 6-membered heterocyclic group;
L¹ is selected from a direct bond and —O—;
R² is —(C₆-C₁₀)aryl, wherein R² is substituted with one, or two occurrences of R^{B},
   wherein each R^{B} is independently selected from —OH, —NH₂, —SF₅, and —O(C₁-C₃)alkyl, wherein each occurrence of —O(C₁-C₃)alkyl is optionally substituted by one or more halo; and
n is 0, or 1.

In embodiments, R¹ is selected from —(C₁-C₆)alkyl, —(C₃-C₇)cycloalkyl, and 4- to 10-membered heterocycloalkyl, wherein R¹ is optionally substituted with one or more occurrences of R^{A} as defined herein.

In embodiments, R¹ is selected from halo (e.g., —Br), —(C₁-C₃)alkyl, —(C₃-C₇)cycloalkyl, and 4- to 7-membered heterocycloalkyl, wherein R¹ is optionally substituted with one or more occurrences of R^{A} as defined herein. In embodiments, R¹ is optionally substituted with one or more occurrences of R^{A}, wherein each R^{A} is independently selected from halo (e.g., —F), —OH, —(C₁-C₂)alkyl, and —OCH₃, wherein each occurrence of —(C₁-C₂)alkyl is optionally substituted by one or more (e.g., one, two, or three) groups independently selected from —F and —OH; and/or wherein two occurrences of R^{A} may be taken together with the atom(s) to which they are attached to form a (C₃-C₆)cycloalkyl group (e.g., cyclopropyl or cyclohexyl) or a 6-membered heterocyclic group comprising one nitrogen or oxygen ring atom (e.g., tetrahydropyran, or piperidine). In embodiments, the (C₃-C₆)cycloalkyl group or 6-membered heterocyclic group formed by two occurrences of R^{A} and the atom(s) to which they are attached is attached to R¹ in a fused or bridged configuration. In embodiments, the 6-membered heterocyclic group is a 6-membered heterocycloalkyl group.

In embodiments, R¹ is selected from —(C₁-C₃)alkyl, —(C₄-C₆)cycloalkyl, 5- to 9-membered heterocycloalkyl (e.g., 5- to 7-membered heterocycloalkyl, or 5- to 6-membered heterocycloalkyl), and 5- to 9-membered heteroaryl (e.g., 5-membered heteroaryl, or 9-membered heteroaryl), wherein R¹ is optionally substituted with one or more occurrences of R^{A} as defined herein. In embodiments, R¹ is optionally substituted with one or more occurrences of R^{A}, wherein each R^{A} is independently selected from halo (e.g., —F), —OH, —(C₁-C₂)alkyl, and —OCH₃, wherein each occurrence of —(C₁-C₂)alkyl is optionally substituted by one or more (e.g., one, or three) groups independently selected from —F and —OH; and/or wherein two occurrences of R^{A} may be taken together with the atom(s) to which they are attached to form a cyclopentanyl group or a 4- to 6-membered heterocyclic group comprising one nitrogen or oxygen ring atom (e.g., oxetane, azetidine, pyrrolidine, or tetrahydropyridine).

In embodiments, R¹ is unsubstituted.

In other embodiments, R¹ is substituted with one, or two occurrences of R^{A} as defined herein.

In embodiments, R¹ is substituted with one occurrence of R^{A}. In embodiments, R¹ is substituted with two occurrences of R^{A}. In embodiments, each R^{A} is independently selected from halo (e.g., —F), —OH, —(C₁-C₃)alkyl, and —OCH₃, wherein each occurrence of —(C₁-C₃)alkyl is optionally substituted by one or more groups independently selected from halo (e.g. —F) and —OH.

In embodiments, R¹ is substituted with one, two, or three occurrences of R^{A} as defined herein. In embodiments, R¹ is substituted with two or three occurrences of R^{A}. In embodiments, R¹ is substituted with three occurrences of R^{A}.

In embodiments, two occurrences of R^{A} are taken together with the atom(s) to which they are attached to form a (C₃-C₆)cycloalkyl group or a 3- to 6-membered heterocyclic group, wherein the cycloalkyl or heterocyclic group is optionally substituted by one or more halo. In embodiments, the 3- to 6-membered heterocyclic group is a 3- to 6-membered heterocycloalkyl group.

In embodiments, R¹ is selected from:

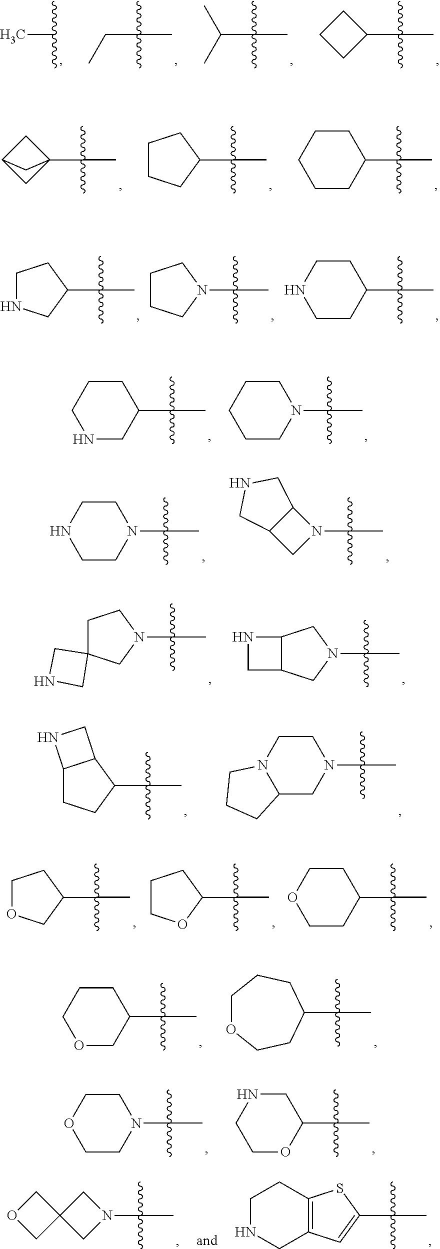

wherein R¹ is optionally substituted by one or more occurrences of R^{A} as defined herein. In embodiments, each R^{A} is independently selected from the group consisting of halo (e.g., —F), —OH, —(C₁-C₃)alkyl (e.g., methyl, or ethyl), and —OCH₃, wherein each occurrence of (C₁-C₃)alkyl may be optionally substituted by one or more substituents selected from halo (e.g., —F) and —OH. In embodiments, R¹ is optionally substituted by one or two occurrences of R^{A}, and each R^{A} is independently selected from the group consisting of halo (e.g., —F), —OH, —(C₁-C₃)alkyl (e.g., methyl, or ethyl), and —OCH₃, wherein each occurrence of (C₁-C₃)alkyl may be optionally substituted by one or more substituents selected from halo (e.g., —F) and —OH.

In embodiments, R¹ is selected from:

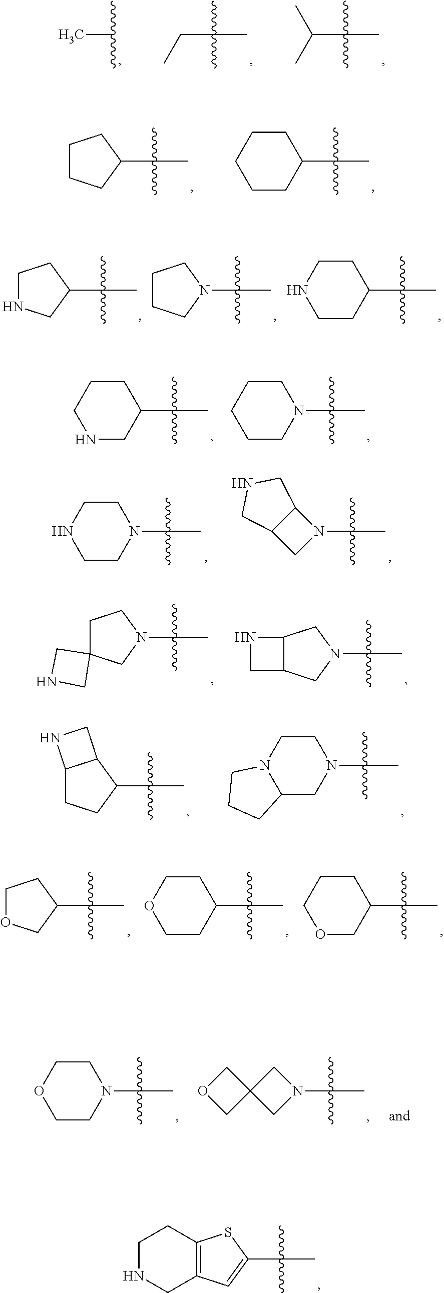

wherein R¹ is optionally substituted by one or more occurrences of R^{A} as defined herein. In embodiments, each R^{A} is independently selected from the group consisting of halo (e.g., —F), —OH, —(C₁-C₃)alkyl (e.g., methyl, or ethyl), and —OCH₃, wherein each occurrence of (C₁-C₃)alkyl may be optionally substituted by one or more substituents selected from halo and —OH.

In embodiments R¹ is selected from:

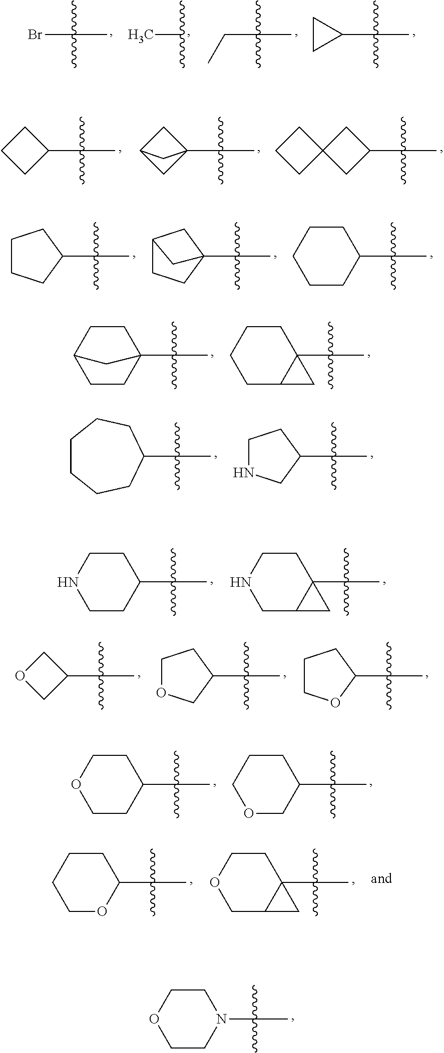

wherein R¹ is optionally substituted by one or two occurrences of R^{A} as defined herein. In embodiments, each R^{A} is independently selected from —F, —OH, oxo, ═N—OH, —OCH₃, and —(C₁-C₂)alkyl optionally substituted by one or more substituents selected from —F and —OH.

In embodiments, R¹ is selected from:

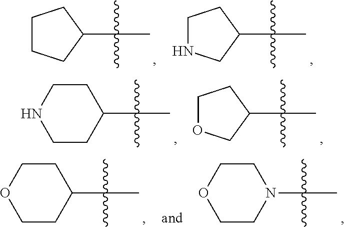

wherein R¹ is optionally substituted by one occurrence of R^{A} as defined herein. In embodiments, R^{A} is methyl optionally substituted by —OH.

In embodiments, R¹ is selected from:

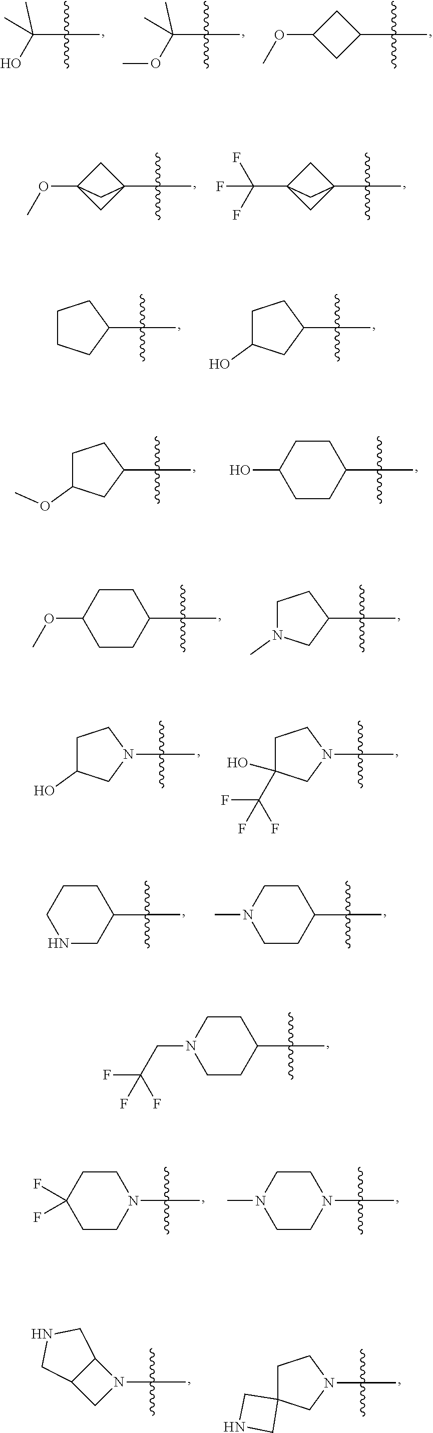

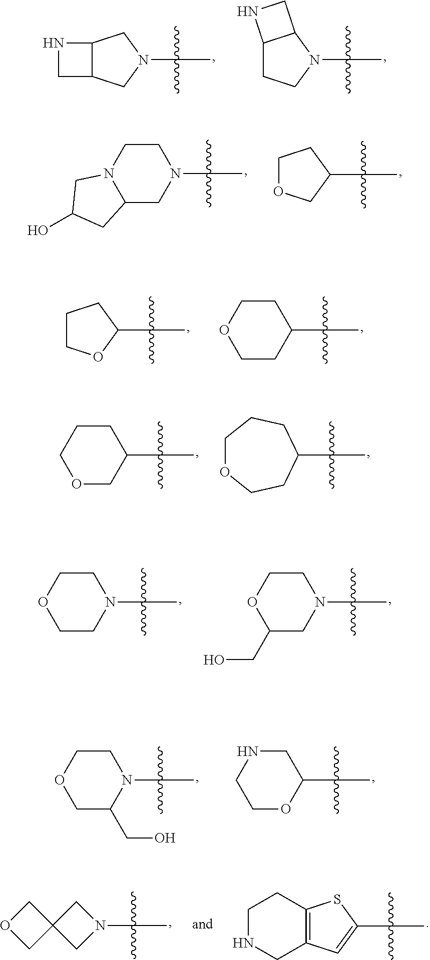

In embodiments, R¹ is selected from:

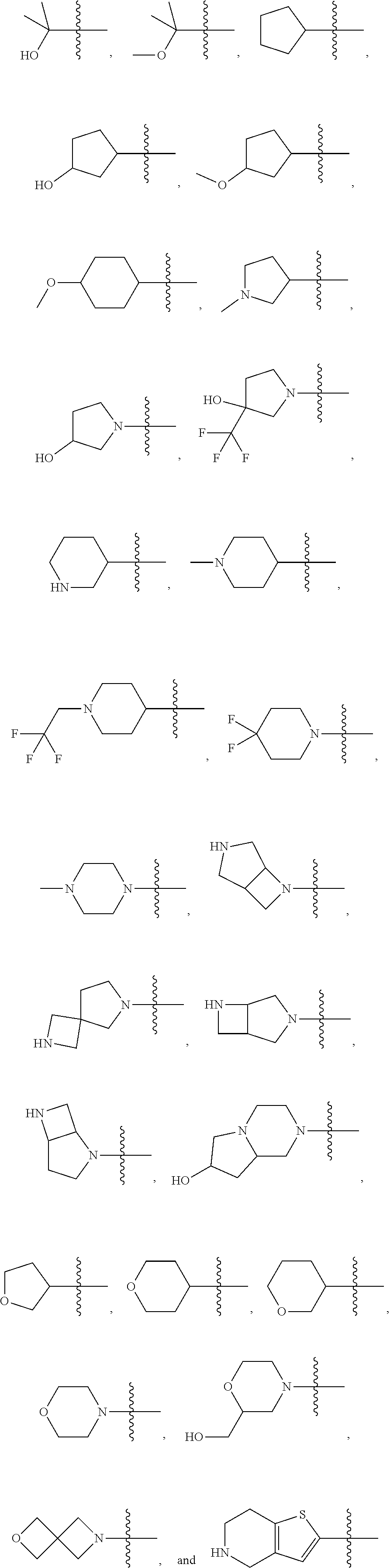

In embodiments, R¹ is 5- to 6-membered heterocycloalkyl, wherein the heterocycloalkyl group comprises one or two atoms independently selected from O and N, and wherein the heterocycloalkyl group is optionally and independently substituted by one or more occurrences of R^{A} as defined herein. In embodiments, each R^{A} is independently selected from —CH₂OH, and —CH₃.

In embodiments, R¹ is selected from:

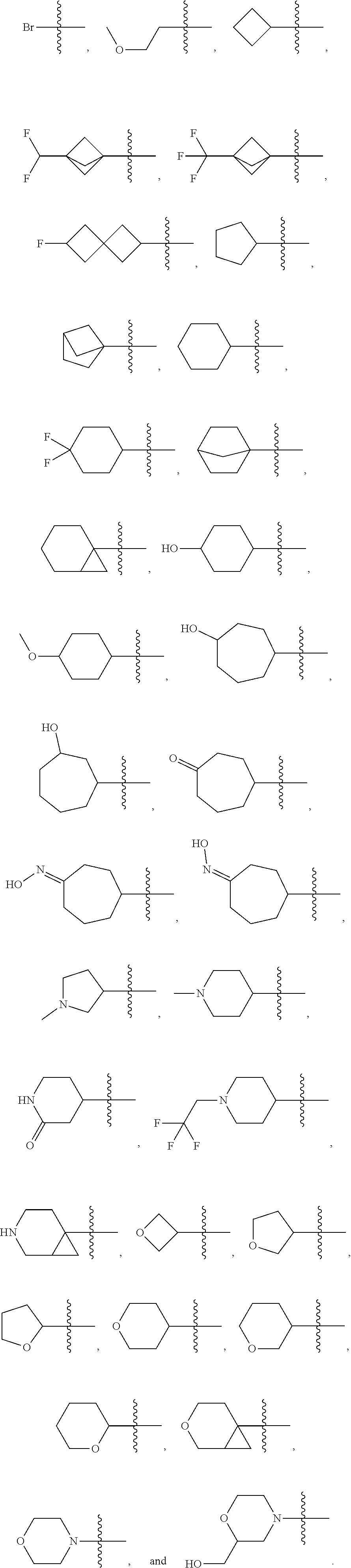

In embodiments, R¹ is selected from:

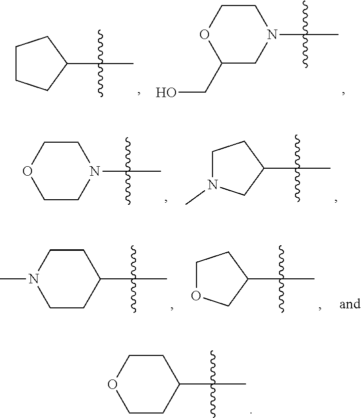

In embodiments, R¹ is multicyclic (e.g., bicyclic) —(C₅-C₇)cycloalkyl, wherein R¹ is optionally substituted with one or more occurrences of R^{A}. In embodiments, R¹ is spiro, bridged, or fused —(C₅-C₇)cycloalkyl, wherein R¹ is optionally substituted with one or more occurrences of R^{A}.

In embodiments, R¹ is 6- to 10-membered multicyclic (e.g., bicyclic) heterocycloalkyl or 7- to 10-membered multicyclic (e.g., bicyclic) heteroaryl, wherein R¹ is optionally substituted with one or more occurrences of R^{A} as defined herein.

In embodiments, R¹ is 6- to 10-membered multicyclic (e.g., bicyclic) heterocycloalkyl optionally substituted with one or more occurrences of R^{A} as defined herein. In embodiments, R¹ is 7- to 9-membered multicyclic (e.g., bicyclic) heterocycloalkyl optionally substituted with one or more occurrences of R^{A}. In embodiments, R¹ is 7- to 9-membered bicyclic heterocycloalkyl comprising one or two (e.g., two) hetero ring atoms selected from nitrogen and oxygen, wherein R¹ is optionally substituted with one or more occurrences of R^{A}. In embodiments, R¹ is 7- to 9-membered bicyclic heterocycloalkyl comprising two nitrogen ring atoms, wherein R¹ is optionally substituted with one or more occurrences of R^{A}.

In embodiments, R¹ is 7- to 10-membered multicyclic (e.g., bicyclic) heteroaryl optionally substituted with one or more occurrences of R^{A} as defined herein. In embodiments, R¹ is 8- to 9-membered (e.g., 9-membered) multicyclic (e.g., bicyclic) heteroaryl optionally substituted with one or more occurrences of R^{A}. In embodiments, R¹ is an unsubstituted 9-membered bicyclic heteroaryl.

In embodiments, R² is phenyl substituted with one, or two occurrences of R^{B}. In embodiments, R² is —(C₆-C₁₀)aryl substituted with one occurrence of R^{B}. In embodiments, R² is phenyl substituted with one occurrence of R^{B}. In embodiments, R² is —(C₆-C₁₀)aryl (e.g., phenyl) substituted with two occurrences of R^{B}.

In embodiments, each R^{B} is independently selected from —OH, —NH₂, —NHC(O)OC(CH₃)₃, —SF₅, and —OCF₃. In embodiments, each R^{B} is independently selected from —OH, —NH₂, —SF₅, and —OCF₃. In embodiments, R² is substituted with two occurrences of R^{B}, and each R^{B} is independently selected from —OH, —NH₂, —SF₅, and —OCF₃.

In embodiments, at least one occurrence of R^{B} is —SF₅. In embodiments, one occurrence of R^{B} is —SF₅. In embodiments, R² is substituted with one occurrence of R^{B}, and R^{B} is —SF₅. In embodiments, R² is substituted with two occurrences of R^{B}, wherein one occurrence of R^{B} is —SF₅, and the other occurrence of R^{B} is selected from —OH and —NH₂.

In embodiments, R² is phenyl substituted with one, or two occurrences of R^{B}, and each R^{B} is independently selected from —OH, —NH₂, —SF₅, and —OCF₃. In embodiments, R² is phenyl substituted with two occurrences of R^{B}, and each R^{B} is independently selected from —OH, —NH₂, —SF₅, and —OCF₃. In embodiments, R² is phenyl substituted with one occurrence of R^{B}, and R^{B} is selected from —SF₅, and —OCF₃. In embodiments, R² is phenyl substituted with two occurrences of R^{B}, wherein one R^{B} is independently selected from —SF₅, and —OCF₃, and the other occurrence of R^{B} is selected from —OH, and —NH₂.

In embodiments, R² is phenyl substituted with one, or two occurrences of R^{B}, and at least one occurrence of R^{B} is —SF₅. In embodiments, R² is phenyl substituted with one, or two occurrences of R^{B}, and one occurrence of R^{B} is —SF₅. In embodiments, R² is phenyl substituted with one, or two occurrences of R^{B}, wherein one occurrence of R^{B} is —SF₅, and the other occurrence of R^{B} (where present) is selected from —OH, and —NH₂. In embodiments, R² is phenyl substituted with two occurrences of R^{B}, wherein one occurrence of R^{B} is —SF₅, and the other occurrence of R^{B} is selected from —OH and —NH₂. In embodiments, R² is phenyl substituted with one occurrence of R^{B}, and R^{B} is —SF₅.

In embodiments, R² is selected from:

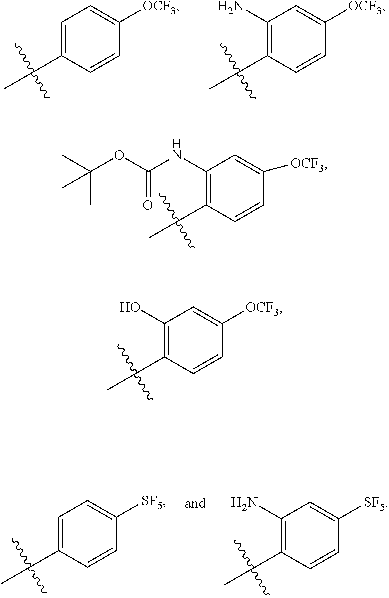

In embodiments, R² is selected from:

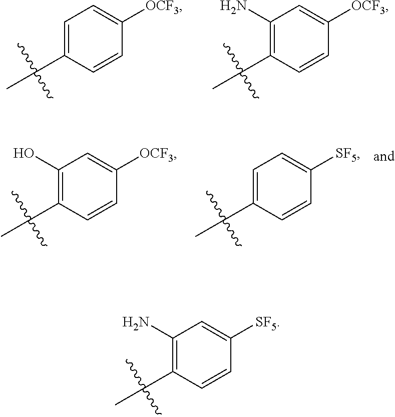

In embodiments, R³ is —CH₃ or —H. In embodiments, R³ is —CH₃. In embodiments, R³ is —H.

In embodiments, L¹ is a direct bond. In other embodiments, L¹ is —O—. In other embodiments, L¹ is —CH₂—. Viewed from this aspect, the disclosure provides a compound of Formula (N-IA), Formula (N-IB), or Formula (N-IC)

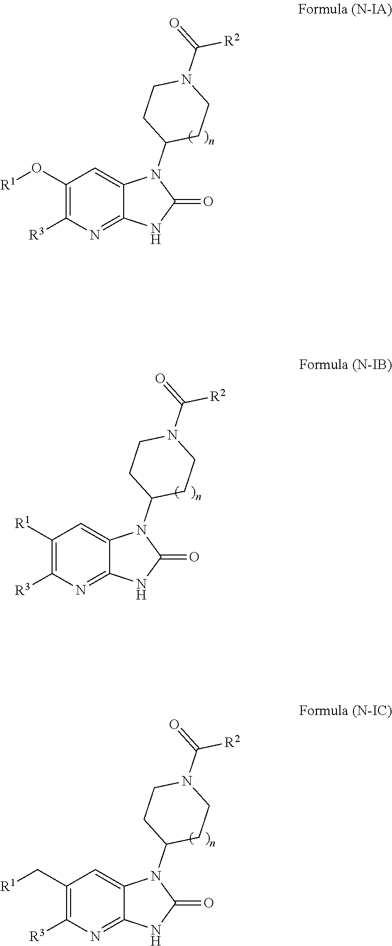

or a pharmaceutically acceptable salt thereof, wherein R¹, R², R³, and n are as defined herein.

In embodiments, the compound is a compound of Formula (N-IA), or a pharmaceutically acceptable salt thereof. In other embodiments, the compound is a compound of Formula (N-IB), or a pharmaceutically acceptable salt thereof. In other embodiments, the compound is a compound of Formula (N-IC), or a pharmaceutically acceptable salt thereof.

In embodiments of Formula (I), L¹ is a direct bond. In other embodiments of Formula (I), L¹ is —O—. Viewed from this aspect, the disclosure provides a compound of Formula (I-A) or Formula (I-B)

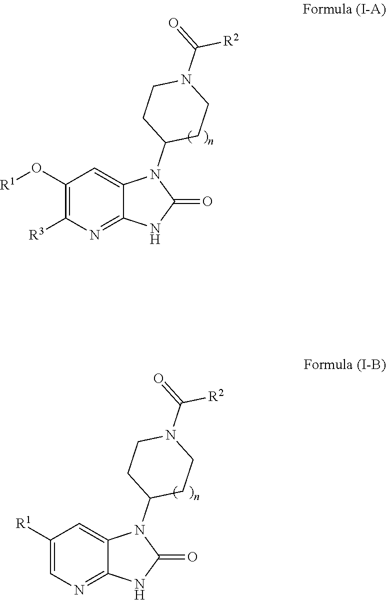

or a pharmaceutically acceptable salt thereof, wherein R¹, R², and n are as defined herein. In embodiments, the compound is a compound of Formula (I-A), or a pharmaceutically acceptable salt thereof. In other embodiments, the compound is a compound of Formula (I-B), or a pharmaceutically acceptable salt thereof.

In other embodiments of Formula (I), L¹ is —CH₂—. Viewed from this aspect, the disclosure provides a compound of Formula (I-C)

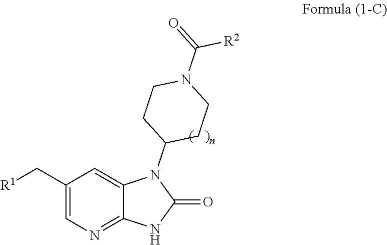

or a pharmaceutically acceptable salt thereof, wherein R¹, R², and n are as defined herein.

In embodiments, n is 1.

In other embodiments, n is 0. Viewed from this aspect, the disclosure provides a compound of Formula (N-ID)

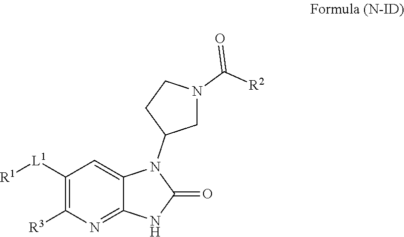

or a pharmaceutically acceptable salt thereof, wherein R¹, R², R³, and L¹ are as defined herein.

In embodiments of Formula (I), n is 0. Viewed from this aspect, the disclosure provides a compound of Formula (I-D)

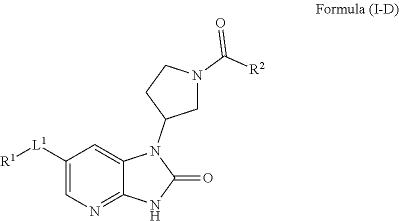

or a pharmaceutically acceptable salt thereof, wherein R¹, R², and L¹ are as defined herein.

In another embodiment, the compound is a compound of Formula (N-II)

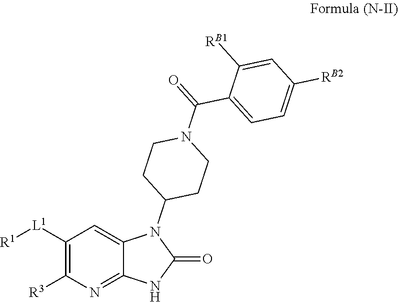

or a pharmaceutically acceptable salt thereof, wherein R¹, R³, and L¹ are as defined herein, and R^{B1} and R^{B2} are independently selected from R^{B} as defined herein.

In embodiments, the compound is a compound of Formula (N-II), or a pharmaceutically acceptable salt thereof, wherein:
R¹ is selected from halo (e.g., —Br), —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo (e.g., —F), —OH, oxo, ═N—OH, —O(C₁-C₃)alkyl, and —(C₁-C₃)alkyl optionally substituted by one or more groups independently selected from halo (e.g., —F) and —OH,
   and/or wherein two occurrences of R^{A} may be taken together with the atom(s) to which they are attached to form a (C₃-C₆)cycloalkyl or 3- to 6-membered heterocycloalkyl group, wherein the cycloalkyl group is optionally substituted by —F;
L¹ is selected from a direct bond, —O—, and —CH₂—;
R^{B1} is selected from —H, —OH, —NH₂, and —NHC(O)OC(CH₃)₃;
R^{B2} is selected from —SF₅, and —OCF₃; and
R³ is selected from —H, and —(C₁-C₃)alkyl (e.g., —CH₃).

In another embodiment, the compound is a compound of Formula (II)

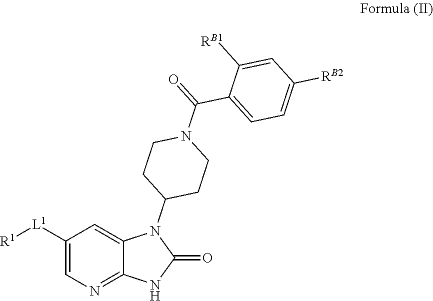

or a pharmaceutically acceptable salt thereof, wherein:
R¹ is selected from halo (e.g., —Br), —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo (e.g., —F), —OH, oxo, ═N—OH, —O(C₁-C₃)alkyl, and —(C₁-C₃)alkyl optionally substituted by one or more groups independently selected from halo (e.g., —F) and —OH,
   and/or wherein two occurrences of R^{A} may be taken together with the atom(s) to which they are attached to form a (C₃-C₆)cycloalkyl or 3- to 6-membered heterocycloalkyl group, wherein the cycloalkyl group is optionally substituted by —F;
L¹ is selected from a direct bond, —O—, and —CH₂—;
R^{B1} is selected from —H, —OH, —NH₂, and —NHC(O)OC(CH₃)₃; and
R^{B2} is selected from —SF₅, and —OCF₃.

In embodiments, the compound is a compound of Formula (II), or a pharmaceutically acceptable salt thereof, wherein:
R¹ is selected from —(C₃-C₇)cycloalkyl, and 4- to 10-membered heterocycloalkyl, wherein R¹ is optionally substituted with one or more occurrences of R^{A},
   wherein each R^{A} is independently —(C₁-C₃)alkyl optionally substituted by —OH;
L¹ is selected from a direct bond and —O—;
R^{B1} is selected from —H, —OH, and —NH₂; and
R^{B2} is selected from —SF₅, and —OCF₃.

In embodiments, R¹ is —(C₄-C₇)cycloalkyl or 4- to 7-membered heterocycloalkyl (e.g., 4- to 6-membered heterocycloalkyl), wherein R¹ is optionally substituted by one or more occurrences of R^{A}. In embodiments, each R^{A} is independently selected from —F, —OH, oxo, ═N—OH, —CH₃, —CHF₂, —CF₃, —CH₂OH, —CH₂CF₃, and —OCH₃.

In embodiments, R¹ is multicyclic (e.g., bicyclic) —(C₅-C₇)cycloalkyl, wherein R¹ is optionally substituted with one or more occurrences of R^{A}. In embodiments, R¹ is spiro, bridged, or fused —(C₅-C₇)cycloalkyl.

In embodiments, R¹ is 6- to 10-membered multicyclic (e.g., bicyclic) heterocycloalkyl, wherein R¹ is optionally substituted with one or more occurrences of R^{A} as defined herein. In embodiments, R¹ is 6- to 8-membered multicyclic (e.g., bicyclic) heterocycloalkyl. In embodiments, R¹ is 7-membered multicyclic (e.g., bicyclic) heterocycloalkyl.

In embodiments, R¹ is 4- to 7-membered heterocycloalkyl (e.g., 4- to 6-membered heterocycloalkyl), wherein the heterocycloalkyl group comprises one or two ring heteroatoms independently selected from O and N, and wherein the heterocycloalkyl group is optionally and independently substituted by one or more occurrences of R^{A}. In embodiments, each R^{A} is independently selected from oxo, —CH₃, —CH₂OH, and —CH₂CF₃.

In embodiments, R¹ is 5- to 6-membered heterocycloalkyl, wherein the heterocycloalkyl group comprises one or two ring heteroatoms independently selected from O and N, and wherein the heterocycloalkyl group is optionally and independently substituted by one or more occurrences of R^{A}. In embodiments, each R^{A} is independently selected from —CH₂OH, and —CH₃.

In embodiments, R¹ is selected from:

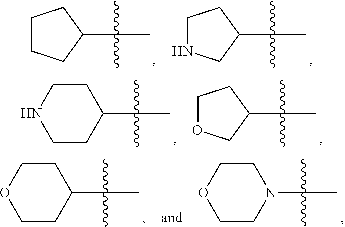

wherein R¹ is optionally substituted by one occurrence of R^{A}, wherein R^{A} is methyl optionally substituted by —OH.

In embodiments, R¹ is selected from:

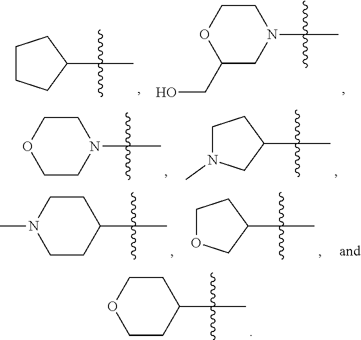

In embodiments, R^{B1} is —H. In embodiments, R^{B1} is selected from —OH, and —NH₂. In embodiments, R^{B1} is —OH. In embodiments, R^{B1} is —NH₂.

In embodiments, R^{B2} is —SF₅. In embodiments, R^{B2} is —OCF₃.

In embodiments, R^{B1} is —H and R^{B2} is —SF₅. In embodiments, R^{B1} is —NH₂ and R^{B2} is —SF₅. In embodiments, R^{B1} is —H and R^{B2} is —OCF₃. In embodiments, R^{B1} is —OH and R^{B2} is —OCF₃. In embodiments, R^{B1} is —NH₂ and R^{B2} is —OCF₃. In embodiments, R^{B1} is —NHC(O)OC(CH₃)₃ and R^{B2} is —OCF₃.

In embodiments, L¹ is a direct bond. In other embodiments, L¹ is —O—. In other embodiments, L¹ is —CH₂—. Viewed from this aspect, the disclosure provides a compound of Formula (N-IIA), Formula (N-IIB), or Formula (N-IIC)

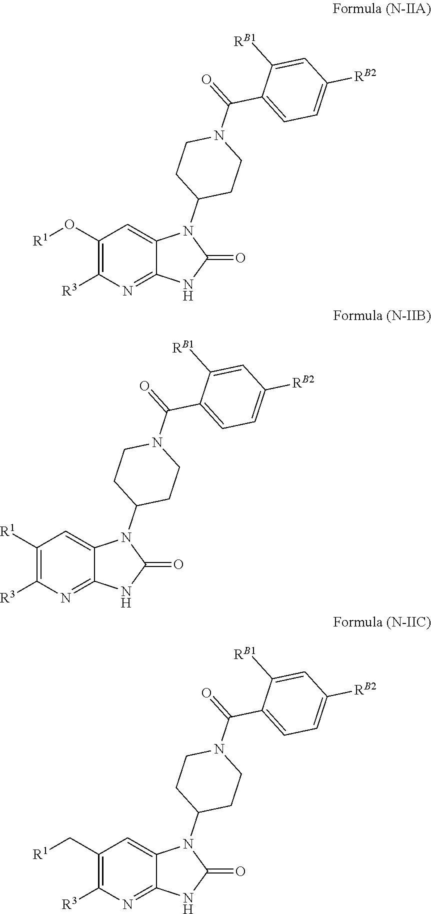

or a pharmaceutically acceptable salt thereof, wherein R¹, R³, R^{B1}, and R^{B2} are as defined herein. In embodiments, the compound is a compound of Formula (N-IIA), or a pharmaceutically acceptable salt thereof. In other embodiments, the compound is a compound of Formula (N-IIB), or a pharmaceutically acceptable salt thereof. In other embodiments, the compound is a compound of Formula (N-IIC), or a pharmaceutically acceptable salt thereof.

In embodiments of Formula (II), L¹ is a direct bond. In other embodiments of Formula (II), L¹ is —O—. Viewed from this aspect, the disclosure provides a compound of Formula (II-A) or Formula (II-B)

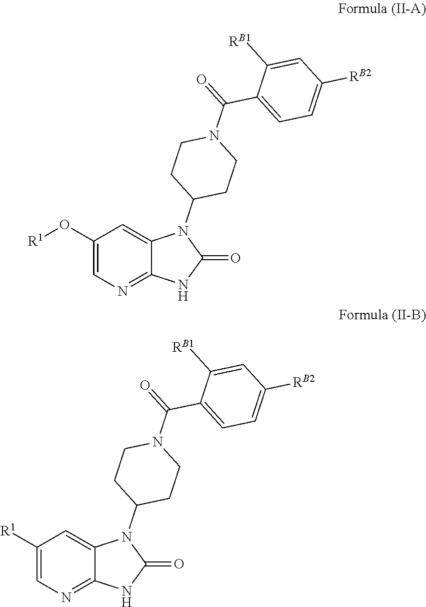

or a pharmaceutically acceptable salt thereof, wherein R¹, R^{B1}, and R^{B2} are as defined herein.

In embodiments, the compound is a compound of Formula (II-A) or (II-B), or a pharmaceutically acceptable salt thereof, wherein:
R¹ is selected from —(C₃-C₇)cycloalkyl, and 4- to 10-membered heterocycloalkyl, wherein R¹ is optionally substituted with one or more occurrences of R^{A},
   wherein each R^{A} is independently —(C₁-C₃)alkyl optionally substituted by —OH;
R^{B1} is selected from —H, —OH, and —NH₂; and
R^{B2} is selected from —SF₅, and —OCF₃.

In embodiments, the compound is a compound of Formula (II-A), or a pharmaceutically acceptable salt thereof. In other embodiments, the compound is a compound of Formula (II-B), or a pharmaceutically acceptable salt thereof.

In other embodiments of Formula (II), L¹ is —CH₂—. Viewed from this aspect, the disclosure provides a compound of Formula (II-C)

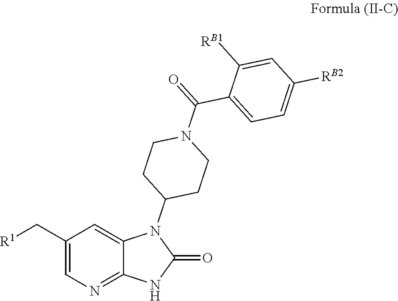

or a pharmaceutically acceptable salt thereof, wherein R¹, R^{B1}, and R^{B2} are as defined herein.

In embodiments, the compound is a compound of Formula (III) or Formula (IV)

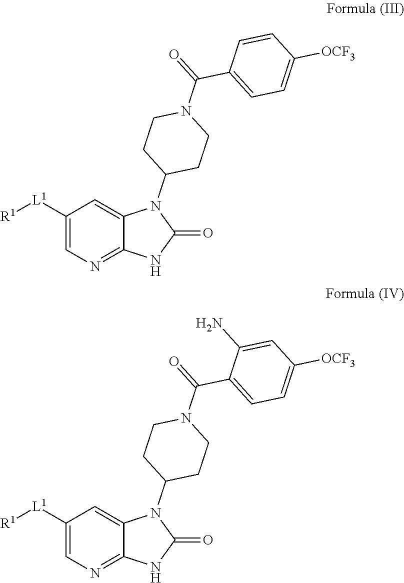

or a pharmaceutically acceptable salt thereof, wherein R¹, and L¹ are as defined herein.

In embodiments, the compound is a compound of Formula (III) or Formula (IV), or a pharmaceutically acceptable salt thereof, wherein:
R¹ is selected from halo (e.g., —Br), —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo (e.g., —F), —OH, oxo, —O(C₁-C₃)alkyl, and —(C₁-C₃)alkyl optionally substituted by —OH; and
L¹ is selected from a direct bond, —O—, and —CH₂—.

In embodiments, the compound is a compound of Formula (III) or Formula (IV), or a pharmaceutically acceptable salt thereof, wherein:
R¹ is selected from —(C₃-C₇)cycloalkyl, and 4- to 10-membered heterocycloalkyl, wherein R¹ is optionally substituted with one or more occurrences of R^{A},
   wherein each R^{A} is independently —(C₁-C₃)alkyl optionally substituted by —OH; and
L¹ is selected from a direct bond and —O—.

In embodiments, the compound is a compound of Formula (III), or a pharmaceutically acceptable salt thereof. In other embodiments, the compound is a compound of Formula (IV), or a pharmaceutically acceptable salt thereof.

In embodiments, the compound is a compound of Formula (III) or a pharmaceutically acceptable salt thereof, and L¹ is a direct bond. In other embodiments, the compound is a compound of Formula (III) or a pharmaceutically acceptable salt thereof, and L¹ is —O—. Viewed from this aspect, the disclosure provides a compound of Formula (III-A) or Formula (III-B)

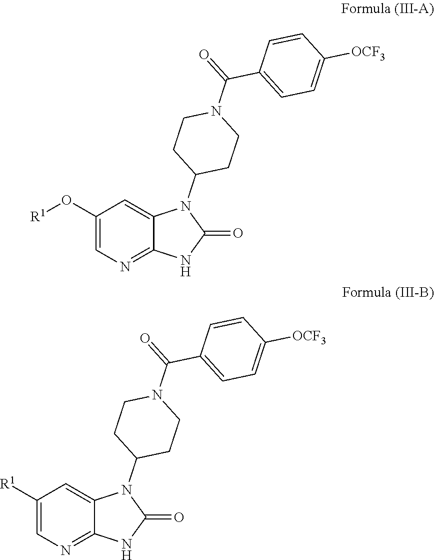

or a pharmaceutically acceptable salt thereof, wherein R¹ is as defined herein.

In embodiments, the compound is a compound of Formula (III-A) or Formula (III-B), or a pharmaceutically acceptable salt thereof, wherein R¹ is selected from —(C₃-C₇)cycloalkyl, and 4- to 10-membered heterocycloalkyl, wherein R¹ is optionally substituted with one or more occurrences of R^{A}, and wherein each R^{A} is independently —(C₁-C₃)alkyl optionally substituted by —OH.

In embodiments, the compound is a compound of Formula (III-A), or a pharmaceutically acceptable salt thereof. In other embodiments, the compound is a compound of Formula (III-B), or a pharmaceutically acceptable salt thereof.

In embodiments, the compound is a compound of Formula (N-IV)

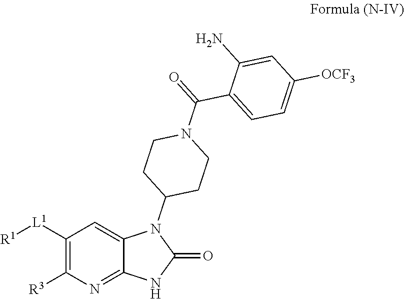

or a pharmaceutically acceptable salt thereof, wherein R¹, R³, and L¹ are as defined herein.

In embodiments, the compound is a compound of Formula (N-IV), or a pharmaceutically acceptable salt thereof, wherein:
R¹ is selected from —(C₁-C₆)alkyl, —(C₃-C₇)cycloalkyl, and 4- to 10-membered heterocycloalkyl, wherein R¹ is optionally substituted with one or more occurrences of R^{A},
   wherein each R^{A} is independently selected from halo (e.g., —F), —OH, oxo, —O(C₁-C₃)alkyl, and —(C₁-C₃)alkyl optionally substituted by one, two, or three groups independently selected from —F and —OH;
R³ is —H or —(C₁-C₃)alkyl; and
L¹ is selected from a direct bond, —O—, and —CH₂—.

In embodiments, L¹ is a direct bond.

In embodiments, the compound is a compound of Formula (IV) or a pharmaceutically acceptable salt thereof, and L¹ is a direct bond. In other embodiments, the compound is a compound of Formula (IV) or a pharmaceutically acceptable salt thereof, and L¹ is —O—.

Viewed from this aspect, the disclosure provides a compound of Formula (IV-A) or Formula (IV-B)

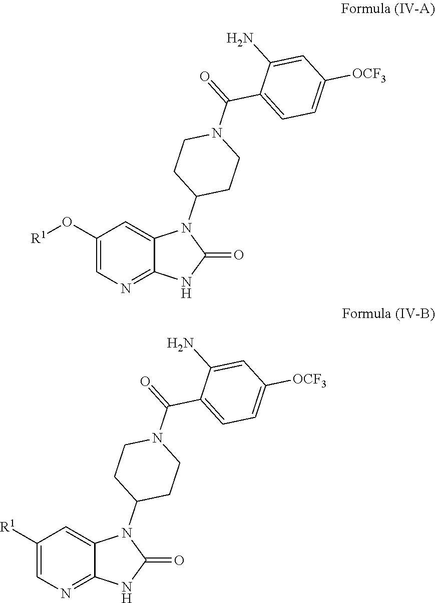

or a pharmaceutically acceptable salt thereof, wherein R¹ is as defined herein.

In embodiments, the compound is a compound of Formula (IV-A) or Formula (IV-B), or a pharmaceutically acceptable salt thereof, wherein R¹ is selected from —(C₃-C₇)cycloalkyl, and 4- to 10-membered heterocycloalkyl, wherein R¹ is optionally substituted with one or more occurrences of R^{A}, and wherein each R^{A} is independently —(C₁-C₃)alkyl optionally substituted by —OH.

In other embodiments, the compound is a compound of Formula (IV) or a pharmaceutically acceptable salt thereof, and L¹ is —CH₂—. Viewed from this aspect, the disclosure provides a compound of Formula (IV-C)

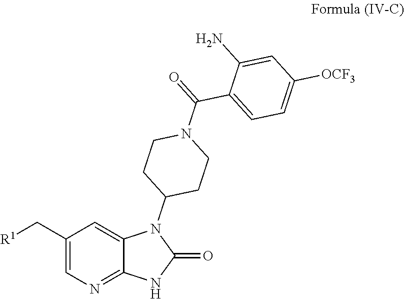

or a pharmaceutically acceptable salt thereof, wherein R¹ is as defined herein.

In embodiments, the compound is a compound of Formula (IV-C), or a pharmaceutically acceptable salt thereof, wherein R¹ is selected from —(C₁-C₃)alkyl, and 4- to 6-membered heterocycloalkyl, wherein R¹ is optionally substituted with one or more occurrences of R^{A}. In embodiments, each R^{A} is independently —O(C₁-C₃)alkyl.

In embodiments, the compound is a compound of Formula (IV-A), or a pharmaceutically acceptable salt thereof. In other embodiments, the compound is a compound of Formula (IV-B), or a pharmaceutically acceptable salt thereof.

In embodiments, the compound is a compound of Formula (V), Formula (VI), or Formula (VII)

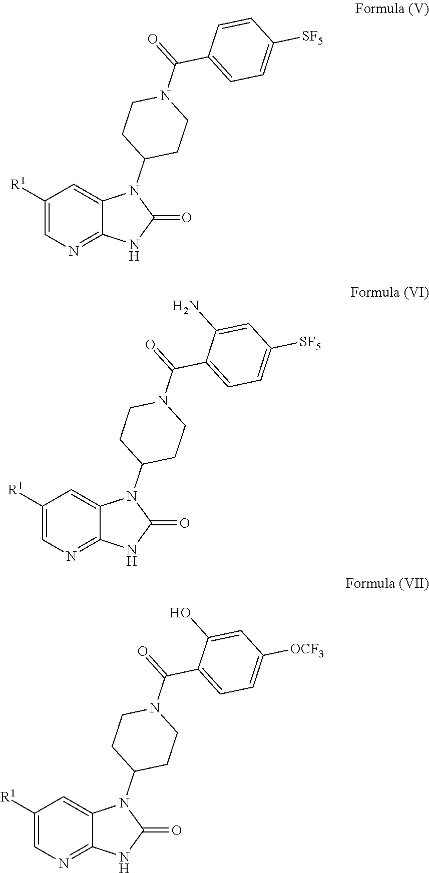

or a pharmaceutically acceptable salt thereof, wherein R¹ is selected from —(C₃-C₇)cycloalkyl, and 4- to 10-membered heterocycloalkyl, wherein R¹ is optionally substituted with one or more occurrences of R^{A}, and wherein each R^{A} is independently —(C₁-C₃)alkyl optionally substituted by —OH.

In embodiments, the compound is a compound of Formula (V), or a pharmaceutically acceptable salt thereof. In other embodiments, the compound is a compound of Formula (VI), or a pharmaceutically acceptable salt thereof. In other embodiments, the compound is a compound of Formula (VII), or a pharmaceutically acceptable salt thereof.

In embodiments, the compound is selected from the group consisting of
1-[1-[2-amino-4-(trifluoromethoxy)benzoyl]-4-piperidyl]-6-tetrahydropyran-4-yloxy-3H-imidazo[4,5-b]pyridin-2-one,
1-[1-[2-amino-4-(trifluoromethoxy)benzoyl]-4-piperidyl]-6-tetrahydropyran-4-yl-3H-imidazo[4,5-b]pyridin-2-one,
1-[1-[2-amino-4-(trifluoromethoxy)benzoyl]-4-piperidyl]-6-N-morpholino-3H-imidazo[4,5-b]pyridin-2-one,
6-tetrahydropyran-4-yloxy-1-[1-[4-(trifluoromethoxy)benzoyl]-4-piperidyl]-3H-imidazo[4,5-b]pyridin-2-one,
6-[(1-methyl-4-piperidyl)oxy]-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-(1-methylpyrrolidin-3-yl)oxy-3H-imidazo[4,5-b]pyridin-2-one,
1-[1-[2-amino-4-(trifluoromethoxy)benzoyl]-4-piperidyl]-6-[(1-methyl-4-piperidyl)oxy]-3H-imidazo[4,5-b]pyridin-2-one,
6-N-morpholino-1-[1-[4-(trifluoromethoxy)benzoyl]-4-piperidyl]-3H-imidazo[4,5-b]pyridin-2-one,
6-(1-methylpyrrolidin-3-yl)oxy-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[2-(hydroxymethyl)morpholin-4-yl]-3H-imidazo[4,5-b]pyridin-2-one,
6-[2-(hydroxymethyl)morpholin-4-yl]-1-[1-[4-(trifluoromethoxy)benzoyl]-4-piperidyl]-3H-imidazo[4,5-b]pyridin-2-one,
1-[1-[2-amino-4-(pentafluoro-λ⁶-sulfanyl)benzoyl]-4-piperidyl]-6-tetrahydropyran-4-yl-3H-imidazo[4,5-b]pyridin-2-one,
6-tetrahydrofuran-3-yl-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-tetrahydrofuran-3-yl-3H-imidazo[4,5-b]pyridin-2-one,
6-cyclopentyl-1-[1-[4-(trifluoromethoxy)benzoyl]-4-piperidyl]-3H-imidazo[4,5-b]pyridin-2-one,
1-[i-[2-hydroxy-4-(trifluoromethoxy)benzoyl]-4-piperidyl]-6-tetrahydropyran-4-yl-3H-imidazo[4,5-b]pyridin-2-one,
1-[1-[4-(pentafluoro-λ⁶-sulfanyl)benzoyl]-4-piperidyl]-6-tetrahydropyran-4-yl-3H-imidazo[4,5-b]pyridin-2-one,
1-[i-[2-amino-4-(trifluoromethoxy)benzoyl]-4-piperidyl]-5-methyl-6-tetrahydropyran-4-yl-3H-imidazo[4,5-b]pyridin-2-one,
1-[1-[2-amino-4-(trifluoromethoxy)benzoyl]-4-piperidyl]-6-(2-fluorospiro[3.3]heptan-6-yl)-3H-imidazo[4,5-b]pyridin-2-one,
1-[1-[2-amino-4-(trifluoromethoxy)benzoyl]-4-piperidyl]-6-(2-oxo-4-piperidyl)-3H-imidazo[4,5-b]pyridin-2-one,
1-[1-[2-amino-4-(trifluoromethoxy)benzoyl]-4-piperidyl]-6-(tetrahydrofuran-3-ylmethyl)-3H-imidazo[4,5-b]pyridin-2-one,
1-[1-[2-amino-4-(trifluoromethoxy)benzoyl]-4-piperidyl]-6-(2-methoxyethyl)-3H-imidazo[4,5-b]pyridin-2-one,
1-[1-[2-amino-4-(trifluoromethoxy)benzoyl]-4-piperidyl]-6-tetrahydrofuran-2-yl-3H-imidazo[4,5-b]pyridin-2-one,
trans-6-(4-methoxycyclohexyl)-1-[1-[4-(trifluoromethoxy)benzoyl]-4-piperidyl]-3H-imidazo[4,5-b]pyridin-2-one,
cis-6-(4-methoxycyclohexyl)-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-(3-azabicyclo[4.1.0]heptan-6-yl)-3H-imidazo[4,5-b]pyridin-2-one,
1-[1-[2-amino-4-(trifluoromethoxy)benzoyl]-4-piperidyl]-6-cyclobutyl-3H-imidazo[4,5-b]pyridin-2-one,
1-[1-[2-amino-4-(trifluoromethoxy)benzoyl]-4-piperidyl]-6-[3-(trifluoromethyl)-1-bicyclo[1.1.1]pentanyl]-3H-imidazo[4,5-b]pyridin-2-one,
1-[1-[2-amino-4-(trifluoromethoxy)benzoyl]-4-piperidyl]-6-(3-oxabicyclo[4.1.0]heptan-6-yl)-3H-imidazo[4,5-b]pyridin-2-one,
1-[1-[2-amino-4-(trifluoromethoxy)benzoyl]-4-piperidyl]-6-(1-bicyclo[2.1.1]hexanyl)-3H-imidazo[4,5-b]pyridin-2-one,
1-[1-[2-amino-4-(trifluoromethoxy)benzoyl]-4-piperidyl]-6-[3-(difluoromethyl)-1-bicyclo[1.1.1]pentanyl]-3H-imidazo[4,5-b]pyridin-2-one,
1-[1-[2-amino-4-(trifluoromethoxy)benzoyl]-4-piperidyl]-6-norcaran-1-yl-3H-imidazo[4,5-b]pyridin-2-one,
1-[1-[2-amino-4-(trifluoromethoxy)benzoyl]-4-piperidyl]-6-(4,4-difluorocyclohexyl)-3H-imidazo[4,5-b]pyridin-2-one,
1-[1-[2-amino-4-(trifluoromethoxy)benzoyl]-4-piperidyl]-6-cyclohexyl-3H-imidazo[4,5-b]pyridin-2-one,
1-[1-[2-amino-4-(trifluoromethoxy)benzoyl]-4-piperidyl]-6-norbornan-2-yl-3H-imidazo[4,5-b]pyridin-2-one,
1-[1-[2-amino-4-(trifluoromethoxy)benzoyl]-4-piperidyl]-6-tetrahydropyran-2-yl-3H-imidazo[4,5-b]pyridin-2-one,
1-[1-[2-amino-4-(trifluoromethoxy)benzoyl]-4-piperidyl]-6-(tetrahydropyran-4-ylmethyl)-3H-imidazo[4,5-b]pyridin-2-one,
1-[1-[2-amino-4-(trifluoromethoxy)benzoyl]-4-piperidyl]-6-tetrahydropyran-3-yl-3H-imidazo[4,5-b]pyridin-2-one,
6-tetrahydropyran-4-yl-1-[1-[4-(trifluoromethoxy)benzoyl]-4-piperidyl]-3H-imidazo[4,5-b]pyridin-2-one,
6-(oxetan-3-ylmethyl)-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[tetrahydrofuran-3-yl]oxy-3H-imidazo[4,5-b]pyridin-2-one,
trans-1-[1-[2-amino-4-(trifluoromethoxy)benzoyl]-4-piperidyl]-6-(4-methoxycyclohexyl)-3H-imidazo[4,5-b]pyridin-2-one,
cis-1-[1-[2-amino-4-(trifluoromethoxy)benzoyl]-4-piperidyl]-6-(4-methoxycyclohexyl)-3H-imidazo[4,5-b]pyridin-2-one,
1-[1-[2-amino-4-(trifluoromethoxy)benzoyl]-4-piperidyl]-6-[1-methylpyrrolidin-3-yl]oxy-3H-imidazo[4,5-b]pyridin-2-one,
1-[1-[2-amino-4-(pentafluoro-λ⁶-sulfanyl)benzoyl]pyrrolidin-3-yl]-6-tetrahydropyran-4-yl-3H-imidazo[4,5-b]pyridin-2-one,
1-[1-[4-(pentafluoro-λ⁶-sulfanyl)benzoyl]pyrrolidin-3-yl]-6-tetrahydropyran-4-yl-3H-imidazo[4,5-b]pyridin-2-one,
trans-1-[1-[2-amino-4-(trifluoromethoxy)benzoyl]-4-piperidyl]-6-(4-hydroxycyclohexyl)-3H-imidazo[4,5-b]pyridin-2-one,
cis-1-[1-[2-amino-4-(trifluoromethoxy)benzoyl]-4-piperidyl]-6-(4-hydroxycyclohexyl)-3H-imidazo[4,5-b]pyridin-2-one,
1-[1-[2-amino-4-(trifluoromethoxy)benzoyl]-4-piperidyl]-6-[1-(2,2,2-trifluoroethyl)-4-piperidyl]-3H-imidazo[4,5-b]pyridin-2-one,
1-[1-[2-amino-4-(trifluoromethoxy)benzoyl]-4-piperidyl]-6-(1-methyl-4-piperidyl)-3H-imidazo[4,5-b]pyridin-2-one,
1-[1-[2-amino-4-(trifluoromethoxy)benzoyl]-4-piperidyl]-6-tetrahydrofuran-3-yloxy-3H-imidazo[4,5-b]pyridin-2-one,
6-tetrahydrofuran-3-yloxy-1-[1-[4-(trifluoromethoxy)benzoyl]-4-piperidyl]-3H-imidazo[4,5-b]pyridin-2-one,
trans-6-(4-hydroxycyclohexyl)-1-[1-[4-(trifluoromethoxy)benzoyl]-4-piperidyl]-3H-imidazo[4,5-b]pyridin-2-one,
cis-6-(4-hydroxycyclohexyl)-1-[1-[4-(trifluoromethoxy)benzoyl]-4-piperidyl]-3H-imidazo[4,5-b]pyridin-2-one,
tert-butyl N-[2-[4-(6-bromo-2-oxo-3H-imidazo[4,5-b]pyridin-1-yl)piperidine-1-carbonyl]-5-(trifluoromethoxy)phenyl]carbamate,
6-bromo-1-[1-[4-(trifluoromethoxy)benzoyl]-4-piperidyl]-3H-imidazo[4,5-b]pyridin-2-one,
trans-6-(4-hydroxycycloheptyl)-1-[1-[4-(trifluoromethoxy)benzoyl]-4-piperidyl]-3H-imidazo[4,5-b]pyridin-2-one,
cis-6-(4-hydroxycycloheptyl)-1-[1-[4-(trifluoromethoxy)benzoyl]-4-piperidyl]-3H-imidazo[4,5-b]pyridin-2-one,
6-(4-oxocycloheptyl)-1-[1-[4-(trifluoromethoxy)benzoyl]-4-piperidyl]-3H-imidazo[4,5-b]pyridin-2-one,
6-[4-hydroxyiminocycloheptyl]-1-[1-[4-(trifluoromethoxy)benzoyl]-4-piperidyl]-3H-imidazo[4,5-b]pyridin-2-one,
trans-6-(3-hydroxycycloheptyl)-1-[1-[4-(trifluoromethoxy)benzoyl]-4-piperidyl]-3H-imidazo[4,5-b]pyridin-2-one,
cis-6-(3-hydroxycycloheptyl)-1-[1-[4-(trifluoromethoxy)benzoyl]-4-piperidyl]-3H-imidazo[4,5-b]pyridin-2-one,
   and the pharmaceutically acceptable salts thereof.

In embodiments, the compound is selected from the group consisting of
1-[1-[2-amino-4-(trifluoromethoxy)benzoyl]-4-piperidyl]-6-tetrahydropyran-4-yloxy-3H-imidazo[4,5-b]pyridin-2-one,
1-[1-[2-amino-4-(trifluoromethoxy)benzoyl]-4-piperidyl]-6-tetrahydropyran-4-yl-3H-imidazo[4,5-b]pyridin-2-one,
1-[1-[2-amino-4-(trifluoromethoxy)benzoyl]-4-piperidyl]-6-N-morpholino-3H-imidazo[4,5-b]pyridin-2-one,
6-tetrahydropyran-4-yloxy-1-[1-[4-(trifluoromethoxy)benzoyl]-4-piperidyl]-3H-imidazo[4,5-b]pyridin-2-one,
6-[(1-methyl-4-piperidyl)oxy]-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-(1-methylpyrrolidin-3-yl)oxy-3H-imidazo[4,5-b]pyridin-2-one,
1-[1-[2-amino-4-(trifluoromethoxy)benzoyl]-4-piperidyl]-6-[(1-methyl-4-piperidyl)oxy]-3H-imidazo[4,5-b]pyridin-2-one,
6-N-morpholino-1-[1-[4-(trifluoromethoxy)benzoyl]-4-piperidyl]-3H-imidazo[4,5-b]pyridin-2-one,
6-(1-methylpyrrolidin-3-yl)oxy-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[2-(hydroxymethyl)morpholin-4-yl]-3H-imidazo[4,5-b]pyridin-2-one,
6-[2-(hydroxymethyl)morpholin-4-yl]-1-[1-[4-(trifluoromethoxy)benzoyl]-4-piperidyl]-3H-imidazo[4,5-b]pyridin-2-one,
1-[1-[2-amino-4-(pentafluoro-λ⁶-sulfanyl)benzoyl]-4-piperidyl]-6-tetrahydropyran-4-yl-3H-imidazo[4,5-b]pyridin-2-one,
6-tetrahydrofuran-3-yl-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-tetrahydrofuran-3-yl-3H-imidazo[4,5-b]pyridin-2-one,
6-cyclopentyl-1-[1-[4-(trifluoromethoxy)benzoyl]-4-piperidyl]-3H-imidazo[4,5-b]pyridin-2-one,
1-[1-[2-hydroxy-4-(trifluoromethoxy)benzoyl]-4-piperidyl]-6-tetrahydropyran-4-yl-3H-imidazo[4,5-b]pyridin-2-one,
1-[1-[4-(pentafluoro-λ⁶-sulfanyl)benzoyl]-4-piperidyl]-6-tetrahydropyran-4-yl-3H-imidazo[4,5-b]pyridin-2-one,
   and the pharmaceutically acceptable salts thereof.

In embodiments where the compound has enantiomeric forms (e.g., where the compound has a chiral centre, such as a chiral carbon atom), the compound is present as a racemic mixture of enantiomers. In embodiments where the compound has a chiral centre (e.g., a chiral carbon atom), the compound is present as the (R) isomer. In other embodiments where the compound has a chiral centre (e.g., a chiral carbon atom), the compound is present as the (S) isomer.

Thus, in embodiments the compound is selected from the group consisting of:
(rac)-1-[1-[2-amino-4-(trifluoromethoxy)benzoyl]-4-piperidyl]-6-(1-methylpyrrolidin-3-yl)oxy-3H-imidazo[4,5-b]pyridin-2-one,
(rac)-6-(1-methylpyrrolidin-3-yl)oxy-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[(2S)-2-(hydroxymethyl)morpholin-4-yl]-3H-imidazo[4,5-b]pyridin-2-one,
6-[(2R)-2-(hydroxymethyl)morpholin-4-yl]-1-[1-[4-(trifluoromethoxy)benzoyl]-4-piperidyl]-3H-imidazo[4,5-b]pyridin-2-one,
6-[(2S)-2-(hydroxymethyl)morpholin-4-yl]-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[(2R)-2-(hydroxymethyl)morpholin-4-yl]-3H-imidazo[4,5-b]pyridin-2-one,
(rac)-6-tetrahydrofuran-3-yl-1-[1-[4-(trifluoromethoxy)benzoyl]-4-piperidyl]-3H-imidazo[4,5-b]pyridin-2-one,
(rac)-1-[1-[2-amino-4-(trifluoromethoxy)benzoyl]-4-piperidyl]-6-tetrahydrofuran-3-yl-3H-imidazo[4,5-b]pyridin-2-one,
   and the pharmaceutically acceptable salts thereof.

In embodiments the compound is selected from the group consisting of:
(rac)-1-[1-[2-amino-4-(trifluoromethoxy)benzoyl]-4-piperidyl]-6-(1-methylpyrrolidin-3-yl)oxy-3H-imidazo[4,5-b]pyridin-2-one,
(rac)-6-(1-methylpyrrolidin-3-yl)oxy-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[(2S)-2-(hydroxymethyl)morpholin-4-yl]-3H-imidazo[4,5-b]pyridin-2-one,
6-[(2R)-2-(hydroxymethyl)morpholin-4-yl]-1-[1-[4-(trifluoromethoxy)benzoyl]-4-piperidyl]-3H-imidazo[4,5-b]pyridin-2-one,
6-[(2S)-2-(hydroxymethyl)morpholin-4-yl]-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[(2R)-2-(hydroxymethyl)morpholin-4-yl]-3H-imidazo[4,5-b]pyridin-2-one,
(rac)-6-tetrahydrofuran-3-yl-1-[1-[4-(trifluoromethoxy)benzoyl]-4-piperidyl]-3H-imidazo[4,5-b]pyridin-2-one,
(rac)-1-[1-[2-amino-4-(trifluoromethoxy)benzoyl]-4-piperidyl]-6-tetrahydrofuran-3-yl-3H-imidazo[4,5-b]pyridin-2-one,
(rac)-1-[1-[2-amino-4-(trifluoromethoxy)benzoyl]-4-piperidyl]-6-(2-oxo-4-piperidyl)-3H-imidazo[4,5-b]pyridin-2-one,
(rac)-1-[1-[2-amino-4-(trifluoromethoxy)benzoyl]-4-piperidyl]-6-(tetrahydrofuran-3-ylmethyl)-3H-imidazo[4,5-b]pyridin-2-one,
(rac)-1-[1-[2-amino-4-(trifluoromethoxy)benzoyl]-4-piperidyl]-6-tetrahydrofuran-2-yl-3H-imidazo[4,5-b]pyridin-2-one,
(rac)-1-[1-[2-amino-4-(trifluoromethoxy)benzoyl]-4-piperidyl]-6-(3-azabicyclo[4.1.0]heptan-6-yl)-3H-imidazo[4,5-b]pyridin-2-one,
(rac)-1-[1-[2-amino-4-(trifluoromethoxy)benzoyl]-4-piperidyl]-6-(3-oxabicyclo[4.1.0]heptan-6-yl)-3H-imidazo[4,5-b]pyridin-2-one,
(rac)-1-[1-[2-amino-4-(trifluoromethoxy)benzoyl]-4-piperidyl]-6-norcaran-1-yl-3H-imidazo[4,5-b]pyridin-2-one,
(rac)-1-[1-[2-amino-4-(trifluoromethoxy)benzoyl]-4-piperidyl]-6-tetrahydropyran-2-yl-3H-imidazo[4,5-b]pyridin-2-one,
(rac)-1-[1-[2-amino-4-(trifluoromethoxy)benzoyl]-4-piperidyl]-6-tetrahydropyran-3-yl-3H-imidazo[4,5-b]pyridin-2-one,
1-[1-[2-amino-4-(trifluoromethoxy)benzoyl]-4-piperidyl]-6-[(3R)-tetrahydrofuran-3-yl]oxy-3H-imidazo[4,5-b]pyridin-2-one,
1-[1-[2-amino-4-(trifluoromethoxy)benzoyl]-4-piperidyl]-6-[(3S)-tetrahydrofuran-3-yl]oxy-3H-imidazo[4,5-b]pyridin-2-one,
1-[1-[2-amino-4-(trifluoromethoxy)benzoyl]-4-piperidyl]-6-[(3R)-1-methylpyrrolidin-3-yl]oxy-3H-imidazo[4,5-b]pyridin-2-one,
1-[1-[2-amino-4-(trifluoromethoxy)benzoyl]-4-piperidyl]-6-[(3S)-1-methylpyrrolidin-3-yl]oxy-3H-imidazo[4,5-b]pyridin-2-one,
(rac)-1-[1-[2-amino-4-(pentafluoro-λ⁶-sulfanyl)benzoyl]pyrrolidin-3-yl]-6-tetrahydropyran-4-yl-3H-imidazo[4,5-b]pyridin-2-one,
(rac)-1-[1-[4-(pentafluoro-λ⁶-sulfanyl)benzoyl]pyrrolidin-3-yl]-6-tetrahydropyran-4-yl-3H-imidazo[4,5-b]pyridin-2-one,
(rac)-1-[1-[2-amino-4-(trifluoromethoxy)benzoyl]-4-piperidyl]-6-tetrahydrofuran-3-yloxy-3H-imidazo[4,5-b]pyridin-2-one,
(rac)-6-tetrahydrofuran-3-yloxy-1-[1-[4-(trifluoromethoxy)benzoyl]-4-piperidyl]-3H-imidazo[4,5-b]pyridin-2-one,
(rac)-trans-6-(4-hydroxycycloheptyl)-1-[1-[4-(trifluoromethoxy)benzoyl]-4-piperidyl]-3H-imidazo[4,5-b]pyridin-2-one,
(rac)-cis-6-(4-hydroxycycloheptyl)-1-[1-[4-(trifluoromethoxy)benzoyl]-4-piperidyl]-3H-imidazo[4,5-b]pyridin-2-one,
(rac)-6-(4-oxocycloheptyl)-1-[1-[4-(trifluoromethoxy)benzoyl]-4-piperidyl]-3H-imidazo[4,5-b]pyridin-2-one,
(rac)-6-[(4E)-4-hydroxyiminocycloheptyl]-1-[1-[4-(trifluoromethoxy)benzoyl]-4-piperidyl]-3H-imidazo[4,5-b]pyridin-2-one,
(rac)-6-[(4Z)-4-hydroxyiminocycloheptyl]-1-[1-[4-(trifluoromethoxy)benzoyl]-4-piperidyl]-3H-imidazo[4,5-b]pyridin-2-one,
(rac)-trans-6-(3-hydroxycycloheptyl)-1-[1-[4-(trifluoromethoxy)benzoyl]-4-piperidyl]-3H-imidazo[4,5-b]pyridin-2-one,
(rac)-cis-6-(3-hydroxycycloheptyl)-1-[1-[4-(trifluoromethoxy)benzoyl]-4-piperidyl]-3H-imidazo[4,5-b]pyridin-2-one,
   and the pharmaceutically acceptable salts thereof.

In embodiments, the compound is selected from the compounds produced in Examples 1 to 67 (i.e., from the group consisting of Compounds 1-67), and the pharmaceutically acceptable salts thereof. In other embodiments, the compound is selected from the compounds obtainable by the synthetic methods described in any one of Examples 1 to 67 (i.e., the methods for synthesising Compounds 1-67), and the pharmaceutically acceptable salts thereof.

In embodiments, the compound is selected from the compounds produced in Examples 1 to 19 (i.e., from the group consisting of Compounds 1-19), and the pharmaceutically acceptable salts thereof. In other embodiments, the compound is selected from the compounds obtainable by the synthetic methods described in any one of Examples 1 to 19 (i.e., the methods for synthesising Compounds 1-19), and the pharmaceutically acceptable salts thereof. In embodiments, the compound is selected from the group consisting of
1-[1-[2-amino-4-(trifluoromethoxy)benzoyl]-4-piperidyl]-6-tetrahydropyran-4-yloxy-3H-imidazo[4,5-b]pyridin-2-one,
1-[1-[2-amino-4-(trifluoromethoxy)benzoyl]-4-piperidyl]-6-tetrahydropyran-4-yl-3H-imidazo[4,5-b]pyridin-2-one,
1-[1-[2-amino-4-(trifluoromethoxy)benzoyl]-4-piperidyl]-6-N-morpholino-3H-imidazo[4,5-b]pyridin-2-one,
6-tetrahydropyran-4-yloxy-1-[1-[4-(trifluoromethoxy)benzoyl]-4-piperidyl]-3H-imidazo[4,5-b]pyridin-2-one,
6-[(1-methyl-4-piperidyl)oxy]-1-[1-[4-(trifluoromethoxy)benzoyl]-4-piperidyl]-3H-imidazo[4,5-b]pyridin-2-one,
(rac)-1-[1-[2-amino-4-(trifluoromethoxy)benzoyl]-4-piperidyl]-6-(1-methylpyrrolidin-3-yl)oxy-3H-imidazo[4,5-b]pyridin-2-one,
1-[1-[2-amino-4-(trifluoromethoxy)benzoyl]-4-piperidyl]-6-[(1-methyl-4-piperidyl)oxy]-3H-imidazo[4,5-b]pyridin-2-one,
6-N-morpholino-1-[1-[4-(trifluoromethoxy)benzoyl]-4-piperidyl]-3H-imidazo[4,5-b]pyridin-2-one,
(rac)-6-(1-methylpyrrolidin-3-yl)oxy-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[(2S)-2-(hydroxymethyl)morpholin-4-yl]-3H-imidazo[4,5-b]pyridin-2-one,
6-[(2R)-2-(hydroxymethyl)morpholin-4-yl]-1-[1-[4-(trifluoromethoxy)benzoyl]-4-piperidyl]-3H-imidazo[4,5-b]pyridin-2-one,
6-[(2S)-2-(hydroxymethyl)morpholin-4-yl]-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[(2R)-2-(hydroxymethyl)morpholin-4-yl]-3H-imidazo[4,5-b]pyridin-2-one,
1-[1-[2-amino-4-(pentafluoro-λ⁶-sulfanyl)benzoyl]-4-piperidyl]-6-tetrahydropyran-4-yl-3H-imidazo[4,5-b]pyridin-2-one,
(rac)-6-tetrahydrofuran-3-yl-1-[1-[4-(trifluoromethoxy)benzoyl]-4-piperidyl]-3H-imidazo[4,5-b]pyridin-2-one,
(rac)-1-[1-[2-amino-4-(trifluoromethoxy)benzoyl]-4-piperidyl]-6-tetrahydrofuran-3-yl-3H-imidazo[4,5-b]pyridin-2-one,
6-cyclopentyl-1-[1-[4-(trifluoromethoxy)benzoyl]-4-piperidyl]-3H-imidazo[4,5-b]pyridin-2-one,
1-[1-[2-hydroxy-4-(trifluoromethoxy)benzoyl]-4-piperidyl]-6-tetrahydropyran-4-yl-3H-imidazo[4,5-b]pyridin-2-one,
1-[1-[4-(pentafluoro-λ⁶-sulfanyl)benzoyl]-4-piperidyl]-6-tetrahydropyran-4-yl-3H-imidazo[4,5-b]pyridin-2-one,
   and the pharmaceutically acceptable salts thereof.

In embodiments, the compound of the disclosure is characterised according to its inhibitory activity against ERK5, e.g., as measured according to the cell-based assay or cell-free assay described in the examples below. In embodiments, the compound has an IC₅₀ value of less than about 10 μM against ERK5 (e.g., when measured according to the cell-free assay described below). In embodiments, the compound has an IC₅₀ value of less than about 5 μM against ERK5 (e.g., when measured according to the cell-free assay described below). In embodiments, the compound has an IC₅₀ value of less than about 2 μM, e.g., less than about 1 μM, 0.5 μM, 0.2 μM, 100 nM, or 50 nM against ERK5 (e.g., when measured according to the cell-free assay described below). In embodiments, the compound has an IC₅₀ value of less than about 10 μM against ERK5 when measured according to the cell-free assay described below. In embodiments, the compound has an IC₅₀ value of less than about 5 μM, e.g., less than about 2 μM, 1 μM, 0.5 μM, 0.2 μM, 100 nM, or 50 nM against ERK5 when measured according to the cell-free assay described below. In embodiments, the compound has an IC₅₀ value of less than about 10 μM against ERK5 when measured according to the cell-based assay described below. In embodiments, the compound has an IC₅₀ value of less than about 5 μM, e.g., less than about 2 μM, 1 μM, 0.5 μM, 0.2 μM, 100 nM, or 50 nM against ERK5 when measured according to the cell-based assay described below.

In embodiments, the compound is selected from the compound of Examples 1, 2, 3, 4, 5, 6, 7, 8, 9, 10, 14, 15, 16, 17, and 19. In other embodiments, the compound is selected from the compound of Examples 1, 2, 3, 4, 5, 6, 7, 8, 9, 14, 15, 16, 17, and 19. In other embodiments, the compound is selected from the compound of Examples 1, 2, 3, 4, 6, 14, 16, 17, and 19. In other embodiments, the compound is selected from the compound of Examples 1, 2, and 3. In other embodiments, the compound is the compound of Example 1.

In embodiments, the compound is selected from the compound of Examples 1, 2, 3, 4, 5, 6, 7, 8, 9, 10, 12, 13, 14, 15, 16, 17, 18, and 19. In other embodiments, the compound is selected from the compound of Examples 1, 2, 3, 4, 5, 6, 7, 9, 10, 12, 13, 14, 15, 16, 17, 18, and 19. In other embodiments, the compound is selected from the compound of Examples 1, 2, 3, 4, 5, 6, 7, 10, 14, 16, 17, and 18. In other embodiments, the compound is selected from the compound of Examples 1, 2, 3, 6, 7, 16, and 18. In other embodiments, the compound is selected from the compound of Examples 1 and 2.

In embodiments, the compound is selected from the compound of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, and 67. In embodiments, the compound is selected from the compound of Examples 1, 2, 3, 4, 5, 6, 7, 8, 9, 10, 14, 15, 16, 17, 18, 19, 20, 21, 22, 23, 24, 25, 26, 27, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 60, 61, 62, 63, 64, 65, 66, and 67. In other embodiments, the compound is selected from the compound of Examples 1, 2, 3, 4, 5, 6, 7, 8, 9, 14, 15, 16, 17, 18, 19, 20, 21, 22, 23, 24, 25, 26, 29, 30, 31, 32, 33, 34, 35, 36, 37, 39, 40, 41, 42, 43, 44, 45, 46, 47, 48, 49, 50, 51, 52, 53, 54, 55, 56, 57, 58, 61, 62, 63, 64, and 67. In other embodiments, the compound is selected from the compound of Examples 1, 2, 3, 4, 6, 14, 16, 17, 18, 19, 21, 22, 23, 24, 25, 26, 31, 33, 37, 39, 40, 41, 42, 43, 44, 45, 46, 48, 53, 54, 55, and 56. In other embodiments, the compound is selected from the compound of Examples 1, 2, 3, 18, 21, 24, 25, 39, 43, 45, 55, and 56. In other embodiments, the compound is selected from the compound of Examples 1, 21, and 45.

In embodiments, the compound is selected from the compound of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, and 60. In embodiments, the compound is selected from the compound of Examples 1, 2, 3, 4, 5, 6, 7, 8, 9, 10, 12, 13, 14, 15, 16, 17, 18, 19, 21, 22, 23, 24, 25, 26, 28, 29, 31, 33, 34, 35, 36, 37, 39, 40, 41, 42, 43, 44, 45, 46, 47, 48, 49, 51, 52, 53, 54, 55, 56, 57, and 58. In other embodiments, the compound is selected from the compound of Examples 1, 2, 3, 4, 5, 6, 7, 9, 10, 12, 13, 14, 15, 16, 17, 18, 19, 21, 23, 25, 26, 29, 31, 33, 39, 40, 42, 43, 44, 45, 47, 48, 51, 52, 53, 54, 55, 56, 57, and 58. In other embodiments, the compound is selected from the compound of Examples 1, 2, 3, 4, 5, 6, 7, 10, 14, 16, 17, 18, 23, 39, 43, 45, 48, 51, 52, 54, 55, and 57. In other embodiments, the compound is selected from the compound of Examples 1, 2, 3, 6, 7, 16, 18, 23, 48, 51, 52, 54, and 55.

### Pharmaceutical Compositions

The present disclosure provides a pharmaceutical composition comprising a compound described herein (e.g., a compound of Formula (I) or a pharmaceutically acceptable salt thereof), and at least one pharmaceutically acceptable excipient or carrier.

In embodiments, the pharmaceutical composition comprises a compound of Formula (N-I) or a pharmaceutically acceptable salt thereof. In embodiments, the pharmaceutical composition comprises a compound of Formula (I) or a pharmaceutically acceptable salt thereof. In embodiments, the pharmaceutical composition comprises a compound of Formula (N-IA) or a pharmaceutically acceptable salt thereof. In embodiments, the pharmaceutical composition comprises a compound of Formula (N-IB) or a pharmaceutically acceptable salt thereof. In embodiments, the pharmaceutical composition comprises a compound of Formula (N-IC) or a pharmaceutically acceptable salt thereof. In embodiments, the pharmaceutical composition comprises a compound of Formula (N-ID) or a pharmaceutically acceptable salt thereof. In embodiments, the pharmaceutical composition comprises a compound of Formula (I-A) or a pharmaceutically acceptable salt thereof. In embodiments, the pharmaceutical composition comprises a compound of Formula (I-B) or a pharmaceutically acceptable salt thereof. In embodiments, the pharmaceutical composition comprises a compound of Formula (I-C) or a pharmaceutically acceptable salt thereof. In embodiments, the pharmaceutical composition comprises a compound of Formula (I-D) or a pharmaceutically acceptable salt thereof. In embodiments, the pharmaceutical composition comprises a compound of Formula (N-II) or a pharmaceutically acceptable salt thereof. In embodiments, the pharmaceutical composition comprises a compound of Formula (N-IIA) or a pharmaceutically acceptable salt thereof. In embodiments, the pharmaceutical composition comprises a compound of Formula (N-IIB) or a pharmaceutically acceptable salt thereof. In embodiments, the pharmaceutical composition comprises a compound of Formula (N-IIC) or a pharmaceutically acceptable salt thereof. In embodiments, the pharmaceutical composition comprises a compound of Formula (II) or a pharmaceutically acceptable salt thereof. In embodiments, the pharmaceutical composition comprises a compound of Formula (II-A) or a pharmaceutically acceptable salt thereof. In embodiments, the pharmaceutical composition comprises a compound of Formula (II-B) or a pharmaceutically acceptable salt thereof. In embodiments, the pharmaceutical composition comprises a compound of Formula (II-C) or a pharmaceutically acceptable salt thereof. In embodiments, the pharmaceutical composition comprises a compound of Formula (III) or a pharmaceutically acceptable salt thereof. In embodiments, the pharmaceutical composition comprises a compound of Formula (III-A) or a pharmaceutically acceptable salt thereof. In embodiments, the pharmaceutical composition comprises a compound of Formula (III-B) or a pharmaceutically acceptable salt thereof. In embodiments, the pharmaceutical composition comprises a compound of Formula (N-IV) or a pharmaceutically acceptable salt thereof. In embodiments, the pharmaceutical composition comprises a compound of Formula (IV) or a pharmaceutically acceptable salt thereof. In embodiments, the pharmaceutical composition comprises a compound of Formula (IV-A) or a pharmaceutically acceptable salt thereof. In embodiments, the pharmaceutical composition comprises a compound of Formula (IV-B) or a pharmaceutically acceptable salt thereof. In embodiments, the pharmaceutical composition comprises a compound of Formula (IV-C) or a pharmaceutically acceptable salt thereof. In embodiments, the pharmaceutical composition comprises a compound of Formula (V) or a pharmaceutically acceptable salt thereof. In embodiments, the pharmaceutical composition comprises a compound of Formula (VI) or a pharmaceutically acceptable salt thereof. In embodiments, the pharmaceutical composition comprises a compound of Formula (VII) or a pharmaceutically acceptable salt thereof.

The pharmaceutical compositions of the disclosure may be formulated for administration in solid or liquid form, e.g., using conventional carriers or excipients. Compositions may be adapted for, e.g., oral administration (e.g., as a solution, suspension, tablet, or capsule), parenteral administration (e.g., as a solution, dispersion, suspension, or emulsion, or as a dry powder for reconstitution), or topical application (e.g., as a cream, ointment, patch, or spray to be applied to the skin) using techniques known in the art.

### Medical Uses

Compounds of the present disclosure act as inhibitors of ERK5, which gives them utility in the treatment of ERK5-associated disorders and conditions. In particular, compounds of the disclosure are useful in the treatment of cancers.

Viewed from this aspect, the disclosure provides a method of treatment comprising administering to a subject in need thereof a therapeutically effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament. In a further related aspect, the disclosure provides a compound of the disclosure (e.g., a compound of Formula (I) or a pharmaceutically acceptable salt thereof) for use in therapy.

Compounds of the present disclosure are useful for treating or preventing: diseases or deleterious conditions in which ERK5, or a variant or mutant thereof, is known to play a role; diseases or disorders associated with increased MAPK7 (i.e., ERK5 gene) expression and/or increased ERK5 activity; and diseases or disorders in which inhibition or antagonism of ERK5 activity is beneficial.

In one aspect, the present disclosure provides a method of treating or preventing a disease or disorder mediated by ERK5, or a disease or disorder in which ERK5 is implicated, in a subject in need thereof, the method comprising administering to the subject an effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament for the treatment or prevention of a disease or disorder mediated by ERK5, or a disease or disorder in which ERK5 is implicated. In a further related aspect, the disclosure provides a compound of the disclosure (e.g., a compound of Formula (I) or a pharmaceutically acceptable salt thereof) for use in the treatment or prevention of a disease or disorder mediated by ERK5, or a disease or disorder in which ERK5 is implicated.

In another aspect, the present disclosure provides a method of treating or preventing a disease or disorder associated with ERK5 (e.g., cancer) in a subject in need thereof, the method comprising administering to the subject an effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament for the treatment or prevention of a disease or disorder associated with ERK5 (e.g., cancer). In a further related aspect, the disclosure provides a compound of the disclosure (e.g., a compound of Formula (I) or a pharmaceutically acceptable salt thereof) for use in the treatment or prevention of a disease or disorder associated with ERK5 (e.g., cancer).

In another aspect, the present disclosure provides a method of treating or preventing cancer in a subject in need thereof, the method comprising administering to the subject an effective amount of a compound of the disclosure (e.g., a compound of Formula (I) or a pharmaceutically acceptable salt thereof). In a related aspect, the disclosure provides the use of a compound of the disclosure (e.g., a compound of Formula (I) or a pharmaceutically acceptable salt thereof) in the manufacture of a medicament for the treatment or prevention of cancer. In a further related aspect, the disclosure provides a compound of the disclosure (e.g., a compound of Formula (I) or a pharmaceutically acceptable salt thereof) for use in the treatment or prevention of cancer.

In embodiments, the compound reduces angiogenesis, reduces or prevents metastasis, reduces inflammation, blocks tumorigenesis (e.g., in part or completely), reduces evasion of growth suppression, reduces or inhibits growth of cancerous or pre-cancerous cells, suppresses proliferation of cancerous or pre-cancerous cells, and/or reduces the survival of cancerous or pre-cancerous cells.

In embodiments, the cancer is characterized by increased MAPK7 (i.e., ERK5 gene) expression and/or increased ERK5 activity. In embodiments, the cancer has elevated ERK5 activity. In embodiments, the cancer overexpresses ERK5. In embodiments, the cancer is characterised by MAPK7 genomic amplification and/or constitutively active ERK5 signalling.

In embodiments, the cancer has genomically amplified ERK5. In embodiments, the cancer has constitutively active ERK5 signalling.

In embodiments, the cancer is a solid tumour (e.g., a melanoma, carcinoma, or blastoma). In other embodiments, the cancer is leukaemia (e.g., chronic lymphocytic leukaemia, CLL; acute myelogenous leukaemia, AML; or chronic myelogenous leukaemia, CML).

In embodiments, the cancer is a primary tumour. In other embodiments, the cancer is a secondary tumour (e.g., a metastatic tumour).

In embodiments, the cancer is selected from breast cancer (e.g., ductal breast carcinoma, or breast adenocarcinoma), liver cancer, kidney cancer (e.g., hepatocellular carcinoma), prostate cancer, colorectal cancer (CRC), lung cancer (e.g., non-small cell lung cancer, NSCLC; lung adenocarcinoma; or lung squamous cell carcinoma), pancreatic cancer (e.g., adenocarcinoma), ovarian cancer, brain cancer (e.g., glioblastoma), cervical cancer (e.g., adenocarcinoma), gastric cancer, skin cancer (e.g., melanoma), bile duct cancer (e.g., cholangiocarcinoma), nervous system cancer (e.g., neuroblastoma), and melanoma.

In embodiments, the cancer is selected from leukaemia (e.g., acute leukaemia, acute lymphocytic leukaemia, acute myelocytic leukaemia, acute myeloblastic leukaemia, acute promyelocytic leukaemia, acute myelomonocytic leukaemia, acute monocytic leukaemia, acute erythroleukemia, chronic leukaemia, chronic myelocytic leukaemia, or chronic lymphocytic leukaemia), polycythemia vera, lymphoma (e.g., Hodgkin's disease or non-Hodgkin's disease), Waldenstrom macroglobulinemia, and multiple myeloma.

In embodiments, the cancer is selected from leukaemia (e.g., chronic myeloid leukaemia), breast cancer, multiple myeloma, colon cancer, colorectal cancer, lung cancer, pancreatic cancer, renal cell carcinoma, mesothelioma, adenocarcinoma, neuroblastoma, melanoma, and hepatocellular carcinoma.

In embodiments, the cancer is selected from leukaemia (e.g., chronic myeloid leukaemia), breast cancer, multiple myeloma, colon cancer, renal cell carcinoma, mesothelioma, adenocarcinoma, neuroblastoma, and hepatocellular carcinoma.

In another aspect, the disclosure provides a method of inhibiting ERK5 activity, the method comprising contacting ERK5 (e.g., a cell comprising ERK5) with a compound of the present disclosure (e.g., a compound of Formula (I) or a pharmaceutically acceptable salt thereof). In embodiments, the method is an in vitro or ex vivo method. In other embodiments the method is an in vivo method. In a related aspect, the disclosure provides an in vitro method of inhibiting ERK5 activity in a cell, the method comprising contacting the cell with a compound of the present disclosure (e.g., a compound of Formula (I) or a pharmaceutically acceptable salt thereof).

Compounds of the present disclosure (e.g., compounds of Formula (I)) and the pharmaceutically acceptable salts thereof may be administered as pharmaceutical compositions, which may optionally comprise one or more pharmaceutically acceptable excipients.

It will be appreciated that the methods and treatments of the various aspects of this disclosure may be effected by administering to a subject an effective amount of a compound of the disclosure (e.g., a compound of Formula (I) or a pharmaceutically acceptable salt thereof), in the form of a pharmaceutical composition, which may optionally comprise one or more pharmaceutically acceptable excipients, as described herein.

The compounds of the disclosure may be used alone (e.g., as a monotherapy) or in combination with one or more cancer therapies.

Having been generally described herein, the follow non-limiting examples are provided to further illustrate this disclosure.

### EXAMPLES

### General Synthetic Schemes

The following scheme, Scheme 1A, illustrates an exemplary way of preparing compounds in accordance with the present disclosure and examples:

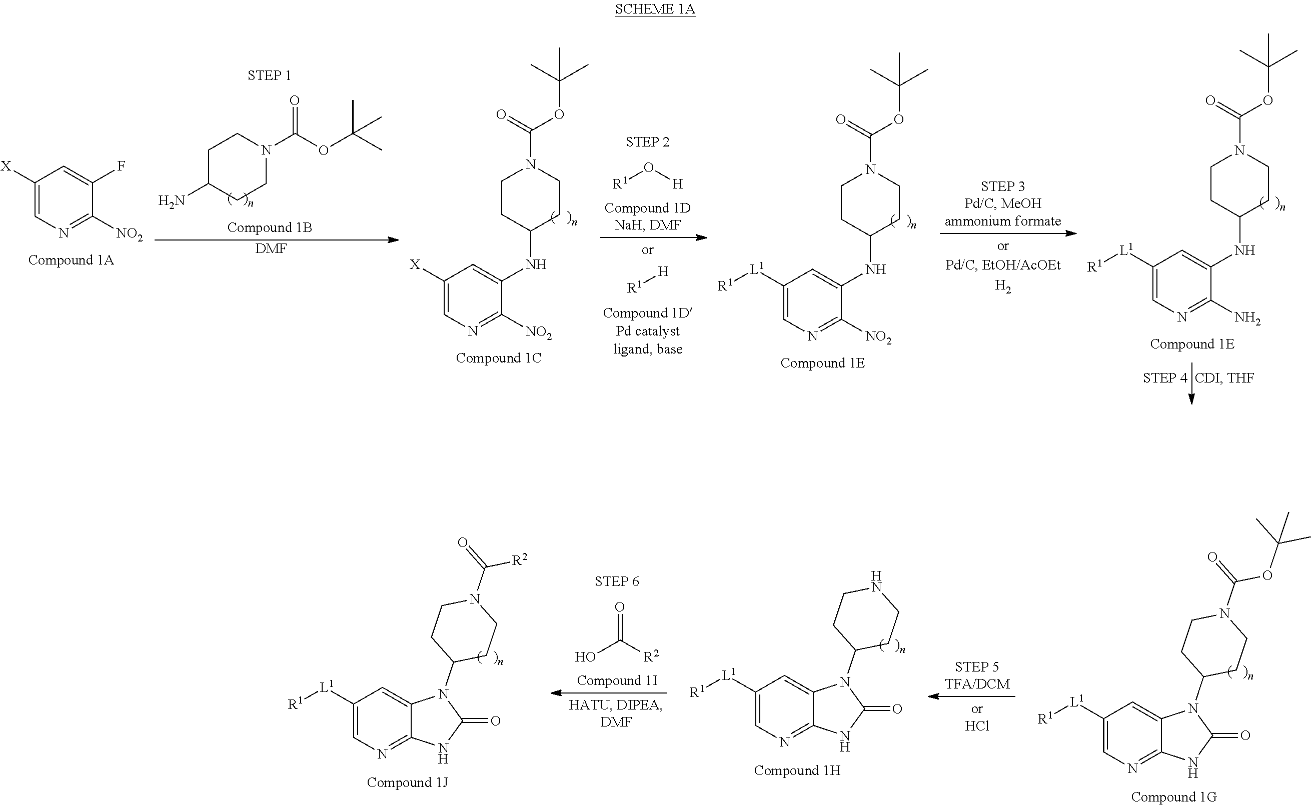

According to Scheme 1A (in which R¹, R², L¹, and n may be, e.g., as described above), Compound 1C can be obtained in STEP 1 by nucleophilic aromatic substitution between Compound 1A (wherein X=I, Br, Cl, F) and Compound 1B. Compound 1E can be prepared by reacting Compound 1C with alcohol Compound 1D by nucleophilic aromatic substitution using, for example, a base such as sodium hydride. Alternatively, Compound 1E can be prepared by reacting Compound 1C with Compound 1D′ (wherein R¹—H denotes the hydrogen atom of a secondary amine) by Buchwald coupling using a palladium catalyst such as Pd(OAc)₂ in the presence of RuPhos and a base such as LiHMDS. Compound 1E can be reduced in STEP 3 to Compound 1F by hydrogenation with a catalyst such as Pd/C under hydrogen pressure (H₂) around 5 bars at 40° C., for example. Compound 1F can then be converted to Compound 1G in STEP 4 by condensation with CDI, for example. Compound 1G can be N-deprotected by using HCl or TFA to give Compound 1H. Compound 1J can then be prepared from Compound 1H in STEP 6 with carboxylic acid Compound 11, using conditions known by the person skilled in the art such as HATU in a solvent like DMF in presence of a base such as DIPEA.

An alternative way to convert Compound 1C into Compound 1F is illustrated in the following scheme, Scheme 1B:

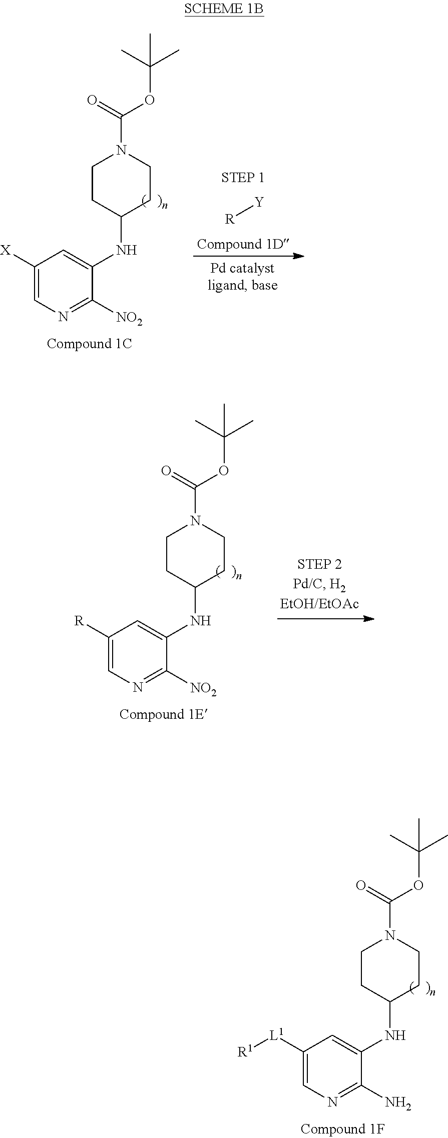

According to Scheme 1B (in which R¹, and n may be, e.g., as described above, and L¹ is a direct bond), Compound 1E′ can be prepared in STEP 1 by reacting Compound 1C with Compound 1D″ (R—Y, wherein R is an oxidized precursor of R¹ and Y=BPin) by Suzuki coupling using a palladium catalyst such as XPhosPdG4 in the presence of XPhos and a base. Compound 1E′ can be reduced in STEP 2 to Compound 1F by hydrogenation with a catalyst such as Pd/C under hydrogen pressure (H₂) around 5 bars at 40° C., for example; this not only reduces the nitro group of the pyridine but also reduces precursor R to form group R¹.

An alternative way to convert Compound 1C into Compound 1G is illustrated in the following scheme, Scheme 1C:

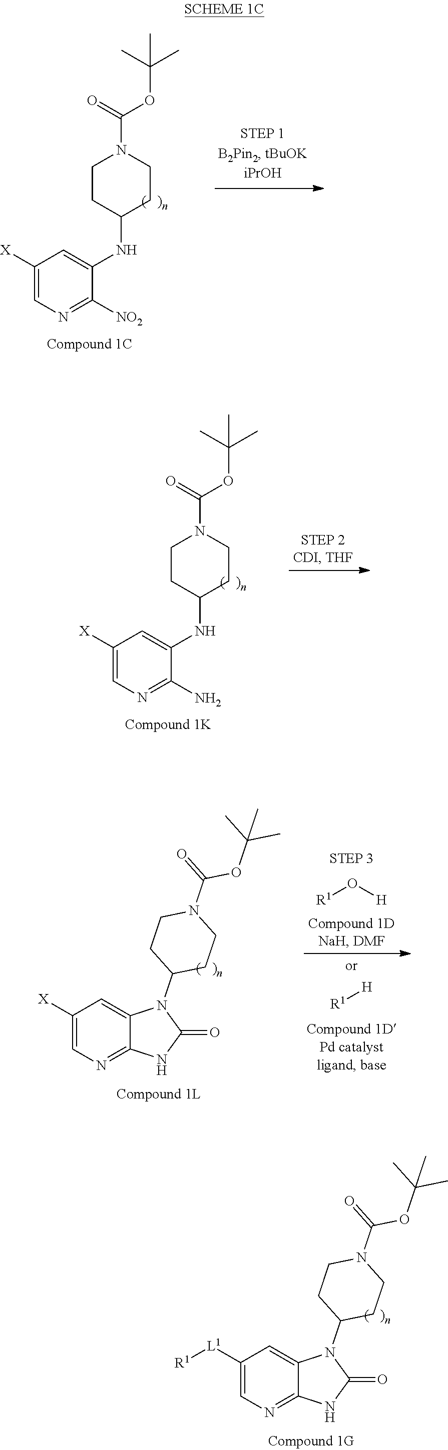

According to Scheme 1C (in which R¹, L¹, and n may be, e.g., as described above), Compound 1K can be obtained in STEP 1 by reduction of Compound 1C (wherein X may be I, Br, Cl, or F) using B₂Pin₂ and t-BuOK, for example. Compound 1L can be prepared from Compound 1K in STEP 2 by condensation with CDI. Compound 1G can be prepared in STEP 3 by analogy to Compound 1E in Scheme 1A. For example, Compound 1L may be reacted with alcohol Compound 1D by nucleophilic aromatic substitution using, for example, a base such as sodium hydride. Alternatively, Compound 1L may be reacted with Compound 1D′ by Buchwald coupling using a palladium catalyst such as Pd(OAc)₂ in the presence of RuPhos and a base such as LiHMDS.

An alternative way to convert Compound 1L into Compound 1G is illustrated in the following scheme, Scheme 1D (analogous to Scheme 1B above):

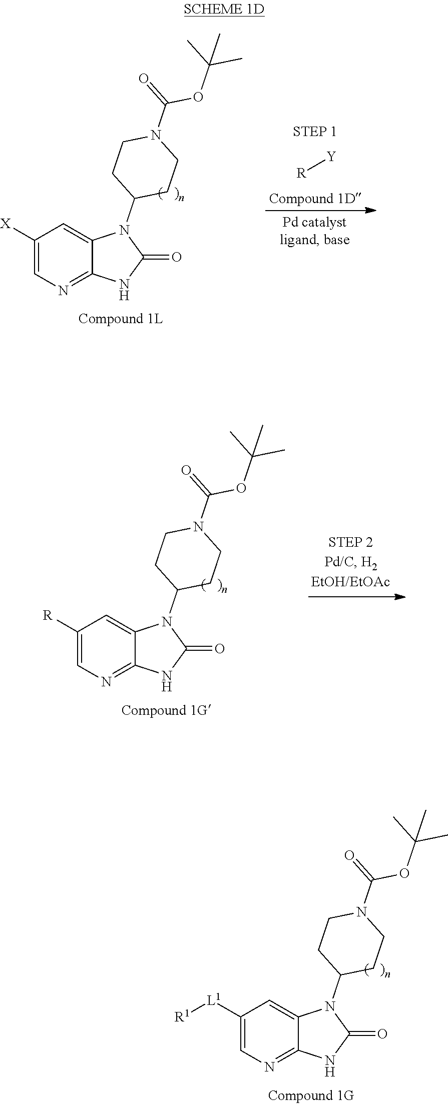

According to Scheme 1D (in which R¹, and n may be, e.g., as described above, and L¹ is a direct bond), Compound 1G′ can be prepared in STEP 1 by reacting Compound 1L with Compound 1D″ (R—Y, wherein R is an oxidized precursor of R¹ and Y=BPin) by Suzuki coupling using a palladium catalyst such as XPhosPdG4 in the presence of XPhos and a base. Compound 1G′ can be reduced in STEP 2 to Compound 1G by hydrogenation with a catalyst such as Pd/C under hydrogen pressure (H₂) around 5 bars at 40° C., for example.

An alternative way to convert Compound 1L into Compound 1J is illustrated in the following scheme, Scheme 1E (analogous to Scheme 1C above):

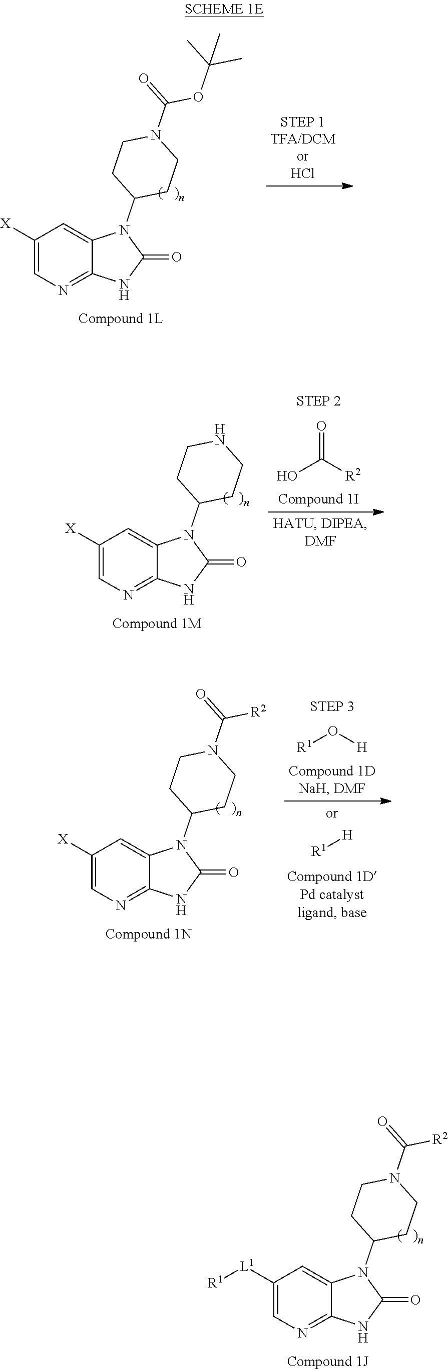

According to Scheme 1E (in which R¹, R², L¹, and n may be, e.g., as described above), Compound 1L (wherein X=I, Br, Cl, F) can be N-deprotected by using HCl or TFA to give Compound 1M in STEP 1. Compound 1N can then be prepared from compound 1M in STEP 2 with carboxylic acid Compound 11, using conditions known by the person skilled in the art such as HATU in a solvent like DMF in presence of a base such as DIPEA. Compound 1J can be prepared in STEP 3 by reacting Compound 1L either with alcohol Compound 1D by nucleophilic aromatic substitution using, for example, a base such as sodium hydride, or with Compound 1D′ by Buchwald coupling using a palladium catalyst such as Pd(OAc)₂ in the presence of RuPhos and a base such as LiHMDS.

An alternative way to convert Compound 1N into Compound 1J is illustrated in the following scheme, Scheme 1F (analogous to Scheme 1B and 1D above):

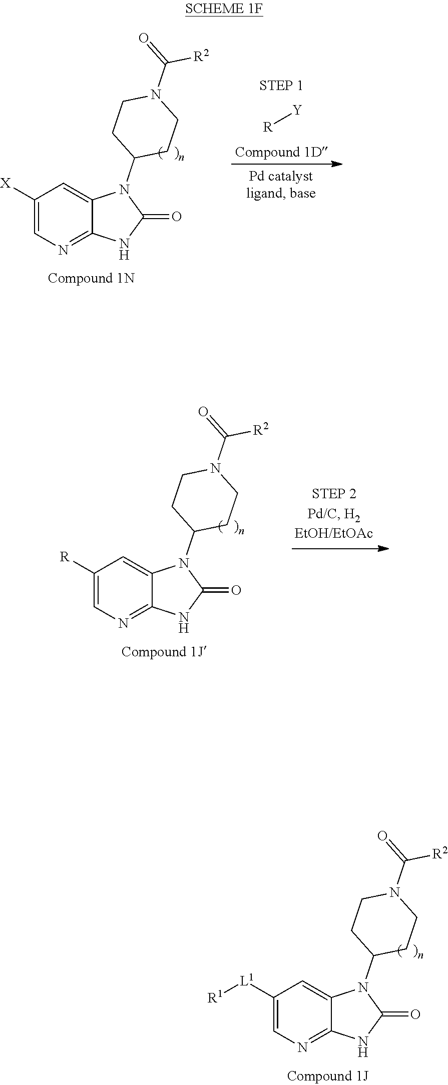

According to Scheme 1F (in which R¹, R², and n may be, e.g., as described above, and L¹ is a direct bond), Compound 1J′ can be prepared in STEP 1 by reacting Compound 1N with Compound 1D″ (R—Y, wherein R is an oxidized precursor of R¹ and Y=BPin) by Suzuki coupling using a palladium catalyst such as XPhosPdG4 in the presence of XPhos and a base. Compound 1J′ can be reduced in STEP 2 to Compound 1J by hydrogenation with a catalyst such as Pd/C under hydrogen pressure (H₂) around 5 bars at 40° C., for example.

Compounds in which R¹ is directly bonded to the pyridoimidazolone core via a tertiary carbon atom (e.g., where R¹ is a bicyclo[1.1.1]pentanyl group) may be obtained, e.g., by a modified version of any one of Schemes 1A to 1F in which a heteroaryl halide such as, e.g., Compound 1C, Compound 1L, or Compound 1N is reacted directly with R¹ in the form of a redox active ester, e.g., as described in Polites et al., Org. Lett. (2021) 23(12):4828-4833. Compounds in which R¹ is directly bonded to the pyridoimidazolone core via a tertiary carbon atom which has one oxygen substituent (e.g., where R^{A} is —OH, or —O(C₁-C₃)alkyl) may be obtained, e.g., by alkylation of a carbonyl group directly bonded to the pyridoimidazolone core at that position (which may be prepared from a heteroaryl halide, e.g., by palladium-assisted carbonylation) using, for example, an alkyl lithium or alkyl Grignard reagent (such as by the procedure described in WO 2021/195781), optionally with further alkylation of the resulting —OH group with an alkylating agent such as Mel in the presence of a base such as NaH. Where necessary in the aforementioned transformations, protecting groups may be used, e.g., on the —NH— group in the pyridoimidazolone core. Suitable protection strategies will be apparent to a person of skill in the art in view of the present disclosure.

The preparation of compounds of the disclosure in which L¹ is —CH₂—, or in which L¹ is a direct bond and R¹ is bonded to the rest of the molecule through an aliphatic carbon atom (e.g. a quaternary carbon), may be effected according to the following scheme, Scheme 1G.

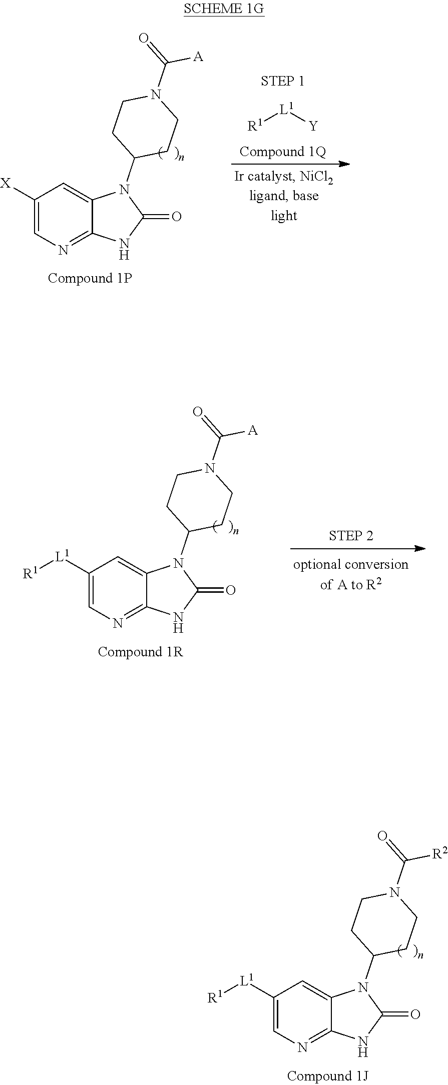

According to Scheme 1G (in which R¹, R², X, and n may be, e.g., as described above; and L¹ is a direct bond or —CH₂—), Compound 1R can be prepared from Compound 1P (in which A is selected from R² as defined herein, a precursor to R² (e.g., having an —NO₂ group in place of an —NH₂ group), and a protected version of R² (e.g., comprising an —NHBoc group), or taken together with the carbonyl to which it is attached forms a protecting group (e.g., Boc where A is —O-tBu, or Cbz where A is —OBn)) by a photochemical reaction with Compound 1Q (in which Y=BPin) using, for example, [Ir(dF(CF₃)ppy)₂(dtbbpy)]PF₆ and nickel(II) chloride in the presence of 4,4′-di-tert-butyl-2,2′-bipyridine, and morpholine or 4-acetamidopiperidine. Compound 1P may be prepared, e.g., analogously to Compound 1L or Compound 1N. Compound 1R may be converted to Compound 1J in STEP 2, e.g. by Boc deprotection using TFA or HCl, or by following STEPS 5 and 6 of Scheme 1A.

Compounds of the disclosure (e.g., compounds of Formula (N-I)) in which R³ is —(C₁-C₃)alkyl or —OH may be synthesized by analogy to the methods shown above, e.g., using a modified version of Scheme 1A. Scheme 2A below depicts how such compounds may be prepared by analogy to Scheme 1A.

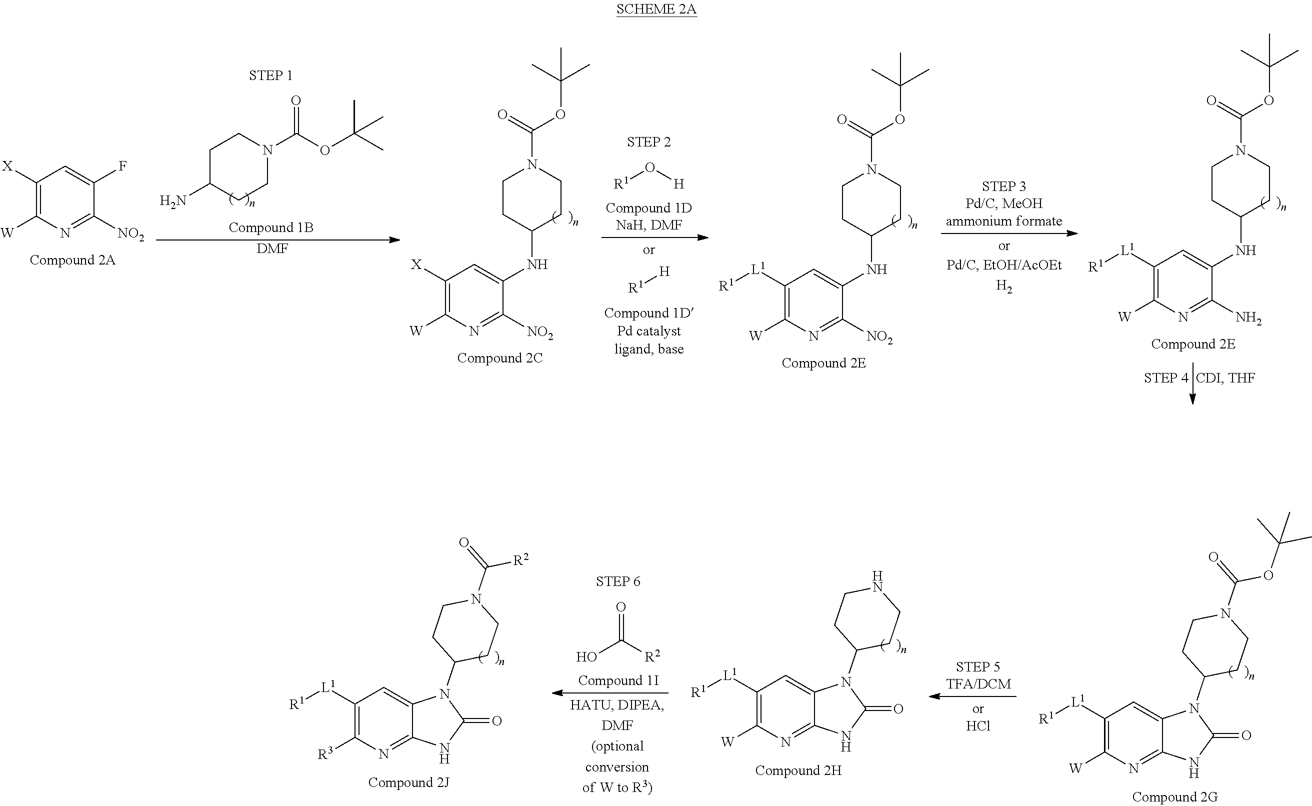

According to Scheme 2A (in which R¹, R², L¹, and n may be, e.g., as described above; and W is either R³, e.g. —(C₁-C₃)alkyl, or a protected version of R³, e.g. —OCH₃), Compound 2C can be obtained in STEP 1 by nucleophilic aromatic substitution between Compound 2A (wherein X=I, Br, Cl, F) and Compound 1B. Compound 2E can be prepared by reacting Compound 2C with alcohol Compound 1D by nucleophilic aromatic substitution using, for example, a base such as sodium hydride. Alternatively, Compound 2E can be prepared by reacting Compound 2C with Compound 1D′ (wherein R¹—H denotes the hydrogen atom of a secondary amine) by Buchwald coupling using a palladium catalyst such as Pd(OAc)₂ in the presence of RuPhos and a base such as LiHMDS. Compound 2E can be reduced in STEP 3 to Compound 2F by hydrogenation with a catalyst such as Pd/C under hydrogen pressure (H₂) around 5 bars at 40° C., for example. Compound 2F can then be converted to Compound 2G in STEP 4 by condensation with CDI, for example. Compound 2G can be N-deprotected by using HCl or TFA to give Compound 2H. Compound 2J can then be prepared from Compound 2H in STEP 6 with carboxylic acid Compound 11, using conditions known by the person skilled in the art such as HATU in a solvent like DMF in presence of a base such as DIPEA. Where W is a protected version of R³, STEP 6 may further comprise deprotection to reveal R³, for example where W is —OCH₃ demethylation conditions such as BBr₃, or HBr in water, or NaI followed by TMSCl may be used to furnish Compound 2J.

An alternative way of converting Compound 2C to Compound 2G is shown in the following scheme, Scheme 2B.

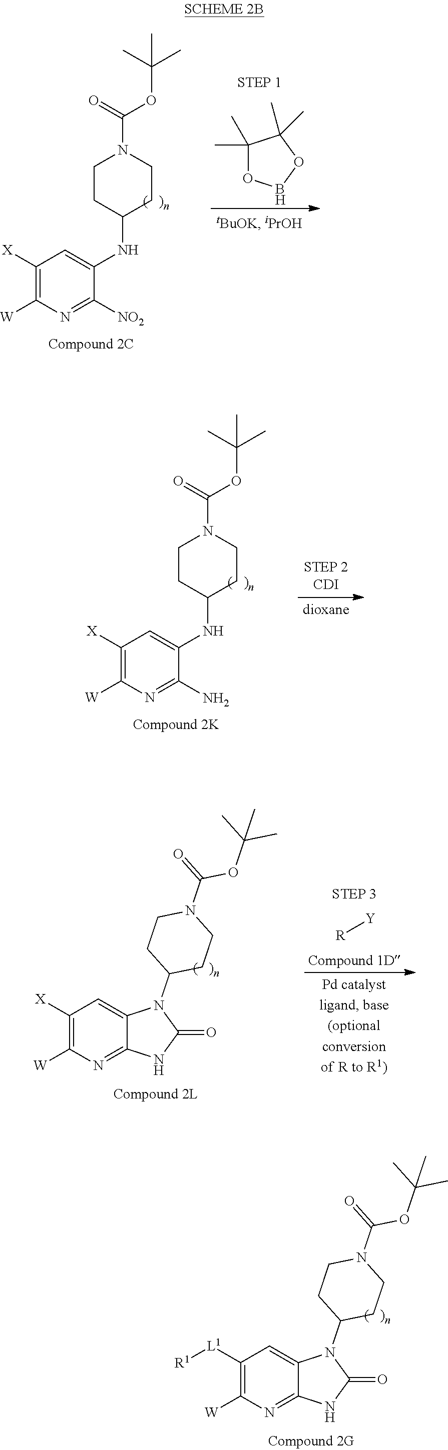

According to Scheme 2B (in which R¹, L¹, n, X, and W may be, e.g., as described above), Compound 2C can be reduced to Compound 2K in STEP 1 using, for example, pinacolborane in the presence of t-BuOK in a solvent such as iPrOH. Compound 2L can then be obtained in STEP 2 by reaction of Compound 2K with CDI in dioxane, for example. Compound 2L can then be reacted with Compound 1D″ (R—Y, wherein R is an oxidized precursor of R¹ and Y=BPin) by Suzuki coupling in STEP 3 using a palladium catalyst such as XPhosPdG4 in the presence of XPhos and a base. STEP 3 can optionally further comprise reduction of R to R¹ by hydrogenation with a catalyst such as Pd/C under hydrogen pressure (H₂) around 5 bars at 40° C., for example.

If not commercially available, Compound 2A may be prepared, for example, by the following scheme, Scheme 2C.

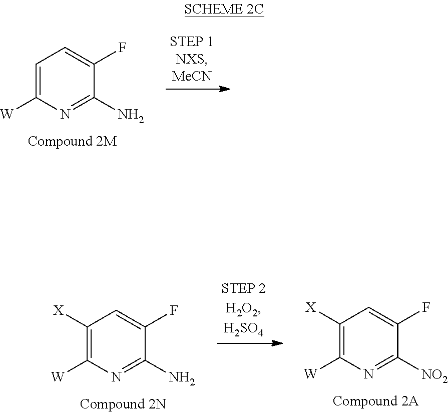

According to Scheme 2C, Compound 2N can be obtained in STEP 1 by halogenation of Compound 2M (wherein W═H or —(C₁-C₃)alkyl) using a halogenation reagent such as NXS (wherein X=I, Br, Cl, F). Compound 2A can be obtained in STEP 2 by oxidation of Compound 2N with H₂O₂ and sulfuric acid, for example.

### Experimental Techniques

¹H NMR Spectra at 400 and 500 MHz were performed on a Bruker Avance DRX-400 and Bruker Avance DPX-500 spectrometer, respectively, with the chemical shifts (6 in ppm) in the solvent dimethyl sulfoxide-d₆ (DMSO-d₆) referenced at 2.5 ppm at the quoted temperatures. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210<1<400 nm and column Acquity UPLC CSH C18 1.7 μm, dimension 2.1×30 mm, mobile phase H₂O+0.1% HCO₂H/CH₃CN+0.1% HCO₂H, or column Acquity BEH C18 1.7 μm, dimension 2.1×50 mm, mobile phase H₂O+0.05% formic acid/CH₃CN+0.035% formic acid.

All synthetic reactions were performed under an inert atmosphere, unless otherwise stated. In the following examples, when the source of the starting products is not specified, it should be understood that said products are known compounds (e.g., commercially available compounds from suppliers such as Sigma-Aldrich).

### Examples 1 to 67—Compounds

Table 1 below lists the compounds synthesized in the following synthetic examples.

TABLE 1

| Example No. | Structure | Name |
|---|---|---|
| 1 | 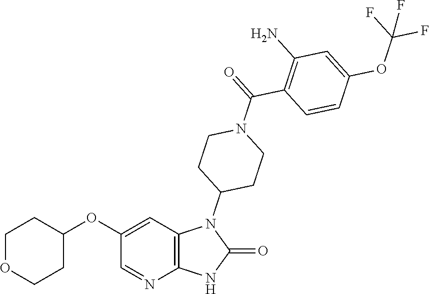 [SMILES: Nc1cc(OC(F)(F)F)ccc1C(=O)N1CCC(n2c(=O)[nH]c3ncc(OC4CCOCC4)cc32)CC1] | 1-[1-[2-amino-4- (trifluoromethoxy)benzoyl]-4- piperidyl]-6-tetrahydropyran-4-yloxy- 3H-imidazo[4,5-b]pyridin-2-one |
| 2 | 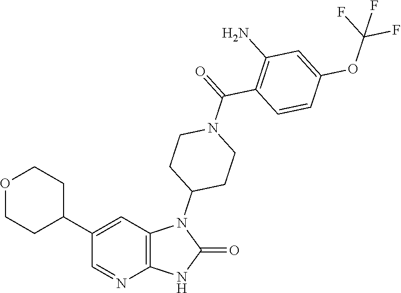 [SMILES: Nc1cc(OC(F)(F)F)ccc1C(=O)N1CCC(n2c(=O)[nH]c3ncc(C4CCOCC4)cc32)CC1] | 1-[1-[2-amino-4- (trifluoromethoxy)benzoyl]-4- piperidyl]-6-tetrahydropyran-4-yl-3H- imidazo[4,5-b]pyridin-2-one |
| 3 | 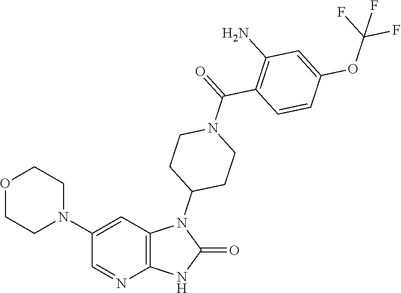 [SMILES: Nc1cc(OC(F)(F)F)ccc1C(=O)N1CCC(n2c(=O)[nH]c3ncc(N4CCOCC4)cc32)CC1] | 1-[1-[2-amino-4- (trifluoromethoxy)benzoyl]-4- piperidyl]-6-N-morpholino-3H- imidazo[4,5-b]pyridin-2-one |
| 4 | 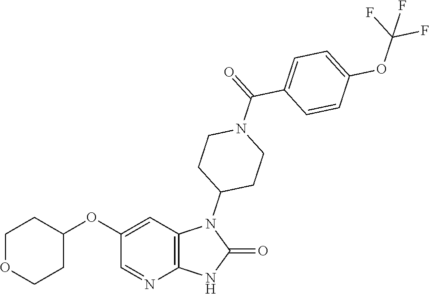 [SMILES: O=C(c1ccc(OC(F)(F)F)cc1)N1CCC(n2c(=O)[nH]c3ncc(OC4CCOCC4)cc32)CC1] | 6-tetrahydropyran-4-yloxy-1-[1-[4- (trifluoromethoxy)benzoyl]-4- piperidyl]-3H-imidazo[4,5-b]pyridin- 2-one |
| 5 | 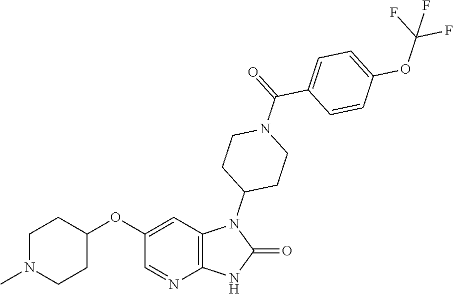 [SMILES: CN1CCC(Oc2cnc3[nH]c(=O)n(C4CCN(C(=O)c5ccc(OC(F)(F)F)cc5)CC4)c3c2)CC1] | 6-[(1-methyl-4-piperidyl)oxy]-1-[1-[4- (trifluoromethoxy)benzoyl]-4- piperidyl]-3H-imidazo[4,5-b]pyridin- 2-one |
| 6 | 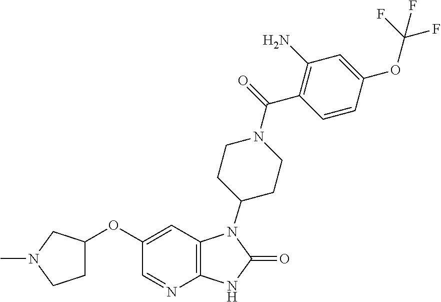 [SMILES: CN1CCC(Oc2cnc3[nH]c(=O)n(C4CCN(C(=O)c5ccc(OC(F)(F)F)cc5N)CC4)c3c2)C1] | (rac)-1-[1-[2-amino-4- (trifluoromethoxy)benzoyl]-4- piperidyl]-6-(1-methylpyrrolidin-3- yl)oxy-3H-imidazo[4,5-b]pyridin-2- one |
| 7 | 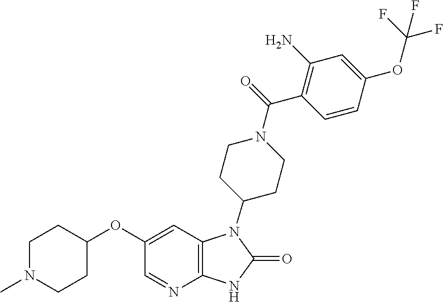 [SMILES: CN1CCC(Oc2cnc3[nH]c(=O)n(C4CCN(C(=O)c5ccc(OC(F)(F)F)cc5N)CC4)c3c2)CC1] | 1-[1-[2-amino-4- (trifluoromethoxy)benzoyl]-4- piperidyl]-6-[(1-methyl-4- piperidyl)oxy]-3H-imidazo[4,5- b]pyridin-2-one |
| 8 | 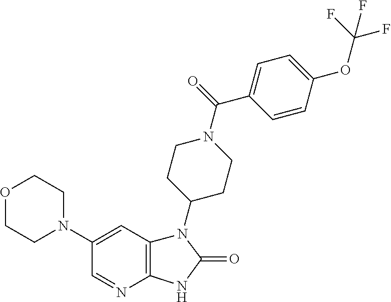 [SMILES: O=C(c1ccc(OC(F)(F)F)cc1)N1CCC(n2c(=O)[nH]c3ncc(N4CCOCC4)cc32)CC1] | 6-N-morpholino-1-[1-[4- (trifluoromethoxy)benzoyl]-4- piperidyl]-3H-imidazo[4,5-b]pyridin- 2-one |
| 9 | 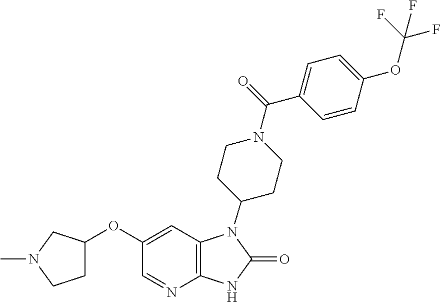 [SMILES: CN1CCC(Oc2cnc3[nH]c(=O)n(C4CCN(C(=O)c5ccc(OC(F)(F)F)cc5)CC4)c3c2)C1] | (rac)-6-(1-methylpyrrolidin-3-yl)oxy- 1-[1-[4-(trifluoromethoxy)benzoyl]-4- piperidyl]-3H-imidazo[4,5-b]pyridin- 2-one |
| 10 | 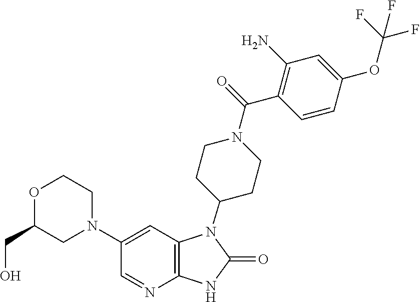 [SMILES: Nc1cc(OC(F)(F)F)ccc1C(=O)N1CCC(n2c(=O)[nH]c3ncc(N4CCO[C@H](CO)C4)cc32)CC1] | 1-[1-[2-amino-4- (trifluoromethoxy)benzoyl]-4- piperidyl]-6-[(2S)-2- (hydroxymethyl)morpholin-4-yl]-3H- imidazo[4,5-b]pyridin-2-one |
| 11 | 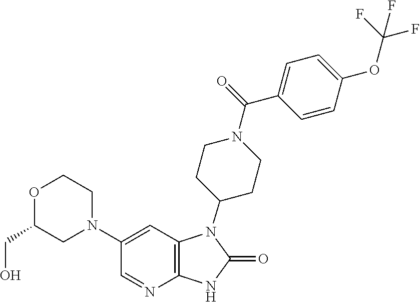 [SMILES: O=C(c1ccc(OC(F)(F)F)cc1)N1CCC(n2c(=O)[nH]c3ncc(N4CCO[C@@H](CO)C4)cc32)CC1] | 6-[(2R)-2-(hydroxymethyl)morpholin- 4-yl]-1-[1-[4- (trifluoromethoxy)benzoyl]-4- piperidyl]-3H-imidazo[4,5-b]pyridin- 2-one |
| 12 | 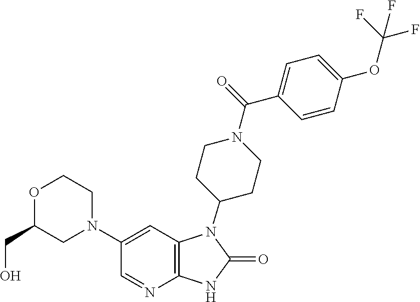 [SMILES: O=C(c1ccc(OC(F)(F)F)cc1)N1CCC(n2c(=O)[nH]c3ncc(N4CCO[C@H](CO)C4)cc32)CC1] | 6-[(2S)-2-(hydroxymethyl)morpholin- 4-yl]-1-[1-[4- (trifluoromethoxy)benzoyl]-4- piperidyl]-3H-imidazo[4,5-b]pyridin- 2-one |
| 13 | 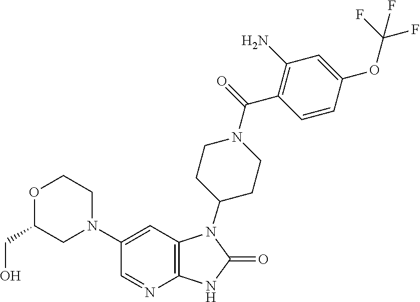 [SMILES: Nc1cc(OC(F)(F)F)ccc1C(=O)N1CCC(n2c(=O)[nH]c3ncc(N4CCO[C@@H](CO)C4)cc32)CC1] | 1-[1-[2-amino-4- (trifluoromethoxy)benzoyl]-4- piperidyl]-6-[(2R)-2- (hydroxymethyl)morpholin-4-yl]-3H- imidazo[4,5-b]pyridin-2-one |
| 14 | 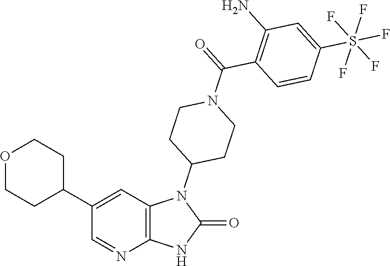 [SMILES: Nc1cc(S(F)(F)(F)(F)F)ccc1C(=O)N1CCC(n2c(=O)[nH]c3ncc(C4CCOCC4)cc32)CC1] | 1-[1-[2-amino-4-(pentafluoro-λ⁶- sulfanyl)benzoyl]-4-piperidyl]-6- tetrahydropyran-4-yl-3H-imidazo[4,5- b]pyridin-2-one |
| 15 | 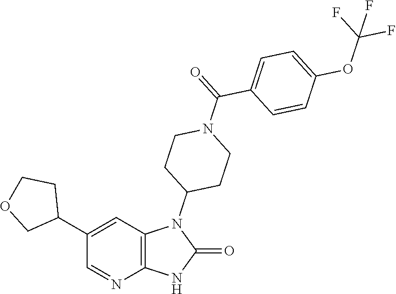 [SMILES: O=C(c1ccc(OC(F)(F)F)cc1)N1CCC(n2c(=O)[nH]c3ncc(C4CCOC4)cc32)CC1] | (rac)-6-tetrahydrofuran-3-yl-1-[1-[4- (trifluoromethoxy)benzoyl]-4- piperidyl]-3H-imidazo[4,5-b]pyridin- 2-one |
| 16 | 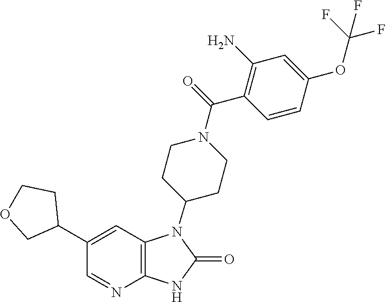 [SMILES: Nc1cc(OC(F)(F)F)ccc1C(=O)N1CCC(n2c(=O)[nH]c3ncc(C4CCOC4)cc32)CC1] | (rac)-1-[1-[2-amino-4- (trifluoromethoxy)benzoyl]-4- piperidyl]-6-tetrahydrofuran-3-yl-3H- imidazo[4,5-b]pyridin-2-one |
| 17 | 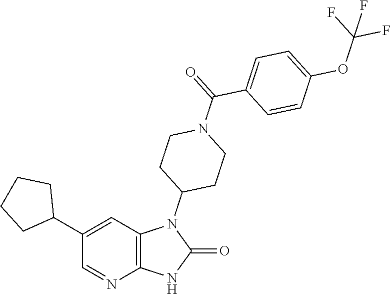 [SMILES: O=C(c1ccc(OC(F)(F)F)cc1)N1CCC(n2c(=O)[nH]c3ncc(C4CCCC4)cc32)CC1] | 6-cyclopenty1-1-[1-[4- (trifluoromethoxy)benzoyl]-4- piperidyl]-3H-imidazo[4,5-b]pyridin- 2-one |
| 18 | 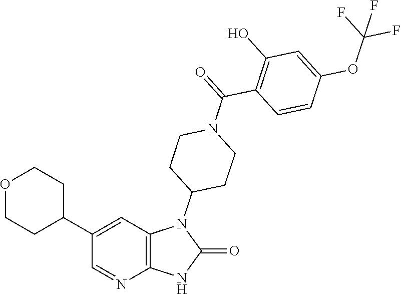 [SMILES: O=C(c1ccc(OC(F)(F)F)cc1O)N1CCC(n2c(=O)[nH]c3ncc(C4CCOCC4)cc32)CC1] | 1-[1-[2-hydroxy-4- (trifluoromethoxy)benzoyl]-4- piperidyl]-6-tetrahydropyran-4-yl-3H- imidazo[4,5-b]pyridin-2-one |
| 19 | 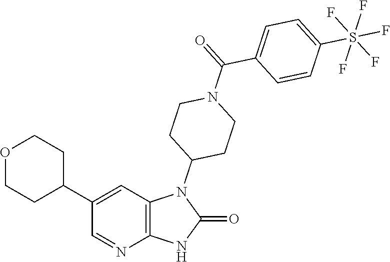 [SMILES: O=C(c1ccc(S(F)(F)(F)(F)F)cc1)N1CCC(n2c(=O)[nH]c3ncc(C4CCOCC4)cc32)CC1] | 1-[1-[4-(pentafluoro-λ⁶- sulfanyl)benzoyl]-4-piperidyl]-6- tetrahydropyran-4-y1-3H-imidazo[4,5- b]pyridin-2-one |
| 20 | 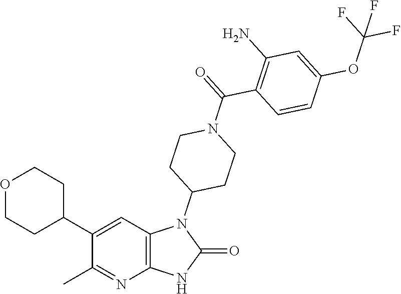 [SMILES: Cc1nc2[nH]c(=O)n(C3CCN(C(=O)c4ccc(OC(F)(F)F)cc4N)CC3)c2cc1C1CCOCC1] | 1-[1-[2-amino-4- (trifluoromethoxy)benzoyl]-4- piperidyl]-5-methyl-6- tetrahydropyran-4-yl-3H-imidazo[4,5- b]pyridin-2-one |
| 21 | 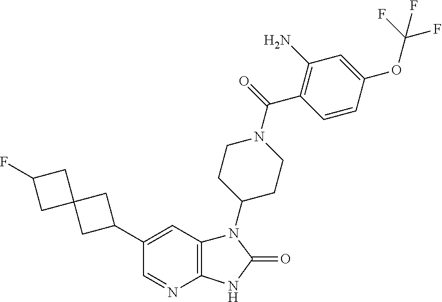 [SMILES: Nc1cc(OC(F)(F)F)ccc1C(=O)N1CCC(n2c(=O)[nH]c3ncc(C4CC5(CC(F)C5)C4)cc32)CC1] | 1-[1-[2-amino-4- (trifluoromethoxy)benzoyl]-4- piperidyl]-6-(2-fluorospiro[3.3]heptan- 6-yl)-3H-imidazo[4,5-b]pyridin-2-one |
| 22 | 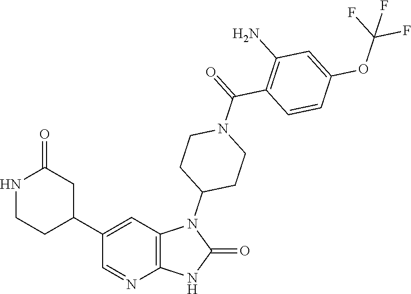 [SMILES: Nc1cc(OC(F)(F)F)ccc1C(=O)N1CCC(n2c(=O)[nH]c3ncc(C4CCNC(=O)C4)cc32)CC1] | (rac)-1-[1-[2-amino-4- (trifluoromethoxy)benzoyl]-4- piperidyl]-6-(2-oxo-4-piperidyl)-3H- imidazo[4,5-b]pyridin-2-one |
| 23 | 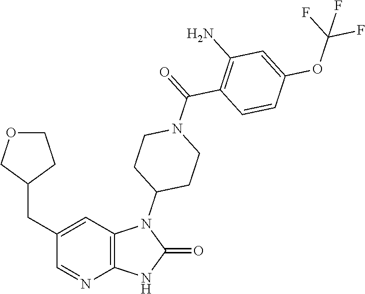 [SMILES: Nc1cc(OC(F)(F)F)ccc1C(=O)N1CCC(n2c(=O)[nH]c3ncc(CC4CCOC4)cc32)CC1] | (rac)-1-[1-[2-amino-4- (trifluoromethoxy)benzoyl]-4- piperidyl]-6-(tetrahydrofuran-3- ylmethyl)-3H-imidazo[4,5-b]pyridin- 2-one |
| 24 | 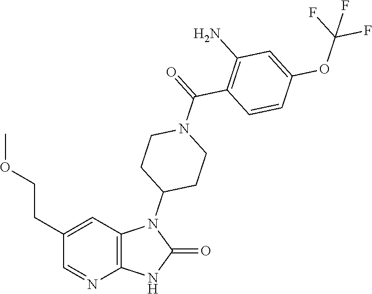 [SMILES: COCCc1cnc2[nH]c(=O)n(C3CCN(C(=O)c4ccc(OC(F)(F)F)cc4N)CC3)c2c1] | 1-[1-[2-amino-4- (trifluoromethoxy)benzoyl]-4- piperidyl]-6-(2-methoxyethyl)-3H- imidazo[4,5-b]pyridin-2-one |
| 25 | 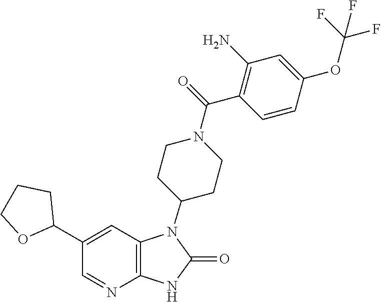 [SMILES: Nc1cc(OC(F)(F)F)ccc1C(=O)N1CCC(n2c(=O)[nH]c3ncc(C4CCCO4)cc32)CC1] | (rac)-1-[1-[2-amino-4- (trifluoromethoxy)benzoyl]-4- piperidyl]-6-tetrahydrofuran-2-yl-3H- imidazo[4,5-b]pyridin-2-one |
| 26 | 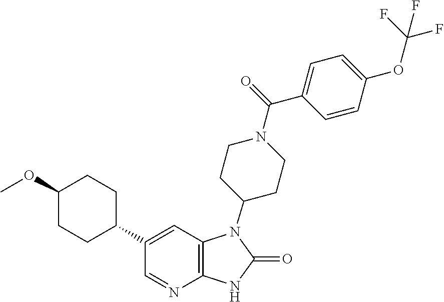 [SMILES: CO[C@H]1CC[C@H](c2cnc3[nH]c(=O)n(C4CCN(C(=O)c5ccc(OC(F)(F)F)cc5)CC4)c3c2)CC1] | trans-6-(4-methoxycyclohexyl)-1-[1- [4-(trifluoromethoxy)benzoyl]-4- piperidyl]-3H-imidazo[4,5-b]pyridin- 2-one |
| 27 | 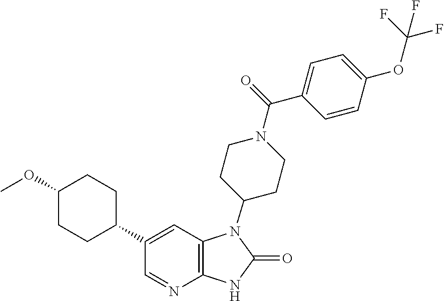 [SMILES: CO[C@H]1CC[C@@H](c2cnc3[nH]c(=O)n(C4CCN(C(=O)c5ccc(OC(F)(F)F)cc5)CC4)c3c2)CC1] | cis-6-(4-methoxycyclohexyl)-1-[1-[4- (trifluoromethoxy)benzoyl]-4- piperidyl]-3H-imidazo[4,5-b]pyridin- 2-one |
| 28 | 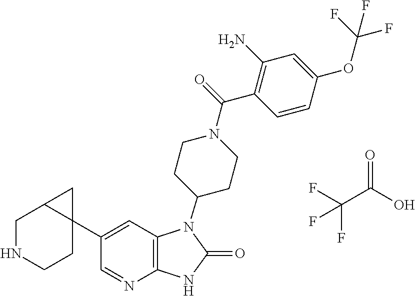 [SMILES: Nc1cc(OC(F)(F)F)ccc1C(=O)N1CCC(n2c(=O)[nH]c3ncc(C45CCNCC4C5)cc32)CC1 ; O=C(O)C(F)(F)F] | (rac)-1-[1-[2-amino-4- (trifluoromethoxy)benzoyl]-4- piperidyl]-6-(3- azabicyclo[4.1.0]heptan-6-yl)-3H- imidazo[4,5-b]pyridin-2-one, 2,2,2- trifluoroacetic acid |
| 29 | 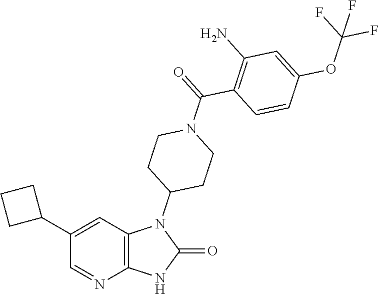 [SMILES: Nc1cc(OC(F)(F)F)ccc1C(=O)N1CCC(n2c(=O)[nH]c3ncc(C4CCC4)cc32)CC1] | 1-[1-[2-amino-4- (trifluoromethoxy)benzoyl]-4- piperidyl]-6-cyclobutyl-3H- imidazo[4,5-b]pyridin-2-one |
| 30 | 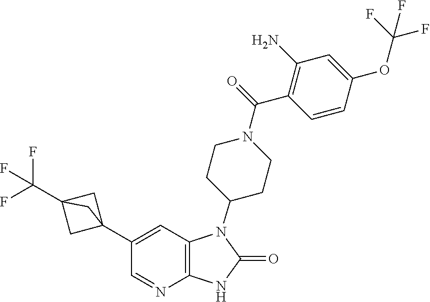 [SMILES: Nc1cc(OC(F)(F)F)ccc1C(=O)N1CCC(n2c(=O)[nH]c3ncc(C45CC(C(F)(F)F)(C4)C5)cc32)CC1] | 1-[1-[2-amino-4- (trifluoromethoxy)benzoyl]-4- piperidyl]-6-[3-(trifluoromethyl)-1- bicyclo[1.1.1]pentanyl]-3H- imidazo[4,5-b]pyridin-2-one |
| 31 | 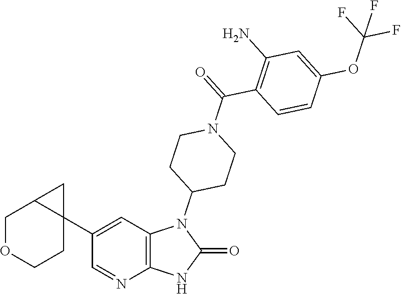 [SMILES: Nc1cc(OC(F)(F)F)ccc1C(=O)N1CCC(n2c(=O)[nH]c3ncc(C45CCOCC4C5)cc32)CC1] | (rac)-1-[1-[2-amino-4- (trifluoromethoxy)benzoyl]-4- piperidyl]-6-(3- oxabicyclo[4.1.0]heptan-6-yl)-3H- imidazo[4,5-b]pyridin-2-one |
| 32 | 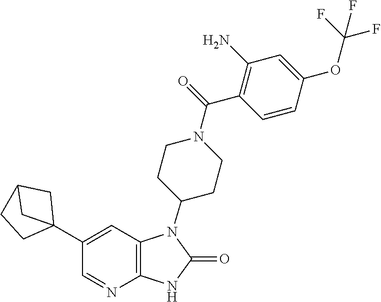 [SMILES: Nc1cc(OC(F)(F)F)ccc1C(=O)N1CCC(n2c(=O)[nH]c3ncc(C45CCC(C4)C5)cc32)CC1] | 1-[1-[2-amino-4- (trifluoromethoxy)benzoyl]-4- piperidyl]-6-(1-bicyclo[2.1.1]hexanyl)- 3H-imidazo[4,5-b]pyridin-2-one |
| 33 | 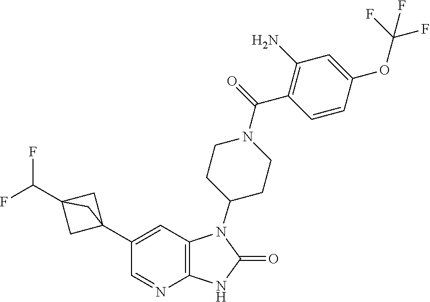 [SMILES: Nc1cc(OC(F)(F)F)ccc1C(=O)N1CCC(n2c(=O)[nH]c3ncc(C45CC(C(F)F)(C4)C5)cc32)CC1] | 1-[1-[2-amino-4- (trifluoromethoxy)benzoyl]-4- piperidyl]-6-[3-(difluoromethyl)-1- bicyclo[1.1.1]pentanyl]-3H- imidazo[4,5-b]pyridin-2-one |
| 34 | 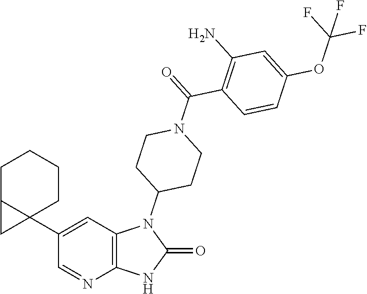 [SMILES: Nc1cc(OC(F)(F)F)ccc1C(=O)N1CCC(n2c(=O)[nH]c3ncc(C45CCCCC4C5)cc32)CC1] | (rac)-1-[1-[2-amino-4- (trifluoromethoxy)benzoyl]-4- piperidyl]-6-norcaran-1-y1-3H- imidazo[4,5-b]pyridin-2-one |
| 35 | 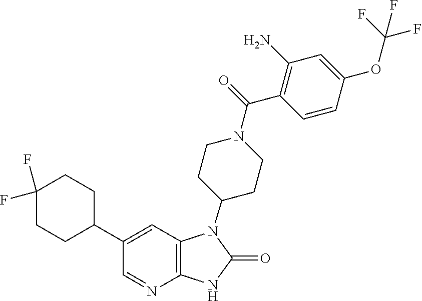 [SMILES: Nc1cc(OC(F)(F)F)ccc1C(=O)N1CCC(n2c(=O)[nH]c3ncc(C4CCC(F)(F)CC4)cc32)CC1] | 1-[1-[2-amino-4- (trifluoromethoxy)benzoyl]-4- piperidyl]-6-(4,4-difluorocyclohexyl)- 3H-imidazo[4,5-b]pyridin-2-one |
| 36 | 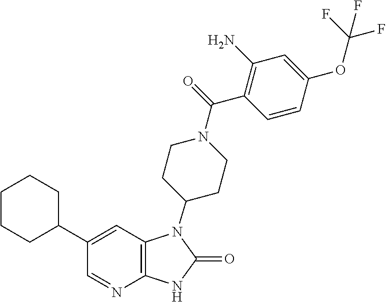 [SMILES: Nc1cc(OC(F)(F)F)ccc1C(=O)N1CCC(n2c(=O)[nH]c3ncc(C4CCCCC4)cc32)CC1] | 1-[1-[2-amino-4- (trifluoromethoxy)benzoyl]-4- piperidyl]-6-cyclohexyl-3H- imidazo[4,5-b]pyridin-2-one |
| 37 | 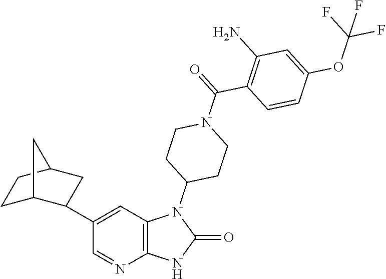 [SMILES: Nc1cc(OC(F)(F)F)ccc1C(=O)N1CCC(n2c(=O)[nH]c3ncc(C4CC5CCC4C5)cc32)CC1] | 1-[1-[2-amino-4- (trifluoromethoxy)benzoyl]-4- piperidyl]-6-norbornan-2-yl-3H- imidazo[4,5-b]pyridin-2-one |
| 38 | 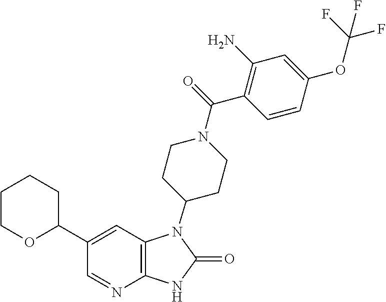 [SMILES: Nc1cc(OC(F)(F)F)ccc1C(=O)N1CCC(n2c(=O)[nH]c3ncc(C4CCCCO4)cc32)CC1] | (rac)-1-[1-[2-amino-4- (trifluoromethoxy)benzoyl]-4- piperidyl]-6-tetrahydropyran-2-yl-3H- imidazo[4,5-b]pyridin-2-one |
| 39 | 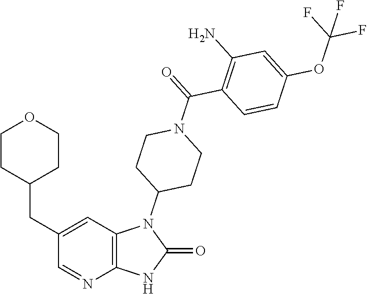 [SMILES: Nc1cc(OC(F)(F)F)ccc1C(=O)N1CCC(n2c(=O)[nH]c3ncc(CC4CCOCC4)cc32)CC1] | 1-[1-[2-amino-4- (trifluoromethoxy)benzoyl]-4- piperidyl]-6-(tetrahydropyran-4- ylmethyl)-3H-imidazo[4,5-b]pyridin- 2-one |
| 40 | 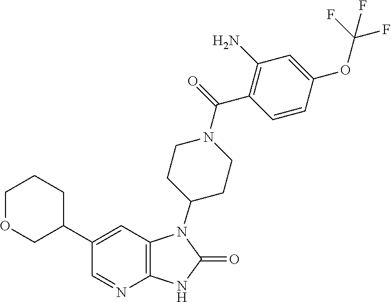 [SMILES: Nc1cc(OC(F)(F)F)ccc1C(=O)N1CCC(n2c(=O)[nH]c3ncc(C4CCCOC4)cc32)CC1] | (rac)-1-[1-[2-amino-4- (trifluoromethoxy)benzoyl]-4- piperidyl]-6-tetrahydropyran-3-yl-3H- imidazo[4,5-b]pyridin-2-one |
| 41 | 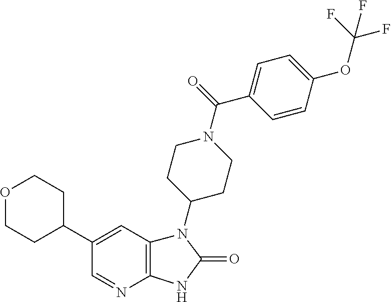 [SMILES: O=C(c1ccc(OC(F)(F)F)cc1)N1CCC(n2c(=O)[nH]c3ncc(C4CCOCC4)cc32)CC1] | 6-tetrahydropyran-4-y1-1-[1-[4- (trifluoromethoxy)benzoyl]-4- piperidyl]-3H-imidazo[4,5-b]pyridin- 2-one |
| 42 | 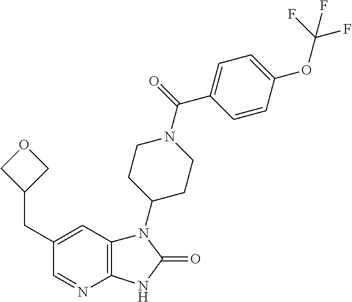 [SMILES: O=C(c1ccc(OC(F)(F)F)cc1)N1CCC(n2c(=O)[nH]c3ncc(CC4COC4)cc32)CC1] | 6-(oxetan-3-ylmethyl)-1-[1-[4- (trifluoromethoxy)benzoyl]-4- piperidyl]-3H-imidazo[4,5-b]pyridin- 2-one |
| 43 | 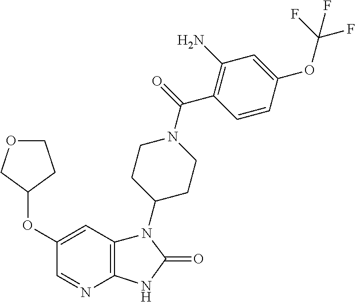 [SMILES: Nc1cc(OC(F)(F)F)ccc1C(=O)N1CCC(n2c(=O)[nH]c3ncc(OC4CCOC4)cc32)CC1] | 1-[1-[2-amino-4- (trifluoromethoxy)benzoyl]-4- piperidyl]-6-[tetrahydrofuran-3- yl]oxy-3H-imidazo[4,5-b]pyridin-2- one, Isomer 1 |
| | Isomer 1 | |
| 44 | 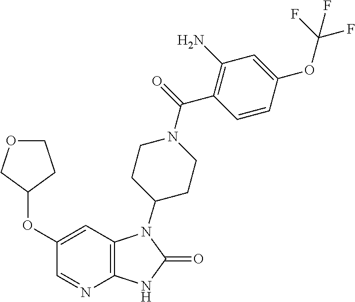 [SMILES: Nc1cc(OC(F)(F)F)ccc1C(=O)N1CCC(n2c(=O)[nH]c3ncc(OC4CCOC4)cc32)CC1] | 1-[1-[2-amino-4- (trifluoromethoxy)benzoyl]-4- piperidyl]-6-[tetrahydrofuran-3- yl]oxy-3H-imidazo[4,5-b]pyridin-2- one, Isomer 2 |
| | Isomer 2 | |
| 45 | 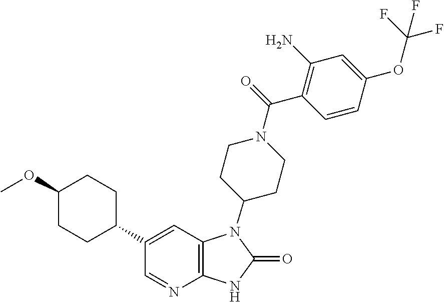 [SMILES: CO[C@H]1CC[C@H](c2cnc3[nH]c(=O)n(C4CCN(C(=O)c5ccc(OC(F)(F)F)cc5N)CC4)c3c2)CC1] | trans-1-[1-[2-amino-4- (trifluoromethoxy)benzoyl]-4- piperidyl]-6-(4-methoxycyclohexyl)- 3H-imidazo[4,5-b]pyridin-2-one |
| 46 | 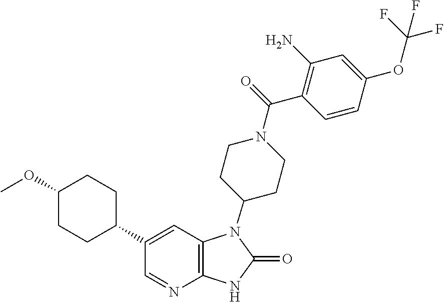 [SMILES: CO[C@H]1CC[C@@H](c2cnc3[nH]c(=O)n(C4CCN(C(=O)c5ccc(OC(F)(F)F)cc5N)CC4)c3c2)CC1] | cis-1-[1-[2-amino-4- (trifluoromethoxy)benzoyl]-4- piperidyl]-6-(4-methoxycyclohexyl)- 3H-imidazo[4,5-b]pyridin-2-one |
| 47 | 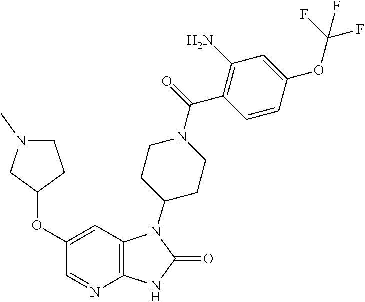 [SMILES: CN1CCC(Oc2cnc3[nH]c(=O)n(C4CCN(C(=O)c5ccc(OC(F)(F)F)cc5N)CC4)c3c2)C1] | 1-[1-[2-amino-4- (trifluoromethoxy)benzoyl]-4- piperidyl]-6-[1-methylpyrrolidin-3- yl]oxy-3H-imidazo[4,5-b]pyridin-2- one, Isomer 1 |
| | Isomer 1 | |
| 48 | 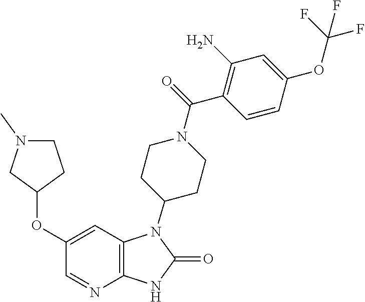 [SMILES: CN1CCC(Oc2cnc3[nH]c(=O)n(C4CCN(C(=O)c5ccc(OC(F)(F)F)cc5N)CC4)c3c2)C1] | 1-[1-[2-amino-4- (trifluoromethoxy)benzoyl]-4- piperidyl]-6-[1-methylpyrrolidin-3- yl]oxy-3H-imidazo[4,5-b]pyridin-2- one, Isomer 2 |
| | Isomer 2 | |
| 49 | 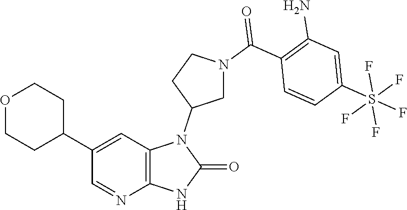 [SMILES: Nc1cc(S(F)(F)(F)(F)F)ccc1C(=O)N1CCC(n2c(=O)[nH]c3ncc(C4CCOCC4)cc32)C1] | (rac)-1-[1-[2-amino-4-(pentafluoro-λ⁶- sulfanyl)benzoyl]pyrrolidin-3-yl]-6- tetrahydropyran-4-yl-3H-imidazo[4,5- b]pyridin-2-one |
| 50 | 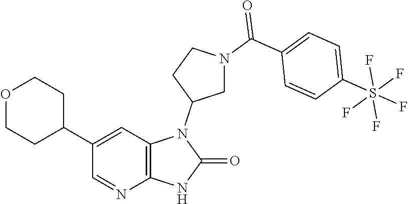 [SMILES: O=C(c1ccc(S(F)(F)(F)(F)F)cc1)N1CCC(n2c(=O)[nH]c3ncc(C4CCOCC4)cc32)C1] | (rac)-1-[1-[4-(pentafluoro-λ⁶- sulfanyl)benzoyl]pyrrolidin-3-yl]-6- tetrahydropyran-4-y1-3H-imidazo[4,5- b]pyridin-2-one |
| 51 | 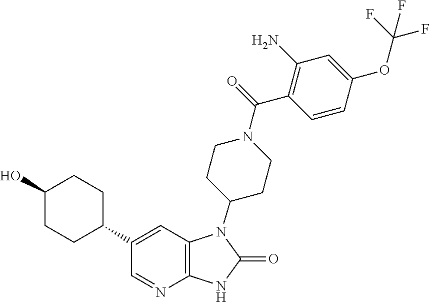 [SMILES: Nc1cc(OC(F)(F)F)ccc1C(=O)N1CCC(n2c(=O)[nH]c3ncc([C@H]4CC[C@H](O)CC4)cc32)CC1] | trans-1-[1-[2-amino-4- (trifluoromethoxy)benzoyl]-4- piperidyl]-6-(4-hydroxycyclohexyl)- 3H-imidazo[4,5-b]pyridin-2-one |
| 52 | 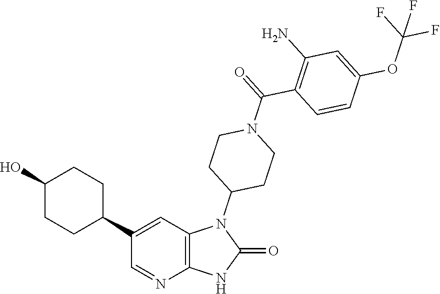 [SMILES: Nc1cc(OC(F)(F)F)ccc1C(=O)N1CCC(n2c(=O)[nH]c3ncc([C@H]4CC[C@@H](O)CC4)cc32)CC1] | cis-1-[1-[2-amino-4- (trifluoromethoxy)benzoyl]-4- piperidyl]-6-(4-hydroxycyclohexyl)- 3H-imidazo[4,5-b]pyridin-2-one |
| 53 | 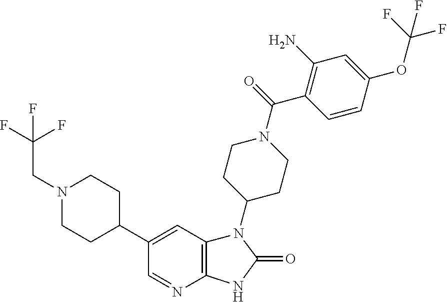 [SMILES: Nc1cc(OC(F)(F)F)ccc1C(=O)N1CCC(n2c(=O)[nH]c3ncc(C4CCN(CC(F)(F)F)CC4)cc32)CC1] | 1-[1-[2-amino-4- (trifluoromethoxy)benzoyl]-4- piperidyl]-6-[1-(2,2,2-trifluoroethyl)- 4-piperidyl]-3H-imidazo[4,5- b]pyridin-2-one |
| 54 | 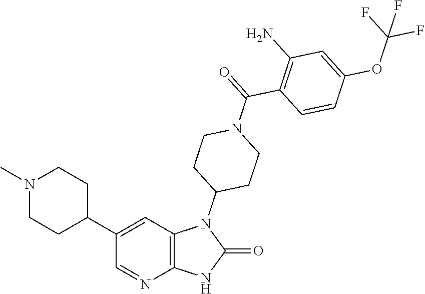 [SMILES: CN1CCC(c2cnc3[nH]c(=O)n(C4CCN(C(=O)c5ccc(OC(F)(F)F)cc5N)CC4)c3c2)CC1] | 1-[1-[2-amino-4- (trifluoromethoxy)benzoyl]-4- piperidyl]-6-(1-methyl-4-piperidyl)- 3H-imidazo[4,5-b]pyridin-2-one |
| 55 | 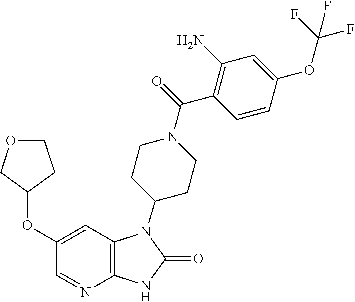 [SMILES: Nc1cc(OC(F)(F)F)ccc1C(=O)N1CCC(n2c(=O)[nH]c3ncc(OC4CCOC4)cc32)CC1] | (rac)-1-[1-[2-amino-4- (trifluoromethoxy)benzoyl]-4- piperidyl]-6-tetrahydrofuran-3-yloxy- 3H-imidazo[4,5-b]pyridin-2-one |
| 56 | 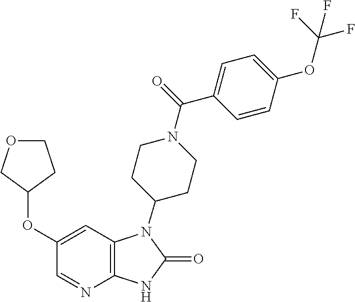 [SMILES: O=C(c1ccc(OC(F)(F)F)cc1)N1CCC(n2c(=O)[nH]c3ncc(OC4CCOC4)cc32)CC1] | (rac)-6-tetrahydrofuran-3-yloxy-1-[1- [4-(trifluoromethoxy)benzoyl]-4- piperidyl]-3H-imidazo[4,5-b]pyridin- 2-one |
| 57 | 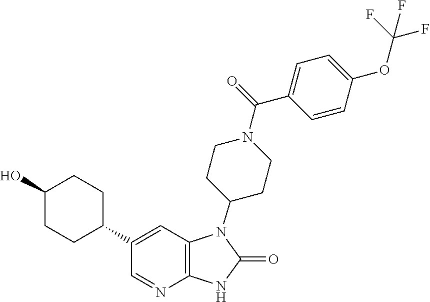 [SMILES: O=C(c1ccc(OC(F)(F)F)cc1)N1CCC(n2c(=O)[nH]c3ncc([C@H]4CC[C@H](O)CC4)cc32)CC1] | trans-6-(4-hydroxycyclohexyl)-1-[1- [4-(trifluoromethoxy)benzoyl]-4- piperidyl]-3H-imidazo[4,5-b]pyridin- 2-one |
| 58 | 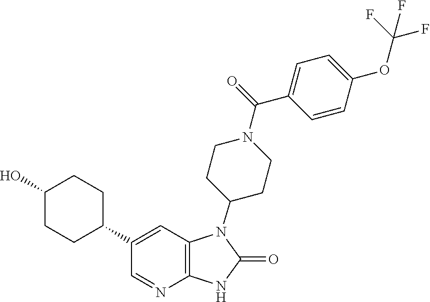 [SMILES: O=C(c1ccc(OC(F)(F)F)cc1)N1CCC(n2c(=O)[nH]c3ncc([C@H]4CC[C@@H](O)CC4)cc32)CC1] | cis-6-(4-hydroxycyclohexyl)-1-[1-[4- (trifluoromethoxy)benzoyl]-4- piperidyl]-3H-imidazo[4,5-b]pyridin- 2-one |
| 59 | 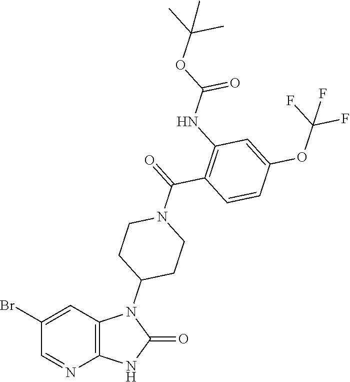 [SMILES: CC(C)(C)OC(=O)Nc1cc(OC(F)(F)F)ccc1C(=O)N1CCC(n2c(=O)[nH]c3ncc(Br)cc32)CC1] | tert-butyl N-[2-[4-(6-bromo-2-oxo-3H- imidazo[4,5-b]pyridin-1-yl)piperidine- 1-carbonyl]-5- (trifluoromethoxy)phenyl]carbamate |
| 60 | 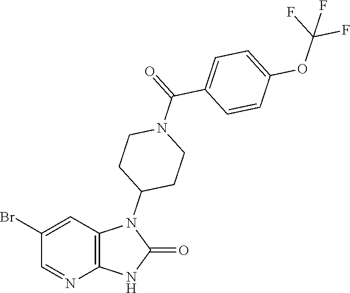 [SMILES: O=C(c1ccc(OC(F)(F)F)cc1)N1CCC(n2c(=O)[nH]c3ncc(Br)cc32)CC1] | 6-bromo-1-[1-[4- (trifluoromethoxy)benzoyl]-4- piperidyl]-3H-imidazo[4,5-b]pyridin- 2-one |
| 61 | 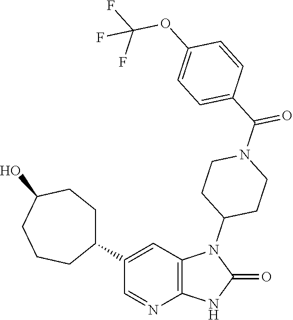 [SMILES: O=C(c1ccc(OC(F)(F)F)cc1)N1CCC(n2c(=O)[nH]c3ncc([C@@H]4CCC[C@@H](O)CC4)cc32)CC1] | (rac)-trans-6-(4-hydroxycycloheptyl)- 1-[1-[4-(trifluoromethoxy)benzoyl]-4- piperidyl]-3H-imidazo[4,5-b]pyridin- 2-one |
| 62 | 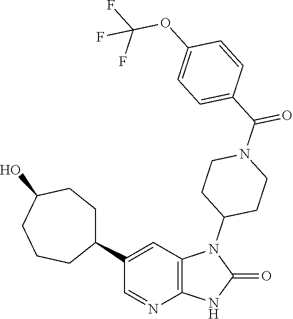 [SMILES: O=C(c1ccc(OC(F)(F)F)cc1)N1CCC(n2c(=O)[nH]c3ncc([C@H]4CCC[C@@H](O)CC4)cc32)CC1] | (rac)-cis-6-(4-hydroxycycloheptyl)-1- [1-[4-(trifluoromethoxy)benzoyl]-4- piperidyl]-3H-imidazo[4,5-b]pyridin- 2-one |
| 63 | 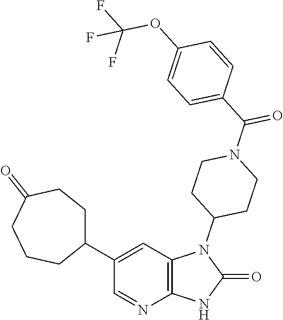 [SMILES: O=C1CCCC(c2cnc3[nH]c(=O)n(C4CCN(C(=O)c5ccc(OC(F)(F)F)cc5)CC4)c3c2)CC1] | (rac)-6-(4-oxocycloheptyl)-1-[1-[4- (trifluoromethoxy)benzoyl]-4- piperidyl]-3H-imidazo[4,5-b]pyridin- 2-one |
| 64 | 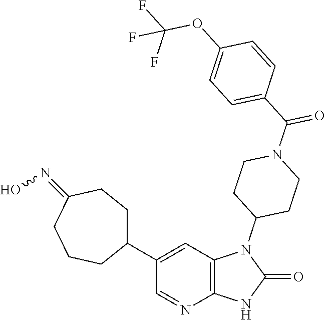 [SMILES: O=C(c1ccc(OC(F)(F)F)cc1)N1CCC(n2c(=O)[nH]c3ncc(C4CCCC(=NO)CC4)cc32)CC1] | (rac)-6-[(4E/Z)-4- hydroxyiminocycloheptyl]-1-[1-[4- (trifluoromethoxy)benzoyl]-4- piperidyl]-3H-imidazo[4,5-b]pyridin- 2-one, Isomer 1 |
| | Isomer 1 | |
| 65 | 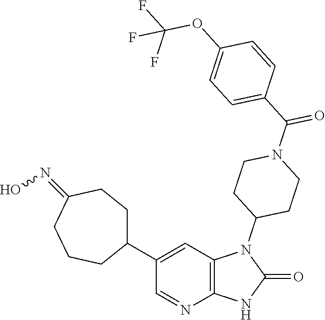 [SMILES: O=C(c1ccc(OC(F)(F)F)cc1)N1CCC(n2c(=O)[nH]c3ncc(C4CCCC(=NO)CC4)cc32)CC1] | (rac)-6-[(4E/Z)-4- hydroxyiminocycloheptyl]-1-[1-[4- (trifluoromethoxy)benzoyl]-4- piperidyl]-3H-imidazo[4,5-b]pyridin- 2-one, Isomer 2 |
| | Isomer 2 | |
| 66 | 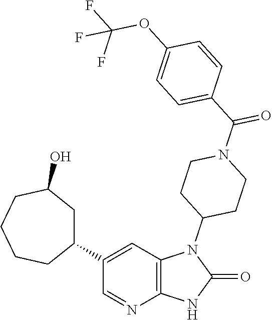 [SMILES: O=C(c1ccc(OC(F)(F)F)cc1)N1CCC(n2c(=O)[nH]c3ncc([C@@H]4CCCC[C@@H](O)C4)cc32)CC1] | (rac)-trans-6-(3-hydroxycycloheptyl)- 1-[1-[4-(trifluoromethoxy)benzoyl]-4- piperidyl]-3H-imidazo[4,5-b]pyridin- 2-one |
| 67 | 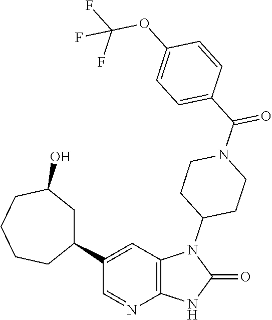 [SMILES: O=C(c1ccc(OC(F)(F)F)cc1)N1CCC(n2c(=O)[nH]c3ncc([C@H]4CCCC[C@@H](O)C4)cc32)CC1] | (rac)-cis-6-(3-hydroxycycloheptyl)-1- [1-[4-(trifluoromethoxy)benzoyl]-4- piperidyl]-3H-imidazo[4,5-b]pyridin- 2-one |

### Example 1: 1-[1-[2-Amino-4-(trifluoromethoxy)benzoyl]-4-piperidyl]-6-tetrahydropyran-4-yloxy-3H-imidazo[4,5-b]pyridin-2-one

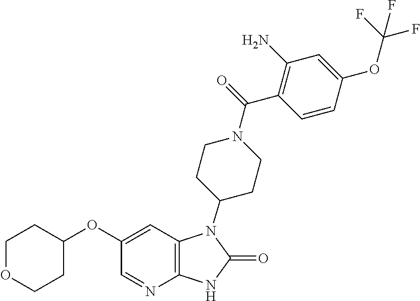 [SMILES: Nc1cc(OC(F)(F)F)ccc1C(=O)N1CCC(n2c(=O)[nH]c3ncc(OC4CCOCC4)cc32)CC1]

### Step 1: tert-Butyl 4-[(5-fluoro-2-nitro-3-pyridyl)amino]piperidine-1-carboxylate

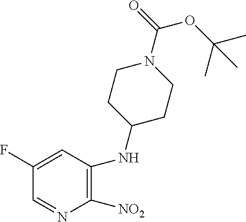 [SMILES: CC(C)(C)OC(=O)N1CCC(Nc2cc(F)cnc2[N+](=O)[O-])CC1]

To a solution of 3,5-difluoro-2-nitro-pyridine (1300 mg, 8.12 mmol) in DMF (20 mL) at 0° C. was dropwise added a solution of tert-butyl 4-aminopiperidine-1-carboxylate (1845 mg, 8.93 mmol) in DMF (30 mL). The reaction mixture was warmed up to room temperature and stirred overnight. The reaction mixture was then diluted with water (50 mL), extracted with ethyl acetate (2×100 mL). The organic layers were combined, dried over Na₂SO₄, filtered, and concentrated in vacuo. The resulting residue was purified by flash chromatography on silica gel (SiO₂ 120 g) eluting with DCM/MeOH/NH₄OH (aq. 30%) (99/1/0.1), to give 2.355 g (85% yield) of tert-butyl 4-[(5-fluoro-2-nitro-3-pyridyl)amino]piperidine-1-carboxylate as a yellow solid. LC/MS (m/z, M+): calc. 340.2, found 340.0.

### Step 2: tert-Butyl 4-[(2-nitro-5-tetrahydropyran-4-yloxy-3-pyridyl)amino]piperidine-1-carboxylate

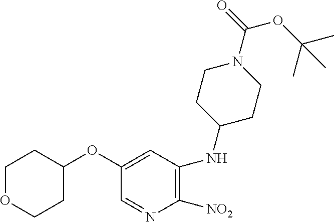 [SMILES: CC(C)(C)OC(=O)N1CCC(Nc2cc(OC3CCOCC3)cnc2[N+](=O)[O-])CC1]

Under argon atmosphere, to a solution of tetrahydropyran-4-ol (210 mg, 2.06 mmol) in DMF (3 mL) at 0° C. was added sodium hydride (60% dispersion in oil, 86 mg, 2.16 mmol). The reaction mixture was stirred for 20 minutes and a solution of tert-butyl 4-[(5-fluoro-2-nitro-3-pyridyl)amino]piperidine-1-carboxylate (350 mg, 1.03 mmol) in DMF (3 mL) was added. The reaction mixture was then warmed up to room temperature and stirred for 1.5 hour. The reaction mixture was diluted with water (50 mL) and extracted with ethyl acetate (2×100 mL). The organic layers were combined, washed with brine, dried over Na₂SO₄, filtered, and concentrated in vacuo to give 434 mg (100% yield) of crude tert-butyl 4-[(2-nitro-5-tetrahydropyran-4-yloxy-3-pyridyl)amino]piperidine-1-carboxylate as a yellow solid which was engaged in the next step without further purification. LC/MS (m/z, M+): calc. 422.2, found 422.0.

### Step 3: tert-Butyl 4-[(2-amino-5-tetrahydropyran-4-yloxy-3-pyridyl)amino]piperidine-1-carboxylate

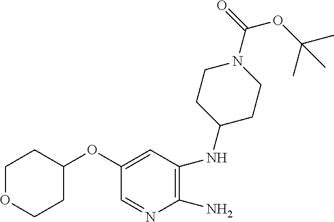 [SMILES: CC(C)(C)OC(=O)N1CCC(Nc2cc(OC3CCOCC3)cnc2N)CC1]

To a solution of tert-butyl 4-[(2-nitro-5-tetrahydropyran-4-yloxy-3-pyridyl)amino]piperidine-1-carboxylate (434 mg, 1.03 mmol) in MeOH (10 mL) was added ammonium formate (1297 mg, 20.57 mmol) and Pd/C 10% (100 mg). The reaction mixture was then stirred at 65° C. for 1 hour, cooled down to room temperature, filtered through celite and washed with MeOH. The filtrate was concentrated in vacuo. The resulting residue was diluted with ethyl acetate (50 mL), washed with 1N NaOH, dried over Na₂SO₄, filtered and concentrated in vacuo. The resulting residue was purified by flash chromatography on silica gel (SiO₂ 24 g) eluting with DCM/MeOH/aq. 30% NH₄OH (95/5/0.5) to give 294 mg (69% yield) of tert-butyl 4-[(2-amino-5-tetrahydropyran-4-yloxy-3-pyridyl)amino]piperidine-1-carboxylate as a brown foam. LC/MS (m/z, M+H): calc. 393.2, found 393.2.

### Step 4: tert-Butyl 4-(2-oxo-6-tetrahydropyran-4-yloxy-3H-imidazo[4,5-b]pyridin-1-yl)piperidine-1-carboxylate

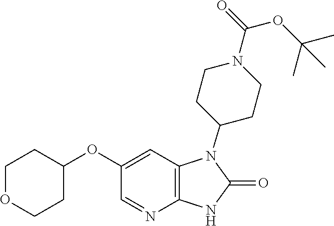 [SMILES: CC(C)(C)OC(=O)N1CCC(n2c(=O)[nH]c3ncc(OC4CCOCC4)cc32)CC1]

To a solution of tert-butyl 4-[(2-amino-5-tetrahydropyran-4-yloxy-3-pyridyl)amino]piperidine-1-carboxylate (290 mg, 0.71 mmol) in THF (8 mL) wad added CDI (345 mg, 2.13 mmol) and the resulting reaction mixture was refluxed for 1.5 hour, then cooled down to room temperature and concentrated. The resulting residue was diluted with a saturated aqueous solution of NaHCO₃ (100 mL) and extracted twice with ethyl acetate (2×50 mL). The organic layers were combined, dried over Na₂SO₄, filtered and concentrated in vacuo. The resulting residue was purified by flash chromatography on silica gel (SiO₂ 12 g) eluting with DCM/MeOH/aq. 30% NH₄OH (95/5/0.5) to give 267 mg (85% yield) of tert-butyl 4-(2-oxo-6-tetrahydropyran-4-yloxy-3H-imidazo[4,5-b]pyridin-1-yl)piperidine-1-carboxylate as a green foam. LC/MS (m/z, M+H): calc. 419.2, found 419.2.

### Step 5: 1-(4-Piperidyl)-6-tetrahydropyran-4-yloxy-3H-imidazo[4,5-b]pyridin-2-one, hydrochloride

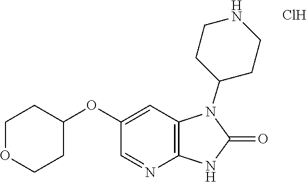 [SMILES: Cl ; O=c1[nH]c2ncc(OC3CCOCC3)cc2n1C1CCNCC1]

To a solution of tert-butyl 4-(2-oxo-6-tetrahydropyran-4-yloxy-3H-imidazo[4,5-b]pyridin-1-yl)piperidine-1-carboxylate (264 mg, 0.6 mmol) in MeOH (4 mL) was added 2.25 mL of a 4N solution of HCl in dioxane (8.99 mmol). The reaction mixture was stirred at 40° C. for one hour, then concentrated in vacuo to give 234 mg (100% yield) of crude 1-(4-piperidyl)-6-tetrahydropyran-4-yloxy-3H-imidazo[4,5-b]pyridin-2-one, hydrochloride as a yellow foam, which was used in the next step without further purification. LC/MS (m/z, M+H): calc. 319.2, found 319.2 (free base).

### Step 6: 1-[1-[2-Amino-4-(trifluoromethoxy)benzoyl]-4-piperidyl]-6-tetrahydropyran-4-yloxy-3H-imidazo[4,5-b]pyridin-2-one

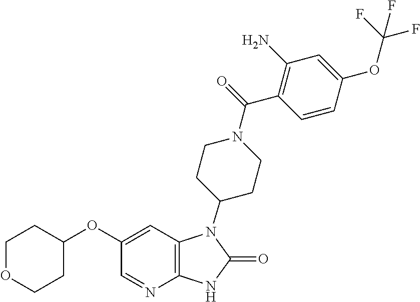 [SMILES: Nc1cc(OC(F)(F)F)ccc1C(=O)N1CCC(n2c(=O)[nH]c3ncc(OC4CCOCC4)cc32)CC1]

To a solution of 1-(4-piperidyl)-6-tetrahydropyran-4-yloxy-3H-imidazo[4,5-b]pyridin-2-one, hydrochloride (234 mg, 0.6 mmol) in DMF (4 mL) was added DIPEA (387 mg, 0.52 mL, 3 mmol) and the resulting mixture was stirred for 5 minutes. Then, 2-amino-4-(trifluoromethoxy)benzoic acid (132 mg, 0.6 mmol) followed by HATU (251 mg, 0.66 mmol) were added, and the resulting solution was stirred at room temperature for one hour, then, diluted with ethyl acetate (50 mL), washed with a 1N solution of NaOH, water and brine. The organic layer was then dried over Na₂SO₄, filtered, concentrated in vacuo. The resulting residue was purified by flash chromatography on Puriflash® interchim PF-15SiHP-F0012 (12 g) column eluting with DCM/MeOH/aq. 30% NH₄OH (96/4/0.4) to give 188 mg (57% yield) of 1-[1-[2-amino-4-(trifluoromethoxy)benzoyl]-4-piperidyl]-6-tetrahydropyran-4-yloxy-3H-imidazo[4,5-b]pyridin-2-one as a white solid. LC/MS (m/z, M+H): calc. 522.2, found 522.3; ¹H NMR (400 MHz, DMSO-d6, 120° C.) δ ppm 1.64 (dtd, J=13, 9, 9, 4 Hz, 2H), 1.74-1.86 (m, 2H), 1.90-2.02 (m, 2H), 2.28 (qd, J=13, 4 Hz, 2H), 3.05 (td, J=13, 3 Hz, 2H), 3.48 (ddd, J=12, 9, 3 Hz, 2H), 3.81-3.98 (m, 2H), 4.17 (br d, J=13 Hz, 2H), 4.33-4.52 (m, 2H), 5.31 (s, 2H), 6.44-6.55 (m, 1H), 6.66-6.73 (m, 1H), 7.13 (d, J=8 Hz, 1H), 7.22 (d, J=2 Hz, 1H), 7.63 (d, J=2 Hz, 1H), 10.53-11.18 (m, 1H)

### Example 2: [1-[1-[2-Amino-4-(trifluoromethoxy)benzoyl]-4-piperidyl]-6-tetrahydropyran-4-yl-3H-imidazo[4,5-b]pyridin-2-one

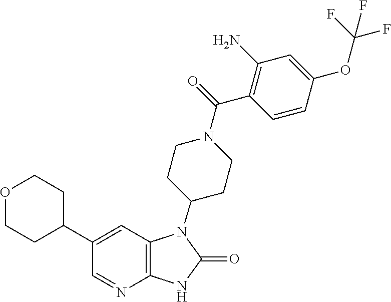 [SMILES: Nc1cc(OC(F)(F)F)ccc1C(=O)N1CCC(n2c(=O)[nH]c3ncc(C4CCOCC4)cc32)CC1]

### Step 1: tert-Butyl 4-[(5-bromo-2-nitro-3-pyridyl)amino]piperidine-1-carboxylate

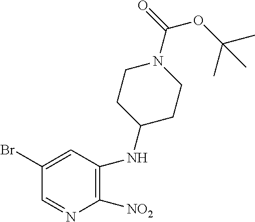 [SMILES: CC(C)(C)OC(=O)N1CCC(Nc2cc(Br)cnc2[N+](=O)[O-])CC1]

To a solution of 5-bromo-3-fluoro-2-nitropyridine (500 mg, 2.26 mmol) in ethanol (15 mL) was added triethylamine (950 μL, 6.82 mmol) followed by 4-amino-1-Boc-piperidine (545 mg, 2.72 mmol). The resulting reaction mixture was then stirred at 90° C. for 75 minutes, then cooled down to room temperature and concentrated in vacuo. The resulting residue was then diluted in 50 mL of ethyl acetate, washed with an aqueous saturated solution of NH₄Cl. The aqueous layer was then extracted twice with ethyl acetate (2×25 mL). The organic layers were combined, dried over Na₂SO₄, filtered, and concentrated in vacuo to give 908 mg (100% yield) of tert-butyl 4-[(5-bromo-2-nitro-3-pyridyl)amino]piperidine-1-carboxylate as a yellow solid. LC/MS (m/z, M+H): calc. 401.1, found 401.3.

### Step 2: tert-Butyl 4-[[5-(3,6-dihydro-2H-pyran-4-yl)-2-nitro-3-pyridyl]amino]piperidine-1-carboxylate

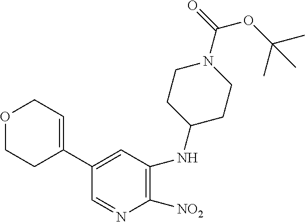 [SMILES: CC(C)(C)OC(=O)N1CCC(Nc2cc(C3=CCOCC3)cnc2[N+](=O)[O-])CC1]

A solution of tert-butyl 4-[(5-bromo-2-nitro-3-pyridyl)amino]piperidine-1-carboxylate (500 mg, 1.25 mmol) in THF (8 mL) and water (2 mL) was bubbled with argon for 5 minutes, and then potassium phosphate tribasic (650 mg, 3.06 mmol), XPhos (30 mg, 0.063 mmol), 3,6-dihydro-2H-pyran-4-boronic acid pinacol ester (315 mg, 1.5 mmol) and XPhosPdG4 (30 mg, 0.035 mmol) were added. The resulting mixture was stirred at 80° C. for one hour, then cooled down to room temperature, and diluted with ethyl acetate (50 mL) and water (50 mL). The organic layer was discarded, and the aqueous layer was extracted twice with ethyl acetate (2×50 mL). The combined organic layers were then dried over Na₂SO₄, filtered and concentrated in vacuo. The resulting residue was purified by flash chromatography on silica gel (SiO₂ 40 g) eluting with cyclohexane/AcOEt (6/4) to give 453 mg (90% yield) of tert-butyl 4-[[5-(3,6-dihydro-2H-pyran-4-yl)-2-nitro-3-pyridyl]amino]piperidine-1-carboxylate as a yellow solid. LC/MS (m/z, M+H): calc. 405.2, found 405.4.

### Step 3: tert-Butyl 4-[(2-amino-5-tetrahydropyran-4-yl-3-pyridyl)amino]piperidine-1-carboxylate

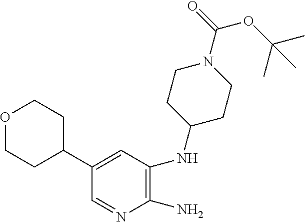 [SMILES: CC(C)(C)OC(=O)N1CCC(Nc2cc(C3CCOCC3)cnc2N)CC1]

A solution of tert-butyl 4-[[5-(3,6-dihydro-2H-pyran-4-yl)-2-nitro-3-pyridyl]amino]piperidine-1-carboxylate (450 mg, 1.11 mmol) in ethanol (6 mL) and ethyl acetate (6 mL) was bubbled with argon for 5 minutes and then Pd/C 10% (50% wet) (250 mg, 2.35 mmol) was added. The resulting reaction mixture was then submitted to hydrogenation at 50° C. under 4 bars of hydrogen for 1.5 hours. The resulting mixture was cooled down to room temperature, filtered through a GF/F filter and washed with EtOH/AcOEt. The resulting filtrate was concentrated in vacuo to give 405 mg (97% yield) of tert-butyl 4-[(2-amino-5-tetrahydropyran-4-yl-3-pyridyl)amino]piperidine-1-carboxylate as a red solid. LC/MS (m/z, M+H): calc. 377.2, found 377.4.

### Step 4: tert-Butyl 4-(2-oxo-6-tetrahydropyran-4-yl-3H-imidazo[4,5-b]pyridin-1-yl)piperidine-1-carboxylate

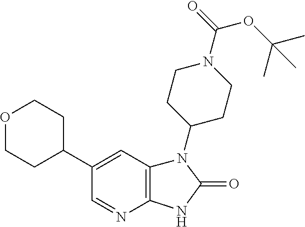 [SMILES: CC(C)(C)OC(=O)N1CCC(n2c(=O)[nH]c3ncc(C4CCOCC4)cc32)CC1]

To a solution of tert-butyl 4-[(2-amino-5-tetrahydropyran-4-yl-3-pyridyl)amino]piperidine-1-carboxylate (400 mg, 1.06 mmol) in acetonitrile (5 mL) was added CDI (350 mg, 2.16 mmol) and the resulting mixture was stirred at 80° C. for 2 hours, then cooled down to room temperature, and the resulting precipitate was filtered to give 260 mg (61% yield) of tert-butyl 4-(2-oxo-6-tetrahydropyran-4-yl-3H-imidazo[4,5-b]pyridin-1-yl)piperidine-1-carboxylate as a grey powder. LC/MS (m/z, M+H): calc. 403.2, found 403.0.

### Step 5: 1-(4-Piperidyl)-6-tetrahydropyran-4-yl-3H-imidazo[4,5-b]pyridin-2-one, dihydrochloride

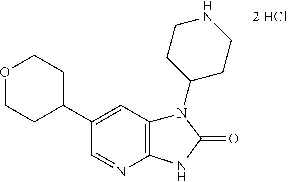 [SMILES: O=c1[nH]c2ncc(C3CCOCC3)cc2n1C1CCNCC1]

To a solution of 4-(2-oxo-6-tetrahydropyran-4-yl-3H-imidazo[4,5-b]pyridin-1-yl)piperidine-1-carboxylate (255 mg, 0.63 mmol) in methanol (6 mL) was added 1.8 mL of a 4N solution of HCl in dioxane (7.2 mmol) and the resulting mixture was stirred overnight at room temperature. The resulting reaction mixture was then concentrated in vacuo, and the resulting residue was triturated in diethyl ether. The solid obtained was filtered to give 245 mg (65% yield) of 1-(4-piperidyl)-6-tetrahydropyran-4-yl-3H-imidazo[4,5-b]pyridin-2-one, dihydrochloride as a grey powder. LC/MS (m/z, M+H): calc. 303.2, found 303.3 (free base).

### Step 6: [1-[1-[2-Amino-4-(trifluoromethoxy)benzoyl]-4-piperidyl]-6-tetrahydropyran-4-yl-3H-imidazo[4,5-b]pyridin-2-one

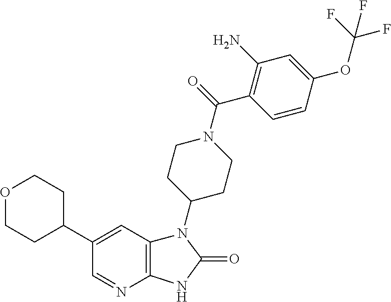 [SMILES: Nc1cc(OC(F)(F)F)ccc1C(=O)N1CCC(n2c(=O)[nH]c3ncc(C4CCOCC4)cc32)CC1]

To a solution of 1-(4-piperidyl)-6-tetrahydropyran-4-yl-3H-imidazo[4,5-b]pyridin-2-one, hydrochloride (150 mg, 0.4 mmol) in DMF (3 mL) was added N,N-diisopropylethylamine (350 μL, 2.01 mmol) followed by 2-amino-4-(trifluoromethoxy)benzoic acid (110 mg, 0.5 mmol) and HATU (190 mg, 0.5 mmol). The resulting reaction mixture was stirred at room temperature for 75 minutes then concentrated in vacuo. The resulting residue was diluted with ethyl acetate (20 mL) and water (20 mL). The mixture was basified to pH 11-12 with a TN aqueous solution of NaOH under stirring, and then the organic layer was discarded. The aqueous layer was extracted with ethyl acetate (20 mL). The combined organic layers were dried over Na₂SO₄, filtered, and concentrated in vacuo. The resulting residue was purified by flash chromatography on silica gel (SiO₂ 12 g) eluting with DCM/MeOH 95/5 to 0/100 to give an orange wax which was triturated in acetonitrile. The resulting white precipitate was filtered off to give 53 mg (26% yield) of [1-[1-[2-amino-4-(trifluoromethoxy)benzoyl]-4-piperidyl]-6-tetrahydropyran-4-yl-3H-imidazo[4,5-b]pyridin-2-one as a white solid. LC/MS (m/z, M+H): calc. 506.5, found 506.2; ¹H NMR (400 MHz, DMSO-d6, 100° C.) δ ppm 1.65-1.88 (m, 6H), 2.30 (qd, J=13, 4 Hz, 2H), 2.82 (tt, J=10, 5 Hz, 1H), 3.06 (td, J=13, 3 Hz, 2H), 3.40-3.56 (m, 2H), 3.91-4.03 (m, 2H), 4.17 (br d, J=13 Hz, 2H), 4.43 (tt, J=12, 4 Hz, 1H), 5.41 (s, 2H), 6.39-6.55 (m, 1H), 6.62-6.74 (m, 1H), 7.14 (d, J=8 Hz, 1H), 7.41 (d, J=2 Hz, 1H), 7.78 (d, J=2 Hz, 1H), 11.03 (br s, 1H)

### Example 3: 1-[1-[2-Amino-4-(trifluoromethoxy)benzoyl]-4-piperidyl]-6-morpholino-3H-imidazo[4,5-b]pyridin-2-one

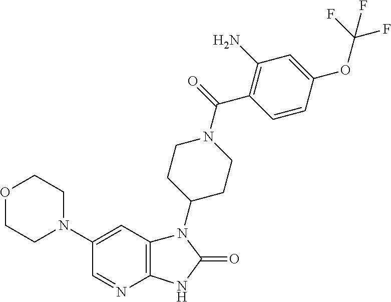 [SMILES: Nc1cc(OC(F)(F)F)ccc1C(=O)N1CCC(n2c(=O)[nH]c3ncc(N4CCOCC4)cc32)CC1]

### Step 1: tert-Butyl 4-[(2-amino-5-bromo-3-pyridyl)amino]piperidine-1-carboxylate

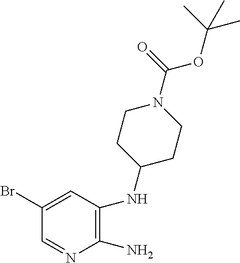 [SMILES: CC(C)(C)OC(=O)N1CCC(Nc2cc(Br)cnc2N)CC1]

In a sealed tube, to a solution of tert-butyl 4-[(5-bromo-2-nitro-3-pyridyl)amino]piperidine-1-carboxylate (7.61 g, 19 mmol) in i-PrOH (75 mL) was added bis(pinacolato)diboron (14.9 g, 58.8 mmol) followed by t-BuOK (2.55 g, 22.8 mmol). The tube was sealed and heated under stirring at 110° C. for 2.5 hours. Then, the reaction mixture was cooled down to room temperature and concentrated in vacuo. The resulting residue was then diluted with ethyl acetate and with an aqueous saturated solution of NaCl. The organic layer was discarded, dried over Na₂SO₄, filtered and concentrated under reduced pressure. The resulting residue was diluted in ethyl acetate, washed twice with a 2N aqueous solution of NaOH. The organic layer was discarded, dried over Na₂SO₄, filtered and concentrated under reduced pressure. The resulting residue was purified by flash chromatography on silica gel (SiO₂ 220 g) eluting with heptane/(AcOEt/EtOH 3/1) 7/3 to give 5.47 g (78% yield) of tert-butyl 4-[(2-amino-5-bromo-3-pyridyl)amino]piperidine-1-carboxylate as a pale brown solid. LC/MS (m/z, M+): calc. 370.1, found 370.0.

### Step 2: tert-Butyl 4-(6-bromo-2-oxo-3H-imidazo[4,5-b]pyridin-1-yl)piperidine-1-carboxylate

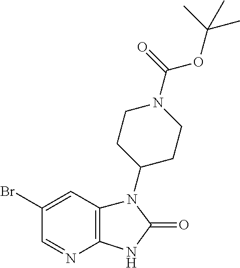 [SMILES: CC(C)(C)OC(=O)N1CCC(n2c(=O)[nH]c3ncc(Br)cc32)CC1]

To a solution of tert-butyl 4-[(2-amino-5-bromo-3-pyridyl)amino]piperidine-1-carboxylate (5370 mg, 14.5 mmol) in THF (145 mL) was added CDI (7040 mg, 43.4 mmol) and the resulting reaction mixture was refluxed for 2.5 hours. The reaction mixture was then cooled down to room temperature, concentrated in vacuo, and the resulting residue was diluted with an aqueous saturated solution of NaHCO₃ and extracted twice with ethyl acetate. The combined organic layers were combined, dried over Na₂SO₄, filtered and concentrated in vacuo. The resulting residue was purified by flash chromatography on silica gel (SiO₂ 120 g) eluting with DCM/MeOH 96/4 to give 5.34 g (93% yield) of tert-butyl 4-(6-bromo-2-oxo-3H-imidazo[4,5-b]pyridin-1-yl)piperidine-1-carboxylate as a pale brown solid. LC/MS (m/z, M+): calc. 396.1, found 396.0.

### Step 3: Tert-butyl 4-(6-morpholino-2-oxo-3H-imidazo[4,5-b]pyridin-1-yl)piperidine-1-carboxylate

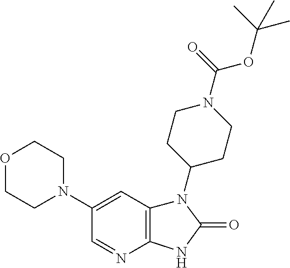 [SMILES: CC(C)(C)OC(=O)N1CCC(n2c(=O)[nH]c3ncc(N4CCOCC4)cc32)CC1]

In a sealed tube, a solution of tert-butyl 4-(6-bromo-2-oxo-3H-imidazo[4,5-b]pyridin-1-yl)piperidine-1-carboxylate (266 mg, 0.67 mmol), morpholine (70 mg, 0.80 mmol) and RuPhos (25 mg, 0.054 mmol) in THF (0.45 mL) was bubbled with argon for 5 minutes. Then, Pd(OAc)₂ (6 mg, 0.027 mmol) and LiHMDS (1.61 mL, 1.61 mmol, 1M solution in THF) were added, the tube was sealed, and the reaction mixture stirred at 75° C. overnight. The reaction mixture was then cooled down to room temperature, diluted with water, and extracted four times with ethyl acetate. The combined organic layers were washed with brine, dried over Na₂SO₄, filtered, and concentrated in vacuo. The resulting residue was purified by flash chromatography on silica gel (SiO₂ 12 g) eluting with DCM/MeOH/aq. 30% NH₄OH (94.5:5.5:0.55) to give 29 mg of a white solid which was then triturated in i-Pr₂O, and filtered to give 216 mg (80% yield) of tert-butyl 4-(6-morpholino-2-oxo-3H-imidazo[4,5-b]pyridin-1-yl)piperidine-1-carboxylate as a white solid. LC/MS (m/z, M+): calc. 403.2, found 403.0.

### Step 4: 6-Morpholino-1-(4-piperidyl)-3H-imidazo[4,5-b]pyridin-2-one, trihydrochloride

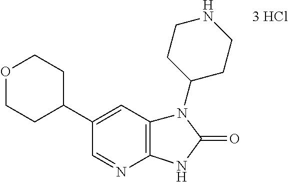 [SMILES: O=c1[nH]c2ncc(C3CCOCC3)cc2n1C1CCNCC1]

To a solution of tert-butyl 4-(6-morpholino-2-oxo-3H-imidazo[4,5-b]pyridin-1-yl)piperidine-1-carboxylate (212 mg, 0.52 mmol) in MeOH (30 mL), was added at 45° C. HCl (4 N solution in dioxane, 1.05 mL, 4.2 mmol), and the reaction mixture was stirred at 45° C. for 3 hours. Then, the reaction mixture was concentrated in vacuo. The resulting residue was triturated in ethyl acetate (20 mL) one night and filtered to give 196 mg (90% yield) of 6-morpholino-1-(4-piperidyl)-3H-imidazo[4,5-b]pyridin-2-one, trihydrochloride as a pale yellow solid. LC/MS (m/z, M+H): calc. 304.2, found 304.0 (free base).

### Step 5: 1-[1-[2-Amino-4-(trifluoromethoxy)benzoyl]-4-piperidyl]-6-morpholino-3H-imidazo[4,5-b]pyridin-2-one

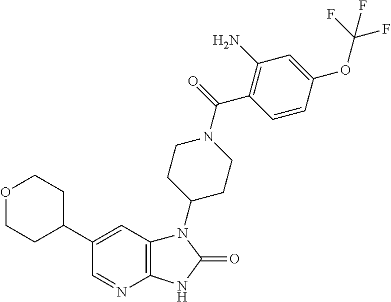 [SMILES: Nc1cc(OC(F)(F)F)ccc1C(=O)N1CCC(n2c(=O)[nH]c3ncc(C4CCOCC4)cc32)CC1]

To a solution of 6-morpholino-1-(4-piperidyl)-3H-imidazo[4,5-b]pyridin-2-one, trihydrochloride (95 mg, 0.23 mmol) in DMF (1.5 mL) was added N,N-diisopropylethylamine (281 μL, 1.61 mmol) followed by 2-amino-4-(trifluoromethoxy)benzoic acid (53 mg, 0.23 mmol) and HATU (96 mg, 0.25 mmol). The resulting reaction mixture was stirred at room temperature for 3 hours, then concentrated in vacuo. The resulting residue was diluted with ethyl acetate (20 mL) and water (20 mL). The mixture was basified to pH 11-12 with a 1N aqueous solution of NaOH under stirring, and then the organic layer was discarded. The aqueous layer was extracted with ethyl acetate (20 mL). The combined organic layers were dried over Na₂SO₄, filtered, and concentrated in vacuo. The resulting residue was purified by flash chromatography on Puriflash® interchim PF-15SiHP-F0012 (12 g) column eluting with AcOEt/MeOH/NH₄OH (94/6/0.6) to give a wax, which was triturated in acetonitrile. The resulting white precipitate was filtered off to give 78 mg (67% yield) of 1-[1-[2-amino-4-(trifluoromethoxy)benzoyl]-4-piperidyl]-6-morpholino-3H-imidazo[4,5-b]pyridin-2-one as a white solid. LC/MS (m/z, M+H): calc. 507.2, found 507.2; ¹H NMR (400 MHz, DMSO-d6, 30° C.) δ ppm 1.63-1.79 (m, 2H), 2.20-2.40 (m, 2H), 2.85-3.42 (m, 2H), 3.04-3.13 (m, 4H), 3.62-4.89 (m, 2H), 3.72-3.80 (m, 4H), 4.44 (ddd, J=12, 9, 4 Hz, 1H), 5.68 (s, 2H), 6.50 (dd, J=8, 1 Hz, 1H), 6.67 (br d, J=1 Hz, 1H), 7.13 (d, J=8 Hz, 1H), 7.32 (d, J=2 Hz, 1H), 7.57 (d, J=2 Hz, 1H), 11.21 (br s, 1H)

### Example 4: 6-Tetrahydropyran-4-yloxy-1-[1-[4-(trifluoromethoxy)benzoyl]-4-piperidyl]-3H-imidazo[4,5-b]pyridin-2-one

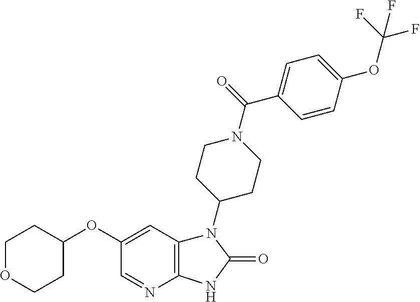 [SMILES: O=C(c1ccc(OC(F)(F)F)cc1)N1CCC(n2c(=O)[nH]c3ncc(OC4CCOCC4)cc32)CC1]

To a solution of 1-(4-piperidyl)-6-tetrahydropyran-4-yloxy-3H-imidazo[4,5-b]pyridin-2-one, dihydrochloride (60.0 mg, 0.15 mmol) in DMF (1 mL) was added DIPEA (99 mg, 0.13 mL, 0.767 mmol) and the reaction mixture was stirred at room temperature for 5 minutes. 4-(trifluoromethoxy)benzoic acid (32 mg, 0.15 mmol) and HATU (64 mg, 0.17 mmol) were added, and the resulting mixture was stirred at room temperature for 1.5 hours. The reaction mixture was diluted with ethyl acetate, washed with a 1N aqueous solution of NaOH and with brine. The organic layer was dried over Na₂SO₄, filtered, and concentrated under reduced pressure. The resulting residue was purified by flash chromatography on silica gel (SiO₂ 12 g) eluting with DCM/MeOH/30% aq. NH₄OH (95/5/0.5) to give 63 mg (81% yield) of 6-tetrahydropyran-4-yloxy-1-[1-[4-(trifluoromethoxy)benzoyl]-4-piperidyl]-3H-imidazo[4,5-b]pyridin-2-one as a pale brown solid. LC/MS (m/z, M+H): calc. 507.2, found 507.3; ¹H NMR (400 MHz, DMSO-d6, 101° C.) δ ppm 1.64 (dtd, J=13, 9, 9, 4 Hz, 2H), 1.80 (br d, J=11 Hz, 2H), 1.90-2.04 (m, 2H), 2.29 (qd, J=12, 4 Hz, 2H), 3.08 (br t, J=13 Hz, 2H), 3.47 (ddd, J=12, 9, 3 Hz, 2H), 3.81-3.93 (m, 2H), 4.07-4.28 (m, 2H), 4.33-4.53 (m, 2H), 7.27 (d, J=2 Hz, 1H), 7.39 (br d, J=8 Hz, 2H), 7.57 (d, J=8 Hz, 2H), 7.64 (d, J=2 Hz, 1H), 10.44-11.38 (m, 1H)

### Example 5: 6-[(1-Methyl-4-piperidyl)oxy]-1-[1-[4-(trifluoromethoxy)benzoyl]-4-piperidyl]-3H-imidazo[4,5-b]pyridin-2-one

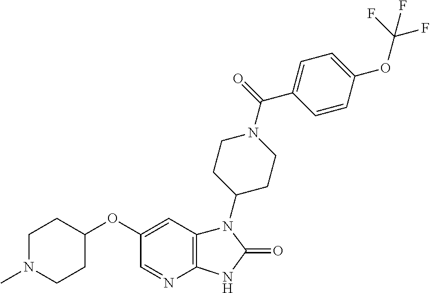 [SMILES: CN1CCC(Oc2cnc3[nH]c(=O)n(C4CCN(C(=O)c5ccc(OC(F)(F)F)cc5)CC4)c3c2)CC1]

### Step 1: tert-Butyl 4-[[5-[(1-methyl-4-piperidyl)oxy]-2-nitro-3-pyridyl]amino]piperidine-1-carboxylate

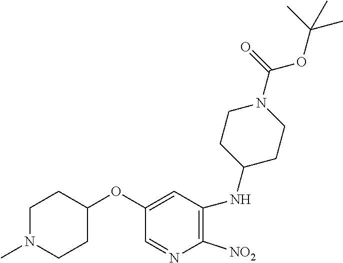 [SMILES: CN1CCC(Oc2cnc([N+](=O)[O-])c(NC3CCN(C(=O)OC(C)(C)C)CC3)c2)CC1]

Under argon atmosphere, to a solution of 1-methylpiperidin-4-ol (414 mg, 3.52 mmol) in DMF (5 mL) at 0° C. was added sodium hydride (60% dispersion in oil, 148 mg, 3.70 mmol) and the resulting reaction mixture was stirred for 20 minutes. Then, a solution of tert-butyl 4-[(5-fluoro-2-nitro-3-pyridyl)amino]piperidine-1-carboxylate (600 mg, 1.76 mmol) in DMF (4 mL) was added dropwise. The resulting mixture was warmed up to room temperature and stirred for 1.5 hours, then diluted with water and extracted with ethyl acetate. The organic layer was washed with brine, dried over Na₂SO₄, filtered, and concentrated under reduced pressure to give 768 mg (100% yield) of tert-butyl 4-[[5-[(1-methyl-4-piperidyl)oxy]-2-nitro-3-pyridyl]amino]piperidine-1-carboxylate as an orange gum which was used in the next step without further purification. LC/MS (m/z, M+): calc. 435.2, found 435.0.

### Step 2: tert-Butyl 4-[[2-amino-5-[(1-methyl-4-piperidyl)oxy]-3-pyridyl]amino]piperidine-1-carboxylate

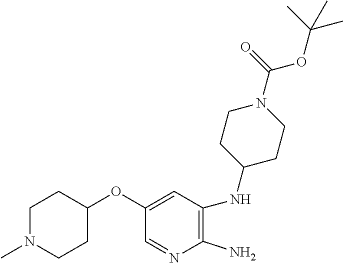 [SMILES: CN1CCC(Oc2cnc(N)c(NC3CCN(C(=O)OC(C)(C)C)CC3)c2)CC1]

To a solution of 4-[[5-[(1-methyl-4-piperidyl)oxy]-2-nitro-3-pyridyl]amino]piperidine-1-carboxylate (768 mg, 1.76 mmol) in MeOH (20 mL) were successively added ammonium formate (2223 mg, 35.26 mmol) and Pd/C (10%) (150 mg) and the resulting mixture was stirred at 65° C. for one hour. After one hour, 556 mg of ammonium formate (8.81 mmol) was added and the reaction mixture was stirred for 30 additional minutes at 65° C., then filtered through celite, washed with MeOH, and the filtrate concentrated in vacuo. The resulting residue was diluted with ethyl acetate, washed with a 1N aqueous solution of NaOH, dried over Na₂SO₄, filtered, and concentrated in vacuo. The resulting dark oil was purified by flash chromatography on silica gel (SiO₂ 24 g) eluting with DCM/MeOH/30% aq. NH₄OH (92/8/0.8) to give 448 mg (57% yield) of tert-butyl 4-[[2-amino-5-[(1-methyl-4-piperidyl)oxy]-3-pyridyl]amino]piperidine-1-carboxylate as a brown foam. LC/MS (m/z, M+H): calc. 406.2, found 406.3.

### Step 3: tert-Butyl 4-[6-[(1-methyl-4-piperidyl)oxy]-2-oxo-3H-imidazo[4,5-b]pyridin-1-yl]piperidine-1-carboxylate

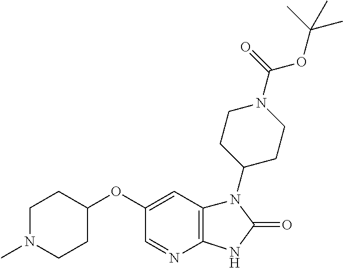 [SMILES: CN1CCC(Oc2cnc3[nH]c(=O)n(C4CCN(C(=O)OC(C)(C)C)CC4)c3c2)CC1]

To a solution of tert-butyl 4-[[2-amino-5-[(1-methyl-4-piperidyl)oxy]-3-pyridyl]amino]piperidine-1-carboxylate (445 mg, 1 mmol) in THF (10 mL) was added CDI (485 mg, 3 mmol) and the resulting mixture was refluxed for one hour. Then, the reaction mixture was cooled down to room temperature, diluted with an aqueous saturated solution of NaHCO₃ and extracted with ethyl acetate. The organic layer was then dried over Na₂SO₄, filtered, and concentrated in vacuo. The resulting dark oil was purified by flash chromatography on silica gel (SiO₂ 24 g) eluting with DCM/MeOH/30% aq. NH₄OH (92/8/0.8) to give 431 mg (100% yield) of tert-butyl 4-[6-[(1-methyl-4-piperidyl)oxy]-2-oxo-3H-imidazo[4,5-b]pyridin-1-yl]piperidine-1-carboxylate as brown foam. LC/MS (m/z, M+H): calc. 432.2, found 432.3.

### Step 4: 6-[(1-Methyl-4-piperidyl)oxy]-1-(4-piperidyl)-3H-imidazo[4,5-b]pyridin-2-one, trihydrochloride

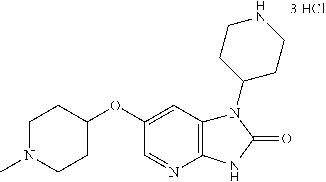 [SMILES: CN1CCC(Oc2cnc3[nH]c(=O)n(C4CCNCC4)c3c2)CC1]

To a solution of tert-butyl 4-[6-[(1-methyl-4-piperidyl)oxy]-2-oxo-3H-imidazo[4,5-b]pyridin-1-yl]piperidine-1-carboxylate (431 mg, 1 mmol) in MeOH (8 mL) was added 3.74 mL of a 4N HCl solution in dioxane (15 mmol) and the resulting mixture was stirred at 40° C. for 1 hour. Then, the reaction mixture was cooled down to room temperature and concentrated under reduced pressure to give 440 mg (100% yield) of 6-[(1-methyl-4-piperidyl)oxy]-1-(4-piperidyl)-3H-imidazo[4,5-b]pyridin-2-one, trihydrochloride as a brown solid which was used in the next step without further purification. LC/MS (m/z, M+H): calc. 332.2, found 332.2 (free base).

### Step 5: 6-[(1-Methyl-4-piperidyl)oxy]-1-[1-[4-(trifluoromethoxy)benzoyl]-4-piperidyl]-3H-imidazo[4,5-b]pyridin-2-one

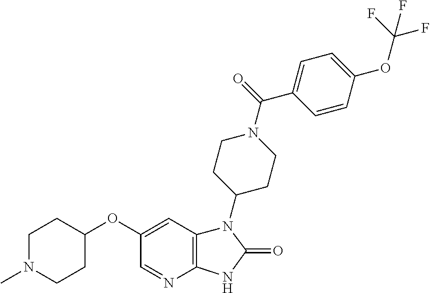 [SMILES: CN1CCC(Oc2cnc3[nH]c(=O)n(C4CCN(C(=O)c5ccc(OC(F)(F)F)cc5)CC4)c3c2)CC1]

To a solution of 6-[(1-methyl-4-piperidyl)oxy]-1-(4-piperidyl)-3H-imidazo[4,5-b]pyridin-2-one, trihydrochloride (220 mg, 0.5 mmol) in DMF (3.3 mL) was added DIPEA (323 mg, 0.44 mL, 2.5 mmol) and the resulting mixture was stirred for 5 minutes. Then, 4-(trifluoromethoxy)benzoic acid (103 mg, 0.5 mmol) and HATU (209 mg, 0.55 mmol) were successively added and the reaction mixture was stirred 1.5 hour at room temperature, then diluted with ethyl acetate, washed with a 1N aqueous solution of NaOH, water and brine. The organic layer was dried over Na₂SO₄, filtered, and concentrated under reduced pressure. The resulting residue was purified by flash chromatography on silica gel (SiO₂ 12 g) eluting with DCM/MeOH/30% aq. NH₄OH (92/8/0.8) to give 14 mg (5% yield) of 6-[(1-methyl-4-piperidyl)oxy]-1-[1-[4-(trifluoromethoxy)benzoyl]-4-piperidyl]-3H-imidazo[4,5-b]pyridin-2-one as a pale yellow solid. LC/MS (m/z, M+H): calc. 520.2, found 520.2; ¹H NMR (400 MHz, DMSO-d6, 120° C.) δ ppm 1.64-1.78 (m, 2H), 1.84 (br d, J=11 Hz, 2H), 1.90-2.01 (m, 2H), 2.15-2.26 (m, 2H), 2.23 (s, 3H), 2.32 (qd, J=12, 4 Hz, 2H), 2.62-2.71 (m, 2H), 3.06-3.17 (m, 2H), 4.20 (br d, J=12 Hz, 2H), 4.28 (tt, J=8, 4 Hz, 1H), 4.42 (tt, J=12, 4 Hz, 1H), 7.24 (d, J=2 Hz, 1H), 7.41 (br d, J=9 Hz, 2H), 7.60 (d, J=9 Hz, 2H), 7.64 (d, J=2 Hz, 1H), 10.43-11.42 (m, 1H)

### Example 6. (rac)-1-[1-[2-Amino-4-(trifluoromethoxy)benzoyl]-4-piperidyl]-6-(1-methylpyrrolidin-3-yl)oxy-3H-imidazo[4,5-b]pyridin-2-one

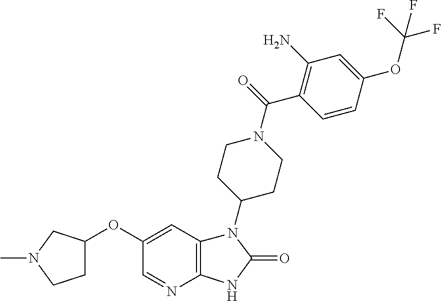 [SMILES: CN1CCC(Oc2cnc3[nH]c(=O)n(C4CCN(C(=O)c5ccc(OC(F)(F)F)cc5N)CC4)c3c2)C1]

### Step 1: (rac)-tert-Butyl 4-[[5-(1-methylpyrrolidin-3-yl)oxy-2-nitro-3-pyridyl]amino]piperidine-1-carboxylate

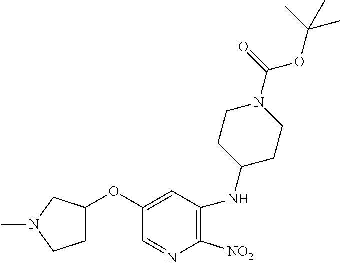 [SMILES: CN1CCC(Oc2cnc([N+](=O)[O-])c(NC3CCN(C(=O)OC(C)(C)C)CC3)c2)C1]

Under argon atmosphere, to a solution of 1-methylpyrrolidin-3-ol (476 mg, 4.7 mmol) in DMF (6 mL) was added at 0° C., sodium hydride (60% dispersion in oil, 197 mg, 4.94 mmol) and the reaction mixture was stirred for 20 minutes. Then, a solution of tert-butyl 4-[(5-fluoro-2-nitro-3-pyridyl)amino]piperidine-1-carboxylate (800 mg, 2.35 mmol) in DMF (6 mL) was added dropwise and the resulting reaction mixture was warmed up to room temperature and stirred for 1.5 hours, then diluted with water and extracted with ethyl acetate. The organic layer was washed with brine, dried over Na₂SO₄, filtered, and concentrated under reduced pressure. The resulting orange oil was purified by flash chromatography on silica gel (SiO₂ 24 g) eluting with DCM/MeOH/30% aq. NH₄OH (95/5/0.5) to give 931 mg (85% yield) of (rac)-tert-butyl 4-[[5-(1-methylpyrrolidin-3-yl)oxy-2-nitro-3-pyridyl]amino]piperidine-1-carboxylate as a yellow solid. LC/MS (m/z, M+): calc. 421.2, found 421.0.

### Step 2: (rac)-tert-Butyl 4-[[2-amino-5-(1-methylpyrrolidin-3-yl)oxy-3-pyridyl]amino]piperidine-1-carboxylate

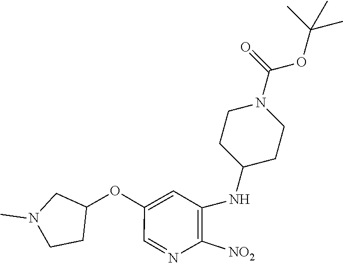 [SMILES: CN1CCC(Oc2cnc([N+](=O)[O-])c(NC3CCN(C(=O)OC(C)(C)C)CC3)c2)C1]

To a solution of (rac)-tert-butyl 4-[[5-(1-methylpyrrolidin-3-yl)oxy-2-nitro-3-pyridyl]amino]piperidine-1-carboxylate (928 mg, 2.00 mmol) in MeOH (20 mL) were added ammonium formate (2527 mg, 40.1 mmol) and Pd/C (10%) (200 mg) and the resulting reaction mixture was stirred at 65° C. for one hour. Then, the reaction mixture was cooled down to room temperature, filtered through celite, washed with MeOH and the filtrate was concentrated under reduced pressure. The resulting residue was diluted with ethyl acetate and washed with a TN aqueous solution of NaOH. The organic layer was dried over Na₂SO₄, filtered, and concentrated in vacuo. The resulting dark oil was purified by flash chromatography on silica gel (SiO₂ 24 g) eluting with DCM/MeOH/30% aq. NH₄OH (90/10/1) to give 458 mg of (rac)-tert-butyl 4-[[2-amino-5-(1-methylpyrrolidin-3-yl)oxy-3-pyridyl]amino]piperidine-1-carboxylate as a brown foam. LC/MS (m/z, M+): calc. 391.2, found 391.0.

### Step 3: (rac)-tert-Butyl 4-[6-(1-methylpyrrolidin-3-yl)oxy-2-oxo-3H-imidazo[4,5-b]pyridin-1-yl]piperidine-1-carboxylate

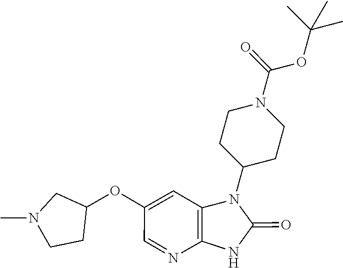 [SMILES: CN1CCC(Oc2cnc3[nH]c(=O)n(C4CCN(C(=O)OC(C)(C)C)CC4)c3c2)C1]

To a solution of (rac)-tert-butyl 4-[[2-amino-5-(1-methylpyrrolidin-3-yl)oxy-3-pyridyl]amino]piperidine-1-carboxylate (455 mg, 1.09 mmol) in THF (10 mL) was added CDI (531 mg, 3.28 mmol) and the resulting reaction mixture was refluxed for 1.5 hours, then cooled down to room temperature and concentrated under reduced pressure. The resulting residue was diluted with ethyl acetate, and washed twice with an aqueous saturated solution of NaHCO₃. The organic layer was dried over Na₂SO₄, filtered, and concentrated in vacuo. The resulting residue was purified by flash chromatography on silica gel (SiO₂ 24 g) eluting with DCM/MeOH/30% aq. NH₄OH (90/10/1) to give a purple powder which was triturated in acetonitrile and filtered to give 305 mg (66% yield) of (rac)-tert-butyl 4-[6-(1-methylpyrrolidin-3-yl)oxy-2-oxo-3H-imidazo[4,5-b]pyridin-1-yl]piperidine-1-carboxylate as a pink powder. LC/MS (m/z, M+): calc. 417.2, found 417.0.

### Step 4: (rac)-6-(1-Methylpyrrolidin-3-yl)oxy-1-(4-piperidyl)-3H-imidazo[4,5-b]pyridin-2-one, trihydrochloride

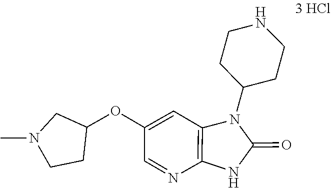 [SMILES: CN1CCC(Oc2cnc3[nH]c(=O)n(C4CCNCC4)c3c2)C1]

To a solution of (rac)-tert-butyl 4-[6-(1-methylpyrrolidin-3-yl)oxy-2-oxo-3H-imidazo[4,5-b]pyridin-1-yl]piperidine-1-carboxylate (300 mg, 0.72 mmol) in MeOH (4 mL) was added 2.7 mL of a 4N HCl solution in dioxane (10.8 mmol). The resulting mixture was stirred at room temperature for 4 hours then concentrated in vacuo to give 307 mg (100% yield) of (rac)-6-(1-methylpyrrolidin-3-yl)oxy-1-(4-piperidyl)-3H-imidazo[4,5-b]pyridin-2-one, trihydrochloride as a brown foam. LC/MS (m/z, M+H): calc. 318.2, found 318.2 (free base).

### Step 5: (rac)-1-[1-[2-Amino-4-(trifluoromethoxy)benzoyl]-4-piperidyl]-6-(1-methylpyrrolidin-3-yl)oxy-3H-imidazo[4,5-b]pyridin-2-one

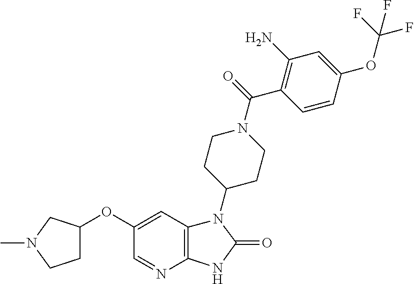 [SMILES: CN1CCC(Oc2cnc3[nH]c(=O)n(C4CCN(C(=O)c5ccc(OC(F)(F)F)cc5N)CC4)c3c2)C1]

To a solution of (rac)-6-(1-methylpyrrolidin-3-yl)oxy-1-(4-piperidyl)-3H-imidazo[4,5-b]pyridin-2-one, trihydrochloride (153 mg, 0.36 mmol) in DMF (2.4 mL) was added DIPEA (232 mg, 0.31 mL, 1.8 mmol) and the resulting mixture was stirred at room temperature for 5 minutes. Then 2-amino-4-(trifluoromethoxy)benzoic acid (79 mg, 0.36 mmol) followed by HATU (150 mg, 0.39 mmol) were added and the resulting reaction mixture was stirred at room temperature for 2 hours, then diluted with ethyl acetate, washed with an aqueous saturated solution of NaHCO₃, water and brine, dried over Na₂SO₄, filtered and concentrated in vacuo. The resulting residue was purified by flash chromatography on silica gel (12 g) eluting with DCM/MeOH/30% aq. NH₄OH (90/10/1) to give a yellow foam which was then triturated in i-PrOH. The resulting white precipitate was filtered off to give 131 mg (70% yield) of (rac)-1-[1-[2-amino-4-(trifluoromethoxy)benzoyl]-4-piperidyl]-6-(1-methylpyrrolidin-3-yl)oxy-3H-imidazo[4,5-b]pyridin-2-one as a white solid. LC/MS (m/z, M+H): calc. 521.2, found 521.2; ¹H NMR (400 MHz, DMSO-d6, 120° C.) δ ppm 1.75-1.91 (m, 3H), 2.12-2.33 (m, 3H), 2.29 (s, 3H), 2.40-2.52 (m partially hidden, 1H), 2.59-2.72 (m, 2H), 2.76-2.87 (m partially hidden, 1H), 3.05 (td, J=13, 3 Hz, 2H), 4.16 (br d, J=14 Hz, 2H), 4.39 (tt, J=12, 4 Hz, 1H), 4.82-4.91 (m, 1H), 5.33 (s, 2H), 6.44-6.54 (m, 1H), 6.67-6.73 (m, 1H), 7.14 (d, J=8 Hz, 1H), 7.17 (d, J=2 Hz, 1H), 7.54 (d, J=2 Hz, 1H), 10.71-11.00 (m, 1H)

### Example 7: 1-[1-[2-Amino-4-(trifluoromethoxy)benzoyl]-4-piperidyl]-6-[(1-methyl-4-piperidyl)oxy]-3H-imidazo[45-h]pyridin-2-one

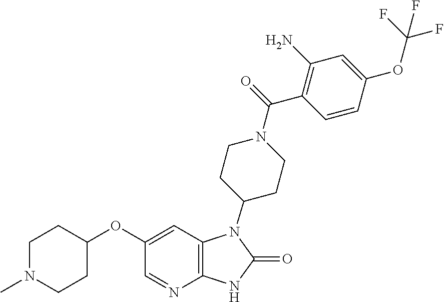 [SMILES: CN1CCC(Oc2cnc3[nH]c(=O)n(C4CCN(C(=O)c5ccc(OC(F)(F)F)cc5N)CC4)c3c2)CC1]

To a solution of 6-[(1-methyl-4-piperidyl)oxy]-1-(4-piperidyl)-3H-imidazo[4,5-b]pyridin-2-one, hydrochloride (220 mg, 0.5 mmol) in DMF (3.3 mL) was added DIPEA (323 mg, 0.44 mL, 2.5 mmol) and the resulting mixture was stirred at room temperature for 5 minutes. Then 2-amino-4-(trifluoromethoxy)benzoic acid (110 mg, 0.5 mmol) followed by HATU (209 mg, 0.55 mmol) were added and the resulting reaction mixture was stirred at room temperature for 1.5 hours, then diluted with ethyl acetate, washed with a TN aqueous solution of NaOH, water and brine. The organic layer was dried over Na₂SO₄, filtered, and concentrated under reduced pressure. The resulting residue was purified by flash chromatography on silica gel (SiO₂ 12 g) eluting with DCM/MeOH/30% aq. NH₄OH (90/10/1) to give a yellow gum which was triturated in acetonitrile. The resulting precipitate was filtered off to give 19 mg (7% yield) of −[1-[2-amino-4-(trifluoromethoxy)benzoyl]-4-piperidyl]-6-[(1-methyl-4-piperidyl)oxy]-3H-imidazo[4,5-b]pyridin-2-one as a pale brown solid. LC/MS (m/z, M+H): calc. 535.2, found 535.3; ¹H NMR (400 MHz, DMSO-d6, 120° C.) δ ppm 1.62-1.75 (m, 2H), 1.80 (br d, J=12 Hz, 2H), 1.87-1.97 (m, 2H), 2.13-2.35 (m, 4H), 2.19 (s, 3H), 2.58-2.67 (m, 2H), 3.05 (br t, J=12 Hz, 2H), 4.16 (br d, J=13 Hz, 2H), 4.24 (tt, J=8, 4 Hz, 1H), 4.33-4.45 (m, 1H), 5.31 (br s, 2H), 6.48 (br d, J=8 Hz, 1H), 6.69 (br s, 1H), 7.14 (d, J=8 Hz, 1H), 7.19 (d, J=2 Hz, 1H), 7.60 (d, J=2 Hz, 1H), 10.61-11.06 (m, 1H)

### Example 8: 6-Morpholino-1-[1-[4-(trifluoromethoxy)benzoyl]-4-piperidyl]-3H-imidazo[4,5-b]pyridin-2-one

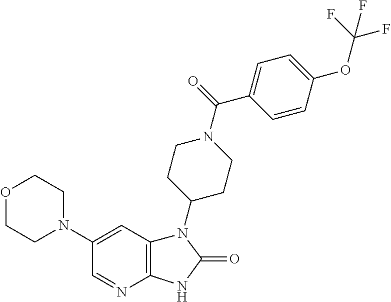 [SMILES: O=C(c1ccc(OC(F)(F)F)cc1)N1CCC(n2c(=O)[nH]c3ncc(N4CCOCC4)cc32)CC1]

To a solution of 6-morpholino-1-(4-piperidyl)-3H-imidazo[4,5-b]pyridin-2-one, trihydrochloride (95 mg, 0.23 mmol) in DMF (1.5 mL) was added DIPEA (208 mg, 0.28 mL, 1.61 mmol) and the resulting mixture was stirred at room temperature for 5 minutes. Then, 4-(trifluoromethoxy)benzoic acid (47 mg, 0.23 mmol) and HATU (96 mg, 0.25 mmol) were successively added and the reaction mixture was stirred 12 hours at room temperature. The reaction mixture was diluted with ethyl acetate and washed with a TN aqueous solution of NaOH (5 mL). The aqueous layer was extracted 4 times with ethyl acetate. The combined organic layers were dried over Na₂SO₄, filtered, and concentrated under reduced pressure. The resulting residue was purified by flash chromatography on Puriflash® interchim PF-15SiHP-F0012 (12 g) column eluting with AcOEt/MeOH/30% aq. NH₄OH (94/6/0.6) to give a solid which was triturated in acetonitrile and filtered to furnish 64 mg (56% yield) of 6-morpholino-1-[1-[4-(trifluoromethoxy)benzoyl]-4-piperidyl]-3H-imidazo[4,5-b]pyridin-2-one as a white solid. LC/MS (m/z, M+H): calc. 492.2, found 492.2; ¹H NMR (400 MHz, DMSO-d6, 30° C.) δ ppm 1.52-1.91 (m, 2H), 2.15-2.40 (m, 2H), 2.75-3.40 (m partially hidden, 2H), 3.03-3.12 (m, 4H), 3.55-3.80 (m, 1H), 3.73-3.83 (m, 4H), 4.44 (tt, J=12, 4 Hz, 1H), 4.53-4.83 (m, 1H), 7.35 (d, J=2 Hz, 1H), 7.46 (br d, J=8 Hz, 2H), 7.58 (d, J=2 Hz, 1H), 7.60 (d, J=9 Hz, 2H), 11.17-11.34 (m, 1H)

### Example 9: (rac)-6-(1-Methylpyrrolidin-3-yl)oxy-1-[1-[4-(trifluoromethoxy)benzoyl]-4-piperidyl]-3H-imidazo[4,5-b]pyridin-2-one

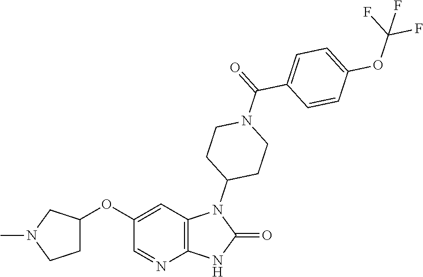 [SMILES: CN1CCC(Oc2cnc3[nH]c(=O)n(C4CCN(C(=O)c5ccc(OC(F)(F)F)cc5)CC4)c3c2)C1]

To a solution of 6-(1-methylpyrrolidin-3-yl)oxy-1-(4-piperidyl)-3H-imidazo[4,5-b]pyridin-2-one, trihydrochloride (153 mg, 0.36 mmol) in DMF (2.4 mL) was added DIPEA (232 mg, 0.31 mL, 1.8 mmol) and the resulting reaction mixture was stirred at room temperature for 5 minutes. Then, 4-(trifluoromethoxy)benzoic acid (74 mg, 0.36 mmol) followed by HATU (150 mg, 0.39 mmol) were added and the resulting mixture was stirred at room temperature for 2 hours, then diluted with ethyl acetate, washed with an aqueous saturated solution of NaHCO₃, water and brine, dried over Na₂SO₄, filtered, and concentrated in vacuo. The resulting residue was purified by flash chromatography on silica gel (SiO₂ 12 g) eluting with DCM/MeOH/30% aq. NH₄OH (90/10/1) to give 143 mg (78% yield) of (rac)-6-(1-methylpyrrolidin-3-yl)oxy-1-[1-[4-(trifluoromethoxy)benzoyl]-4-piperidyl]-3H-imidazo[4,5-b]pyridin-2-one as a pale brown solid. LC/MS (m/z, M+H): calc. 506.2, found 506.2; ¹H NMR (400 MHz, DMSO-d6, 120° C.) δ ppm 1.75-1.93 (m, 3H), 2.16-2.36 (m, 3H), 2.29 (s, 3H), 2.41-2.50 (m partially hidden, 1H), 2.61-2.72 (m, 2H), 2.78-2.87 (m partially hidden, 1H), 3.08 (td, J=13, 3 Hz, 2H), 4.11-4.25 (m, 2H), 4.40 (tt, J=12, 4 Hz, 1H), 4.83-4.89 (m, 1H), 7.18 (d, J=2 Hz, 1H), 7.38 (br d, J=8 Hz, 2H), 7.55 (d, J=2 Hz, 1H), 7.57 (d, J=8 Hz, 2H), 10.58-11.11 (m, 1H)

### Example 10: 1-[1-[2-Amino-4-(trifluoromethoxy)benzoyl]-4-piperidyl]-6-[(2S)-2-(hydroxymethyl)morpholin-4-yl]-3H-imidazo[4,5-b]pyridin-2-one

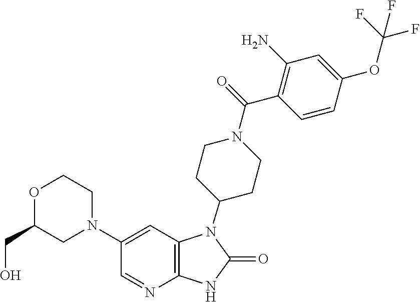 [SMILES: Nc1cc(OC(F)(F)F)ccc1C(=O)N1CCC(n2c(=O)[nH]c3ncc(N4CCO[C@H](CO)C4)cc32)CC1]

### Step 1: (2S)-2-(Tetrahydropyran-2-yloxymethyl)morpholine

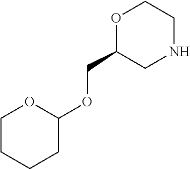 [SMILES: C1CCC(OC[C@@H]2CNCCO2)OC1]

To a suspension of [(2S)-morpholin-2-yl]methanol, hydrochloride (2.16 g, 13.6 mmol) in DCM (140 mL) under argon atmosphere were successively added DHP (2.48 mL, 2290 mg, 27.3 mmol) and PPTS (343 mg, 1.36 mmol) and the resulting reaction mixture was stirred at room temperature for 20 hours. Then, the reaction mixture was diluted with 100 mL of an aqueous saturated solution of NaHCO₃, extracted with DCM. The organic layer was then dried over Na₂SO₄, filtered, and concentrated in vacuo. The resulting residue was purified by flash chromatography on silica gel (SiO₂ 40 g) eluting with DCM/MeOH/aq. 30% NH₄OH (95/5/0.5) to give 1.13 g (41% yield) of (2S)-2-(tetrahydropyran-2-yloxymethyl)morpholine as a yellow oil. LC/MS (m/z, M+): calc. 201.2, found 201.1.

### Step 2: tert-Butyl 4-[2-oxo-6-[(2S)-2-(tetrahydropyran-2-yloxymethyl)morpholin-4-yl]-3H-imidazo[4,5-b]pyridin-1-yl]piperidine-1-carboxylate

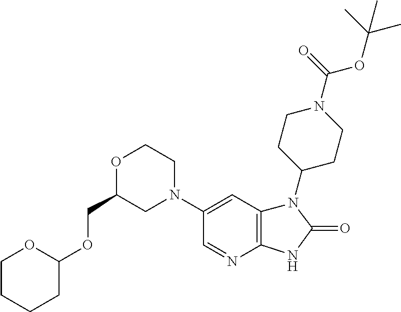 [SMILES: CC(C)(C)OC(=O)N1CCC(n2c(=O)[nH]c3ncc(N4CCO[C@H](COC5CCCCO5)C4)cc32)CC1]

In a sealed tube, to a solution of (2S)-2-(tetrahydropyran-2-yloxymethyl)morpholine (319 mg, 1.58 mmol), tert-butyl 4-(6-bromo-2-oxo-3H-imidazo[4,5-b]pyridin-1-yl)piperidine-1-carboxylate (525 mg, 1.32 mmol) and RuPhos (49 mg, 0.11 mmol) in THF (0.45 mL) was added LiHMDS (1 M solution in THF, 3.17 mL, 3.17 mmol) and the resulting mixture was bubbled with argon for 5 minutes. Pd(OAc)₂ (12 mg, 0.053 mmol) was then added, the tube was sealed and heated at 75° C. under stirring overnight. After one night, the reaction mixture was cooled down to room temperature, diluted with a saturated aqueous solution of ammonium chloride, extracted with DCM. The organic layer was then dried over Na₂SO₄, filtered, and concentrated in vacuo. The resulting residue was purified by flash chromatography on silica gel (SiO₂ 40 g) eluting with DCM/MeOH/30% aq. NH₄OH (94.5/5.5/0.55) to give a solid which was triturated in 15 mL of i-Pr₂O. The resulting precipitate was filtered off to give 544 mg (79% yield) of tert-butyl 4-[2-oxo-6-[(2S)-2-(tetrahydropyran-2-yloxymethyl)morpholin-4-yl]-3H-imidazo[4,5-b]pyridin-1-yl]piperidine-1-carboxylate as a white solid. LC/MS (m/z, M+): calc. 517.3, found 517.0.

### Step 3: 6-[(2S)-2-(Hydroxymethyl)morpholin-4-yl]-1-(4-piperidyl)-3H-imidazo[4,5-b]pyridin-2-one, hydrochloride

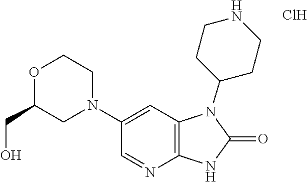 [SMILES: Cl ; O=c1[nH]c2ncc(N3CCO[C@H](CO)C3)cc2n1C1CCNCC1]

To a solution of tert-butyl 4-[2-oxo-6-[(2S)-2-(tetrahydropyran-2-yloxymethyl)morpholin-4-yl]-3H-imidazo[4,5-b]pyridin-1-yl]piperidine-1-carboxylate (541 mg, 1.05 mmol) in MeOH (5 mL) was added 3.13 mL of a 4N HCl solution in dioxane (12.5 mmol). The resulting reaction mixture was stirred at room temperature overnight, then concentrated in vacuo. The resulting residue was diluted in MeOH and concentrated in vacuo three times, and the resulting residue was triturated in i-PrOH (20 mL). The precipitate was filtered off and washed with ethyl acetate to give 367 mg (94% yield) of 6-[(2S)-2-(hydroxymethyl)morpholin-4-yl]-1-(4-piperidyl)-3H-imidazo[4,5-b]pyridin-2-one, hydrochloride as pale brown solid. LC/MS (m/z, M+H): calc. 334.2, found 334 (free base).

### Step 4:1-[1-[2-Amino-4-(trifluoromethoxy)benzoyl]-4-piperidyl]-6-[(2S)-2-(hydroxymethyl)morpholin-4-yl]-3H-imidazo[4,5-b]pyridin-2-one

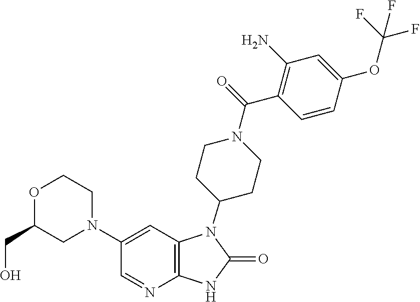 [SMILES: Nc1cc(OC(F)(F)F)ccc1C(=O)N1CCC(n2c(=O)[nH]c3ncc(N4CCO[C@H](CO)C4)cc32)CC1]

To a suspension of 6-[(2S)-2-(hydroxymethyl)morpholin-4-yl]-1-(4-piperidyl)-3H-imidazo[4,5-b]pyridin-2-one, hydrochloride (180 mg, 0.49 mmol) in DMF (3.2 mL) was added DIPEA (0.59 mL, 440 mg, 3.41 mmol) and the resulting mixture was stirred at room temperature for 5 minutes. Then, 2-amino-4-(trifluoromethoxy)benzoic acid (108 mg, 0.49 mmol) followed by HATU (185 mg, 0.49 mmol) were added and the resulting solution was stirred at room temperature overnight. After one night, the reaction mixture was diluted with a TN aqueous solution of NaOH (5 mL), MeOH (5 mL) and toluene (5 mL), and concentrated in vacuo. The resulting residue was diluted with MeOH and toluene and concentrated to dryness three times. The resulting residue was then purified by flash chromatography on Puriflash® interchim PF-15SiHP-F0012 (12 g) column eluting with DCM/MeOH/30% aq. NH₄OH (91.5/8.5/0.85) to give a solid which was triturated in i-Pr₂O. The precipitate was filtered off and furnished 148 mg (56% yield) of 1-[1-[2-amino-4-(trifluoromethoxy)benzoyl]-4-piperidyl]-6-[(2S)-2-(hydroxymethyl)morpholin-4-yl]-3H-imidazo[4,5-b]pyridin-2-one as a white solid. LC/MS (m/z, M+H): calc. 537.2, found 537.2; ¹H NMR (400 MHz, DMSO-d6, 120° C.) δ ppm 1.75-1.84 (m, 2H), 2.24-2.36 (m, 2H), 2.56 (dd, J=12, 10 Hz, 1H), 2.74-2.84 (m partially hidden, 1H), 3.01-3.11 (m, 2H), 3.32 (br dd, J=12, 2 Hz, 1H), 3.40-3.51 (m, 2H), 3.51-3.59 (m, 1H), 3.59-3.66 (m, 1H), 3.69 (td, J=11, 3 Hz, 1H), 3.90-3.97 (m, 1H), 4.16 (br d, J=13 Hz, 2H), 4.20-4.27 (m, 1H), 4.41 (tt, J=12, 4 Hz, 1H), 5.32 (s, 2H), 6.45-6.55 (m, 1H), 6.66-6.70 (m, 1H), 7.12 (d, J=8 Hz, 1H), 7.15 (d, J=2 Hz, 1H), 7.57 (d, J=2 Hz, 1H), 10.74 (br s, 1H)

### Example 11: 6-[(2R)-2-(Hydroxymethyl)morpholin-4-yl]-1-[1-[4-(trifluoromethoxy)benzoyl]-4-piperidyl]-3H-imidazo[4,5-b]pyridin-2-one

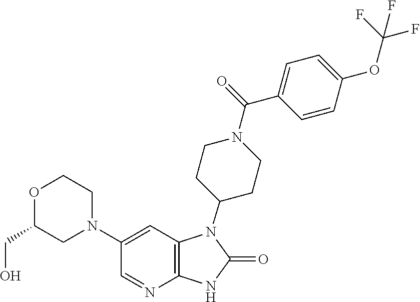 [SMILES: O=C(c1ccc(OC(F)(F)F)cc1)N1CCC(n2c(=O)[nH]c3ncc(N4CCO[C@@H](CO)C4)cc32)CC1]

### Step 1: (2R)-2-(Tetrahydropyran-2-yloxymethyl)morpholine

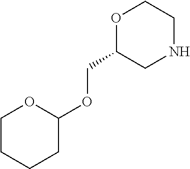 [SMILES: C1CCC(OC[C@H]2CNCCO2)OC1]

To a suspension of [(2R)-morpholin-2-yl]methanol, hydrochloride (1.08 g, 7.03 mmol) in DCM (70 mL) under argon atmosphere were successively added DHP (1.28 mL, 1180 mg, 14.1 mmol) and PPTS (177 mg, 0.7 mmol) and the resulting reaction mixture was stirred at room temperature for 40 hours. The reaction mixture was then concentrated to an approximative volume of 20 mL, and slowly poured onto a solution of KOH (585 mg, 10.4 mmol) in MeOH (100 mL). The whole mixture was then concentrated to dryness, diluted with DCM, filtered and the filtrate was concentrated in vacuo. The resulting residue was purified by flash chromatography on silica gel (SiO₂ 40 g) eluting with DCM/MeOH/aq. 30% NH₄OH (95/5/0.5) to give 1.22 g (86% yield) of (2R)-2-(tetrahydropyran-2-yloxymethyl)morpholine as a brown oil. LC/MS (m/z, M+H): calc. 202.1, found 202.0.

### Step 2: 6-Bromo-1-(4-piperidyl)-3H-imidazo[4,5-b]pyridin-2-one, dihydrochloride

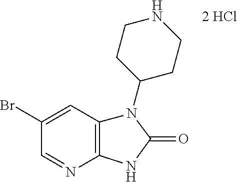 [SMILES: O=c1[nH]c2ncc(Br)cc2n1C1CCNCC1]

To a solution of tert-butyl 4-(6-bromo-2-oxo-3H-imidazo[4,5-b]pyridin-1-yl)piperidine-1-carboxylate (2.01 g, 5.06 mmol) in MeOH (60 mL) and DCM (20 mL) at 45° C. was added 10.1 mL of a 4N HCl solution in dioxane (40.5 mmol) and the resulting mixture was stirred for 3 hours at 45° C. The reaction mixture was then concentrated and the resulting residue was triturated in ethyl acetate (50 mL). The precipitate was filtered off and washed with ethyl acetate to give 1.87 g (100% yield) of 6-bromo-1-(4-piperidyl)-3H-imidazo[4,5-b]pyridin-2-one, dihydrochloride as a pale brown solid. LC/MS (m/z, M+): calc. 296.0, found 296.0 (free base).

### Step 3: 6-Bromo-1-[1-[4-(trifluoromethoxy)benzoyl]-4-piperidyl]-3H-imidazo[4,5-b]pyridin-2-one (Example 60)

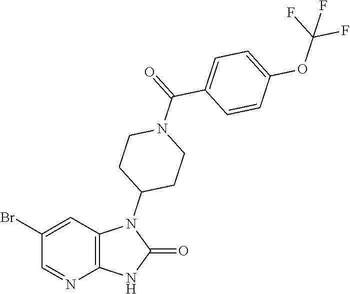 [SMILES: O=C(c1ccc(OC(F)(F)F)cc1)N1CCC(n2c(=O)[nH]c3ncc(Br)cc32)CC1]

To a suspension of 6-bromo-1-(4-piperidyl)-3H-imidazo[4,5-b]pyridin-2-one, dihydrochloride (918 mg, 2.48 mmol) in DMF (17 mL) was added DIPEA (2244 mg, 3.02 mL, 17.4 mmol) and the resulting mixture was stirred at room temperature for 5 minutes. Then, 4-(trifluoromethoxy)benzoic acid (511 mg, 2.48 mmol) followed by HATU (1037 mg, 2.73 mmol) were added and the resulting mixture was stirred at room temperature overnight. After one night, the resulting mixture was concentrated in vacuo, diluted with ethyl acetate, washed with a 1N aqueous solution of NaOH (50 mL). The aqueous layer was then extracted twice with ethyl acetate. The combined organic layers were dried over Na₂SO₄, filtered, and concentrated in vacuo. The resulting residue was purified by flash chromatography on Puriflash® interchim PF-15SiHP/40G (40 g) column eluting with DCM/MeOH/30% aq. NH₄OH (96/4/0.4) to give a solid which was triturated in i-PrOH (20 mL) to furnish 1.04 g (86% yield) of 6-bromo-1-[1-[4-(trifluoromethoxy)benzoyl]-4-piperidyl]-3H-imidazo[4,5-b]pyridin-2-one as a pale brown solid. LC/MS (m/z, M+H): calc. 484.0, found 484.0; ¹H NMR (400 MHz, DMSO-d6, 100° C.) d ppm 1.81 (br d, J=12.7 Hz, 2H), 2.22-2.33 (m, 2H), 3.01-3.12 (m, 2H), 4.09-4.26 (m, 2H), 4.38-4.47 (m, 1H), 7.39 (br d, J=8.4 Hz, 2H), 7.58 (br d, J=8.4 Hz, 2H), 7.83 (d, J=1.8 Hz, 1H), 7.96 (d, J=1.8 Hz, 1H), 11.39-11.49 (m, 1H)

### Step 4: 6-[(2R)-2-(Tetrahydropyran-2-yloxymethyl)morpholin-4-yl]-1-[1-[4-(trifluoromethoxy)benzoyl]-4-piperidyl]-3H-imidazo[4,5-b]pyridin-2-one

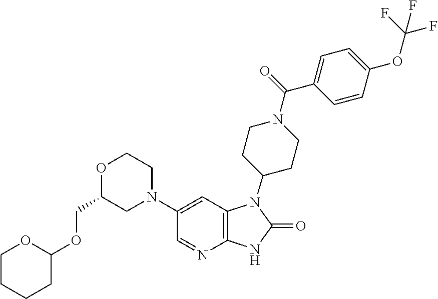 [SMILES: O=C(c1ccc(OC(F)(F)F)cc1)N1CCC(n2c(=O)[nH]c3ncc(N4CCO[C@@H](COC5CCCCO5)C4)cc32)CC1]

In a sealed tube, to a solution of (2R)-2-(tetrahydropyran-2-yloxymethyl)morpholine (151 mg, 0.75 mmol), 6-bromo-1-[1-[4-(trifluoromethoxy)benzoyl]-4-piperidyl]-3H-imidazo[4,5-b]pyridin-2-one (305 mg, 0.63 mmol) and RuPhos (25 mg, 0.05 mmol) in THF (0.42 mL) was added LiHMDS (1 M solution in THF, 1.51 mL, 1.51 mmol) and the resulting mixture was bubbled with argon for 5 minutes. Pd(OAc)₂ (6 mg, 0.03 mmol) was then added, the tube was sealed and heated at 70° C. under stirring overnight. After one night, the reaction mixture was cooled down to room temperature, diluted with a saturated aqueous solution of ammonium chloride and extracted with DCM. The organic layer was then dried over Na₂SO₄, filtered, and concentrated in vacuo. The resulting residue was purified by flash chromatography on silica gel (SiO₂ 40 g) eluting with DCM/MeOH/30% aq. NH₄OH (95/5/0.5) to give a solid which was triturated in 10 mL of i-Pr₂O. The resulting precipitate was filtered off to give 210 mg (55% yield) of 6-[(2R)-2-(tetrahydropyran-2-yloxymethyl)morpholin-4-yl]-1-[1-[4-(trifluoromethoxy)benzoyl]-4-piperidyl]-3H-imidazo[4,5-b]pyridin-2-one as a white solid. LC/MS (m/z, M+): calc. 605.2, found 605.0.

### Step 5: 6-[(2R)-2-(Hydroxymethyl)morpholin-4-yl]-1-[1-[4-(trifluoromethoxy)benzoyl]-4-piperidyl]-3H-imidazo[4,5-b]pyridin-2-one

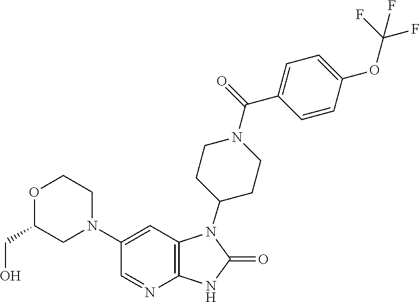 [SMILES: O=C(c1ccc(OC(F)(F)F)cc1)N1CCC(n2c(=O)[nH]c3ncc(N4CCO[C@@H](CO)C4)cc32)CC1]

To a solution of 6-[(2R)-2-(tetrahydropyran-2-yloxymethyl)morpholin-4-yl]-1-[1-[4-(trifluoromethoxy)benzoyl]-4-piperidyl]-3H-imidazo[4,5-b]pyridin-2-one (205 mg, 0.34 mmol) in MeOH (15 mL) and DCM (5 mL) was added 1.02 mL of a 4N HCl solution in dioxane (4.06 mmol) and the resulting mixture was stirred at 45° C. overnight. After one night, the whole mixture was concentrated in vacuo and the resulting residue was diluted with MeOH, basified with a 15% aqueous solution of NH₄OH and concentrated to dryness. The resulting residue was diluted with MeOH and toluene and concentrated to dryness, then purified by flash chromatography on silica gel (SiO₂ 12 g) eluting with DCM/MeOH (94/6) to give a solid which was triturated in i-Pr₂O. The precipitate was filtered off to give 131 mg (74% yield) of 6-[(2R)-2-(hydroxymethyl)morpholin-4-yl]-1-[1-[4-(trifluoromethoxy)benzoyl]-4-piperidyl]-3H-imidazo[4,5-b]pyridin-2-one as a white powder. LC/MS (m/z, M+H): calc. 522.2, found 522.2; ¹H NMR (400 MHz, DMSO-d6, 120° C.) δ ppm 1.77-1.85 (m, 2H), 2.31 (qd, J=12, 4 Hz, 2H), 2.57 (dd, J=12, 10 Hz, 1H), 2.74-2.84 (m, 1H), 3.03-3.15 (m, 2H), 3.27-3.36 (m, 1H), 3.41-3.50 (m, 2H), 3.51-3.58 (m, 1H), 3.58-3.66 (m, 1H), 3.69 (td, J=11, 3 Hz, 1H), 3.90-3.99 (m, 1H), 4.11-4.28 (m, 3H), 4.40 (tt, J=12, 4 Hz, 1H), 7.17 (d, J=2 Hz, 1H), 7.38 (d, J=9 Hz, 2H), 7.55 (d, J=9 Hz, 2H), 7.57 (d, J=2 Hz, 1H), 10.64-10.91 (m, 1H)

### Example 12: (6-[(2S)-2-(Hydroxymethyl)morpholin-4-yl]-1-[1-[4-(trifluoromethoxy)benzoyl]-4-piperidyl]-3H-imidazo[4,5-b]pyridin-2-one

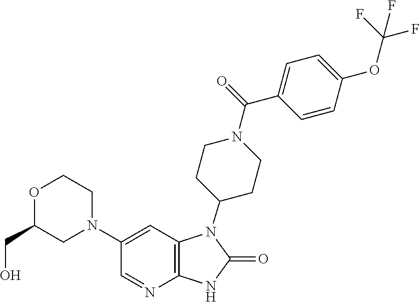 [SMILES: O=C(c1ccc(OC(F)(F)F)cc1)N1CCC(n2c(=O)[nH]c3ncc(N4CCO[C@H](CO)C4)cc32)CC1]

### Step 1: 6-[(2S)-2-(Tetrahydropyran-2-yloxymethyl)morpholin-4-yl]-1-[1-[4-(trifluoromethoxy)benzoyl]-4-piperidyl]-3H-imidazo[4,5-b]pyridin-2-one

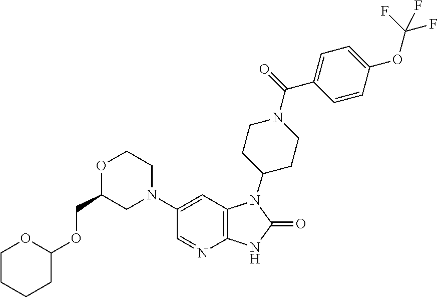 [SMILES: O=C(c1ccc(OC(F)(F)F)cc1)N1CCC(n2c(=O)[nH]c3ncc(N4CCO[C@H](COC5CCCCO5)C4)cc32)CC1]

In a sealed tube, to a solution of (2S)-2-(tetrahydropyran-2-yloxymethyl)morpholine (158 mg, 0.79 mmol), 6-bromo-1-[1-[4-(trifluoromethoxy)benzoyl]-4-piperidyl]-3H-imidazo[4,5-b]pyridin-2-one (3018 mg, 0.66 mmol) and RuPhos (26 mg, 0.056 mmol) in THF (1.6 mL) was added LiHMDS (1 M solution in THF, 1.57 mL, 1.57 mmol) and the resulting mixture was bubbled with argon for 5 minutes. Pd(OAc)₂ (6 mg, 0.03 mmol) was then added. The tube was sealed and heated at 70° C. under stirring overnight. After one night, the reaction mixture was cooled down to room temperature, diluted with a saturated aqueous solution of ammonium chloride, extracted with DCM. The organic layer was then dried over Na₂SO₄, filtered, and concentrated in vacuo. The resulting residue was purified by flash chromatography on silica gel (SiO₂ 40 g) eluting with DCM/MeOH/30% aq. NH₄OH (94.5/5.5/0.55) to give a solid which was triturated in 10 mL of i-Pr₂O. The resulting precipitate was filtered off to give 247 mg (62% yield) of 6-[(2S)-2-(tetrahydropyran-2-yloxymethyl)morpholin-4-yl]-1-[1-[4-(trifluoromethoxy)benzoyl]-4-piperidyl]-3H-imidazo[4,5-b]pyridin-2-one as a white solid. LC/MS (m/z, M+): calc. 605.2, found 605.0.

### Step 2: (6-[(2S)-2-(Hydroxymethyl)morpholin-4-yl]-1-[1-[4-(trifluoromethoxy)benzoyl]-4-piperidyl]-3H-imidazo[4,5-b]pyridin-2-one

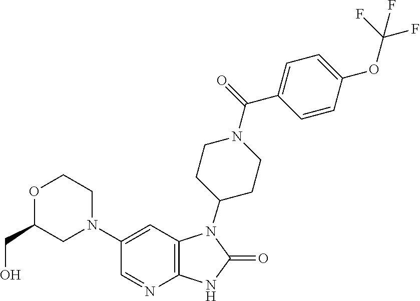 [SMILES: O=C(c1ccc(OC(F)(F)F)cc1)N1CCC(n2c(=O)[nH]c3ncc(N4CCO[C@H](CO)C4)cc32)CC1]

To a solution of 6-[(2S)-2-(tetrahydropyran-2-yloxymethyl)morpholin-4-yl]-1-[1-[4-(trifluoromethoxy)benzoyl]-4-piperidyl]-3H-imidazo[4,5-b]pyridin-2-one (243 mg, 0.40 mmol) in MeOH (10 mL) and DCM (5 mL) was added 1.2 mL of a 4N HCl solution in dioxane (4.82 mmol) and the resulting mixture was stirred at 45° C. for 4 hours. After 4 hours, the whole mixture was concentrated in vacuo and the resulting residue was diluted with MeOH, basified with a 15% aqueous solution of NH₄OH and concentrated to dryness. The resulting residue was diluted with MeOH and toluene and concentrated to dryness, then purified by flash chromatography on silica gel (SiO₂ 12 g) eluting with DCM/MeOH (94/6) to give a solid which was triturated in i-Pr₂O. The precipitate was filtered off to give 142 mg (68% yield) of 6-[(2S)-2-(hydroxymethyl)morpholin-4-yl]-1-[1-[4-(trifluoromethoxy)benzoyl]-4-piperidyl]-3H-imidazo[4,5-b]pyridin-2-one as a white powder. LC/MS (m/z, M+H): calc. 522.2, found 522.2; ¹H NMR (400 MHz, DMSO-d6, 120° C.) δ ppm 1.81 (br d, J=11 Hz, 2H), 2.31 (qd, J=12, 4 Hz, 2H), 2.57 (br dd, J=12, 10 Hz, 1H), 2.71-2.84 (m partially hidden, 1H), 3.03-3.18 (m, 2H), 3.27-3.36 (m, 1H), 3.40-3.81 (m, 5H), 3.88-4.01 (m, 1H), 4.17 (br d, J=12 Hz, 3H), 4.40 (tt, J=12, 4 Hz, 1H), 7.17 (d, J=2 Hz, 1H), 7.38 (br d, J=8 Hz, 2H), 7.55 (br d, J=9 Hz, 2H), 7.57 (d, J=2 Hz, 1H), 10.62-10.93 (m, 1H)

### Example 13: 1-[1-[2-Amino-4-(trifluoromethoxy)benzoyl]-4-piperidyl]-6-[(2R)-2-(hydroxymethyl)morpholin-4-yl]-3H-imidazo[4,5-b]pyridin-2-one

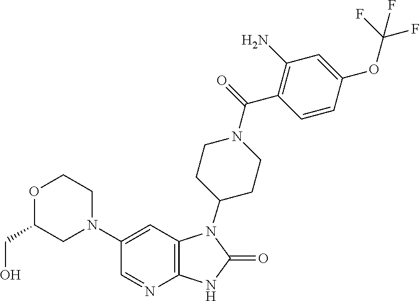 [SMILES: Nc1cc(OC(F)(F)F)ccc1C(=O)N1CCC(n2c(=O)[nH]c3ncc(N4CCO[C@@H](CO)C4)cc32)CC1]

### Step 1: tert-Butyl 4-[2-oxo-6-[(2R)-2-(tetrahydropyran-2-yloxymethyl)morpholin-4-yl]-3H-imidazo[4,5-b]pyridin-1-yl]piperidine-1-carboxylate

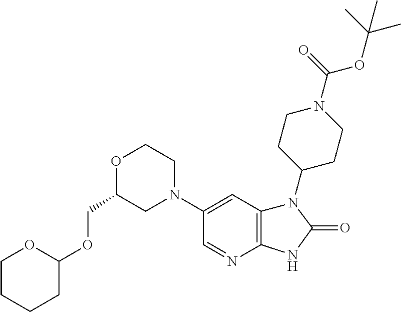 [SMILES: CC(C)(C)OC(=O)N1CCC(n2c(=O)[nH]c3ncc(N4CCO[C@@H](COC5CCCCO5)C4)cc32)CC1]

In a sealed tube, to a solution of (2R)-2-(tetrahydropyran-2-yloxymethyl)morpholine (232 mg, 1.15 mmol), tert-butyl 4-(6-bromo-2-oxo-3H-imidazo[4,5-b]pyridin-1-yl)piperidine-1-carboxylate (382 mg, 0.96 mmol) and RuPhos (36 mg, 0.08 mmol) in THF (0.64 mL) was added LiHMDS (1 M solution in THF, 2.31 mL, 2.31 mmol) and the resulting mixture was bubbled with argon for 5 minutes. Pd(OAc)₂ (9 mg, 0.04 mmol) was then added, the tube was sealed and heated at 75° C. under stirring overnight. After one night, the reaction mixture was cooled down to room temperature, diluted with a saturated aqueous solution of ammonium chloride, extracted with DCM. The organic layer was then dried over Na₂SO₄, filtered, and concentrated in vacuo. The resulting residue was purified by flash chromatography on silica gel (SiO₂ 40 g) eluting with DCM/MeOH/30% aq. NH₄OH (95/5/0.5) to give a solid which was triturated in 15 mL of i-Pr₂O. The resulting precipitate was filtered off to give 396 mg (79% yield) of tert-butyl 4-[2-oxo-6-[(2R)-2-(tetrahydropyran-2-yloxymethyl)morpholin-4-yl]-3H-imidazo[4,5-b]pyridin-1-yl]piperidine-1-carboxylate as a white solid. LC/MS (m/z, M+): calc. 517.3, found 517.0.

### Step 2: 6-[(2R)-2-(Hydroxymethyl)morpholin-4-yl]-1-(4-piperidyl)-3H-imidazo[4,5-b]pyridin-2-one, hydrochloride

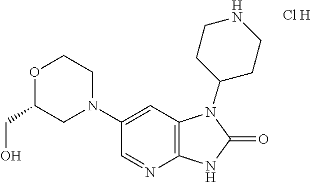

To a solution of tert-butyl 4-[2-oxo-6-[(2R)-2-(tetrahydropyran-2-yloxymethyl)morpholin-4-yl]-3H-imidazo[4,5-b]pyridin-1-yl]piperidine-1-carboxylate (390 mg, 0.75 mmol) in MeOH (30 mL) and DCM (5 mL) was added 2.82 mL of a 4N HCl solution in dioxane (11.3 mmol). The resulting reaction mixture was stirred at room temperature overnight, then concentrated in vacuo. The resulting residue was diluted in MeOH and concentrated in vacuo three times, and the resulting residue was triturated in i-PrOH (20 mL). The precipitate was filtered off and washed with ethyl acetate to give 284 mg (92% yield) of 6-[(2R)-2-(hydroxymethyl)morpholin-4-yl]-1-(4-piperidyl)-3H-imidazo[4,5-b]pyridin-2-one, hydrochloride as pale brown solid. LC/MS (m/z, M+): calc. 333.2, found 333.0 (free base).

### Step 3: 1-[1-[2-Amino-4-(trifluoromethoxy)benzoyl]-4-piperidyl]-6-[(2R)-2-(hydroxymethyl)morpholin-4-yl]-3H-imidazol[4,5-b]pyridin-2-one

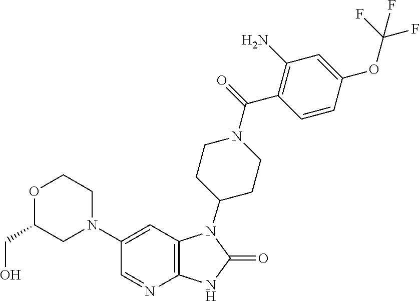 [SMILES: Nc1cc(OC(F)(F)F)ccc1C(=O)N1CCC(n2c(=O)[nH]c3ncc(N4CCO[C@@H](CO)C4)cc32)CC1]

To a suspension of 6-[(2R)-2-(hydroxymethyl)morpholin-4-yl]-1-(4-piperidyl)-3H-imidazo[4,5-b]pyridin-2-one, hydrochloride (180 mg, 0.44 mmol) in DMF (3 mL) was added DIPEA (0.54 mL, 400 mg, 3.1 mmol) and the resulting mixture was stirred at room temperature for 5 minutes. Then, 2-amino-4-(trifluoromethoxy)benzoic acid (98 mg, 0.44 mmol) followed by HATU (185 mg, 0.49 mmol) were added and the resulting solution was stirred at room temperature for 48 hours. After 48 hours, the reaction mixture was diluted with a 1 N aqueous solution of NaOH (5 mL), MeOH (5 mL) and toluene (5 mL), concentrated in vacuo. The resulting residue was diluted with MeOH and toluene and concentrated to dryness three times; the resulting residue was then purified by flash chromatography on Puriflash® interchim PF-15SiHP-F0012 (12 g) column eluting with DCM/MeOH/30% aq. NH₄OH (91.5/8.5/0.85) to give a solid which was triturated in i-Pr₂O. The precipitate was filtered off to furnish 164 mg (69% yield) of 1-[1-[2-amino-4-(trifluoromethoxy)benzoyl]-4-piperidyl]-6-[(2R)-2-(hydroxymethyl)morpholin-4-yl]-3H-imidazo[4,5-b]pyridin-2-one as a white solid. LC/MS (m/z, M+H): calc. 537.2, found 537.2; ¹H NMR (400 MHz, DMSO-d6, 30° C.) δ ppm 1.73 (br d, J=11 Hz, 2H), 2.21-2.40 (m, 2H), 2.40-2.52 (m partially hidden, 1H), 2.63-2.77 (m, 1H), 2.88-3.15 (m, 2H), 3.29-3.56 (m, 4H), 3.56-3.64 (m, 1H), 3.60-4.70 (m, 2H), 3.67 (td, J=11, 2 Hz, 1H), 3.95 (br dd, J=11, 1 Hz, 1H), 4.37-4.55 (m, 1H), 4.84 (t, J=6 Hz, 1H), 5.67 (s, 2H), 6.51 (br d, J=8 Hz, 1H), 6.66 (br s, 1H), 7.12 (d, J=8 Hz, 1H), 7.33 (d, J=2 Hz, 1H), 7.56 (d, J=2 Hz, 1H), 11.26 (br s, 1H)

### Example 14: 1-[1-[2-Amino-4-(pentafluoro-λ⁶-sulfanyl)benzoyl]-4-piperidyl]-6-tetrahydropyran-4-yl-3H-imidazo[4,5-b]pyridin-2-one

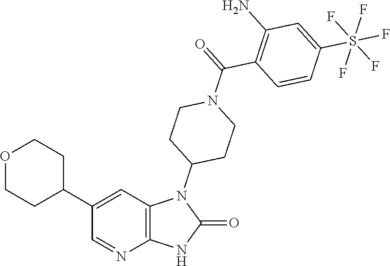 [SMILES: Nc1cc(S(F)(F)(F)(F)F)ccc1C(=O)N1CCC(n2c(=O)[nH]c3ncc(C4CCOCC4)cc32)CC1]

Example 14 was prepared following the procedure described in STEP 6 of Example 2, using 1-(4-piperidyl)-6-tetrahydropyran-4-yl-3H-imidazo[4,5-b]pyridin-2-one;hydrochloride (90 mg, 0.26 mmol) in DMF (1 mL), triethylamine (204 μL, 1.46 mmol), 2-amino-4-(pentafluoro-λ⁶-sulfanyl)benzoic acid; hydrochloride (81 mg, 0.27 mmol), TATU (103 mg, 0.32 mmol), stirred for 0.5 hours, to give 55 mg (38% yield) of 1-[1-[2-amino-4-(pentafluoro-λ⁶-sulfanyl)benzoyl]-4-piperidyl]-6-tetrahydropyran-4-yl-3H-imidazo[4,5-b]pyridin-2-one as a white solid. LC/MS (m/z, M+H): calc. 548.2, found 548.3; ¹H NMR (400 MHz, DMSO-d6, 100° C.) δ ppm 1.70-1.84 (m, 6H), 2.31 (m, 2H), 2.76-2.87 (m, 1H), 3.02-3.13 (m, 2H), 3.39-3.53 (m, 2H), 3.94-4.00 (m, 2H), 4.09-4.20 (m, 2H), 4.37-4.51 (m, 1H), 5.53 (br s, 2H), 7.01 (dd, J=8.4, 2.3 Hz, 1H), 7.23 (br d, J=8.4 Hz, 1H), 7.28 (d, J=2.3 Hz, 1H), 7.42 (d, J=1.8 Hz, 1H), 7.79 (d, J=1.8 Hz, 1H), 11.00-11.09 (m, 1H)

### Example 15: (rac)-6-Tetrahydrofuran-3-yl-1-[1-[4-(trifluoromethoxy)benzoyl]-4-piperidyl]-3H-imidazo[4,5-b]pyridin-2-one

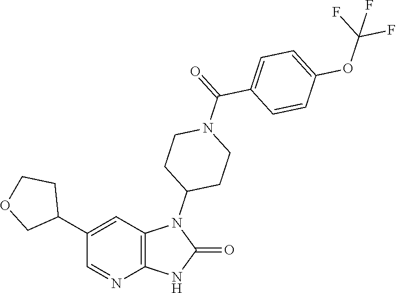 [SMILES: O=C(c1ccc(OC(F)(F)F)cc1)N1CCC(n2c(=O)[nH]c3ncc(C4CCOC4)cc32)CC1]

### Step 1: (rac)-tert-Butyl 4-[6-(2,5-dihydrofuran-3-yl)-2-oxo-3H-imidazo[4,5-b]pyridin-1-yl]piperidine-1-carboxylate

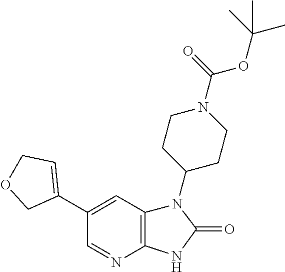 [SMILES: CC(C)(C)OC(=O)N1CCC(n2c(=O)[nH]c3ncc(C4=CCOC4)cc32)CC1]

To a solution of tert-butyl 4-(6-bromo-2-oxo-3H-imidazo[4,5-b]pyridin-1-yl)piperidine-1-carboxylate (400 mg, 1.01 mmol) and 2-(2,5-dihydrofuran-3-yl)-4,4,5,5-tetramethyl-1,3,2-dioxaborolane (237 mg, 1.21 mmol) in a dioxane/water mixture (8 mL/2 mL) was added K₃PO₄ (470 mg, 2.21 mmol), and the resulting reaction mixture was bubbled with argon for 5 minutes. Then, chloro(2-dicyclohexylphosphino-2′,6′-dimethoxy-1,1′-biphenyl)[2-(2′-amino-1,1′-biphenyl)]palladium(II) (73 mg, 0.10 mmol) was added and the resulting mixture was refluxed for 6 hours, cooled to room temperature, diluted with ethyl acetate, transferred to a separating funnel, and washed with a 1N aqueous solution of NaOH. The aqueous layer was then extracted with ethyl acetate. The combined organic layers were dried over Na₂SO₄, filtered, and concentrated in vacuo. The resulting residue was purified by flash chromatography (SiO₂ 12 g) eluting with DCM/MeOH 97/3 to give 273 mg (64% yield) of (rac)-tert-butyl 4-[6-(2,5-dihydrofuran-3-yl)-2-oxo-3H-imidazo[4,5-b]pyridin-1-yl]piperidine-1-carboxylate as a white solid. LC/MS (m/z, M+H): calc. 387.4, found 387.3.

### Step 2: (rac)-tert-Butyl 4-(2-oxo-6-tetrahydrofuran-3-yl-3H-imidazo[4,5-b]pyridin-1-yl)piperidine-1-carboxylate

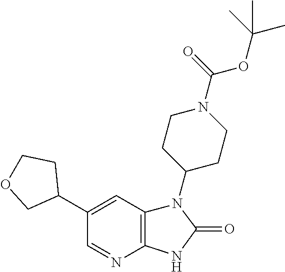 [SMILES: CC(C)(C)OC(=O)N1CCC(n2c(=O)[nH]c3ncc(C4CCOC4)cc32)CC1]

To a solution of (rac)-tert-butyl 4-[6-(2,5-dihydrofuran-3-yl)-2-oxo-3H-imidazo[4,5-b]pyridin-1-yl]piperidine-1-carboxylate (270 mg, 0.64 mmol) in a mixture of MeOH (10 mL) and DCM (10 mL) was added Pd/C (10%)-50% wet (40 mg). The whole mixture was then hydrogenated with 4 bars of H₂ at room temperature for 18 hours. The reaction mixture was then filtered and concentrated in vacuo. The resulting residue was purified by flash chromatography (SiO₂ 12 g) eluting with DCM/MeOH/NH₄OH (95/5/0.5) to give 240 mg (91% yield) of (rac)-tert-butyl 4-(2-oxo-6-tetrahydrofuran-3-yl-3H-imidazo[4,5-b]pyridin-1-yl)piperidine-1-carboxylate as a white solid. LC/MS (m/z, M+H): calc. 389.5, found 389.3.

### Step 3: (rac)-1-(4-Piperidyl)-6-tetrahydrofuran-3-yl-3H-imidazo[4,5-b]pyridin-2-one;hydrochloride

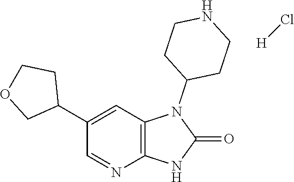

To a solution of (rac)-tert-butyl 4-(2-oxo-6-tetrahydrofuran-3-yl-3H-imidazo[4,5-b]pyridin-1-yl)piperidine-1-carboxylate (235 mg, 0.57 mmol) in MeOH (4 mL), was added 2.15 mL of a 4N solution of HCl in dioxane (8.62 mmol). The resulting mixture was stirred at room temperature for 1 hour, then concentrated in vacuo to give 208 mg (100% yield) of (rac)-1-(4-piperidyl)-6-tetrahydrofuran-3-yl-3H-imidazo[4,5-b]pyridin-2-one;hydrochloride as a white solid. LC/MS (m/z, M+H-HCl): calc. 289.2, found 289.3.

### Step 4: (rac)-6-Tetrahydrofuran-3-yl-1-[1-[4-(trifluoromethoxy)benzoyl]-4-piperidyl]-3H-imidazo[4,5-b]pyridin-2-one

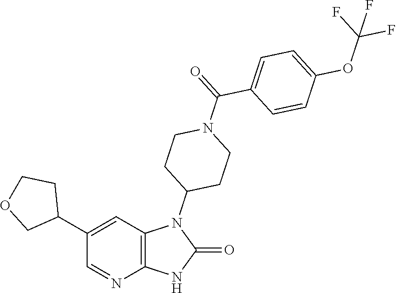 [SMILES: O=C(c1ccc(OC(F)(F)F)cc1)N1CCC(n2c(=O)[nH]c3ncc(C4CCOC4)cc32)CC1]

To a solution of (rac)-1-(4-piperidyl)-6-tetrahydrofuran-3-yl-3H-imidazo[4,5-b]pyridin-2-one;hydrochloride (103 mg, 0.28 mmol) in DMF (1.9 mL) was added DIPEA (148 mg, 0.20 mL, 1.14 mmol) and the resulting mixture was stirred for 5 minutes. Then, 4-(trifluoromethoxy)benzoic acid (65 mg, 0.31 mmol), TBTU (110 mg, 0.34 mmol) were successively added. The resulting mixture was then stirred at room temperature for 1.5 hours. The reaction mixture was diluted with ethyl acetate, washed with an aqueous saturated solution of NaHCO₃, then with water and brine. The combined organic layers were dried over Na₂SO₄, filtered, and concentrated in vacuo. The resulting residue was purified by flash chromatography (SiO₂ 12 g) eluting with DCM/MeOH (95/5) to give 89 mg (65% yield) of (rac)-6-tetrahydrofuran-3-yl-1-[1-[4-(trifluoromethoxy)benzoyl]-4-piperidyl]-3H-imidazo[4,5-b]pyridin-2-one as a white solid. LC/MS (m/z, M+H): calc. 477.2, found 477.2; ¹H NMR (400 MHz, DMSO-d6, 30° C.) δ ppm 1.64-1.90 (m, 2H), 1.92-2.04 (m, 1H), 2.16-2.36 (m, 3H), 2.7-3.8 (m, 3H), 3.35-3.49 (m, 1H), 3.59 (t, J=8.0 Hz, 1H), 3.77-3.87 (m, 1H), 3.95-4.07 (m, 2H), 4.41-4.54 (m, 1H), 4.59-4.73 (m, 1H), 7.47 (br d, J=8.8 Hz, 2H), 7.57 (d, J=1.8 Hz, 1H), 7.61 (br d, J=8.8 Hz, 2H), 7.84 (d, J=1.8 Hz, 1H), 11.48 (br s, 1H)

### Example 16: (rac)-1-[1-[2-Amino-4-(trifluoromethoxy)benzoyl]-4-piperidyl]-6-tetrahydrofuran-3-yl-3H-imidazo[4,5-b]pyridin-2-one

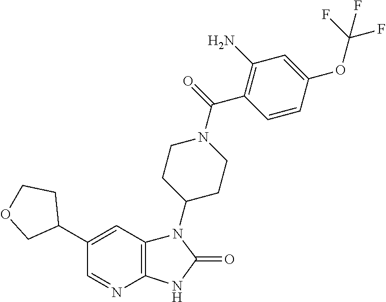 [SMILES: Nc1cc(OC(F)(F)F)ccc1C(=O)N1CCC(n2c(=O)[nH]c3ncc(C4CCOC4)cc32)CC1]

Example 16 was prepared following the procedure described in STEP 4 of Example 15, using (rac)-1-(4-piperidyl)-6-tetrahydrofuran-3-yl-3H-imidazo[4,5-b]pyridin-2-one;hydrochloride (103 mg, 0.28 mmol) in DMF (1.9 mL), DIPEA (148 mg, 0.2 mL, 1.14 mmol), and 2-amino-4-(trifluoromethoxy)benzoic acid (69 mg, 0.31 mmol) and TBTU (110 mg, 0.34 mmol) to give 92 mg (65% yield) of (rac)-1-[1-[2-amino-4-(trifluoromethoxy)benzoyl]-4-piperidyl]-6-tetrahydrofuran-3-yl-3H-imidazo[4,5-b]pyridin-2-one as a pale brown solid. LC/MS (m/z, M+H): calc. 492.2, found 492.3; ¹H NMR (400 MHz, DMSO-d6, 100° C.) δ ppm 1.77-1.88 (m, 2H), 1.94-2.05 (m, 1H), 2.22-2.44 (m, 3H), 3.09 (br td, J=13.0, 1.5 Hz, 2H), 3.40-3.51 (m, 1H), 3.61-3.67 (m, 1H), 3.81-3.89 (m, 1H), 3.97-4.10 (m, 2H), 4.19 (br d, J=13.0 Hz, 2H), 4.40-4.59 (m, 1H), 5.43 (br s, 2H), 6.47-6.59 (m, 1H), 6.72 (m, 1H), 7.16 (br d, J=8.4 Hz, 1H), 7.43 (d, J=1.8 Hz, 1H), 7.83 (br d, J=1.8 Hz, 1H), 11.05-11.19 (m, 1H)

### Example 17: 6-Cyclopentyl-1-[1-[4-(trifluoromethoxy)benzoyl]-4-piperidyl]-3H-imidazo[4,5-b]pyridin-2-one

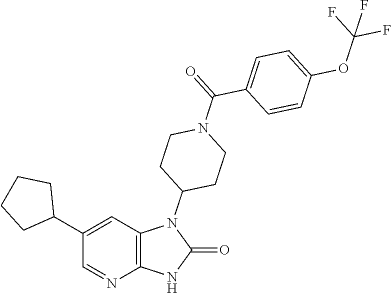 [SMILES: O=C(c1ccc(OC(F)(F)F)cc1)N1CCC(n2c(=O)[nH]c3ncc(C4CCCC4)cc32)CC1]

### Step 1: 6-(Cyclopenten-1-yl)-1-[1-[4-(trifluoromethoxy)benzoyl]-4-piperidyl]-3H-imidazo[4,5-b]pyridin-2-one

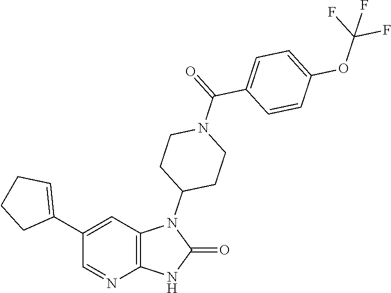 [SMILES: O=C(c1ccc(OC(F)(F)F)cc1)N1CCC(n2c(=O)[nH]c3ncc(C4=CCCC4)cc32)CC1]

A solution of 6-bromo-1-[1-[4-(trifluoromethoxy)benzoyl]-4-piperidyl]-3H-imidazo[4,5-b]pyridin-2-one (100 mg, 0.21 mmol), cyclopent-1-en-1-ylboronic acid (28 mg, 0.25 mmol) in dioxane (2 mL) and water (0.5 mL) was bubbled with argon for 5 minutes. Then potassium phosphate tribasic (109 mg, 0.51 mmol) and chloro(2-dicyclohexylphosphino-2′,6′-dimethoxy-1,1′-biphenyl)[2-(2′-amino-1,1′-biphenyl)]palladium(II) (15 mg, 0.021 mmol) were added and the resulting mixture was refluxed for 2 hours. Then, chloro(2-dicyclohexylphosphino-2′,6′-dimethoxy-1,1′-biphenyl)[2-(2′-amino-1,1′-biphenyl)]palladium(II) (5 mg, 0.007 mmol) was added, and the resulting solution was refluxed for one additional hour, then cooled to room temperature, diluted with ethyl acetate and water, and transferred into a separating funnel. The aqueous layer was extracted with ethyl acetate, and the combined organic layers were dried over Na₂SO₄, filtered, and concentrated in vacuo. The resulting residue was purified by flash chromatography (SiO₂ 12 g), eluting with DCM/MeOH 95/5, to give 120 mg (62% yield) of 6-(cyclopenten-1-yl)-1-[1-[4-(trifluoromethoxy)benzoyl]-4-piperidyl]-3H-imidazo[4,5-b]pyridin-2-one as a white solid. LC/MS (m/z, M+H): calc. 473.4, found 473.3.

### Step 2: 6-Cyclopentyl-1-[1-[4-(trifluoromethoxy)benzoyl]-4-piperidyl]-3H-imidazo[4,5-b]pyridin-2-one

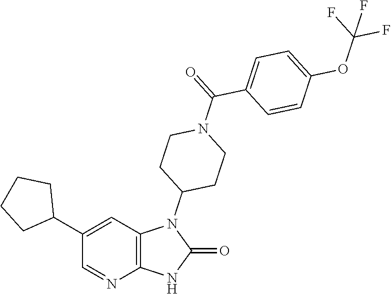 [SMILES: O=C(c1ccc(OC(F)(F)F)cc1)N1CCC(n2c(=O)[nH]c3ncc(C4CCCC4)cc32)CC1]

A solution of 6-(cyclopenten-1-yl)-1-[1-[4-(trifluoromethoxy)benzoyl]-4-piperidyl]-3H-imidazo[4,5-b]pyridin-2-one (120 mg, 0.25 mmol) in MeOH (4 mL) and DCM (4 mL) was bubbled with argon for 5 minutes. Then, Pd/C 10% wet (27 mg, 0.025 mmol) was added and the resulting mixture was hydrogenated under 3 bars of H₂ at room temperature for 18 hours. Then, Pd/C 10% wet (10 mg, 0.009 mmol) was added, and the resulting solution was hydrogenated under 3 bars of H₂ for 48 additional hours, filtered, washed with MeOH and DCM, and concentrated in vacuo to give 106 mg (88% yield) of 6-cyclopentyl-1-[1-[4-(trifluoromethoxy)benzoyl]-4-piperidyl]-3H-imidazo[4,5-b]pyridin-2-one as a white solid. LC/MS (m/z, M+H): calc. 475.5, found 475.2; ¹H NMR (400 MHz, DMSO-d6, 100° C.) δ ppm 1.51-1.89 (m, 8H), 1.98-2.14 (m, 2H), 2.19-2.40 (m, 2H), 2.99-3.19 (m, 3H), 4.08-4.28 (m, 2H), 4.38-4.49 (m, 1H), 7.36 (d, J=1.8 Hz, 1H), 7.40 (br d, J=8.6 Hz, 2H), 7.57 (br d, J=8.6 Hz, 2H), 7.78 (d, J=1.8 Hz, 1H), 10.88-11.16 (m, 1H)

### Example 18: 1-[1-[2-Hydroxy-4-(trifluoromethoxy)benzoyl]-4-piperidyl]-6-tetrahydropyran-4-yl-3H-imidazo[4,5-b]pyridin-2-one

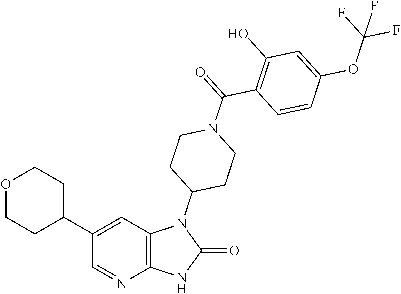 [SMILES: O=C(c1ccc(OC(F)(F)F)cc1O)N1CCC(n2c(=O)[nH]c3ncc(C4CCOCC4)cc32)CC1]

Under argon atmosphere, to a solution of 2-hydroxy-4-(trifluoromethoxy)benzoic acid (94 mg, 0.42 mmol) in DMF (3 mL) was added TBTU (148 mg, 0.46 mmol) and the resulting solution was stirred at room temperature for 2 hours. Then, this solution was added on a solution of 1-(4-piperidyl)-6-tetrahydropyran-4-yl-3H-imidazo[4,5-b]pyridin-2-one;hydrochloride (130 mg, 0.38 mmol) in DMF (2 mL) and the resulting reaction mixture was stirred at room temperature for 4 hours, then diluted with ethyl acetate and water, and transferred to a separating funnel. The aqueous layer was extracted with ethyl acetate 3 times. The combined organic layers were washed with water and with an aqueous saturated solution of NaHCO₃, dried over MgSO₄, filtered, and concentrated in vacuo. The resulting residue was purified by flash chromatography (SiO₂ 24 g) eluting with DCM/isopropanol 85/15 to give 55 mg (28% yield) of 1-[1-[2-hydroxy-4-(trifluoromethoxy)benzoyl]-4-piperidyl]-6-tetrahydropyran-4-yl-3H-imidazo[4,5-b]pyridin-2-one as a white solid. LC/MS (m/z, M+H): calc. 507.2, found 507.2; ¹H NMR (400 MHz, DMSO-d6, 100° C.) δ ppm 1.70-1.84 (m, 6H), 2.22-2.36 (m, 2H), 2.77-2.87 (m, 1H), 2.98-3.10 (m, 2H), 3.41-3.53 (m, 2H), 3.93-4.01 (m, 2H), 4.07-4.20 (m, 2H), 4.37-4.47 (m, 1H), 6.78-6.84 (m, 2H), 7.28 (d, J=8.1 Hz, 1H), 7.36 (d, J=1.8 Hz, 1H), 7.79 (d, J=1.8 Hz, 1H), 9.98-10.32 (m, 1H), 10.88-11.23 (m, 1H)

### Example 19: 1-[1-[4-(Pentafluoro-λ⁶-sulfanyl)benzoyl]-4-piperidyl]-6-tetrahydropyran-4-yl-3H-imidazo[4,5-b]pyridin-2-one

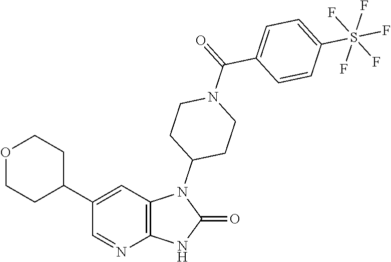 [SMILES: O=C(c1ccc(S(F)(F)(F)(F)F)cc1)N1CCC(n2c(=O)[nH]c3ncc(C4CCOCC4)cc32)CC1]

Example 19 was prepared following the procedure described in STEP 6 of Example 2, using 1-(4-piperidyl)-6-tetrahydropyran-4-yl-3H-imidazo[4,5-b]pyridin-2-one;hydrochloride (100 mg, 0.29 mmol) in DMF (1 mL), triethylamine (206 μL, 1.47 mmol), and 4-(pentafluoro-_⁶-sulfanyl)benzoic acid (75 mg, 0.3 mmol), TATU (114 mg, 0.35 mmol), stirred for 0.5 hours, to give 57 mg (36% yield) of 1-[1-[4-(pentafluoro-λ⁶-sulfanyl)benzoyl]-4-piperidyl]-6-tetrahydropyran-4-yl-3H-imidazo[4,5-b]pyridin-2-one as a white solid. LC/MS (m/z, M+H): calc. 533.2, found 533.2; ¹H NMR (400 MHz, DMSO-d6, 30° C.) δ ppm 1.64-1.90 (m, 6H), 2.19-2.41 (partially hidden m, 2H), 2.78-2.86 (m, 1H), 2.89-2.98 (m, 1H), 3.18-3.27 (partially hidden m, 1H), 3.44 (br td, J=11.5, 11.5, 2.0 Hz, 2H), 3.53-3.71 (m, 1H), 3.91-4.03 (m, 2H), 4.40-4.60 (m, 1H), 4.59-4.74 (m, 1H), 7.59 (d, J=1.8 Hz, 1H), 7.70 (br d, J=8.5 Hz, 2H), 7.81 (br d, J=1.8 Hz, 1H), 8.02 (br d, J=8.5 Hz, 2H), 11.41-11.47 (m, 1H)

### Example 20: 1-[1-[2-amino-4-(trifluoromethoxy)benzoyl]-4-piperidyl]-5-methyl-6-tetrahydropyran-4-yl-3H-imidazo[4,5-b]pyridin-2-one

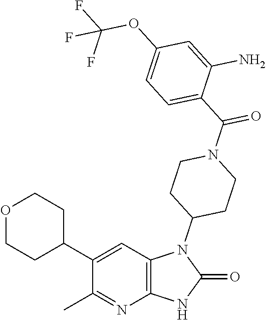 [SMILES: Cc1nc2[nH]c(=O)n(C3CCN(C(=O)c4ccc(OC(F)(F)F)cc4N)CC3)c2cc1C1CCOCC1]

### Step 1: 5-bromo-3-fluoro-6-methyl-pyridin-2-amine

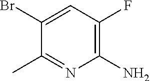

To a stirred solution of 3-fluoro-6-methyl-pyridin-2-amine (3 g, 23.8 mmol) in acetonitrile (90 mL) was added 1-bromopyrrolidine-2,5-dione (4.23 g, 23.8 mmol) at 0° C. The reaction mixture was stirred at room temperature for 1 hour under argon atmosphere. After one hour, the reaction mixture was diluted with ice water (100 mL) and extracted with ethyl acetate (100 mL×2). The combined organic layers were dried over Na₂SO₄, filtered, and concentrated under reduced pressure. The resulting residue was purified by flash chromatography to give 3 g (62% yield) of 5-bromo-3-fluoro-6-methyl-pyridin-2-amine as an off white solid. LC/MS (m/z, M+H): calc. 206.0, found 206.7.

### Step 2: 3-bromo-5-fluoro-2-methyl-6-nitro-pyridine

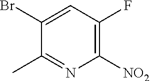

To a stirred solution of hydrogen peroxide (2.49 g, 73.2 mmol) was added sulfuric acid (60 mL) at 0° C. The reaction mixture was then stirred at room temperature for 1.5 hours under argon atmosphere. After 1.5 hours, 5-bromo-3-fluoro-6-methyl-pyridin-2-amine (3 g, 14.6 mmol) was added at 0° C. The reaction mixture was stirred for 2 hours at room temperature and then was diluted with ice water (500 mL) and extracted with ethyl acetate (100 mL×2). The combined organic layers were dried over Na₂SO₄, filtered and concentrated under the reduced pressure. The resulting residue was purified by flash chromatography to give 1.5 g (44% yield) of 3-bromo-5-fluoro-2-methyl-6-nitro-pyridine as a yellow solid.

### Step 3: tert-butyl 4-[(5-bromo-6-methyl-2-nitro-3-pyridyl)amino]piperidine-1-carboxylate

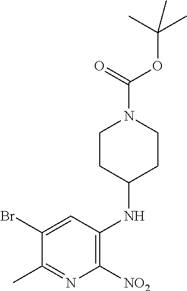 [SMILES: Cc1nc([N+](=O)[O-])c(NC2CCN(C(=O)OC(C)(C)C)CC2)cc1Br]

To a stirred solution of tert-butyl 4-aminopiperidine-1-carboxylate (1.5 g, 7.49 mmol) and 3-bromo-5-fluoro-2-methyl-6-nitro-pyridine (1.5 g, 6.38 mmol) in tetrahydrofuran (30 mL) was added potassium carbonate (3.11 g, 22.5 mmol) at room temperature. The reaction mixture was stirred at 90° C. for 16 hours. After 16 hours, the reaction mixture was cooled down to room temperature, concentrated under reduced pressure and the resulting residue was diluted with ice water (30 mL) and extracted with ethyl acetate (20 mL×2). The combined organic layers were dried over Na₂SO₄, filtered and concentrated under reduced pressure. The resulting residue was purified by flash chromatography to give 1.5 g (56% yield) of tert-butyl 4-[(5-bromo-6-methyl-2-nitro-3-pyridyl)amino]piperidine-1-carboxylate as a yellow solid. LC/MS (m/z, M+H): calc. 416.3, found 416.8.

### Step 4: tert-butyl 4-[(2-amino-5-bromo-6-methyl-3-pyridyl)amino]piperidine-1-carboxylate

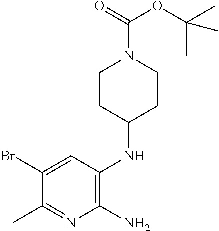 [SMILES: Cc1nc(N)c(NC2CCN(C(=O)OC(C)(C)C)CC2)cc1Br]

To a stirred solution of tert-butyl 4-[(5-bromo-6-methyl-2-nitro-3-pyridyl)amino]piperidine-1-carboxylate (1.50 g, 3.61 mmol) in propan-2-ol (60 mL) was added 4,4,5,5-tetramethyl-2-(4,4,5,5-tetramethyl-1,3,2-dioxaborolan-2-yl)-1,3,2-dioxaborolane (4.59 g, 18.1 mmol) followed by potassium 2-methylpropan-2-olate (1.22 g, 10.8 mmol) at 0° C. The reaction mixture was then stirred for 2 hours at 110° C. After 2 hours, the reaction mixture was cooled down to room temperature and concentrated under reduced pressure. The resulting residue was diluted with ice water (100 mL) and extracted with ethyl acetate (100 mL×2). The combined organic layers were dried over Na₂SO₄, filtered and concentrated under reduced pressure. The resulting residue was purified by flash chromatography to give 1.39 g (100% yield) of tert-butyl 4-[(2-amino-5-bromo-6-methyl-3-pyridyl)amino]piperidine-1-carboxylate as a blue colour solid. LC/MS (m/z, M+H): calc. 386.3, found 387.0.

### Step 5: tert-butyl 4-(6-bromo-5-methyl-2-oxo-3H-imidazo[4,5-b]pyridin-1-yl)piperidine-1-carboxylate

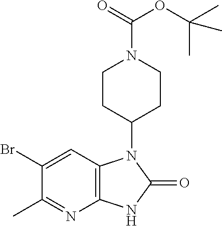 [SMILES: Cc1nc2[nH]c(=O)n(C3CCN(C(=O)OC(C)(C)C)CC3)c2cc1Br]

A stirred solution of tert-butyl 4-[(2-amino-5-bromo-3-pyridyl)amino]piperidine-1-carboxylate (1.50 g, 3.88 mmol) and di(imidazol-1-yl)methanone (3.28 g, 20.2 mmol) in 1,4-dioxane (20 mL) was stirred for 2 hours at 90° C. After 2 hours, the reaction mixture was cooled down to room temperature and concentrated under reduced pressure. The resulting residue was diluted with ice water (100 mL) and extracted with ethyl acetate (200 mL×2).

The combined organic layers were dried over Na₂SO₄, filtered and concentrated under reduced pressure. The resulting residue was purified by flash chromatography to give 1.5 g (94% yield) of tert-butyl 4-(6-bromo-2-oxo-3H-imidazo[4,5-b]pyridin-1-yl)piperidine-1-carboxylate as an off white solid. LC/MS (m/z, M+H): calc. 411.1, found 410.9.

### Step 6: tert-butyl 4-[6-(3,6-dihydro-2H-pyran-4-yl)-5-methyl-2-oxo-3H-imidazo[4,5-b]pyridin-1-yl]piperidine-1-carboxylate

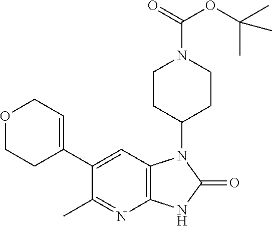 [SMILES: Cc1nc2[nH]c(=O)n(C3CCN(C(=O)OC(C)(C)C)CC3)c2cc1C1=CCOCC1]

To a stirred solution of 2-(3,6-dihydro-2H-pyran-4-yl)-4,4,5,5-tetramethyl-1,3,2-dioxaborolane (750 mg, 3.57 mmol) and tert-butyl 4-(6-bromo-5-methyl-2-oxo-3H-imidazo[4,5-b]pyridin-1-yl)piperidine-1-carboxylate (750 mg, 1.82 mmol) in 1,4-dioxane (10 mL) and water (2 mL) was added disodium carbonate (378 mg, 3.57 mmol) and the resulting mixture was degassed under argon flux for 15 minutes. Then, PdCl₂(dppf) (261 mg, 0.357 mmol) was added and the reaction mixture was stirred at 90° C. for 16 hours. After 16 hours, the reaction mixture was cooled down to room temperature and then filtered through celite bed. The filtrate was diluted with ethyl acetate (50 mL), washed with water (50 mL), and the combined organic layers were dried over anhydrous Na₂SO₄ and concentrated under reduced pressure. The crude compound (30 g) was adsorbed over silica gel and purified by flash chromatography to give 350 mg (46% yield) of tert-butyl 4-[6-(3,6-dihydro-2H-pyran-4-yl)-5-methyl-2-oxo-3H-imidazo[4,5-b]pyridin-1-yl]piperidine-1-carboxylate as an off white solid. LC/MS (m/z, M+H): calc. 415.2, found 415.3.

### Step 7: 6-(3,6-dihydro-2H-pyran-4-yl)-5-methyl-1-(4-piperidyl)-3H-imidazo[4,5-b]pyridin-2-one, dihydrochloride

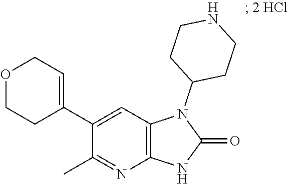 [SMILES: Cc1nc2[nH]c(=O)n(C3CCNCC3)c2cc1C1=CCOCC1]

To a stirred solution of tert-butyl 4-[6-(3,6-dihydro-2H-pyran-4-yl)-5-methyl-2-oxo-3H-imidazo[4,5-b]pyridin-1-yl]piperidine-1-carboxylate (300 mg, 0.72 mmol) in dichloromethane (5 mL) was added a 4 M hydrogen chloride solution in dioxane (4.00 mol/L, 1.81 mL, 7.24 mmol) under argon atmosphere and then the reaction mixture was stirred at room temperature for 1 hour. After 1 hour, the reaction mixture was concentrated under reduced pressure and the resulting residue was triturated with diethyl ether (50 mL), filtered, to give 300 mg (quantitative yield) of 6-(3,6-dihydro-2H-pyran-4-yl)-5-methyl-1-(4-piperidyl)-3H-imidazo[4,5-b]pyridin-2-one, dihydrochloride as an off white solid. LC/MS (m/z, M+H-2 HCl): calc. 315.2, found 315.0.

### Step 8: 1-[1-[2-amino-4-(trifluoromethoxy)benzoyl]-4-piperidyl]-6-(3,6-dihydro-2H-pyran-4-yl)-5-methyl-3H-imidazo[4,5-b]pyridin-2-one

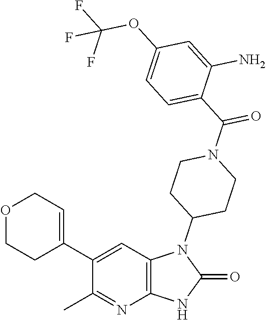 [SMILES: Cc1nc2[nH]c(=O)n(C3CCN(C(=O)c4ccc(OC(F)(F)F)cc4N)CC3)c2cc1C1=CCOCC1]

To a stirred solution of 2-amino-4-(trifluoromethoxy)benzoic acid (200 mg, 0.90 mmol) and 6-(3,6-dihydro-2H-pyran-4-yl)-5-methyl-1-(4-piperidyl)-3H-imidazo[4,5-b]pyridin-2-one, dihydrochloride (0.284 g, 0.73 mmol) in N,N-dimethylformamide (5 mL) was added N,N-diethylethanamine (275 mg, 2.71 mmol) and [benzotriazol-1-yloxy(dimethylamino)methylene]-dimethyl-ammonium;tetrafluoroborate (348 mg, 1.09 mmol) at 0° C. The reaction mixture was stirred at room temperature for 1 hour under argon atmosphere. After one hour, the reaction mixture was concentrated under reduced pressure and the resulting residue was diluted with ice water (100 mL) and extracted with ethyl acetate (100 mL×2). The combined organic layers were dried over Na₂SO₄, filtered and concentrated under reduced pressure. The resulting residue was purified by flash chromatography to give 200 mg (53% yield) of (1-[1-[2-amino-4-(trifluoromethoxy)benzoyl]-4-piperidyl]-6-(3,6-dihydro-2H-pyran-4-yl)-5-methyl-3H-imidazo[4,5-b]pyridin-2-one as a yellow solid. LC/MS (m/z, M+H): calc. 518.2, found 518.4.

### Step 9: 1-[1-[2-amino-4-(trifluoromethoxy)benzoyl]-4-piperidyl]-5-methyl-6-tetrahydropyran-4-yl-3H-imidazo[4,5-b]pyridin-2-one

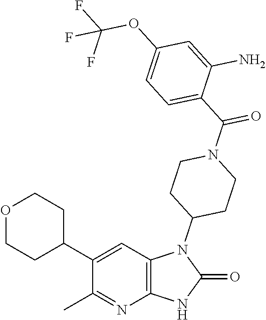 [SMILES: Cc1nc2[nH]c(=O)n(C3CCN(C(=O)c4ccc(OC(F)(F)F)cc4N)CC3)c2cc1C1CCOCC1]

To a stirred solution of 1-[1-[2-amino-4-(trifluoromethoxy)benzoyl]-4-piperidyl]-6-(3,6-dihydro-2H-pyran-4-yl)-5-methyl-3H-imidazo[4,5-b]pyridin-2-one (200 mg, 0.39 mmol) in methanol (10 mL) was added Pd/C (206 mg, 1.93 mmol) and a catalytic amount of acetic acid (2.3 mg, 0.039 mmol) under an inert atmosphere and then the reaction mixture was stirred under 1 atmosphere of hydrogen at room temperature for 16 hours. After 16 hours, the reaction mixture was filtered through a celite bed and washed with ethanol and concentrated to dryness. The resulting residue was purified by preparative HPLC to give 67 mg (33% yield) of 1-[1-[2-amino-4-(trifluoromethoxy)benzoyl]-4-piperidyl]-5-methyl-6-tetrahydropyran-4-yl-3H-imidazo[4,5-b]pyridin-2-one as an off white solid. LC/MS (m/z, M+H): calc. 520.2, found 520.1. ¹H NMR (400 MHz, DMSO-d6) δ ppm 11.25-11.34 (m, 1H) 7.37-7.52 (m, 1H) 7.07-7.18 (m, 1H) 6.63-6.73 (m, 1H) 6.42-6.57 (m, 1H) 5.64-5.79 (m, 2H) 4.37-4.55 (m, 1H) 3.89-4.05 (m, 2H) 3.40-3.56 (m, 2H) 2.86-3.12 (m, 3H) 2.42-2.46 (m, 3H) 2.30-2.35 (m, 1H) 1.76-1.87 (m, 2H) 1.69-1.76 (m, 2H) 1.54-1.63 (m, 2H).

### Example 21: 1-[1-[2-amino-4-(trifluoromethoxy)benzoyl]-4-piperidyl]-6-(2-fluorospiro[3.3]heptan-6-yl)-3H-imidazo[4,5-b]pyridin-2-one

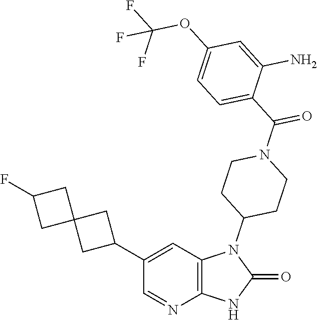 [SMILES: Nc1cc(OC(F)(F)F)ccc1C(=O)N1CCC(n2c(=O)[nH]c3ncc(C4CC5(CC(F)C5)C4)cc32)CC1]

Example 21 was prepared following the procedure described for the preparation of Example 40 using 2-(2-fluorospiro[3.3]heptan-6-yl)-4,4,5,5-tetramethyl-1,3,2-dioxaborolane (60 mg, 0.25 mmol in step 1) to give 51 mg (38% yield over two steps) of (1-[1-[2-amino-4-(trifluoromethoxy)benzoyl]-4-piperidyl]-6-(2-fluorospiro[3.3]heptan-6-yl)-3H-imidazo[4,5-b]pyridin-2-one as a solid. LC/MS (m/z, M+H): calc. 534.2, found 534.3; ¹H NMR (400 MHz, DMSO-d6, 27° C.) δ ppm 1.75 (br d, J=11.1 Hz, 2H) 2.09-2.43 (m, 10H) 2.60-2.69 (m, 1H) 2.95-3.13 (m, 2H) 3.40-3.43 (m partially hidden, 2H) 4.41-4.51 (m, 1H) 5.00 (dquin, J=55.9, 6.7, 6.7, 6.7, 6.7 Hz, 1H) 6.51 (dd, J=8.4, 1.2 Hz, 1H) 6.69 (d, J=1.2 Hz, 1H) 7.14 (d, J=8.4 Hz, 1H) 7.48 (d, J=1.5 Hz, 1H) 7.76 (d, J=1.5 Hz, 1H) 11.44 (br s, 1H).

### Example 22: (rac)-1-[1-[2-amino-4-(trifluoromethoxy)benzoyl]-4-piperidyl]-6-(2-oxo-4-piperidyl)-3H-imidazo[4,5-b]pyridin-2-one

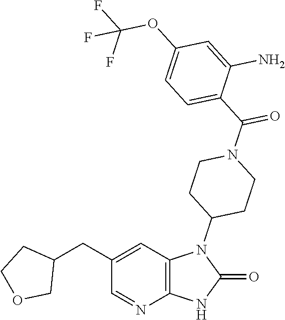 [SMILES: Nc1cc(OC(F)(F)F)ccc1C(=O)N1CCC(n2c(=O)[nH]c3ncc(CC4CCOC4)cc32)CC1]

Example 22 was prepared following the procedure described for the preparation of Example 40 using 4-(4,4,5,5-tetramethyl-1,3,2-dioxaborolan-2-yl)piperidin-2-one (56 mg, 0.25 mmol in step 1) to give 51 mg (39% yield over two steps) of (rac)-1-[1-[2-amino-4-(trifluoromethoxy)benzoyl]-4-piperidyl]-6-(2-oxo-4-piperidyl)-3H-imidazo[4,5-b]pyridin-2-one as a solid. LC/MS (m/z, M+H): calc. 519.2, found 519.3; ¹H NMR (400 MHz, DMSO-d6, 27° C.) δ ppm 1.75 (br d, J=11.1 Hz, 2H) 1.86-1.96 (m, 2H) 2.20-2.37 (m, 2H) 2.37-2.45 (m, 2H) 2.94-3.13 (m, 3H) 3.23-3.28 (m, 2H) 3.39-3.43 (m hidden, 2H) 4.41-4.51 (m, 1H) 6.51 (br d, J=8.4 Hz, 1H) 6.67 (br s, 1H) 7.14 (d, J=8.4 Hz, 1H) 7.56-7.59 (m, 1H) 7.60 (d, J=1.5 Hz, 1H) 7.83 (d, J=1.5 Hz, 1H) 11.49 (br s, 1H).

### Example 23: (rac)-1-[1-[2-amino-4-(trifluoromethoxy)benzoyl]-4-piperidyl]-6-(tetrahydrofuran-3-ylmethyl)-3H-imidazo[4,5-b]pyridin-2-one

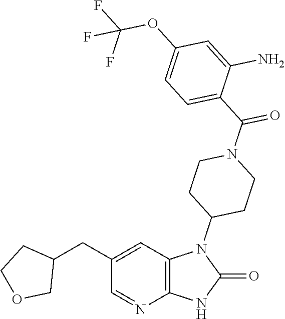 [SMILES: Nc1cc(OC(F)(F)F)ccc1C(=O)N1CCC(n2c(=O)[nH]c3ncc(CC4CCOC4)cc32)CC1]

Example 23 was prepared following the procedure described for the preparation of Example 40 using 4,4,5,5-tetramethyl-2-(tetrahydrofuran-3-ylmethyl)-1,3,2-dioxaborolane (53 mg, 0.25 mmol in step 1) to give 50 mg (40% yield over two steps) of (rac)-1-[1-[2-amino-4-(trifluoromethoxy)benzoyl]-4-piperidyl]-6-(tetrahydrofuran-3-ylmethyl)-3H-imidazo[4,5-b]pyridin-2-one as a solid. LC/MS (m/z, M+H): calc. 506.2, found 506.3; ¹H NMR (400 MHz, DMSO-d6, 27° C.) δ ppm 1.50-1.61 (m, 1H) 1.75 (br d, J=11.1 Hz, 2H) 1.85-1.94 (m, 1H) 2.19-2.34 (m, 2H) 2.42-2.48 (m, 1H) 2.60-2.70 (m, 2H) 2.93-3.12 (m, 2H) 3.32-3.38 (m partially hidden, 3H) 3.63-3.67 (m, 1H) 3.68-3.74 (m, 1H) 3.74-3.81 (m, 1H) 4.38-4.52 (m, 1H) 6.51 (br d, J=8.4 Hz, 1H) 6.67 (br s, 1H) 7.15 (d, J=8.4 Hz, 1H) 7.58 (d, J=1.6 Hz, 1H) 7.76 (d, J=1.6 Hz, 1H) 11.46 (br s, 1H).

### Example 24: 1-[1-[2-amino-4-(trifluoromethoxy)benzoyl]-4-piperidyl]-6-(2-methoxyethyl)-3H-imidazo[4,5-b]pyridin-2-one

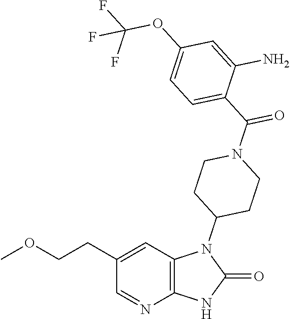 [SMILES: COCCc1cnc2[nH]c(=O)n(C3CCN(C(=O)c4ccc(OC(F)(F)F)cc4N)CC3)c2c1]

Example 24 was prepared following the procedure described for the preparation of Example 40 using 2-(2-methoxyethyl)-4,4,5,5-tetramethyl-1,3,2-dioxaborolane (46 mg, 0.25 mmol in step 1) to give 60 mg (50% yield over two steps) of 1-[1-[2-amino-4-(trifluoromethoxy)benzoyl]-4-piperidyl]-6-(2-methoxyethyl)-3H-imidazo[4,5-b]pyridin-2-one as a solid. LC/MS (m/z, M+H): calc. 480.2, found 480.2; ¹H NMR (400 MHz, DMSO-d6, 27° C.) δ ppm 1.75 (br d, J=11.1 Hz, 2H) 2.19-2.34 (m, 2H) 2.82 (t, J=6.8 Hz, 2H) 2.94-3.13 (m, 2H) 3.26 (s, 3H) 3.40-3.43 (m hidden, 2H) 3.55-3.56 (m partially hidden, 2H) 4.41-4.51 (m, 1H) 6.51 (br d, J=8.4 Hz, 1H) 6.67 (br s, 1H) 7.15 (d, J=8.4 Hz, 1H) 7.60 (d, J=1.5 Hz, 1H) 7.78 (d, J=1.5 Hz, 1H) 11.45 (br s, 1H).

### Example 25: (rac)-1-[1-[2-amino-4-(trifluoromethoxy)benzoyl]-4-piperidyl]-6-tetrahydrofuran-2-yl-3H-imidazo[4,5-b]pyridin-2-one

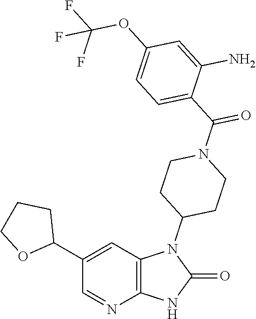 [SMILES: Nc1cc(OC(F)(F)F)ccc1C(=O)N1CCC(n2c(=O)[nH]c3ncc(C4CCCO4)cc32)CC1]

Example 25 was prepared following the procedure described for the preparation of Example 40 using 4,4,5,5-tetramethyl-2-tetrahydrofuran-2-yl-1,3,2-dioxaborolane (50 mg, 0.25 mmol in step 1) to give 56 mg (45% yield over two steps) of (rac)-1-[1-[2-amino-4-(trifluoromethoxy)benzoyl]-4-piperidyl]-6-tetrahydrofuran-2-yl-3H-imidazo[4,5-b]pyridin-2-one as a solid. LC/MS (m/z, M+H): calc. 492.2, found 492.2; ¹H NMR (400 MHz, DMSO-d6, 27° C.) δ ppm 1.68-1.85 (m, 3H) 1.90-2.06 (m, 2H) 2.16-2.37 (m, 3H) 2.95-3.15 (m, 2H) 3.40-3.46 (m hidden, 2H) 3.78-3.83 (m, 1H) 3.99-4.05 (m, 1H) 4.42-4.52 (m, 1H) 4.81 (t, J=7.2 Hz, 1H) 6.51 (br d, J=8.4 Hz, 1H) 6.67 (br s, 1H) 7.14 (d, J=8.4 Hz, 1H) 7.54 (d, J=1.5 Hz, 1H) 7.88 (d, J=1.5 Hz, 1H) 11.53 (br s, 1H).

### Examples 26 & 27: trans-6-(4-methoxycyclohexyl)-1-[1-[4-(trifluoromethoxy)benzoyl]-4-piperidyl]-3H-imidazo[4,5-b]pyridin-2-one & cis-6-(4-methoxycyclohexyl)-1-[1-[4-(trifluoromethoxy)benzoyl]-4-piperidyl]-3H-imidazo[4,5-b]pyridin-2-one

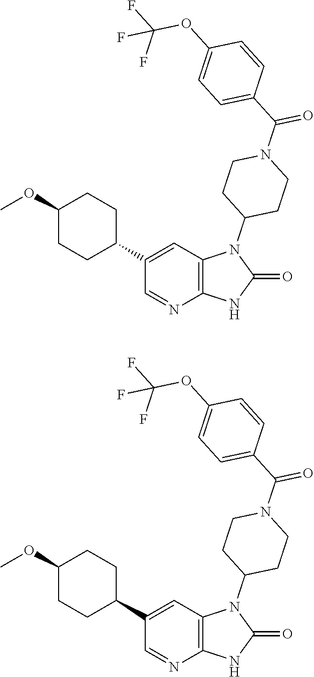 [SMILES: CO[C@H]1CC[C@H](c2cnc3[nH]c(=O)n(C4CCN(C(=O)c5ccc(OC(F)(F)F)cc5)CC4)c3c2)CC1 ; CO[C@H]1CC[C@@H](c2cnc3[nH]c(=O)n(C4CCN(C(=O)c5ccc(OC(F)(F)F)cc5)CC4)c3c2)CC1]

### Step 1: 6-(4-methoxycyclohexen-1-yl)-1-[1-[4-(trifluoromethoxy)benzoyl]-4-piperidyl]-3H-imidazo[4,5-b]pyridin-2-one

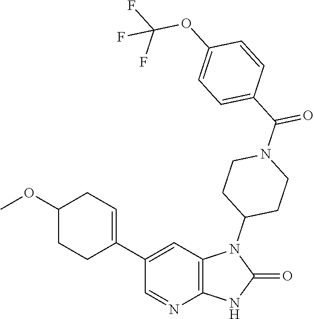 [SMILES: COC1CC=C(c2cnc3[nH]c(=O)n(C4CCN(C(=O)c5ccc(OC(F)(F)F)cc5)CC4)c3c2)CC1]

Step 1 of Examples 26 & 27 was performed following the protocol described in step 1 of Example 17 using 6-bromo-1-[1-[4-(trifluoromethoxy)benzoyl]-4-piperidyl]-3H-imidazo[4,5-b]pyridin-2-one (200 mg, 0.41 mmol, Example 60) and 2-(4-methoxycyclohex-1-en-1-yl)-4,4,5,5-tetramethyl-1,3,2-dioxaborolane (118 mg, 0.49 mmol) to give 166 mg (78% yield) of 6-(4-methoxycyclohexen-1-yl)-1-[1-[4-(trifluoromethoxy)benzoyl]-4-piperidyl]-3H-imidazo[4,5-b]pyridin-2-one as a white solid. LC/MS (m/z, M+H-TFA): calc. 517.2, found 517.2.

### Step 2: cis-6-(4-methoxycyclohexyl)-1-[1-[4-(trifluoromethoxy)benzoyl]-4-piperidyl]-3H-imidazo[4,5-b]pyridin-2-one & trans-6-(4-methoxycyclohexyl)-1-[1-[4-(trifluoromethoxy)benzoyl]-4-piperidyl]-3H-imidazo[4,5-b]pyridin-2-one

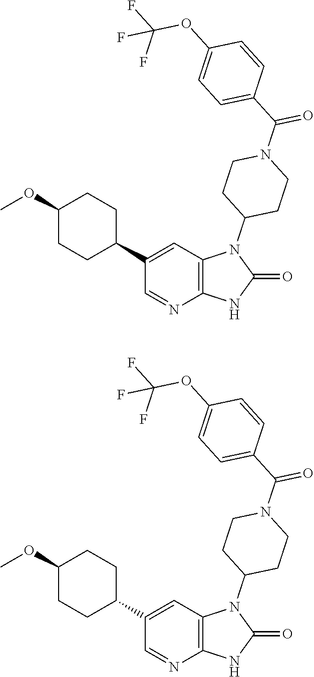 [SMILES: CO[C@H]1CC[C@@H](c2cnc3[nH]c(=O)n(C4CCN(C(=O)c5ccc(OC(F)(F)F)cc5)CC4)c3c2)CC1 ; CO[C@H]1CC[C@H](c2cnc3[nH]c(=O)n(C4CCN(C(=O)c5ccc(OC(F)(F)F)cc5)CC4)c3c2)CC1]

Step 2 of Examples 26 & 27 was performed following the protocol described in step 2 of Example 17 using 6-(4-methoxycyclohexen-1-yl)-1-[1-[4-(trifluoromethoxy)benzoyl]-4-piperidyl]-3H-imidazo[4,5-b]pyridin-2-one (164 mg, 0.32 mmol) to give: 9 mg (5% yield) of trans-6-(4-methoxycyclohexyl)-1-[1-[4-(trifluoromethoxy)benzoyl]-4-piperidyl]-3H-imidazo[4,5-b]pyridin-2-one as a white solid. LC/MS (m/z, M+H-TFA): calc. 519.2, found 519.5; ¹H NMR (400 MHz, DMSO-d6, 100° C.) d ppm 1.25-1.36 (m, 2H), 1.51-1.63 (m, 2H), 1.82 (br d, J=12.7 Hz, 2H), 1.90 (br d, J=12.7 Hz, 2H), 2.13 (br d, J=12.7 Hz, 2H), 2.26-2.36 (m, 2H), 2.54-2.63 (m, 1H), 3.07-3.15 (m, 2H), 3.18-3.28 (m, 1H), 3.30 (s, 3H), 4.13-4.30 (m, 2H), 4.39-4.48 (m, 1H), 7.39 (d, J=1.8 Hz, 1H), 7.42 (br d, J=8.4 Hz, 2H), 7.59 (d, J=8.4 Hz, 2H), 7.79 (d, J=1.8 Hz, 1H), 10.99-11.08 (m, 1H); and

43 mg (26% yield) of cis-6-(4-methoxycyclohexyl)-1-[1-[4-(trifluoromethoxy)benzoyl]-4-piperidyl]-3H-imidazo[4,5-b]pyridin-2-one as a white solid. LC/MS (m/z, M+H-TFA): calc. 519.2, found 519.5; ¹H NMR (400 MHz, DMSO-d6, 100° C.) d ppm 1.51-1.65 (m, 4H), 1.70-1.86 (m, 4H), 1.99 (br d, J=12.7 Hz, 2H), 2.25-2.35 (m, 2H), 2.59-2.67 (m, 1H), 3.06-3.17 (m, 2H), 3.28 (s, 3H), 3.46-3.52 (m, 1H), 4.09-4.30 (m, 2H), 4.41-4.51 (m, 1H), 7.34 (d, J=1.8 Hz, 1H), 7.41 (br d, J=8.4 Hz, 2H), 7.58 (br d, J=8.4 Hz, 2H), 7.77 (d, J=1.8 Hz, 1H), 10.92-11.13 (m, 1H).

### Example 28: (rac)-1-[1-[2-amino-4-(trifluoromethoxy)benzoyl]-4-piperidyl]-6-(3-azabicyclo[4.1.0]heptan-6-yl)-3H-imidazo[4,5-b]pyridin-2-one, 2,2,2-trifluoroacetic acid

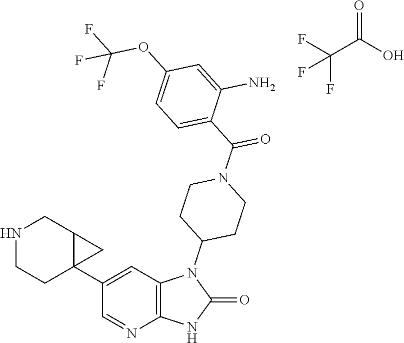 [SMILES: Nc1cc(OC(F)(F)F)ccc1C(=O)N1CCC(n2c(=O)[nH]c3ncc(C45CCNCC4C5)cc32)CC1 ; O=C(O)C(F)(F)F]

Example 28 was prepared following the procedure described for the preparation of Example 34 using 6-(4,4,5,5-tetramethyl-1,3,2-dioxaborolan-2-yl)-3-azabicyclo[4.1.0]heptane, hydrochloride (65 mg, 0.25 mmol in step 1) to give 3 mg (2% yield over two steps) of (rac)-1-[1-[2-amino-4-(trifluoromethoxy)benzoyl]-4-piperidyl]-6-(3-azabicyclo[4.1.0]heptan-6-yl)-3H-imidazo[4,5-b]pyridin-2-one, 2,2,2-trifluoroacetic acid as a solid. LC/MS (m/z, M+H-TFA): calc. 517.2, found 517.3; ¹H NMR (400.23 MHz, DMSO-d6) δ ppm 8.36 (br s, 2H), 7.14 (br d, J=8.44 Hz, 2H), 6.68 (s, 2H), 6.52 (br d, J=9.78 Hz, 1H), 3.67 (br d, J=7.70 Hz, 4H), 3.62 (br d, J=4.16 Hz, 1H), 3.15 (m, 2H), 2.50 (u), 2.33 (s, 1H), 2.25 (br d, J=11.74 Hz, 2H), 2.18 (s, 1H), 1.79 (br d, J=14.06 Hz, 3H), 1.25 (m, 5H), 1.16 (br d, J=3.18 Hz, 1H), 1.11 (br dd, J=17.24, 11.49 Hz, 2H).

### Example 29: 1-[1-[2-amino-4-(trifluoromethoxy)benzoyl]-4-piperidyl]-6-cyclobutyl-3H-imidazo[4,5-b]pyridin-2-one

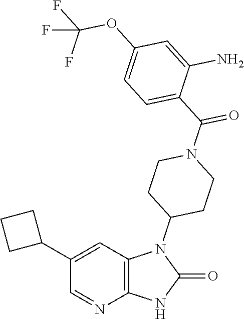 [SMILES: Nc1cc(OC(F)(F)F)ccc1C(=O)N1CCC(n2c(=O)[nH]c3ncc(C4CCC4)cc32)CC1]

Example 29 was prepared following the procedure described for the preparation of Example 34 using 2-cyclobutyl-4,4,5,5-tetramethyl-1,3,2-dioxaborolane (45 mg, 0.25 mmol in step 1) to give 56 mg (47% yield over two steps) of 1-[1-[2-amino-4-(trifluoromethoxy)benzoyl]-4-piperidyl]-6-cyclobutyl-3H-imidazo[4,5-b]pyridin-2-one as a solid. LC/MS (m/z, M+H): calc. 476.2, found 476.2; ¹H NMR (400 MHz, DMSO-d6, 27° C.) δ ppm 1.75 (br d, J=11.1 Hz, 2H) 1.80-1.88 (m, 1H) 1.91-2.02 (m, 1H) 2.10-2.22 (m, 2H) 2.23-2.36 (m, 4H) 2.97-3.13 (m, 2H) 3.50-3.58 (m partially hidden, 3H) 4.42-4.53 (m, 1H) 6.51 (dd, J=8.4, 1.2 Hz, 1H) 6.68 (d, J=1.2 Hz, 1H) 7.14 (d, J=8.4 Hz, 1H) 7.54 (d, J=1.5 Hz, 1H) 7.79 (d, J=1.5 Hz, 1H) 11.44 (br s, 1H).

### Example 30: 1-[1-[2-amino-4-(trifluoromethoxy)benzoyl]-4-piperidyl]-6-[3-(trifluoromethyl)-1-bicyclo[1.1.1]pentanyl]-3H-imidazo[4,5-b]pyridin-2-one

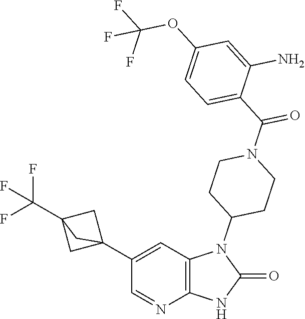 [SMILES: Nc1cc(OC(F)(F)F)ccc1C(=O)N1CCC(n2c(=O)[nH]c3ncc(C45CC(C(F)(F)F)(C4)C5)cc32)CC1]

Example 30 was prepared following the procedure described for the preparation of Example 34 using 4,4,5,5-tetramethyl-2-[3-(trifluoromethyl)-1-bicyclo[1.1.1]pentanyl]-1,3,2-dioxaborolane (65 mg, 0.25 mmol in step 1) to give 12 mg (9% yield over two steps) of 1-[1-[2-amino-4-(trifluoromethoxy)benzoyl]-4-piperidyl]-6-[3-(trifluoromethyl)-1-bicyclo[1.1.1]pentanyl]-3H-imidazo[4,5-b]pyridin-2-one as a solid. LC/MS (m/z, M+H): calc. 556.2, found 556.2; ¹H NMR (400 MHz, DMSO-d6, 27° C.) δ ppm 1.75 (br d, J=11.1 Hz, 2H) 2.24-2.39 (m, 2H) 2.32 (s, 6H) 2.95-3.10 (m, 2H) 3.39-3.39 (m hidden, 2H) 4.40-4.51 (m, 1H) 6.51 (br d, J=8.4 Hz, 1H) 6.68 (br s, 1H) 7.12 (d, J=8.4 Hz, 1H) 7.50 (d, J=1.5 Hz, 1H) 7.81 (d, J=1.5 Hz, 1H) 11.58 (br s, 1H).

### Example 31: (rac)-1-[1-[2-amino-4-(trifluoromethoxy)benzoyl]-4-piperidyl]-6-(3-oxabicyclo[4.1.0]heptan-6-yl)-3H-imidazo[4,5-b]pyridin-2-one

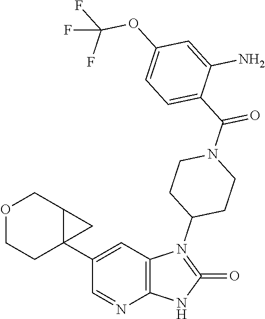

Example 31 was prepared following the procedure described for the preparation of Example 34 using 4,4,5,5-tetramethyl-2-(3-oxabicyclo[4.1.0]heptan-6-yl)-1,3,2-dioxaborolane (56 mg, 0.25 mmol in step 1) to give 10 mg (8% yield over two steps) of (rac)-1-[1-[2-amino-4-(trifluoromethoxy)benzoyl]-4-piperidyl]-6-(3-oxabicyclo[4.1.0]heptan-6-yl)-3H-imidazo[4,5-b]pyridin-2-one as a solid. LC/MS (m/z, M+H): calc. 518.2, found 518.3; ¹H NMR (400 MHz, DMSO-d6, 27° C.) δ ppm 0.84-0.89 (m, 1H) 1.03 (dd, J=9.1, 4.5 Hz, 1H) 1.35-1.40 (m, 1H) 1.75 (br d, J=11.1 Hz, 2H) 1.87-1.94 (m, 1H) 2.05-2.14 (m, 1H) 2.22-2.39 (m, 2H) 2.97-3.10 (m, 2H) 3.47-3.50 (m partially hidden, 4H) 3.82 (d, J=11.3 Hz, 1H) 4.03 (dd, J=11.3, 4.6 Hz, 1H) 4.40-4.52 (m, 1H) 6.51 (br d, J=8.4 Hz, 1H) 6.68 (br s, 1H) 7.13 (d, J=8.3 Hz, 1H) 7.49 (d, J=1.8 Hz, 1H) 7.86 (d, J=1.8 Hz, 1H) 11.45 (br s, 1H).

### Example 32: 1-[1-[2-amino-4-(trifluoromethoxy)benzoyl]-4-piperidyl]-6-(1-bicyclo[2.1.1]hexanyl)-3H-imidazo[4,5-b]pyridin-2-one

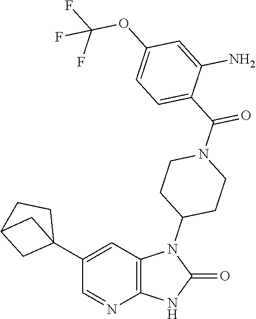 [SMILES: Nc1cc(OC(F)(F)F)ccc1C(=O)N1CCC(n2c(=O)[nH]c3ncc(C45CCC(C4)C5)cc32)CC1]

Example 32 was prepared following the procedure described for the preparation of Example 34 using 2-(1-bicyclo[2.1.1]hexanyl)-4,4,5,5-tetramethyl-1,3,2-dioxaborolane (52 mg, 0.25 mmol in step 1) to give 6 mg (5% yield over two steps) of 1-[1-[2-amino-4-(trifluoromethoxy)benzoyl]-4-piperidyl]-6-(1-bicyclo[2.1.1]hexanyl)-3H-imidazo[4,5-b]pyridin-2-one as a solid. LC/MS (m/z, M+H): calc. 502.2, found 502.3; ¹H NMR (400.23 MHz, DMSO-d6) δ ppm 11.45 (br s, 1H), 7.79 (d, J=1.71 Hz, 1H), 7.46 (d, J=1.71 Hz, 1H), 7.12 (d, J=8.31 Hz, 1H), 6.68 (s, 1H), 6.51 (br d, J=8.56 Hz, 1H), 4.46 (br s, 1H), 3.05 (br s, 2H), 2.89 (br s, 1H), 2.67 (s, 1H), 2.50 (u), 2.45 (br s, 1H), 2.33 (br d, J=1.83 Hz, 2H), 2.09 (m, 1H), 1.80 (m, 7H), 1.55 (br d, J=8.07 Hz, 3H), 1.45 (dd, J=3.73, 1.77 Hz, 1H), 1.41 (br d, J=4.16 Hz, 1H), 1.26 (m, 1H), 0.81 (dd, J=4.03, 1.96 Hz, 1H).

### Example 33: 1-[1-[2-amino-4-(trifluoromethoxy)benzoyl]-4-piperidyl]-6-[3-(difluoromethyl)-1-bicyclo[1.1.1]pentanyl]-3H-imidazo[4,5-b]pyridin-2-one

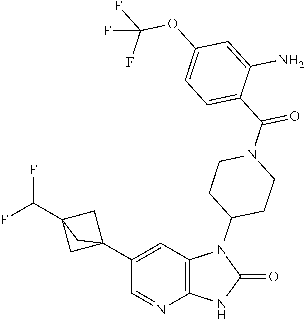 [SMILES: Nc1cc(OC(F)(F)F)ccc1C(=O)N1CCC(n2c(=O)[nH]c3ncc(C45CC(C(F)F)(C4)C5)cc32)CC1]

Example 33 was prepared following the procedure described for the preparation of Example 34 using 2-[3-(difluoromethyl)-1-bicyclo[1.1.1]pentanyl]-4,4,5,5-tetramethyl-1,3,2-dioxaborolane (61 mg, 0.25 mmol in step 1) to give 12 mg (9% yield over two steps) of 1-[1-[2-amino-4-(trifluoromethoxy)benzoyl]-4-piperidyl]-6-[3-(difluoromethyl)-1-bicyclo[1.1.1]pentanyl]-3H-imidazo[4,5-b]pyridin-2-one as a solid. LC/MS (m/z, M+H): calc. 538.2, found 538.2; ¹H NMR (400 MHz, DMSO-d6, 27° C.) δ ppm 1.75 (br d, J=11.1 Hz, 2H) 2.15 (s, 6H) 2.22-2.40 (m, 2H) 2.94-3.11 (m, 2H) 3.41-3.41 (m hidden, 2H) 4.40-4.51 (m, 1H) 6.15 (t, J=56.4 Hz, 1H) 6.51 (br d, J=8.4 Hz, 1H) 6.68 (br s, 1H) 7.12 (d, J=8.4 Hz, 1H) 7.48 (d, J=1.7 Hz, 1H) 7.79 (d, J=1.7 Hz, 1H) 11.55 (br s, 1H).

### Example 34: (rac)-1-[1-[2-amino-4-(trifluoromethoxy)benzoyl]-4-piperidyl]-6-norcaran-1-yl-3H-imidazo[4,5-b]pyridin-2-one

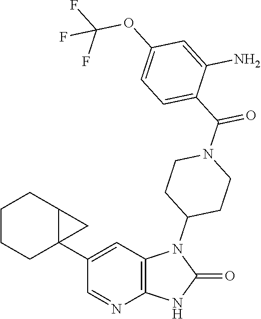 [SMILES: Nc1cc(OC(F)(F)F)ccc1C(=O)N1CCC(n2c(=O)[nH]c3ncc(C45CCCCC4C5)cc32)CC1]

### Step 1: (rac)-tert-butyl N-[2-[4-(6-norcaran-1-yl-2-oxo-3H-imidazo[4,5-b]pyridin-1-yl)piperidine-1-carbonyl]-5-(trifluoromethoxy)phenyl]carbamate

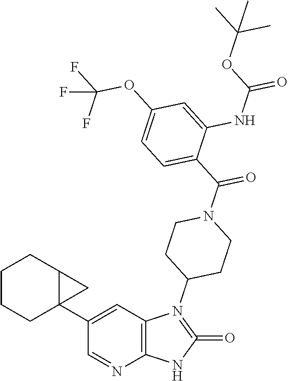

In a 4 mL glassware vial, equipped with a magnetic stir bar and screw cap, (rac)-4,4,5,5-tetramethyl-2-norcaran-1-yl-1,3,2-dioxaborolane (55 mg, 0.25 mmol) was weighed, followed by the addition of 1 mL of stock solution A (prepared according to Example 40, step 1), 0.5 mL of stock solution B (prepared according to Example 40, step 1) and 4-acetamidopiperidine (27 mg, 0.19 mmol). The vial was closed with a septum-screw cap. The mixture was degassed (argon throughflow) for two minutes, using a needle on argon connection and a second needle (pressure compensation). The reaction mixture was then stirred under irradiation of blue LEDs (445 nm, 190 mW) for 6 hours. After 6 hours, the reaction mixture was filtered over a 0.45 μm syringe filter, the vial/filter was rinsed with 0.5 mL of DMF and the filtrate was submitted to preparative reverse phase HPLC purification (column: Waters Sunfire C18 OBD 5 μm, 50×50 mm, eluting with water +0.1% TFA/acetonitrile, Flow rate 2 mL/min) to give 80 mg (52% yield) of (rac)-tert-butyl N-[2-[4-(6-norcaran-1-yl-2-oxo-3H-imidazo[4,5-b]pyridin-1-yl)piperidine-1-carbonyl]-5-(trifluoromethoxy)phenyl]carbamate as a solid, which was engaged in the next step. LC/MS (m/z, M+): calc. 615.3, found 614.2.

### Step 2: (rac)-1-[1-[2-amino-4-(trifluoromethoxy)benzoyl]-4-piperidyl]-6-norcaran-1-yl-3H-imidazo[4,5-b]pyridin-2-one

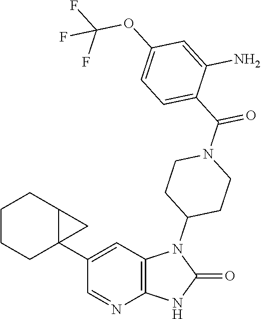 [SMILES: Nc1cc(OC(F)(F)F)ccc1C(=O)N1CCC(n2c(=O)[nH]c3ncc(C45CCCCC4C5)cc32)CC1]

(rac)-1-[1-[2-amino-4-(trifluoromethoxy)benzoyl]-4-piperidyl]-6-norcaran-1-yl-3H-imidazo[4,5-b]pyridin-2-one (80 mg, 0.13 mmol) was placed in a RG24 tube (thick walled test tube with 24 mL volume) and dissolved with 3 mL of dichloromethane. 1 mL of trifluoroacetic acid was added, and the vial was closed with screw caps and shaken overnight at room temperature. After one night, the reaction mixture was evaporated with 3 mL each of fluorobenzene as an entrainer to give 5 mg (7% yield) of ((rac)-1-[1-[2-amino-4-(trifluoromethoxy)benzoyl]-4-piperidyl]-6-norcaran-1-yl-3H-imidazo[4,5-b]pyridin-2-one as a solid. LC/MS (m/z, M+H): calc. 516.2, found 516.3; ¹H NMR (400 MHz, DMSO-d6, 27° C.) δ ppm 0.66 (t, J=5.0 Hz, 1H) 0.93 (dd, J=9.5, 5.0 Hz, 1H) 1.18-1.24 (m, 2H) 1.30-1.40 (m, 2H) 1.42-1.51 (m, 1H) 1.59-1.69 (m, 1H) 1.75 (br d, J=11.1 Hz, 2H) 1.80-1.88 (m, 1H) 2.01-2.12 (m, 2H) 2.19-2.37 (m, 2H) 2.97-3.11 (m, 2H) 3.43-3.44 (m hidden, 2H) 4.40-4.50 (m, 1H) 6.51 (br d, J=8.4 Hz, 1H) 6.69 (br s, 1H) 7.13 (d, J=8.4 Hz, 1H) 7.44 (d, J=1.8 Hz, 1H) 7.83 (d, J=1.8 Hz, 1H) 11.41 (br s, 1H).

### Example 35: 1-[1-[2-amino-4-(trifluoromethoxy)benzoyl]-4-piperidyl]-6-(4,4-difluorocyclohexyl)-3H-imidazo[4,5-b]pyridin-2-one

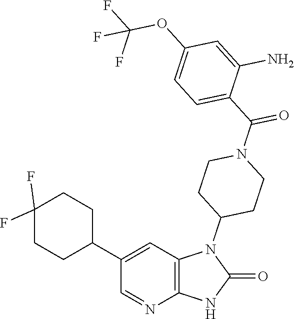 [SMILES: Nc1cc(OC(F)(F)F)ccc1C(=O)N1CCC(n2c(=O)[nH]c3ncc(C4CCC(F)(F)CC4)cc32)CC1]

Example 35 was prepared following the procedure described for the preparation of Example 40 using 2-(4,4-difluorocyclohexyl)-4,4,5,5-tetramethyl-1,3,2-dioxaborolane (62 mg, 0.25 mmol in step 1) to give 34 mg (25% yield over two steps) of 1-[1-[2-amino-4-(trifluoromethoxy)benzoyl]-4-piperidyl]-6-(4,4-difluorocyclohexyl)-3H-imidazo[4,5-b]pyridin-2-one as a solid. LC/MS (m/z, M+H): calc. 540.2, found 540.2; ¹H NMR (400 MHz, DMSO-d6, 27° C.) δ ppm 1.67-1.82 (m, 4H) 1.84-2.05 (m, 4H) 2.10-2.19 (m, 2H) 2.23-2.40 (m, 2H) 2.66-2.80 (m, 1H) 3.04 (s, 2H) 3.21-3.43 (m partially hidden, 2H) 4.42-4.52 (m, 1H) 6.51 (dd, J=8.4, 1.2 Hz, 1H) 6.69 (d, J=1.2 Hz, 1H) 7.14 (d, J=8.4 Hz, 1H) 7.54 (d, J=1.7 Hz, 1H) 7.80 (d, J=1.7 Hz, 1H) 11.45 (br s, 1H).

### Example 36: 1-[1-[2-amino-4-(trifluoromethoxy)benzoyl]-4-piperidyl]-6-cyclohexyl-3H-imidazo[4,5-b]pyridin-2-one

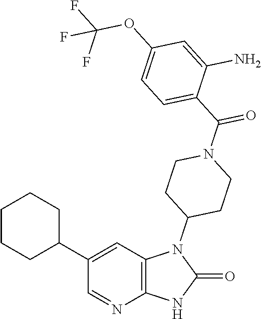 [SMILES: Nc1cc(OC(F)(F)F)ccc1C(=O)N1CCC(n2c(=O)[nH]c3ncc(C4CCCCC4)cc32)CC1]

Example 36 was prepared following the procedure described for the preparation of Example 40 using 2-cyclohexyl-4,4,5,5-tetramethyl-1,3,2-dioxaborolane (52 mg, 0.25 mmol in step 1) to give 29 mg (23% yield over two steps) of 1-[1-[2-amino-4-(trifluoromethoxy)benzoyl]-4-piperidyl]-6-cyclohexyl-3H-imidazo[4,5-b]pyridin-2-one as a solid. LC/MS (m/z, M+H): calc. 504.2, found 504.3; ¹H NMR (400 MHz, DMSO-d6, 27° C.) δ ppm 1.24-1.55 (m, 5H) 1.67-1.85 (m, 7H) 2.22-2.38 (m, 2H) 2.52-2.58 (m, 1H) 2.95-3.14 (m, 2H) 3.44-3.46 (m hidden, 2H) 4.40-4.51 (m, 1H) 6.51 (dd, J=8.3, 1.2 Hz, 1H) 6.68 (d, J=1.2 Hz, 1H) 7.14 (d, J=8.3 Hz, 1H) 7.52 (d, J=1.7 Hz, 1H) 7.77 (d, J=1.7 Hz, 1H) 11.41 (br s, 1H).

### Example 37: 1-[1-[2-amino-4-(trifluoromethoxy)benzoyl]-4-piperidyl]-6-norbornan-2-yl-3H-imidazo[4,5-b]pyridin-2-one

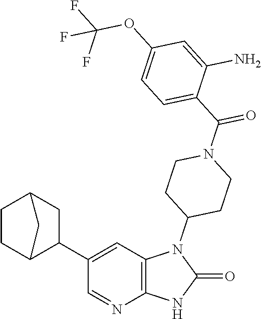 [SMILES: Nc1cc(OC(F)(F)F)ccc1C(=O)N1CCC(n2c(=O)[nH]c3ncc(C4CC5CCC4C5)cc32)CC1]

Example 37 was prepared following the procedure described for the preparation of Example 40 using 4,4,5,5-tetramethyl-2-norbornan-2-yl-1,3,2-dioxaborolane (55 mg, 0.25 mmol in step 1) to give 32 mg (25% yield over two steps) of 1-[1-[2-amino-4-(trifluoromethoxy)benzoyl]-4-piperidyl]-6-norbornan-2-yl-3H-imidazo[4,5-b]pyridin-2-one as a solid. LC/MS (m/z, M+H): calc. 516.2, found 516.3; ¹H NMR (400 MHz, DMSO-d6, 27° C.) δ ppm 1.15-1.20 (m, 1H) 1.24-1.29 (m, 1H) 1.35-1.42 (m, 1H) 1.50-1.61 (m, 3H) 1.61-1.68 (m, 1H) 1.71-1.81 (m, 3H) 2.20-2.38 (m, 4H) 2.73-2.80 (m, 1H) 2.97-3.14 (m, 2H) 3.31-3.42 (m hidden, 2H) 4.42-4.53 (m, 1H) 6.51 (br d, J=8.3 Hz, 1H) 6.69 (br s, 1H) 7.13 (d, J=8.3 Hz, 1H) 7.44 (d, J=1.7 Hz, 1H) 7.78 (d, J=1.7 Hz, 1H) 11.41 (br s, 1H).

### Example 38: (rac)-1-[1-[2-amino-4-(trifluoromethoxy)benzoyl]-4-piperidyl]-6-tetrahydropyran-2-yl-3H-imidazo[4,5-b]pyridin-2-one

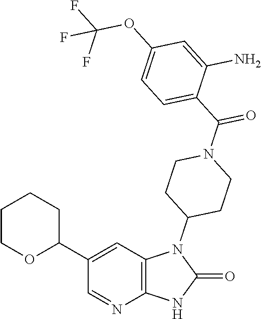 [SMILES: Nc1cc(OC(F)(F)F)ccc1C(=O)N1CCC(n2c(=O)[nH]c3ncc(C4CCCCO4)cc32)CC1]

Example 38 was prepared following the procedure described for the preparation of Example 40 using 4,4,5,5-tetramethyl-2-tetrahydropyran-2-yl-1,3,2-dioxaborolane (53 mg, 0.25 mmol in step 1) to give 48 mg (38% yield over two steps) of (rac)-1-[1-[2-amino-4-(trifluoromethoxy)benzoyl]-4-piperidyl]-6-tetrahydropyran-2-yl-3H-imidazo[4,5-b]pyridin-2-one as a solid. LC/MS (m/z, M+H): calc. 506.2, found 506.2; ¹H NMR (400 MHz, DMSO-d6, 27° C.) δ ppm 1.52-1.67 (m, 4H) 1.70-1.92 (m, 4H) 2.16-2.36 (m, 2H) 2.98-3.16 (m, 2H) 3.52-3.58 (m partially hidden, 3H) 4.03 (br d, J=10.8 Hz, 1H) 4.35 (br d, J=10.8 Hz, 1H) 4.40-4.54 (m, 1H) 6.51 (dd, J=8.4, 1.2 Hz, 1H) 6.68 (d, J=1.2 Hz, 1H) 7.14 (d, J=8.4 Hz, 1H) 7.56 (d, J=1.7 Hz, 1H) 7.88 (d, J=1.7 Hz, 1H) 11.52 (br s, 1H).

### Example 39: 1-[1-[2-amino-4-(trifluoromethoxy)benzoyl]-4-piperidyl]-6-(tetrahydropyran-4-ylmethyl)-3H-imidazo[4,5-b]pyridin-2-one

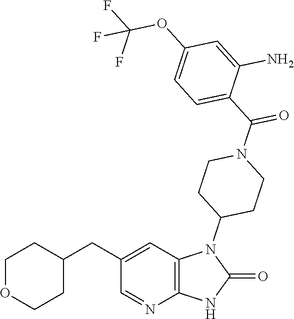 [SMILES: Nc1cc(OC(F)(F)F)ccc1C(=O)N1CCC(n2c(=O)[nH]c3ncc(CC4CCOCC4)cc32)CC1]

Example 39 was prepared following the procedure described for the preparation of Example 40 using 4,4,5,5-tetramethyl-2-(tetrahydropyran-4-ylmethyl)-1,3,2-dioxaborolane (56 mg, 0.25 mmol in step 1) to give 46 mg (35% yield over two steps) of 1-[1-[2-amino-4-(trifluoromethoxy)benzoyl]-4-piperidyl]-6-(tetrahydropyran-4-ylmethyl)-3H-imidazo[4,5-b]pyridin-2-one as a solid. LC/MS (m/z, M+H): calc. 520.2, found 520.3; ¹H NMR (400.23 MHz, DMSO-d6) δ ppm 11.45 (br s, 1H), 7.72 (d, J=1.47 Hz, 1H), 7.53 (d, J=1.47 Hz, 1H), 7.15 (d, J=8.31 Hz, 1H), 6.68 (d, J=1.10 Hz, 1H), 6.50 (d, J=7.81 Hz, 1H), 4.45 (br t, J=12.04, 12.04 Hz, 2H), 3.83 (br dd, J=11.13, 3.06 Hz, 5H), 3.76 (br s, 1H), 3.54 (br s, 101H), 3.24 (br t, J=10.88, 10.88 Hz, 4H), 3.15 (u), 3.03 (br s, 2H), 2.50 (u), 2.33 (br s, 1H), 2.25 (br s, 2H), 1.75 (m, 4H), 1.49 (br d, J=12.72 Hz, 2H), 1.21 (m, 4H).

### Example 40: (rac)-1-[1-[2-amino-4-(trifluoromethoxy)benzoyl]-4-piperidyl]-6-tetrahydropyran-3-yl-3H-imidazo[4,5-b]pyridin-2-one

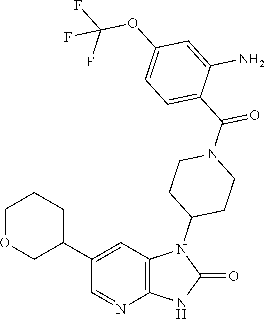 [SMILES: Nc1cc(OC(F)(F)F)ccc1C(=O)N1CCC(n2c(=O)[nH]c3ncc(C4CCCOC4)cc32)CC1]

### Step 1: (rac)-tert-butyl N-[2-[4-(2-oxo-6-tetrahydropyran-3-yl-3H-imidazo[4,5-b]pyridin-1-yl)piperidine-1-carbonyl]-5-(trifluoromethoxy)phenyl]carbamate

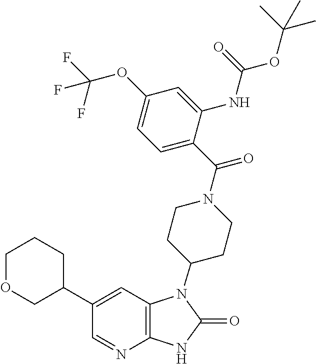 [SMILES: CC(C)(C)OC(=O)Nc1cc(OC(F)(F)F)ccc1C(=O)N1CCC(n2c(=O)[nH]c3ncc(C4CCCOC4)cc32)CC1]

A stock solution A was prepared as follows: In a glass-stoppered conical flask tert-butyl N-[2-[4-(6-bromo-2-oxo-3H-imidazo[4,5-b]pyridin-1-yl)piperidine-1-carbonyl]-5-(trifluoromethoxy)phenyl]carbamate (1651 mg, 2.75 mmol) and [Ir(dF(CF₃)ppy)₂(dtbbpy)]PF₆ (31 mg, 0.028 mmol) was dissolved in dry DMF and adjusted to a total volume of 22 mL.

A stock solution B was prepared as follows: In a conical flask equipped with a septum nickel(II) chloride ethylene glycol dimethyl ether complex (30.2 mg, 0.138 mmol) and 4,4′-di-tert-butyl-2,2′-bipyridine (36.9 mg, 0.138 mmol) were weighed. Then, 11 mL DMF was added and the resulting mixture was warmed to 70-100° C. using a heat gun, until the solution appeared clear and green coloured.

Then, in a 4 mL glass-ware vial, equipped with a magnetic stir bar and screw cap, 4,4,5,5-tetramethyl-2-tetrahydropyran-3-yl-1,3,2-dioxaborolane (53 mg, 0.25 mmol) was weighed, followed by the addition of 1 mL stock solution A, 0.5 mL of stock solution B and morpholine (16 μL, 0.19 mmol). The vial was closed with a septum-screw cap. The reaction mixture was stirred under irradiation of blue LEDs (445 nm, 190 mW) for 4 hours. After 4 hours, the reaction mixture was filtered over a 0.45 μm syringe filter, the vial/filter was rinsed with 0.5 mL of DMF and the filtrate was purified by preparative RP-HPLC purification (column Waters Sunfire C18 OBD 5 μm, 50×50 mm, eluting with Water +0.1% TFA/Acetonitrile, Flow rate 2 mL/min) to give 75 mg (50% yield) of (rac)-tert-butyl N-[2-[4-(2-oxo-6-tetrahydropyran-3-yl-3H-imidazo[4,5-b]pyridin-1-yl)piperidine-1-carbonyl]-5-(trifluoromethoxy)phenyl]carbamate as a white solid which was engaged in the next step. LC/MS (m/z, M+H-BOC): calc. 505.2, found 505.2.

### Step 2: (rac)-1-[1-[2-amino-4-(trifluoromethoxy)benzoyl]-4-piperidyl]-6-tetrahydropyran-3-yl-3H-imidazo[4,5-b]pyridin-2-one

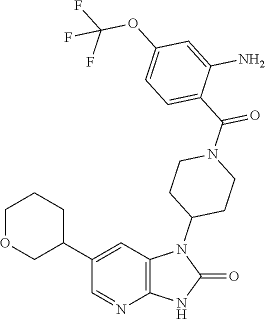 [SMILES: Nc1cc(OC(F)(F)F)ccc1C(=O)N1CCC(n2c(=O)[nH]c3ncc(C4CCCOC4)cc32)CC1]

(rac)-tert-butyl N-[2-[4-(2-oxo-6-tetrahydropyran-3-yl-3H-imidazo[4,5-b]pyridin-1-yl)piperidine-1-carbonyl]-5-(trifluoromethoxy)phenyl]carbamate (75 mg, 0.12 mmol) was placed in a RG24 tube (thick walled test tube with 24 mL volume) and dissolved with 3 mL of dichloromethane. 1 mL of trifluoroacetic acid was added, and the vial was closed with screw caps and shaken overnight at room temperature. After one night, the reaction mixture was evaporated with 3 mL each of fluorobenzene as an entrainer to give 18 mg (29% yield) of (rac)-1-[1-[2-amino-4-(trifluoromethoxy)benzoyl]-4-piperidyl]-6-tetrahydropyran-3-yl-3H-imidazo[4,5-b]pyridin-2-one as a solid. LC/MS (m/z, M+H): calc. 506.2, found 506.3; ¹H NMR (400 MHz, DMSO-d6, 27° C.) δ ppm 1.58-1.98 (m, 6H) 2.17-2.38 (m, 2H) 2.78-2.87 (m, 1H) 2.95-3.11 (m, 2H) 3.35-3.37 (m partially hidden, 4H) 3.80-3.92 (m, 2H) 4.46 (br t, J=12.1 Hz, 1H) 6.51 (br d, J=8.4 Hz, 1H) 6.68 (br s, 1H) 7.14 (d, J=8.4 Hz, 1H) 7.57 (d, J=1.7 Hz, 1H) 7.82 (d, J=1.7 Hz, 1H) 11.47 (br s, 1H).

### Example 41: 6-tetrahydropyran-4-yl-1-[1-[4-(trifluoromethoxy)benzoyl]-4-piperidyl]-3H-imidazo[4,5-b]pyridin-2-one

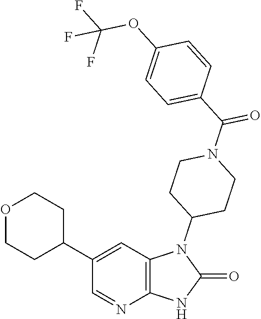 [SMILES: O=C(c1ccc(OC(F)(F)F)cc1)N1CCC(n2c(=O)[nH]c3ncc(C4CCOCC4)cc32)CC1]

Example 41 was prepared following the protocol described for the preparation of Example 42 using 6-bromo-1-[1-[4-(trifluoromethoxy)benzoyl]-4-piperidyl]-3H-imidazo[4,5-b]pyridin-2-one (61 mg, 0.13 mmol) and 4,4,5,5-tetramethyl-2-tetrahydropyran-4-yl-1,3,2-dioxaborolane (53 mg, 0.25 mmol) to give 31 mg (50% yield) of 6-tetrahydropyran-4-yl-1-[1-[4-(trifluoromethoxy)benzoyl]-4-piperidyl]-3H-imidazo[4,5-b]pyridin-2-one as a white solid. LC/MS (m/z, M+H): calc. 491.2, found 491.2; ¹H NMR (400 MHz, DMSO-d6, 27° C.) δ ppm 1.66-1.88 (m, 6H) 2.18-2.40 (m, 2H) 2.78-2.87 (m, 1H) 2.88-3.06 (m, 1H) 3.12-3.27 (m, 1H) 3.44 (br td, J=11.6, 2.1 Hz, 2H) 3.59-3.80 (m, 1H) 3.97 (br dd, J=11.1, 3.5 Hz, 2H) 4.43-4.53 (m, 1H) 4.59-4.75 (m, 1H) 7.47 (br d, J=8.4 Hz, 2H) 7.58 (d, J=1.7 Hz, 1H) 7.62 (br d, J=8.4 Hz, 2H) 7.81 (d, J=1.7 Hz, 1H) 11.44 (br s, 1H).

### Example 42: 6-(oxetan-3-ylmethyl)-1-[1-[4-(trifluoromethoxy)benzoyl]-4-piperidyl]-3H-imidazo[4,5-b]pyridin-2-one

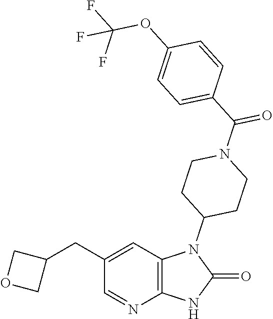 [SMILES: O=C(c1ccc(OC(F)(F)F)cc1)N1CCC(n2c(=O)[nH]c3ncc(CC4COC4)cc32)CC1]

In a 4 mL glass vial, (Ir[dF(CF₃)ppy]₂(dtbpy))PF₆ (1.42 mg, 0.0013 mmol), 6-bromo-1-[1-[4-(trifluoromethoxy)benzoyl]-4-piperidyl]-3H-imidazo[4,5-b]pyridin-2-one (61 mg, 0.13 mmol, Example 60) and 4,4,5,5-tetramethyl-2-(oxetan-3-ylmethyl)-1,3,2-dioxaborolane (50 mg, 0.25 mmol) were dissolved in 0.75 mL of DMF. In a second vial nickel(II) chloride ethylene glycol dimethyl ether complex (1.4 mg, 0.0063 mmol) and 4,4′-di-tert-butyl-2,2′-bipyridine (1.7 mg, 0.0063 mmol) were dissolved in 0.5 mL of DMF. The mixture was sonicated for 1 minute and afterwards heated to 100° C. until complete complexation (clear green solution). Both mixtures were combined, morpholine (16 mg, 0.189 mmol) was added and the mixture was irradiated with blue LEDs (Lumidox 96 LED Array; 445 nm (indigo); 180 mW, 5 cm distance) for 5 hours. After 5 hours, the reaction mixture was purified by preparative HPLC (Conditions: mobile phase A: water +0.1% TFA; mobile phase B: acetonitrile; column: Waters Sunfire C18 OBD 5 μm, 50×50 mm; GRADIENT: time (minutes) % B, 0 min 5%, 2.5 min 25%, 10.5 min 65%, 11 min 99%, 13.5 min 5%; flow rate: 2 mL/min). Afterwards 20 mL 10% NaHCO₃ (aqueous) was added to the collected fractions and the water phase was extracted three times with ethyl acetate. The combined organic phases were dried over MgSO₄. After filtration, the solvent was removed under reduced pressure to give 27 mg (45% yield) of 6-(oxetan-3-ylmethyl)-1-[1-[4-(trifluoromethoxy)benzoyl]-4-piperidyl]-3H-imidazo[4,5-b]pyridin-2-one as a colourless oil. LC/MS (m/z, M+H): calc. 477.2, found 477.2; ¹H NMR (400 MHz, DMSO-d6, 27° C.) δ ppm 1.64-1.88 (m, 2H) 2.13-2.36 (m, 2H) 2.85-3.02 (m, 1H) 2.97 (d, J=7.7 Hz, 2H) 3.15-3.29 (m, 2H) 3.57-3.76 (m, 1H) 4.36 (t, J=5.9 Hz, 2H) 4.40-4.52 (m, 1H) 4.57-4.77 (m, 1H) 4.62 (dd, J=7.7, 5.9 Hz, 2H) 7.47 (br d, J=8.4 Hz, 2H) 7.57 (d, J=1.5 Hz, 1H) 7.63 (br d, J=8.4 Hz, 2H) 7.75 (d, J=1.5 Hz, 1H) 11.46 (br s, 1H).

### Examples 55, 43 & 44: (rac)-1-[1-[2-amino-4-(trifluoromethoxy)benzoyl]-4-piperidyl]-6-[tetrahydrofuran-3-yl]oxy-3H-imidazo[4,5-b]pyridin-2-one, and 1-[1-[2-amino-4-(trifluoromethoxy)benzoyl]-4-piperidyl]-6-[tetrahydrofuran-3-yl]oxy-3H-imidazo[4,5-b]pyridin-2-one Isomers 1 & 2

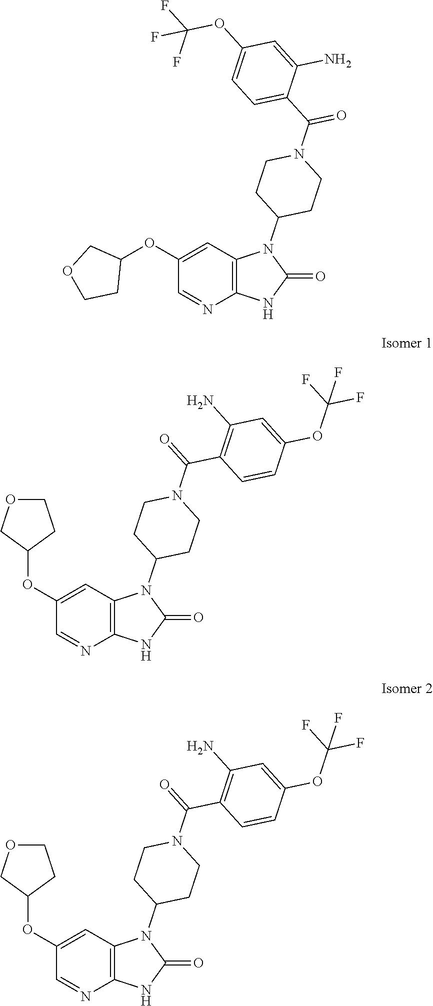 [SMILES: Nc1cc(OC(F)(F)F)ccc1C(=O)N1CCC(n2c(=O)[nH]c3ncc(OC4CCOC4)cc32)CC1 ; Nc1cc(OC(F)(F)F)ccc1C(=O)N1CCC(n2c(=O)[nH]c3ncc(OC4CCOC4)cc32)CC1 ; Nc1cc(OC(F)(F)F)ccc1C(=O)N1CCC(n2c(=O)[nH]c3ncc(OC4CCOC4)cc32)CC1]

### Step 1 (Example 55): (rac)-1-[1-[2-amino-4-(trifluoromethoxy)benzoyl]-4-piperidyl]-6-[tetrahydrofuran-3-yl]oxy-3H-imidazo[4,5-b]pyridin-2-one

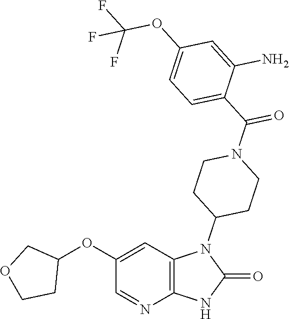 [SMILES: Nc1cc(OC(F)(F)F)ccc1C(=O)N1CCC(n2c(=O)[nH]c3ncc(OC4CCOC4)cc32)CC1]

Step 1 of Examples 43 & 44 was performed following the protocol described in step 4 of Example 15 using (rac)-1-(4-piperidyl)-6-tetrahydrofuran-3-yloxy-3H-imidazo[4,5-b]pyridine-2-one, dihydrochloride (113 mg, 0.3 mmol, prepared in step 4 of Example 56) and 2-amino-4-(trifluoromethoxy)benzoic acid (73 mg, 0.33 mmol) to give 98 mg (64% yield) of (rac)-1-[1-[2-amino-4-(trifluoromethoxy)benzoyl]-4-piperidyl]-6-[tetrahydrofuran-3-yl]oxy-3H-imidazo[4,5-b]pyridin-2-one as a pale yellow solid. LC/MS (m/z, M+H): calc. 508.2, found 508.1; ¹H NMR (400 MHz, DMSO-d6, 100° C.) d ppm 1.81 (br d, J=12.7 Hz, 2H), 1.99-2.07 (m, 1H), 2.16-2.25 (m, 1H), 2.25-2.35 (m, 2H), 3.01-3.11 (m, 2H), 3.76-3.93 (m, 4H), 4.18 (br d, J=12.7 Hz, 2H), 4.37-4.47 (m, 1H), 5.04 (ddt, J=2.2 and 4.4 and 6.4 Hz, 1H), 5.43 (br s, 2H), 6.50 (br d, J=8.4 Hz, 1H), 6.70 (br s, 1H), 7.16 (d, J=8.4 Hz, 1H), 7.28 (d, J=2.4 Hz, 1H), 7.61 (d, J=2.4 Hz, 1H), 11.03 (br s, 1H).

### Step 2 (Examples 43 & 44): 1-[1-[2-amino-4-(trifluoromethoxy)benzoyl]-4-piperidyl]-6-[tetrahydrofuran-3-yl]oxy-3H-imidazo[4,5-b]pyridin-2-one, Isomers 1 & 2

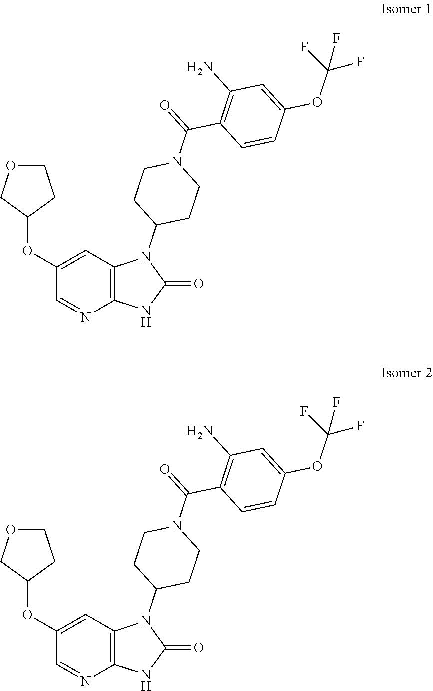 [SMILES: Nc1cc(OC(F)(F)F)ccc1C(=O)N1CCC(n2c(=O)[nH]c3ncc(OC4CCOC4)cc32)CC1 ; Nc1cc(OC(F)(F)F)ccc1C(=O)N1CCC(n2c(=O)[nH]c3ncc(OC4CCOC4)cc32)CC1]

Chiral separation of (rac)-1-[1-[2-amino-4-(trifluoromethoxy)benzoyl]-4-piperidyl]-6-[tetrahydrofuran-3-yl]oxy-3H-imidazo[4,5-b]pyridin-2-one (85 mg, 0.17 mmol) was performed using a Chiralpak AD column (20 μm, 250×4.6 mm), eluting with (heptane 50/EtOH 50)+0.1% TEA (flow rate 1 mL/min, UV detection at 254 nm) to give: 46 mg (54% yield) of 1-[1-[2-amino-4-(trifluoromethoxy)benzoyl]-4-piperidyl]-6-[tetrahydrofuran-3-yl]oxy-3H-imidazo[4,5-b]pyridin-2-one, Isomer 1. LC/MS (m/z, M+H): calc. 508.2, found 508.1; ¹H NMR (400 MHz, DMSO-d6, 100° C.) d ppm 1.78 (br d, J=12.7 Hz, 2H), 1.97-2.06 (m, 1H), 2.15-2.24 (m, 1H), 2.24-2.33 (m, 2H), 3.03-3.08 (m partially hidden, 2H), 3.74-3.90 (m, 4H), 4.16 (br d, J=12.7 Hz, 2H), 4.36-4.45 (m, 1H), 5.01 (ddt, J=2.2 and 4.4 and 6.4 Hz, 1H), 5.41 (br s, 2H), 6.48 (br d, J=8.4 Hz, 1H), 6.68 (br s, 1H), 7.14 (d, J=8.4 Hz, 1H), 7.26 (d, J=2.4 Hz, 1H), 7.59 (d, J=2.4 Hz, 1H), 10.87-11.13 (m, 1H); and 32 mg (38% yield) of 11-[1-[2-amino-4-(trifluoromethoxy)benzoyl]-4-piperidyl]-6-[tetrahydrofuran-3-yl]oxy-3H-imidazo[4,5-b]pyridin-2-one, Isomer 2. LC/MS (m/z, M+H): calc. 508.2, found 508.1; ¹H NMR (400 MHz, DMSO-d6, 100° C.) d ppm 1.78 (br d, J=12.7 Hz, 2H), 1.97-2.05 (m, 1H), 2.14-2.23 (m, 1H), 2.23-2.34 (m, 2H), 3.00-3.09 (m, 2H), 3.74-3.91 (m, 4H), 4.16 (br d, J=12.7 Hz, 2H), 4.36-4.45 (m, 1H), 5.01 (ddt, J=2.2 and 4.4 and 6.4 Hz, 1H), 5.41 (br s, 2H), 6.48 (br d, J=8.4 Hz, 1H), 6.68 (br s, 1H), 7.14 (d, J=8.4 Hz, 1H), 7.25 (d, J=2.4 Hz, 1H), 7.59 (d, J=2.4 Hz, 1H), 10.70-11.20 (m, 1H).

### Examples 45 & 46: trans-1-[1-[2-amino-4-(trifluoromethoxy)benzoyl]-4-piperidyl]-6-(4-methoxycyclohexyl)-3H-imidazo[4,5-b]pyridin-2-one and cis-1-[1-[2-amino-4-(trifluoromethoxy)benzoyl]-4-piperidyl]-6-(4-methoxycyclohexyl)-3H-imidazo[4,5-b]pyridin-2-one

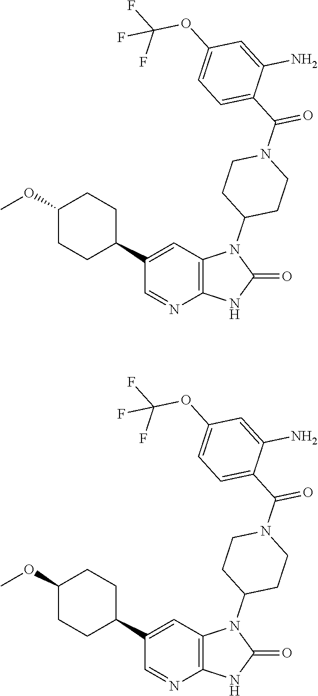 [SMILES: CO[C@H]1CC[C@H](c2cnc3[nH]c(=O)n(C4CCN(C(=O)c5ccc(OC(F)(F)F)cc5N)CC4)c3c2)CC1 ; CO[C@H]1CC[C@@H](c2cnc3[nH]c(=O)n(C4CCN(C(=O)c5ccc(OC(F)(F)F)cc5N)CC4)c3c2)CC1]

### Step 1: (rac)-tert-butyl N-[2-[4-[6-(4-methoxycyclohexen-1-yl)-2-oxo-3H-imidazo[4,5-b]pyridin-1-yl]piperidine-1-carbonyl]-5-(trifluoromethoxy)phenyl]carbamate

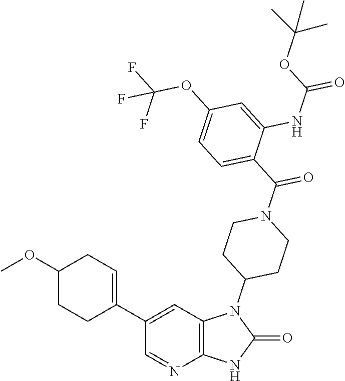 [SMILES: COC1CC=C(c2cnc3[nH]c(=O)n(C4CCN(C(=O)c5ccc(OC(F)(F)F)cc5NC(=O)OC(C)(C)C)CC4)c3c2)CC1]

Step 1 of Examples 45 & 46 was performed following the protocol described in step 1 of Example 15 using tert-butyl N-[2-[4-(6-bromo-2-oxo-3H-imidazo[4,5-b]pyridin-1-yl)piperidine-1-carbonyl]-5-(trifluoromethoxy)phenyl]carbamate (300 mg, 0.5 mmol, Example 59) and 2-(4-methoxycyclohex-1-en-1-yl)-4,4,5,5-tetramethyl-1,3,2-dioxaborolane (142 mg, 0.6 mmol) to give 217 mg (69% yield) of (rac)-tert-butyl N-[2-[4-[6-(4-methoxycyclohexen-1-yl)-2-oxo-3H-imidazo[4,5-b]pyridin-1-yl]piperidine-1-carbonyl]-5-(trifluoromethoxy)phenyl]carbamate as a white solid. LC/MS (m/z, M+H): calc. 632.3, found 632.3.

### Step 2: cis/trans mixture of tert-butyl N-[2-[4-[6-(4-methoxycyclohexyl)-2-oxo-3H-imidazo[4,5-b]pyridin-1-yl]piperidine-1-carbonyl]-5-(trifluoromethoxy)phenyl]carbamate

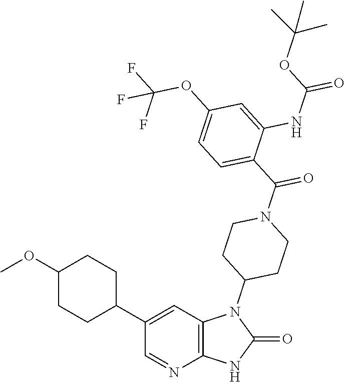 [SMILES: COC1CCC(c2cnc3[nH]c(=O)n(C4CCN(C(=O)c5ccc(OC(F)(F)F)cc5NC(=O)OC(C)(C)C)CC4)c3c2)CC1]

Step 2 of Examples 45 & 46 was performed following the protocol described in step 2 of Examples 57 & 58 using (rac)-tert-butyl N-[2-[4-[6-(4-methoxycyclohexen-1-yl)-2-oxo-3H-imidazo[4,5-b]pyridin-1-yl]piperidine-1-carbonyl]-5-(trifluoromethoxy)phenyl]carbamate (214 mg, 0.34 mmol) in DCM/MeOH (8 mL/8 mL) as solvents, to give 215 mg (100% yield) of a cis/trans mixture of tert-butyl N-[2-[4-[6-(4-methoxycyclohexyl)-2-oxo-3H-imidazo[4,5-b]pyridin-1-yl]piperidine-1-carbonyl]-5-(trifluoromethoxy)phenyl]carbamate as a colorless oil. LC/MS (m/z, M+H): calc. 634.3, found 634.3.

### Step 3: cis/trans mixture of 1-[1-[2-amino-4-(trifluoromethoxy)benzoyl]-4-piperidyl]-6-(4-methoxycyclohexyl)-3H-imidazo[4,5-b]pyridin-2-one, hydrochloride

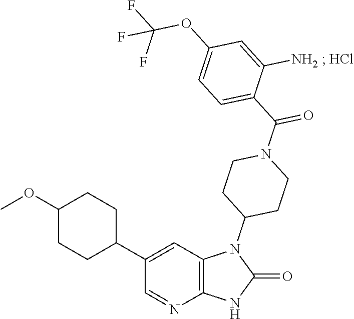

Step 3 of Examples 45 & 46 was performed following the protocol described in step 5 of Example 1 using a cis/trans mixture of tert-butyl N-[2-[4-[6-(4-methoxycyclohexyl)-2-oxo-3H-imidazo[4,5-b]pyridin-1-yl]piperidine-1-carbonyl]-5-(trifluoromethoxy)phenyl]carbamate (290 mg, 0.46 mmol) to give 235 mg (85% yield) of a cis/trans mixture of 1-[1-[2-amino-4-(trifluoromethoxy)benzoyl]-4-piperidyl]-6-(4-methoxycyclohexyl)-3H-imidazo[4,5-b]pyridin-2-one, hydrochloride as a white solid. LC/MS (m/z, M+H-HCl): calc. 534.2, found 534.2.

### Step 4: trans-1-[1-[2-amino-4-(trifluoromethoxy)benzoyl]-4-piperidyl]-6-(4-methoxycyclohexyl)-3H-imidazo[4,5-b]pyridin-2-one and cis-1-[1-[2-amino-4-(trifluoromethoxy)benzoyl]-4-piperidyl]-6-(4-methoxycyclohexyl)-3H-imidazo[4,5-b]pyridin-2-one

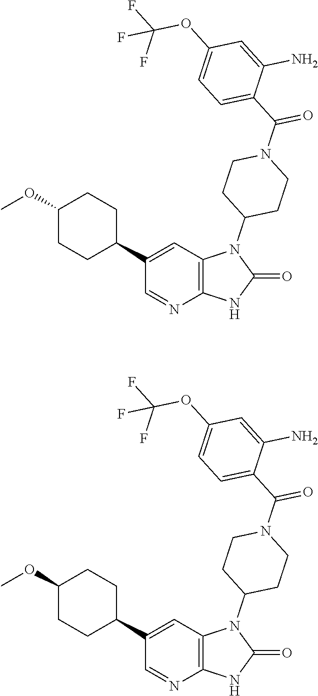 [SMILES: CO[C@H]1CC[C@H](c2cnc3[nH]c(=O)n(C4CCN(C(=O)c5ccc(OC(F)(F)F)cc5N)CC4)c3c2)CC1 ; CO[C@H]1CC[C@@H](c2cnc3[nH]c(=O)n(C4CCN(C(=O)c5ccc(OC(F)(F)F)cc5N)CC4)c3c2)CC1]

Separation of the cis/trans mixture of 1-[1-[2-amino-4-(trifluoromethoxy)benzoyl]-4-piperidyl]-6-(4-methoxycyclohexyl)-3H-imidazo[4,5-b]pyridin-2-one;hydrochloride (161 mg, 0.26 mmol) was performed using a Chiralcel OD-I column (20 μm, 350×76.5 mm), eluting with (Heptane 80/EtOH 20)+0.1% TEA (flow rate 400 mL/min, UV detection at 280 nm) to give: 20 mg (12% yield) of trans-1-[1-[2-amino-4-(trifluoromethoxy)benzoyl]-4-piperidyl]-6-(4-methoxycyclohexyl)-3H-imidazo[4,5-b]pyridin-2-one. LC/MS (m/z, M+H): calc. 534.2, found 534.2; ¹H NMR (400 MHz, DMSO-d6, 100° C.) d ppm 1.29-1.36 (m, 2H), 1.50-1.62 (m, 2H), 1.80 (br d, J=12.7 Hz, 2H), 1.89 (br d, J=12.7 Hz, 2H), 2.13 (br d, J=12.7 Hz, 2H), 2.23-2.37 (m, 2H), 2.52-2.62 (m, 1H), 3.03-3.13 (m, 2H), 3.17-3.28 (m, 1H), 3.30 (s, 3H), 4.19 (br d, J=12.7 Hz, 2H), 4.39-4.49 (m, 1H), 5.44 (br s, 2H), 6.51 (br d, J=8.4 Hz, 1H), 6.72 (br s, 1H), 7.16 (d, J=8.4 Hz, 1H), 7.39 (d, J=1.8 Hz, 1H), 7.78 (d, J=1.8 Hz, 1H), 10.97-11.12 (m, 1H); and

99 mg (61% yield) of cis-1-[1-[2-amino-4-(trifluoromethoxy)benzoyl]-4-piperidyl]-6-(4-methoxycyclohexyl)-3H-imidazo[4,5-b]pyridin-2-one. LC/MS (m/z, M+H): calc. 534.2, found 534.2; ¹H NMR (400 MHz, DMSO-d6, 100° C.) d ppm 1.51-1.64 (m, 4H), 1.71-1.84 (m, 4H), 2.00 (br d, J=12.7 Hz, 2H), 2.24-2.35 (m, 2H), 2.58-2.67 (m, 1H), 3.05-3.14 (m, 2H), 3.29 (s, 3H), 3.48-3.52 (m, 1H), 4.18 (br d, J=12.7 Hz, 2H), 4.42-4.51 (m, 1H), 5.41 (br s, 2H), 6.52 (br d, J=8.4 Hz, 1H), 6.71 (br s, 1H), 7.16 (d, J=8.4 Hz, 1H), 7.35 (d, J=1.8 Hz, 1H), 7.77 (d, J=1.8 Hz, 1H), 11.03 (br s, 1H).

### Examples 47 & 48: 1-[1-[2-amino-4-(trifluoromethoxy)benzoyl]-4-piperidyl]-6-[1-methylpyrrolidin-3-yl]oxy-3H-imidazo[4,5-b]pyridin-2-one, Isomers 1 & 2

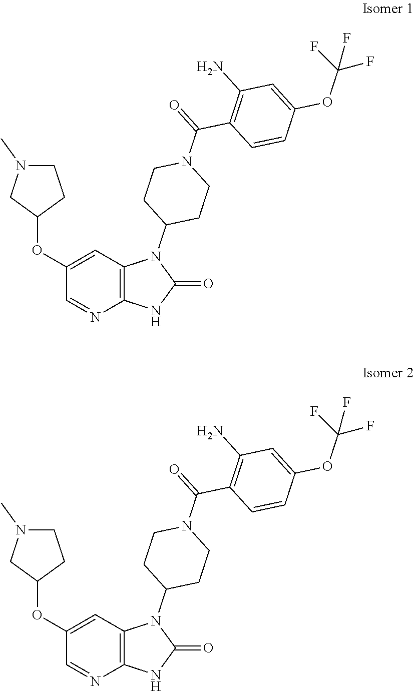 [SMILES: CN1CCC(Oc2cnc3[nH]c(=O)n(C4CCN(C(=O)c5ccc(OC(F)(F)F)cc5N)CC4)c3c2)C1 ; CN1CCC(Oc2cnc3[nH]c(=O)n(C4CCN(C(=O)c5ccc(OC(F)(F)F)cc5N)CC4)c3c2)C1]

Chiral separation of (rac)-1-[1-[2-amino-4-(trifluoromethoxy)benzoyl]-4-piperidyl]-6-(1-methylpyrrolidin-3-yl)oxy-3H-imidazo[4,5-b]pyridin-2-one (Example 6; 111 mg, 0.21 mmol) was performed using a Chiralpak IE column (5 μm, 30×250 mm), eluting with (heptane 30/EtOH 70)+0.1% TEA (flow rate 400 mL/min, UV detection at 254 nm) to give: 40 mg (36% yield) of 1-[i-[2-amino-4-(trifluoromethoxy)benzoyl]-4-piperidyl]-6-(1-methylpyrrolidin-3-yl)oxy-3H-imidazo[4,5-b]pyridin-2-one, Isomer 1. LC/MS (m/z, M+H): calc. 521.2, found 521.1; ¹H NMR (400 MHz, DMSO-d6, 100° C.) d ppm 1.78 (br d, J=12.7 Hz, 2H), 1.81-1.89 (m, 1H), 2.17-2.34 (m, 3H), 2.28 (s, 3H), 2.40-2.45 (m, 1H), 2.60-2.70 (m, 2H), 2.81 (dd, J=6.0 and 10.3 Hz, 1H), 2.99-3.09 (m, 2H), 4.16 (br d, J=12.7 Hz, 2H), 4.35-4.45 (m, 1H), 4.84-4.90 (m, 1H), 5.41 (br s, 2H), 6.48 (br d, J=8.4 Hz, 1H), 6.68 (br s, 1H), 7.14 (d, J=8.4 Hz, 1H), 7.21 (d, J=2.4 Hz, 1H), 7.54 (d, J=2.4 Hz, 1H), 10.89-11.06 (m, 1H); and 46 mg (41% yield) of 1-[1-[2-amino-4-(trifluoromethoxy)benzoyl]-4-piperidyl]-6-(1-methylpyrrolidin-3-yl)oxy-3H-imidazo[4,5-b]pyridin-2-one, Isomer 2. LC/MS (m/z, M+H): calc. 521.2, found 521.1; ¹H NMR (400 MHz, DMSO-d6, 100° C.) d ppm 1.78 (br d, J=12.7 Hz, 2H), 1.81-1.87 (m, 1H), 2.18-2.33 (m, 3H), 2.28 (s, 3H), 2.40-2.45 (m, 1H), 2.59-2.70 (m, 2H), 2.81 (dd, J=6.0 and 10.3 Hz, 1H), 2.99-3.10 (m, 2H), 4.16 (br d, J=12.7 Hz, 2H), 4.35-4.44 (m, 1H), 4.84-4.90 (m, 1H), 5.41 (br s, 2H), 6.48 (br d, J=8.4 Hz, 1H), 6.68 (br s, 1H), 7.14 (d, J=8.4 Hz, 1H), 7.21 (d, J=2.4 Hz, 1H), 7.54 (d, J=2.4 Hz, 1H), 10.90-11.03 (m, 1H).

### Example 49: (rac)-1-[1-[2-amino-4-(pentafluoro-λ⁶-sulfanyl)benzoyl]pyrrolidin-3-yl]-6-tetrahydropyran-4-yl-3H-imidazo[4,5-b]pyridin-2-one

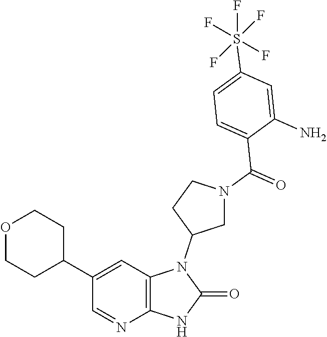 [SMILES: Nc1cc(S(F)(F)(F)(F)F)ccc1C(=O)N1CCC(n2c(=O)[nH]c3ncc(C4CCOCC4)cc32)C1]

Example 49 was prepared following the protocol described in step 4 of Example 15 using (rac)-1-pyrrolidin-3-yl-6-tetrahydropyran-4-yl-3H-imidazo[4,5-b]pyridin-2-one;dihydrochloride (112 mg, 0.31 mmol) and 2-amino-4-(pentafluoro-λ⁶-sulfanyl)benzoic acid (89 mg, 0.34 mmol) to give 122 mg (74% yield) of (rac)-1-[1-[2-amino-4-(pentafluoro-λ⁶-sulfanyl)benzoyl]pyrrolidin-3-yl]-6-tetrahydropyran-4-yl-3H-imidazo[4,5-b]pyridin-2-one as a pale brown solid. LC/MS (m/z, M+H): calc. 534.2, found 534.1; ¹H NMR (400 MHz, DMSO-d6, 100° C.) d ppm 1.69-1.82 (m, 4H), 2.22-2.33 (m, 1H), 2.51-2.56 (m partially hidden, 1H), 2.77-2.90 (m, 1H), 3.43-3.51 (m, 2H), 3.55-3.63 (m, 1H), 3.74-3.86 (m, 2H), 3.90-4.01 (m, 3H), 5.02 (quin, J=7.7 Hz, 1H), 5.70 (br s, 2H), 6.99 (dd, J=2.3 and 8.4 Hz, 1H), 7.28 (d, J=2.3 Hz, 1H), 7.32 (br d, J=8.4 Hz, 1H), 7.37 (d, J=1.8 Hz, 1H), 7.83 (d, J=1.8 Hz, 1H), 11.16 (br s, 1H).

### Example 50: (rac)-1-[1-[4-(pentafluoro-λ⁶-sulfanyl)benzoyl]pyrrolidin-3-yl]-6-tetrahydropyran-4-yl-3H-imidazo[4,5-b]pyridin-2-one

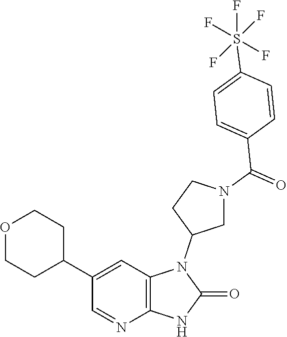 [SMILES: O=C(c1ccc(S(F)(F)(F)(F)F)cc1)N1CCC(n2c(=O)[nH]c3ncc(C4CCOCC4)cc32)C1]

### Step 1: (rac)-tert-butyl 3-[(5-bromo-2-nitro-3-pyridyl)amino]pyrrolidine-1-carboxylate

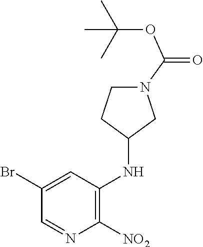 [SMILES: CC(C)(C)OC(=O)N1CCC(Nc2cc(Br)cnc2[N+](=O)[O-])C1]

To a solution of 5-bromo-3-fluoro-2-nitro-pyridine (3 g, 13.6 mmol) in EtOH (91 mL) was added triethylamine (5.68 mL, 40.7 mmol) and (rac)-tert-butyl 3-aminopyrrolidine-1-carboxylate (3.03 g, 16.3 mmol) and the resulting reaction mixture was refluxed for 1.5 hours, cooled down to room temperature, concentrated in vacuo, diluted with ethyl acetate and washed with an aqueous saturated solution of NH₄Cl. The organic layer was dried over sodium sulfate, filtered and concentrated to give 5.284 g (89% yield) of (rac)-tert-butyl 3-[(5-bromo-2-nitro-3-pyridyl)amino]pyrrolidine-1-carboxylate as a yellow solid. LC/MS (m/z, M+H-tBu): calc. 331.0, found 331.1.

### Step 2: (rac)-tert-butyl 3-[(2-amino-5-bromo-3-pyridyl)amino]pyrrolidine-1-carboxylate

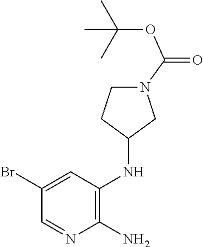 [SMILES: CC(C)(C)OC(=O)N1CCC(Nc2cc(Br)cnc2N)C1]

To a solution of (rac)-tert-butyl 3-[(5-bromo-2-nitro-3-pyridyl)amino]pyrrolidine-1-carboxylate (5 g, 11.5 mmol) and 4,4,5,5-tetramethyl-2-(4,4,5,5-tetramethyl-1,3,2-dioxaborolan-2-yl)-1,3,2-dioxaborolane (9.05 g, 35.6 mmol) in isopropanol (50 mL) was added at room temperature, potassium tert-butoxide (1.55 g, 13.8 mmol) and the resulting reaction mixture was refluxed for 1.5 hours, then cooled down to room temperature, concentrated in vacuo, diluted with ethyl acetate, and washed twice with a 1 N solution of NaOH. The organic layer was then dried over sodium sulfate, filtered and concentrated in vacuo. The resulting residue was triturated in diisopropylether, filtered, washed with diisopropylether and dried under vacuum to give 3.505 g (82% yield) of (rac)-tert-butyl 3-[(2-amino-5-bromo-3-pyridyl)amino]pyrrolidine-1-carboxylate as a brown solid. LC/MS (m/z, M+H-tBu): calc. 302.1, found 303.0.

### Step 3: (rac)-tert-butyl 3-(6-bromo-2-oxo-3H-imidazo[4,5-b]pyridin-1-yl)pyrrolidine-1-carboxylate

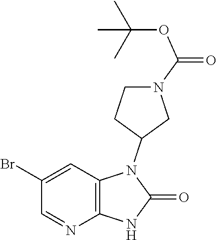 [SMILES: CC(C)(C)OC(=O)N1CCC(n2c(=O)[nH]c3ncc(Br)cc32)C1]

Step 3 of Example 50 was performed following the protocol described in step 4 of Example 1 using (rac)-tert-butyl 3-[(2-amino-5-bromo-3-pyridyl)amino]pyrrolidine-1-carboxylate (3.5 g, 9.41 mmol) to give 3.733 g (97% yield) of (rac)-tert-butyl 3-(6-bromo-2-oxo-3H-imidazo[4,5-b]pyridin-1-yl)pyrrolidine-1-carboxylate as a yellow solid. LC/MS (m/z, M+H): calc. 383.1, found 383.0.

### Step 4: (rac)-tert-butyl 3-[6-(3,6-dihydro-2H-pyran-4-yl)-2-oxo-3H-imidazo[4,5-b]pyridin-1-yl]pyrrolidine-1-carboxylate

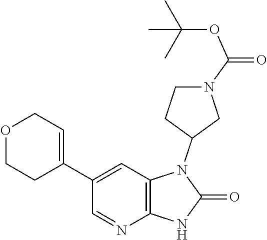 [SMILES: CC(C)(C)OC(=O)N1CCC(n2c(=O)[nH]c3ncc(C4=CCOCC4)cc32)C1]

Step 4 of Example 50 was performed following the protocol described in step 1 of Examples 57 & 58 using tert-butyl (rac)-3-(6-bromo-2-oxo-3H-imidazo[4,5-b]pyridin-1-yl)pyrrolidine-1-carboxylate (400 mg, 0.98 mmol) and 2-(3,6-dihydro-2H-pyran-4-yl)-4,4,5,5-tetramethyl-1,3,2-dioxaborolane (247 mg, 1.18 mmol) to give 264 mg (70% yield) of (rac)-tert-butyl 3-[6-(3,6-dihydro-2H-pyran-4-yl)-2-oxo-3H-imidazo[4,5-b]pyridin-1-yl]pyrrolidine-1-carboxylate as a pale brown solid. LC/MS (m/z, M+H): calc. 387.2, found 387.2.

### Step 5: (rac)-tert-butyl 3-(2-oxo-6-tetrahydropyran-4-yl-3H-imidazo[4,5-b]pyridin-1-yl)pyrrolidine-1-carboxylate

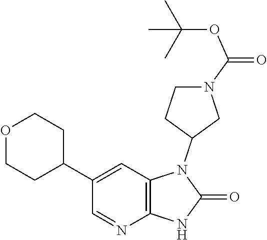 [SMILES: CC(C)(C)OC(=O)N1CCC(n2c(=O)[nH]c3ncc(C4CCOCC4)cc32)C1]

Step 5 of Example 50 was performed following the protocol described in step 2 of Examples 57 & 58 using (rac)-tert-butyl 3-[6-(3,6-dihydro-2H-pyran-4-yl)-2-oxo-3H-imidazo[4,5-b]pyridin-1-yl]pyrrolidine-1-carboxylate (261 mg, 0.67 mmol) to give 246 mg (94% yield) of (rac)-tert-butyl 3-(2-oxo-6-tetrahydropyran-4-yl-3H-imidazo[4,5-b]pyridin-1-yl)pyrrolidine-1-carboxylate as a white solid. LC/MS (m/z, M+H): calc. 389.2, found 389.2.

### Step 6: (rac)-1-pyrrolidin-3-yl-6-tetrahydropyran-4-yl-3H-imidazo[4,5-b]pyridin-2-one, dihydrochloride

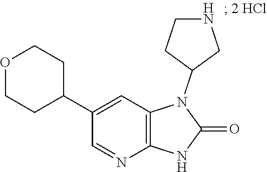 [SMILES: O=c1[nH]c2ncc(C3CCOCC3)cc2n1C1CCNC1]

Step 6 of Example 50 was performed following the protocol described in step 5 of Example 1 using (rac)-tert-butyl 3-(2-oxo-6-tetrahydropyran-4-yl-3H-imidazo[4,5-b]pyridin-1-yl)pyrrolidine-1-carboxylate (245 mg, 0.63 mmol) to give 227 mg (99% yield) of (rac)-1-pyrrolidin-3-yl-6-tetrahydropyran-4-yl-3H-imidazo[4,5-b]pyridin-2-one;dihydrochloride as a yellow solid. LC/MS (m/z, M+H-2HCl): calc. 289.2, found 289.1.

### Step 7: (rac)-1-[1-[4-(pentafluoro-λ⁶-sulfanyl)benzoyl]pyrrolidin-3-yl]-6-tetrahydropyran-4-yl-3H-imidazo[4,5-b]pyridin-2-one

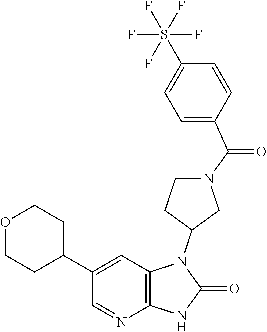 [SMILES: O=C(c1ccc(S(F)(F)(F)(F)F)cc1)N1CCC(n2c(=O)[nH]c3ncc(C4CCOCC4)cc32)C1]

Step 7 of Example 50 was performed following the protocol described in step 4 of Example 15 using (rac)-1-pyrrolidin-3-yl-6-tetrahydropyran-4-yl-3H-imidazo[4,5-b]pyridin-2-one, dihydrochloride (112 mg, 0.31 mmol) and 4-(pentafluoro-λ⁶-sulfanyl)benzoic acid (85 mg, 0.34 mmol) to give 112 mg (70% yield) of (rac)-1-[1-[4-(pentafluoro-λ⁶-sulfanyl)benzoyl]pyrrolidin-3-yl]-6-tetrahydropyran-4-yl-3H-imidazo[4,5-b]pyridin-2-one as a pale brown solid. LC/MS (m/z, M+H): calc. 519.1, found 519.1; ¹H NMR (400 MHz, DMSO-d6, 100° C.) d ppm 1.70-1.81 (m, 4H), 2.25-2.34 (m, 1H), 2.52-2.60 (m partially hidden, 1H), 2.78-2.88 (m, 1H), 3.43-3.52 (m, 2H), 3.57-3.67 (m, 1H), 3.76-3.89 (m, 2H), 3.91-4.02 (m, 3H), 4.98-5.07 (m, 1H), 7.37 (d, J=1.8 Hz, 1H), 7.76 (br d, J=8.5 Hz, 2H), 7.82 (d, J=1.8 Hz, 1H), 7.95 (br d, J=8.5 Hz, 2H), 11.14 (br s, 1H).

### Examples 51 & 52: trans-1-[1-[2-amino-4-(trifluoromethoxy)benzoyl]-4-piperidyl]-6-(4-hydroxycyclohexyl)-3H-imidazo[4,5-b]pyridin-2-one & cis-1-[1-[2-amino-4-(trifluoromethoxy)benzoyl]-4-piperidyl]-6-(4-hydroxycyclohexyl)-3H-imidazo[4,5-b]pyridin-2-one

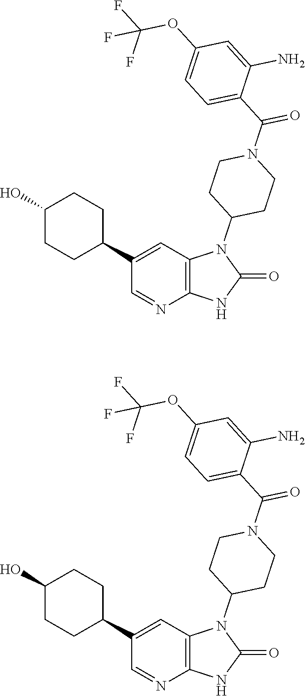 [SMILES: Nc1cc(OC(F)(F)F)ccc1C(=O)N1CCC(n2c(=O)[nH]c3ncc([C@H]4CC[C@H](O)CC4)cc32)CC1 ; Nc1cc(OC(F)(F)F)ccc1C(=O)N1CCC(n2c(=O)[nH]c3ncc([C@H]4CC[C@@H](O)CC4)cc32)CC1]

### Step 1: tert-butyl N-[2-[4-[6-(4-hydroxycyclohexen-1-yl)-2-oxo-3H-imidazo[4,5-b]pyridin-1-yl]piperidine-1-carbonyl]-5-(trifluoromethoxy)phenyl]carbamate

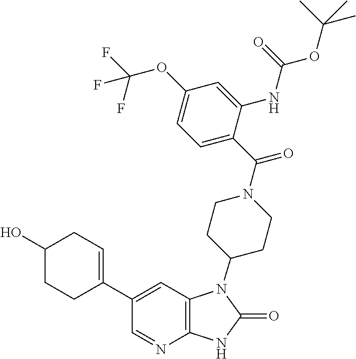 [SMILES: CC(C)(C)OC(=O)Nc1cc(OC(F)(F)F)ccc1C(=O)N1CCC(n2c(=O)[nH]c3ncc(C4=CCC(O)CC4)cc32)CC1]

Step 1 of Examples 51 & 52 was performed following the protocol described in step 1 of Examples 57 & 58 using tert-butyl N-[2-[4-(6-bromo-2-oxo-3H-imidazo[4,5-b]pyridin-1-yl)piperidine-1-carbonyl]-5-(trifluoromethoxy)phenyl]carbamate (185 mg, 0.31 mmol) and 4-(4,4,5,5-tetramethyl-1,3,2-dioxaborolan-2-yl)cyclohex-3-en-1-ol (83 mg, 0.37 mmol) to give 135 mg (71% yield) of tert-butyl N-[2-[4-[6-(4-hydroxycyclohexen-1-yl)-2-oxo-3H-imidazo[4,5-b]pyridin-1-yl]piperidine-1-carbonyl]-5-(trifluoromethoxy)phenyl]carbamate as a pale brown solid. LC/MS (m/z, M+H): calc. 618.2, found 618.2.

### Step 2: cis/trans mixture of tert-butyl N-[2-[4-[6-(4-hydroxycyclohexyl)-2-oxo-3H-imidazo[4,5-b]pyridin-1-yl]piperidine-1-carbonyl]-5-(trifluoromethoxy)phenyl]carbamate

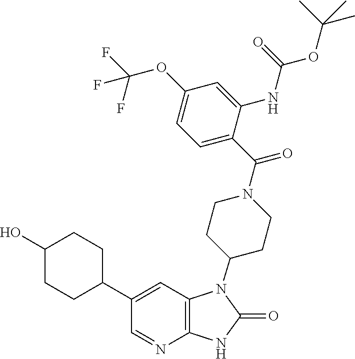 [SMILES: CC(C)(C)OC(=O)Nc1cc(OC(F)(F)F)ccc1C(=O)N1CCC(n2c(=O)[nH]c3ncc(C4CCC(O)CC4)cc32)CC1]

Step 2 of Examples 51 & 52 was performed following the protocol described in step 2 of Examples 57 & 58 using tert-butyl N-[2-[4-[6-(4-hydroxycyclohexen-1-yl)-2-oxo-3H-imidazo[4,5-b]pyridin-1-yl]piperidine-1-carbonyl]-5-(trifluoromethoxy)phenyl]carbamate (132 mg, 0.21 mmol) to give 88 mg (66% yield) of a cis/trans mixture of tert-butyl N-[2-[4-[6-(4-hydroxycyclohexyl)-2-oxo-3H-imidazo[4,5-b]pyridin-1-yl]piperidine-1-carbonyl]-5-(trifluoromethoxy)phenyl]carbamate as a white solid. LC/MS (m/z, M+H): calc. 620.3, found 620.3.

### Step 3: cis/trans mixture of 1-[1-[2-amino-4-(trifluoromethoxy)benzoyl]-4-piperidyl]-6-(4-hydroxycyclohexyl)-3H-imidazo[4,5-b]pyridin-2-one

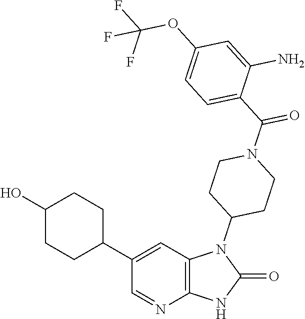 [SMILES: Nc1cc(OC(F)(F)F)ccc1C(=O)N1CCC(n2c(=O)[nH]c3ncc(C4CCC(O)CC4)cc32)CC1]

To a solution of a cis/trans mixture of tert-butyl N-[2-[4-[6-(4-hydroxycyclohexyl)-2-oxo-3H-imidazo[4,5-b]pyridin-1-yl]piperidine-1-carbonyl]-5-(trifluoromethoxy)phenyl]carbamate (85 mg, 0.14 mmol) in MeOH (2 mL) was added a 4N HCl solution in dioxane (0.51 mL, 2.06 mmol) and the resulting mixture was stirred at room temperature for 1.5 hours, followed by 1.5 hours at 40° C. The reaction mixture was then concentrated to dryness, diluted with 4 mL of 7 N solution of ammonia in MeOH, and concentrated again to dryness. The resulting residue was purified by flash chromatography on silica gel (SiO₂ 12 g) eluting with DCM/MeOH/NH₄OH 95/5/0.5 to give 68 mg (95% yield) of a cis/trans mixture of 1-[1-[2-amino-4-(trifluoromethoxy)benzoyl]-4-piperidyl]-6-(4-hydroxycyclohexyl)-3H-imidazo[4,5-b]pyridin-2-one as a white solid. LC/MS (m/z, M+H): calc. 520.2, found 520.2.

### Step 4: trans-1-[1-[2-amino-4-(trifluoromethoxy)benzoyl]-4-piperidyl]-6-(4-hydroxycyclohexyl)-3H-imidazo[4,5-b]pyridin-2-one & cis-1-[1-[2-amino-4-(trifluoromethoxy)benzoyl]-4-piperidyl]-6-(4-hydroxycyclohexyl)-3H-imidazo[4,5-b]pyridin-2-one

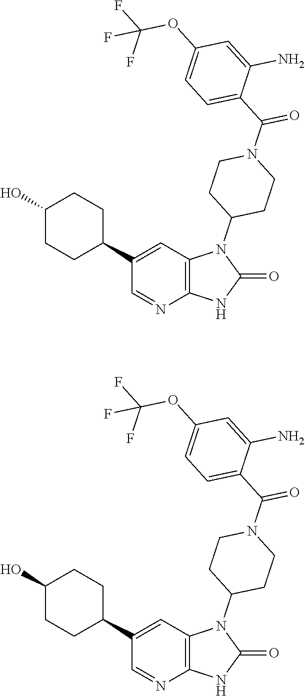 [SMILES: Nc1cc(OC(F)(F)F)ccc1C(=O)N1CCC(n2c(=O)[nH]c3ncc([C@H]4CC[C@H](O)CC4)cc32)CC1 ; Nc1cc(OC(F)(F)F)ccc1C(=O)N1CCC(n2c(=O)[nH]c3ncc([C@H]4CC[C@@H](O)CC4)cc32)CC1]

Separation of the cis/trans mixture of 1-[1-[2-amino-4-(trifluoromethoxy)benzoyl]-4-piperidyl]-6-(4-hydroxycyclohexyl)-3H-imidazo[4,5-b]pyridin-2-one (68 mg, 0.13 mmol) was performed using a Chiralpak IC column (5 μm, 250×30 mm), eluting with (heptane 50/EtOH 50)+0.1% TEA (flow rate 40 mL/min, UV detection at 265 nm) to give: 12 mg (18% yield) of trans-1-[1-[2-amino-4-(trifluoromethoxy)benzoyl]-4-piperidyl]-6-(4-hydroxycyclohexyl)-3H-imidazo[4,5-b]pyridin-2-one. LC/MS (m/z, M+H): calc. 520.2, found 520.1; ¹H NMR (400 MHz, DMSO-d6, 100° C.) d ppm 1.28-1.37 (m, 2H), 1.47-1.59 (m, 2H), 1.73-1.86 (m, 4H), 1.91-2.01 (m, 2H), 2.21-2.34 (m, 2H), 2.52-2.57 (m, 1H), 3.00-3.10 (m, 2H), 3.42-3.58 (m, 1H), 4.12 (br d, J=4.6 Hz, 1H), 4.13-4.20 (m, 2H), 4.37-4.46 (m, 1H), 5.41 (br s, 2H), 6.49 (br d, J=8.4 Hz, 1H), 6.69 (br s, 1H), 7.13 (d, J=8.4 Hz, 1H), 7.36 (d, J=1.8 Hz, 1H), 7.75 (d, J=1.8 Hz, 1H), 10.84-11.14 (m, 1H); and 36 mg (53% yield) of cis-1-[1-[2-amino-4-(trifluoromethoxy)benzoyl]-4-piperidyl]-6-(4-hydroxycyclohexyl)-3H-imidazo[4,5-b]pyridin-2-one. LC/MS (m/z, M+H): calc. 520.2, found 520.1; ¹H NMR (400 MHz, DMSO-d6, 100° C.) d ppm 1.50-1.63 (m, 4H), 1.79 (br d, J=12.7 Hz, 4H), 1.84-1.96 (m, 2H), 2.21-2.32 (m, 2H), 2.52-2.60 (m, 1H), 3.02-3.12 (m, 2H), 3.91-3.95 (m, 1H), 4.07 (br d, J=3.9 Hz, 1H), 4.16 (br d, J=12.7 Hz, 2H), 4.39-4.50 (m, 1H), 5.41 (br s, 2H), 6.50 (br d, J=8.4 Hz, 1H), 6.70 (br s, 1H), 7.14 (d, J=8.4 Hz, 1H), 7.35 (d, J=1.8 Hz, 1H), 7.76 (d, J=1.8 Hz, 1H), 10.89-11.10 (m, 1H).

### Example 53: 1-[1-[2-amino-4-(trifluoromethoxy)benzoyl]-4-piperidyl]-6-[1-(2,2,2-trifluoroethyl)-4-piperidyl]-3H-imidazo[4,5-b]pyridin-2-one

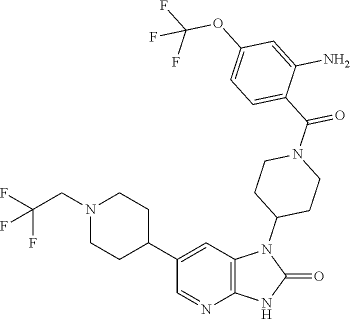 [SMILES: Nc1cc(OC(F)(F)F)ccc1C(=O)N1CCC(n2c(=O)[nH]c3ncc(C4CCN(CC(F)(F)F)CC4)cc32)CC1]

### Step 1: tert-butyl 4-[2-oxo-6-[1-(2,2,2-trifluoroethyl)-3,6-dihydro-2H-pyridin-4-yl]-3H-imidazo[4,5-b]pyridin-1-yl]piperidine-1-carboxylate

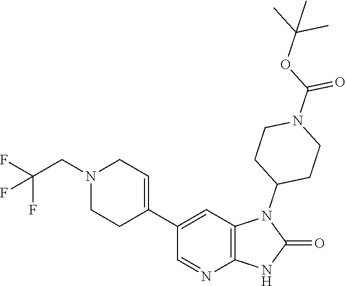 [SMILES: CC(C)(C)OC(=O)N1CCC(n2c(=O)[nH]c3ncc(C4=CCN(CC(F)(F)F)CC4)cc32)CC1]

Step 1 of Example 53 was performed the protocol described in step 1 of Example 15 using tert-butyl 4-(6-bromo-2-oxo-3H-imidazo[4,5-b]pyridin-1-yl)piperidine-1-carboxylate (400 mg, 1.007 mmol, prepared in step 2 of Example 3) and 4-(4,4,5,5-tetramethyl-1,3,2-dioxaborolan-2-yl)-1-(2,2,2-trifluoroethyl)-1,2,3,6-tetrahydropyridine (322 mg, 1.11 mmol) to give 250 mg (52% yield) of tert-butyl 4-[2-oxo-6-[1-(2,2,2-trifluoroethyl)-3,6-dihydro-2H-pyridin-4-yl]-3H-imidazo[4,5-b]pyridin-1-yl]piperidine-1-carboxylate as a solid.

### Step 2: tert-butyl 4-[2-oxo-6-[1-(2,2,2-trifluoroethyl)-4-piperidyl]-3H-imidazo[4,5-b]pyridin-1-yl]piperidine-1-carboxylate

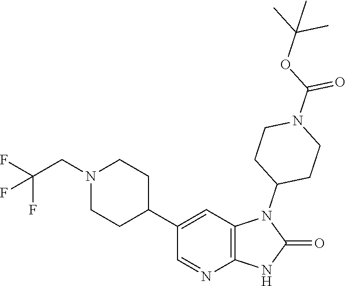 [SMILES: CC(C)(C)OC(=O)N1CCC(n2c(=O)[nH]c3ncc(C4CCN(CC(F)(F)F)CC4)cc32)CC1]

Step 2 of Example 53 was performed following the protocol described in step 2 of Examples 57 & 58 using tert-butyl 4-[2-oxo-6-[1-(2,2,2-trifluoroethyl)-3,6-dihydro-2H-pyridin-4-yl]-3H-imidazo[4,5-b]pyridin-1-yl]piperidine-1-carboxylate (250 mg, 0.52 mmol) to give 240 mg (95% yield) of tert-butyl 4-[2-oxo-6-[1-(2,2,2-trifluoroethyl)-4-piperidyl]-3H-imidazo[4,5-b]pyridin-1-yl]piperidine-1-carboxylate as a white solid. LC/MS (m/z, M+H): calc. 484.2, found 484.2.

### Step 3: 1-(4-piperidyl)-6-[1-(2,2,2-trifluoroethyl)-4-piperidyl]-3H-imidazo[4,5-b]pyridin-2-one, hydrochloride

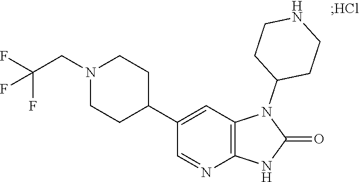 [SMILES: O=c1[nH]c2ncc(C3CCN(CC(F)(F)F)CC3)cc2n1C1CCNCC1]

Step 3 of Example 53 was performed following the protocol described in step 5 of Example 1 using tert-butyl 4-[2-oxo-6-[1-(2,2,2-trifluoroethyl)-4-piperidyl]-3H-imidazo[4,5-b]pyridin-1-yl]piperidine-1-carboxylate (230 mg, 0.44 mmol) to give 185 mg (100% yield) of 1-(4-piperidyl)-6-[1-(2,2,2-trifluoroethyl)-4-piperidyl]-3H-imidazo[4,5-b]pyridin-2-one, hydrochloride as a solid. LC/MS (m/z, M+H-HCl): calc. 384.2, found 384.2.

### Step 4: 1-[1-[2-amino-4-(trifluoromethoxy)benzoyl]-4-piperidyl]-6-[1-(2,2,2-trifluoroethyl)-4-piperidyl]-3H-imidazo[4,5-b]pyridin-2-one

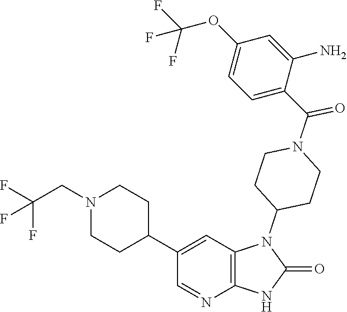 [SMILES: Nc1cc(OC(F)(F)F)ccc1C(=O)N1CCC(n2c(=O)[nH]c3ncc(C4CCN(CC(F)(F)F)CC4)cc32)CC1]

Step 4 of Example 53 was performed following the protocol described in step 4 of Example 15 using 1-(4-piperidyl)-6-[1-(2,2,2-trifluoroethyl)-4-piperidyl]-3H-imidazo[4,5-b]pyridin-2-one, hydrochloride (120 mg, 0.28 mmol), 2-amino-4-(trifluoromethoxy)benzoic acid (69 mg, 0.31 mmol) and triethylamine (0.16 mL, 1.14 mmol) instead of DIPEA, to give 61 mg (37% yield) of 1-[1-[2-amino-4-(trifluoromethoxy)benzoyl]-4-piperidyl]-6-[1-(2,2,2-trifluoroethyl)-4-piperidyl]-3H-imidazo[4,5-b]pyridin-2-one as a solid. LC/MS (m/z, M+H): calc. 587.2, found 587.1; ¹H NMR (400 MHz, DMSO-d6, 100° C.) d ppm 1.71-1.82 (m, 6H), 2.24-2.36 (m, 2H), 2.49-2.59 (m partially hidden, 3H), 3.00-3.11 (m, 4H), 3.16 (q, J=10.2 Hz, 2H), 4.16 (br d, J=12.7 Hz, 2H), 4.39-4.48 (m, 1H), 5.40 (br s, 2H), 6.49 (br d, J=8.4 Hz, 1H), 6.69 (br s, 1H), 7.14 (d, J=8.4 Hz, 1H), 7.42 (d, J=1.8 Hz, 1H), 7.77 (d, J=1.8 Hz, 1H), 11.03 (br s, 1H).

### Example 54: 1-[1-[2-amino-4-(trifluoromethoxy)benzoyl]-4-piperidyl]-6-(1-methyl-4-piperidyl)-3H-imidazo[4,5-b]pyridin-2-one

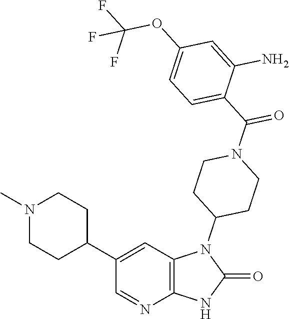 [SMILES: CN1CCC(c2cnc3[nH]c(=O)n(C4CCN(C(=O)c5ccc(OC(F)(F)F)cc5N)CC4)c3c2)CC1]

### Step 1: tert-butyl 4-[6-(1-methyl-3,6-dihydro-2H-pyridin-4-yl)-2-oxo-3H-imidazo[4,5-b]pyridin-1-yl]piperidine-1-carboxylate

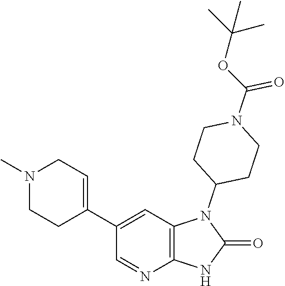 [SMILES: CN1CC=C(c2cnc3[nH]c(=O)n(C4CCN(C(=O)OC(C)(C)C)CC4)c3c2)CC1]

Step 1 of Example 54 was performed the protocol described in step 1 of Example 15 using tert-butyl 4-(6-bromo-2-oxo-3H-imidazo[4,5-b]pyridin-1-yl)piperidine-1-carboxylate (400 mg, 1.007 mmol, prepared in step 2 of Example 3) and 1-methyl-4-(4,4,5,5-tetramethyl-1,3,2-dioxaborolan-2-yl)-1,2,3,6-tetrahydropyridine (247 mg, 1.11 mmol) to give 138 mg (33% yield) of tert-butyl 4-[6-(1-methyl-3,6-dihydro-2H-pyridin-4-yl)-2-oxo-3H-imidazo[4,5-b]pyridin-1-yl]piperidine-1-carboxylate as a solid.

### Step 2: tert-butyl 4-[6-(1-methyl-4-piperidyl)-2-oxo-3H-imidazo[4,5-b]pyridin-1-yl]piperidine-1-carboxylate

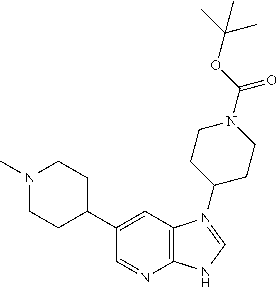

Step 2 of Example 54 was performed following the protocol described in step 2 of Examples 57 & 58 using tert-butyl 4-[6-(1-methyl-3,6-dihydro-2H-pyridin-4-yl)-2-oxo-3H-imidazo[4,5-b]pyridin-1-yl]piperidine-1-carboxylate (130 mg, 0.27 mmol) to give 112 mg (100% yield) of tert-butyl 4-[6-(1-methyl-4-piperidyl)-2-oxo-3H-imidazo[4,5-b]pyridin-1-yl]piperidine-1-carboxylate as a white solid. LC/MS (m/z, M+H): calc. 416.2, found 416.2.

### Step 3: 6-(1-methyl-4-piperidyl)-1-(4-piperidyl)-3H-imidazo[4,5-b]pyridin-2-one, hydrochloride

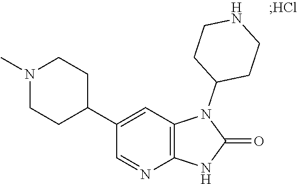 [SMILES: CN1CCC(c2cnc3[nH]c(=O)n(C4CCNCC4)c3c2)CC1]

Step 3 of Example 54 was performed following the protocol described in step 5 of Example 1 using tert-butyl 4-[6-(1-methyl-4-piperidyl)-2-oxo-3H-imidazo[4,5-b]pyridin-1-yl]piperidine-1-carboxylate (140 mg, 0.29 mmol) to give 100 mg (96% yield) of 6-(1-methyl-4-piperidyl)-1-(4-piperidyl)-3H-imidazo[4,5-b]pyridin-2-one, hydrochloride as a solid. LC/MS (m/z, M+H-HCl): calc. 316.2, found 316.2.

### Step 4: 1-[1-[2-amino-4-(trifluoromethoxy)benzoyl]-4-piperidyl]-6-(1-methyl-4-piperidyl)-3H-imidazo[4,5-b]pyridin-2-one

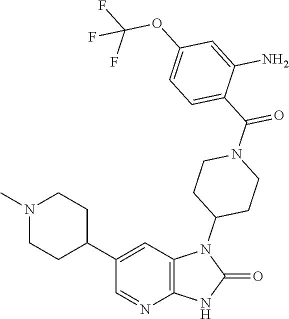 [SMILES: CN1CCC(c2cnc3[nH]c(=O)n(C4CCN(C(=O)c5ccc(OC(F)(F)F)cc5N)CC4)c3c2)CC1]

Step 4 of Example 54 was performed following the protocol described in step 4 of Example 15 using 6-(1-methyl-4-piperidyl)-1-(4-piperidyl)-3H-imidazo[4,5-b]pyridin-2-one, hydrochloride (100 mg, 0.28 mmol), 2-amino-4-(trifluoromethoxy)benzoic acid (69 mg, 0.31 mmol) and triethylamine (0.16 mL, 1.14 mmol) instead of DIPEA, to give 47 mg (32% yield) of 1-[1-[2-amino-4-(trifluoromethoxy)benzoyl]-4-piperidyl]-6-(1-methyl-4-piperidyl)-3H-imidazo[4,5-b]pyridin-2-one as a solid. LC/MS (m/z, M+H): calc. 519.2, found 519.2; ¹H NMR (400 MHz, DMSO-d6, 100° C.) d ppm 1.70-1.82 (m, 6H), 1.96-2.06 (m, 2H), 2.22 (s, 3H), 2.29 (qd, J=4.6 and 12.7 Hz, 2H), 2.50-2.55 (m partially hidden, 1H), 2.85-2.90 (m partially hidden, 2H), 3.01-3.10 (m, 2H), 4.16 (br d, J=12.7 Hz, 2H), 4.39-4.47 (m, 1H), 5.42 (br s, 2H), 6.49 (br d, J=8.4 Hz, 1H), 6.69 (br s, 1H), 7.14 (d, J=8.4 Hz, 1H), 7.40 (d, J=1.8 Hz, 1H), 7.77 (d, J=1.8 Hz, 1H), 11.03 (br s, 1H).

### Example 56: (rac)-6-tetrahydrofuran-3-yloxy-1-[1-[4-(trifluoromethoxy)benzoyl]-4-piperidyl]-3H-imidazo[4,5-b]pyridin-2-one

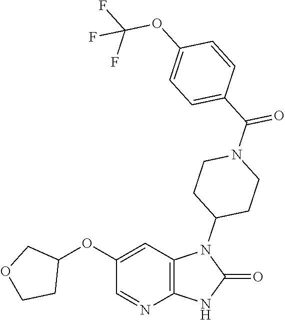 [SMILES: O=C(c1ccc(OC(F)(F)F)cc1)N1CCC(n2c(=O)[nH]c3ncc(OC4CCOC4)cc32)CC1]

### Step 1: (rac)-tert-butyl 4-[(2-nitro-5-tetrahydrofuran-3-yloxy-3-pyridyl)amino]piperidine-1-carboxylate

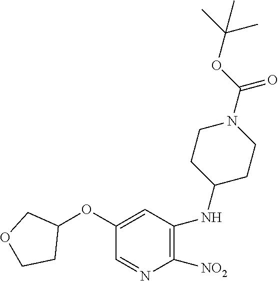 [SMILES: CC(C)(C)OC(=O)N1CCC(Nc2cc(OC3CCOC3)cnc2[N+](=O)[O-])CC1]

Step 1 of Example 56 was performed following the protocol described in step 2 of Example 1 using tert-butyl 4-[(5-fluoro-2-nitro-3-pyridyl)amino]piperidine-1-carboxylate (400 mg, 1.17 mmol) and (rac)-tetrahydrofuran-3-ol (207 mg, 2.35 mmol) to give 466 mg (97% yield) of (rac)-tert-butyl 4-[(2-nitro-5-tetrahydrofuran-3-yloxy-3-pyridyl)amino]piperidine-1-carboxylate as a yellow solid. LC/MS (m/z, M+H-BOC): calc. 309.2, found 309.1 (M+H).

### Step 2: (rac)-tert-butyl 4-[(2-amino-5-tetrahydrofuran-3-yloxy-3-pyridyl)amino]piperidine-1-carboxylate

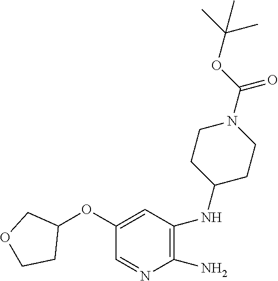 [SMILES: CC(C)(C)OC(=O)N1CCC(Nc2cc(OC3CCOC3)cnc2N)CC1]

Step 2 of Example 56 was performed following the protocol described in step 3 of Example 1 using (rac)-tert-butyl 4-[(2-nitro-5-tetrahydrofuran-3-yloxy-3-pyridyl)amino]piperidine-1-carboxylate (463 mg, 1.13 mmol) heating the reaction mixture at 80° C. instead of 65° C., to give 325 mg (76% yield) of (rac)-tert-butyl 4-[(2-amino-5-tetrahydrofuran-3-yloxy-3-pyridyl)amino]piperidine-1-carboxylate as a brown solid. LC/MS (m/z, M+H): calc. 379.2, found 379.2.

### Step 3: (rac)-tert-butyl 4-(2-oxo-6-tetrahydrofuran-3-yloxy-3H-imidazo[4,5-b]pyridin-1-yl)piperidine-1-carboxylate

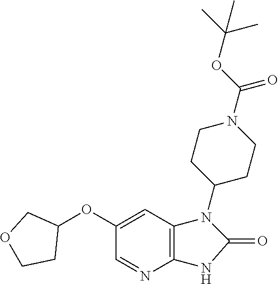 [SMILES: CC(C)(C)OC(=O)N1CCC(n2c(=O)[nH]c3ncc(OC4CCOC4)cc32)CC1]

Step 3 of Example 56 was performed following the protocol described in step 4 of Example 1 using (rac)-tert-butyl 4-[(2-amino-5-tetrahydrofuran-3-yloxy-3-pyridyl)amino]piperidine-1-carboxylate (322 mg, 0.85 mmol) to give 246 mg (71% yield) of (rac)-tert-butyl 4-(2-oxo-6-tetrahydrofuran-3-yloxy-3H-imidazo[4,5-b]pyridin-1-yl)piperidine-1-carboxylate as a white solid. LC/MS (m/z, M+H): calc. 405.2, found 405.2.

### Step 4: (rac)-1-(4-piperidyl)-6-tetrahydrofuran-3-yloxy-3H-imidazo[4,5-b]pyridin-2-one, dihydrochloride

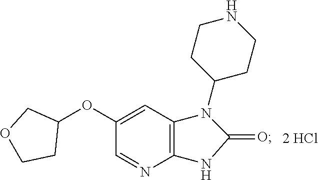 [SMILES: O=c1[nH]c2ncc(OC3CCOC3)cc2n1C1CCNCC1]

Step 4 of Example 56 was performed following the protocol described in step 5 of Example 1 using (rac)-tert-butyl 4-(2-oxo-6-tetrahydrofuran-3-yloxy-3H-imidazo[4,5-b]pyridin-1-yl)piperidine-1-carboxylate (243 mg, 0.6 mmol) to give 226 mg (100% yield) of (rac)-1-(4-piperidyl)-6-tetrahydrofuran-3-yloxy-3H-imidazo[4,5-b]pyridin-2-one, dihydrochloride as a grey solid. LC/MS (m/z, M+H-2HCl): calc. 305.2, found 305.1.

### Step 5: (rac)-6-tetrahydrofuran-3-yloxy-1-[1-[4-(trifluoromethoxy)benzoyl]-4-piperidyl]-3H-imidazo[4,5-b]pyridin-2-one

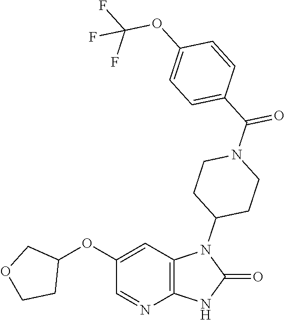 [SMILES: O=C(c1ccc(OC(F)(F)F)cc1)N1CCC(n2c(=O)[nH]c3ncc(OC4CCOC4)cc32)CC1]

Step 5 of Example 56 was performed following the protocol described in step 4 of Example 15 using (rac)-1-(4-piperidyl)-6-tetrahydrofuran-3-yloxy-3H-imidazo[4,5-b]pyridin-2-one, dihydrochloride (113 mg, 0.30 mmol) and 4-(trifluoromethoxy)benzoic acid (68 mg, 0.33 mmol) to give 114 mg (77% yield) of (rac)-6-tetrahydrofuran-3-yloxy-1-[1-[4-(trifluoromethoxy)benzoyl]-4-piperidyl]-3H-imidazo[4,5-b]pyridin-2-one as a white solid. LC/MS (m/z, M+H): calc. 493.2, found 493.1; ¹H NMR (400 MHz, DMSO-d6, 100° C.) d ppm 1.82 (br d, J=12.7 Hz, 2H), 2.00-2.08 (m, 1H), 2.17-2.24 (m, 1H), 2.24-2.36 (m, 2H), 3.04-3.14 (m, 2H), 3.76-3.93 (m, 4H), 4.12-4.27 (m, 2H), 4.38-4.47 (m, 1H), 5.04 (ddt, J=2.2 and 4.4 and 6.4 Hz, 1H), 7.29 (d, J=2.4 Hz, 1H), 7.41 (br d, J=8.4 Hz, 2H), 7.58-7.63 (m, 3H), 11.03 (br s, 1H).

### Examples 57 & 58: trans-6-(4-hydroxycyclohexyl)-1-[1-[4-(trifluoromethoxy)benzoyl]-4-piperidyl]-3H-imidazo[4,5-b]pyridine-2-one and cis-6-(4-hydroxycyclohexyl)-1-[1-[4-(trifluoromethoxy)benzoyl]-4-piperidyl]-3H-imidazo[4,5-b]pyridin-2-one

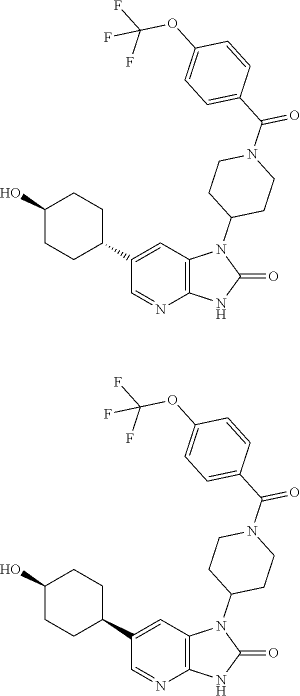 [SMILES: O=C(c1ccc(OC(F)(F)F)cc1)N1CCC(n2c(=O)[nH]c3ncc([C@H]4CC[C@H](O)CC4)cc32)CC1 ; O=C(c1ccc(OC(F)(F)F)cc1)N1CCC(n2c(=O)[nH]c3ncc([C@H]4CC[C@@H](O)CC4)cc32)CC1]

### Step 1: (rac)-6-(4-hydroxycyclohexen-1-yl)-1-[1-[4-(trifluoromethoxy)benzoyl]-4-piperidyl]-3H-imidazo[4,5-b]pyridin-2-one

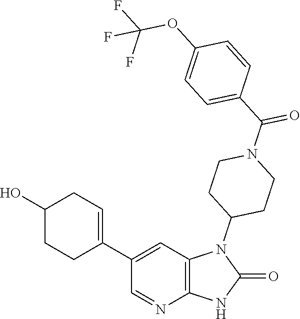 [SMILES: O=C(c1ccc(OC(F)(F)F)cc1)N1CCC(n2c(=O)[nH]c3ncc(C4=CCC(O)CC4)cc32)CC1]

To a solution of 6-bromo-1-[1-[4-(trifluoromethoxy)benzoyl]-4-piperidyl]-3H-imidazo[4,5-b]pyridin-2-one (160 mg, 0.33 mmol, Example 60) and 4-(4,4,5,5-tetramethyl-1,3,2-dioxaborolan-2-yl)cyclohex-3-en-1-ol (89 mg, 0.39 mmol) in dioxane (8 mL) and water (2 mL) was added K₃PO₄ (154 mg, 0.72 mmol) and the resulting mixture was bubbled with argon for five minutes. Then, PdG2SPhos (24 mg, 0.033 mmol) was added and the resulting mixture was refluxed for 12 hours. After 12 hours, the reaction mixture was cooled down to room temperature, diluted with ethyl acetate, washed with a 1N solution of NaOH, with brine. The combined aqueous layers were extracted with ethyl acetate, and the combined organic layers were dried over sodium sulfate, filtered and concentrated in vacuo. The resulting residue was purified by flash chromatography on silica gel (SiO₂ 10 g) eluting with DCM/MeOH 95/5 to give 146 mg (88% yield) of (rac)-6-(4-hydroxycyclohexen-1-yl)-1-[1-[4-(trifluoromethoxy)benzoyl]-4-piperidyl]-3H-imidazo[4,5-b]pyridin-2-one as a yellow solid. LC/MS (m/z, M+H): calc. 503.2, found 503.2.

### Step 2: cis/trans mixture of 6-(4-hydroxycyclohexyl)-1-[1-[4-(trifluoromethoxy)benzoyl]-4-piperidyl]-3H-imidazo[4,5-b]pyridin-2-one

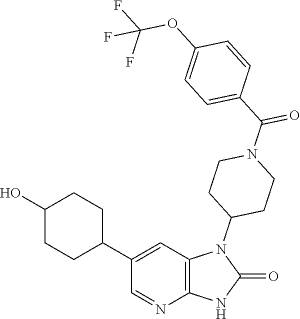 [SMILES: O=C(c1ccc(OC(F)(F)F)cc1)N1CCC(n2c(=O)[nH]c3ncc(C4CCC(O)CC4)cc32)CC1]

To a solution of (rac)-6-(4-hydroxycyclohexen-1-yl)-1-[1-[4-(trifluoromethoxy)benzoyl]-4-piperidyl]-3H-imidazo[4,5-b]pyridin-2-one (143 mg, 0.28 mmol) in MeOH (4 mL) and DCM (4 mL) was added Pd/C (10%)-50% wet (20 mg) and the whole mixture was submitted to hydrogenation with 4 bars of hydrogen at room temperature for 22 hours. After 22 hours, the reaction mixture was filtered, Pd/C (10%)-50% wet (80 mg) was added and the resulting mixture was submitted to hydrogenation with 4 bars of hydrogen at room temperature for 48 additional hours. After 48 hours, the reaction mixture was filtered and concentrated to dryness. The resulting residue was purified by flash chromatography on silica gel (SiO₂ 25 g) eluting with EtOAc/MeOH 92/8 to give 100 mg (69% yield) of a cis/trans mixture of 6-(4-hydroxycyclohexyl)-1-[1-[4-(trifluoromethoxy)benzoyl]-4-piperidyl]-3H-imidazo[4,5-b]pyridin-2-one as a pale brown solid. LC/MS (m/z, M+H): calc. 505.2, found 505.2.

### Step 3: trans-6-(4-hydroxycyclohexyl)-1-[1-[4-(trifluoromethoxy)benzoyl]-4-piperidyl]-3H-imidazo[4,5-b]pyridin-2-one and cis-6-(4-hydroxycyclohexyl)-1-[1-[4-(trifluoromethoxy)benzoyl]-4-piperidyl]-3H-imidazo[4,5-b]pyridine-2-one

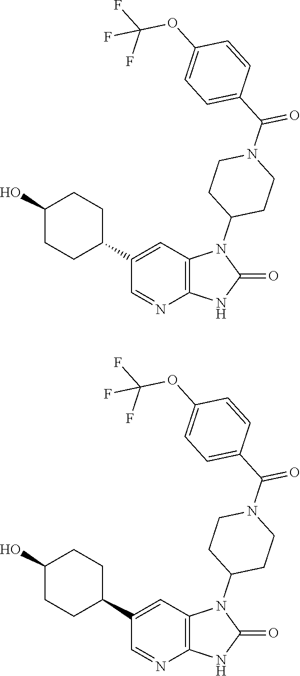 [SMILES: O=C(c1ccc(OC(F)(F)F)cc1)N1CCC(n2c(=O)[nH]c3ncc([C@H]4CC[C@H](O)CC4)cc32)CC1 ; O=C(c1ccc(OC(F)(F)F)cc1)N1CCC(n2c(=O)[nH]c3ncc([C@H]4CC[C@@H](O)CC4)cc32)CC1]

Separation of the cis/trans mixture of 6-(4-hydroxycyclohexyl)-1-[1-[4-(trifluoromethoxy)benzoyl]-4-piperidyl]-3H-imidazo[4,5-b]pyridin-2-one (100 mg, 0.2 mmol) was performed using a Chiralpak IC column (20 μm, 350×76.5 mm), eluting with (heptane 50/EtOH 50)+0.1% TEA (flow rate 400 mL/min, UV detection at 265 nm) to give: 14 mg (14% yield) of trans-6-(4-hydroxycyclohexyl)-1-[1-[4-(trifluoromethoxy)benzoyl]-4-piperidyl]-3H-imidazo[4,5-b]pyridine-2-one. LC/MS (m/z, M+H): calc. 505.2, found 505.1; ¹H NMR (400 MHz, DMSO-d6, 100° C.) d ppm 1.28-1.38 (m, 2H), 1.47-1.60 (m, 2H), 1.76-1.86 (m, 4H), 1.91-2.00 (m, 2H), 2.24-2.34 (m, 2H), 2.51-2.56 (m, 1H), 3.03-3.15 (m, 2H), 3.45-3.54 (m, 1H), 4.07-4.26 (m, 2H), 4.14 (br d, J=4.6 Hz, 1H), 4.37-4.46 (m, 1H), 7.36 (d, J=1.8 Hz, 1H), 7.40 (br d, J=8.4 Hz, 2H), 7.57 (d, J=8.4 Hz, 2H), 7.76 (d, J=1.8 Hz, 1H), 10.78-11.14 (m, 1H); and

66 mg (66% yield) of cis-6-(4-hydroxycyclohexyl)-1-[1-[4-(trifluoromethoxy)benzoyl]-4-piperidyl]-3H-imidazo[4,5-b]pyridine-2-one. LC/MS (m/z, M+H): calc. 505.2, found 505.1; ¹H NMR (400 MHz, DMSO-d6, 100° C.) d ppm 1.51-1.64 (m, 4H), 1.73-1.84 (m, 4H), 1.85-1.96 (m, 2H), 2.21-2.31 (m, 2H), 2.54-2.61 (m, 1H), 3.06-3.15 (m, 2H), 3.90-3.94 (m, 1H), 3.99 (br d, J=3.9 Hz, 1H), 4.09-4.25 (m, 2H), 4.36-4.50 (m, 1H), 7.34 (br s, 1H), 7.40 (br d, J=8.4 Hz, 2H), 7.57 (br d, J=8.4 Hz, 2H), 7.77 (br s, 1H), 10.86-11.13 (m, 1H).

### Example 59: tert-butyl N-[2-[4-(6-bromo-2-oxo-3H-imidazo[4,5-b]pyridin-1-yl)piperidine-1-carbonyl]-5-(trifluoromethoxy)phenyl]carbamate

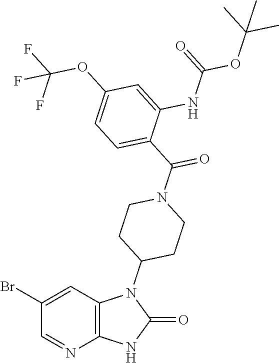 [SMILES: CC(C)(C)OC(=O)Nc1cc(OC(F)(F)F)ccc1C(=O)N1CCC(n2c(=O)[nH]c3ncc(Br)cc32)CC1]

At room temperature, to a solution of 6-bromo-1-(4-piperidyl)-3H-imidazo[4,5-b]pyridin-2-one, hydrochloride (8 g, 23.98 mmol, prepared in step 2 of Examples 60 & 11) in DMF (80 mL) were successively added triethylamine (23.4 mL, 8.04 mmol), 2-(tert-butoxycarbonylamino)-4-(trifluoromethoxy)benzoic acid (7.703 g, 23.98 mmol) and TBTU (8.469 g, 26.38 mmol) and the resulting mixture was stirred for 3 hours, then poured on water (800 mL), extracted twice with AcOEt (2×300 mL) and the combined organic layers were dried over magnesium sulfate, filtered and concentrated in vacuo. The resulting residue was purified by flash chromatography on silica gel (SiO₂ 400 g) eluting with DCM/[DCM/MeOH 95/5]90/10 to give 4.83 g (34% yield) of tert-butyl N-[2-[4-(6-bromo-2-oxo-3H-imidazo[4,5-b]pyridin-1-yl)piperidine-1-carbonyl]-5-(trifluoromethoxy)phenyl]carbamate as a white solid. LC/MS (m/z, M+H): calc. 600.1, found 600.1; ¹H NMR (400 MHz, DMSO-d6, 100° C.) d ppm 1.50 (s, 9H), 1.84 (br d, J=12.7 Hz, 2H), 2.27-2.38 (m, 2H), 3.02-3.13 (m, 2H), 4.10-4.27 (m, 2H), 4.39-4.49 (m, 1H), 7.09 (br d, J=8.4 Hz, 1H), 7.48 (d, J=8.4 Hz, 1H), 7.72 (br s, 1H), 7.83 (d, J=1.8 Hz, 1H), 7.98 (d, J=1.8 Hz, 1H), 8.66 (br s, 1H), 11.28-11.65 (m, 1H).

### Examples 63, 61 and 62: (rac)-6-(4-oxocycloheptyl)-1-[1-[4-(trifluoromethoxy)benzoyl]-4-piperidyl]-3H-imidazo[4,5-b]pyridin-2-one, (rac)-trans-6-(4-hydroxycycloheptyl)-1-[1-[4-(trifluoromethoxy)benzoyl]-4-piperidyl]-3H-imidazo[4,5-b]pyridin-2-one & (rac)-cis-6-(4-hydroxycycloheptyl)-1-[1-[4-(trifluoromethoxy)benzoyl]-4-piperidyl]-3H-imidazo[4,5-b]pyridin-2-one

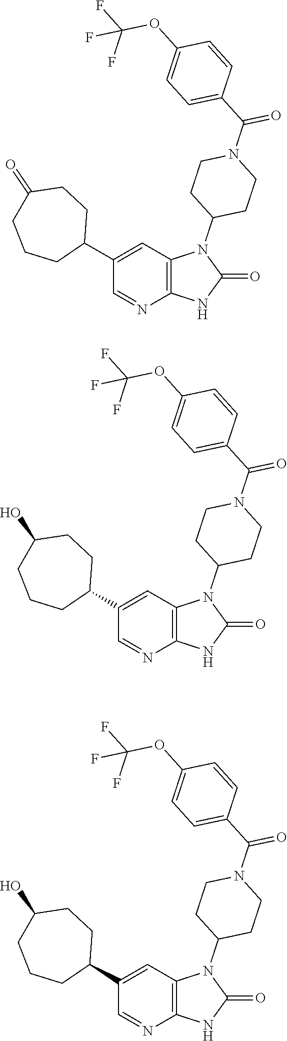 [SMILES: O=C1CCCC(c2cnc3[nH]c(=O)n(C4CCN(C(=O)c5ccc(OC(F)(F)F)cc5)CC4)c3c2)CC1 ; O=C(c1ccc(OC(F)(F)F)cc1)N1CCC(n2c(=O)[nH]c3ncc([C@@H]4CCC[C@@H](O)CC4)cc32)CC1 ; O=C(c1ccc(OC(F)(F)F)cc1)N1CCC(n2c(=O)[nH]c3ncc([C@H]4CCC[C@@H](O)CC4)cc32)CC1]

### Step 1: 6-bromo-1-[1-[4-(trifluoromethoxy)benzoyl]-4-piperidyl]-3-(2-trimethylsilylethoxymethyl)imidazo[4,5-b]pyridin-2-one

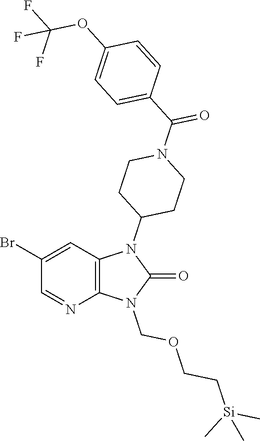 [SMILES: C[Si](C)(C)CCOCn1c(=O)n(C2CCN(C(=O)c3ccc(OC(F)(F)F)cc3)CC2)c2cc(Br)cnc21]

To a stirred solution of 6-bromo-1-[1-[4-(trifluoromethoxy)benzoyl]-4-piperidyl]-3H-imidazo[4,5-b]pyridin-2-one (1.80 g, 3.71 mmol, Example 60) in N,N-dimethylformamide (10 mL) at 0° C. was added sodium hydride (60% dispersion in oil, 356 mg, 14.8 mmol). Then, the reaction mixture was stirred at room temperature for 15 minutes under argon atmosphere. After 15 minutes, 2-(chloromethoxy)ethyl-trimethyl-silane (1.86 g, 11.1 mmol) was added at 0° C. The resulting reaction mixture was stirred for 2 hours at room temperature. After 2 hours, the reaction mixture was diluted with ice water (50 mL) and extracted with ethyl acetate (50 mL×2). The combined organic layers were separated, dried over Na₂SO₄, filtered, and concentrated under reduced pressure. The resulting residue was purified by flash chromatography, eluting with 30% EtOAc in hexane to give 1.7 g (66% yield) of 6-bromo-1-[1-[4-(trifluoromethoxy)benzoyl]-4-piperidyl]-3-(2-trimethylsilylethoxymethyl)imidazo[4,5-b]pyridin-2-one as a brown solid. LC/MS (m/z, M+H): calc. 615.1, found 615.2.

### Step 2: [2-oxo-1-[1-[4-(trifluoromethoxy)benzoyl]-4-piperidyl]-3-(2-trimethylsilylethoxymethyl)imidazo[4,5-b]pyridin-6-yl]boronic acid

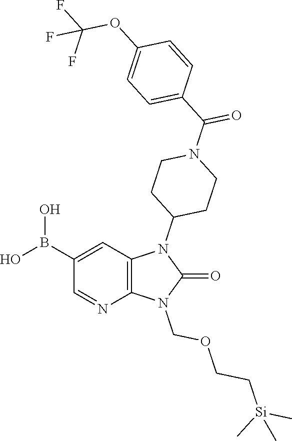 [SMILES: C[Si](C)(C)CCOCn1c(=O)n(C2CCN(C(=O)c3ccc(OC(F)(F)F)cc3)CC2)c2cc(B(O)O)cnc21]

To a stirred solution of 6-bromo-1-[1-[4-(trifluoromethoxy)benzoyl]-4-piperidyl]-3-(2-trimethylsilylethoxymethyl)imidazo[4,5-b]pyridin-2-one (1.70 g, 2.76 mmol) and 4,4,5,5-tetramethyl-2-(4,4,5,5-tetramethyl-1,3,2-dioxaborolan-2-yl)-1,3,2-dioxaborolane (1.05 g, 4.14 mmol) in 1,4-dioxane (20 mL), was added KOAc (542 mg, 5.52 mmol) and the resulting mixture was degassed with argon for 15 minutes. Then, PdCl₂(dppf). DCM complex (226 mg, 0.28 mmol) was added and the reaction mixture was stirred at 80° C. for 12 hours. After 12 hours, the reaction mixture was cooled down to room temperature and then filtered through a celite bed. The filtrate was concentrated under reduced pressure. The resulting residue was purified by flash chromatography eluting with 40% EtOAc in hexane to give 1.5 g (83% yield) of [2-oxo-1-[1-[4-(trifluoromethoxy)benzoyl]-4-piperidyl]-3-(2-trimethylsilylethoxymethyl)imidazo[4,5-b]pyridin-6-yl]boronic acid as an off white solid. LC/MS (m/z, M+H): calc. 581.2, found 581.1.

### Step 3: 1,4-dioxaspiro[4.6]undec-7-en-8-yl trifluoromethanesulfonate

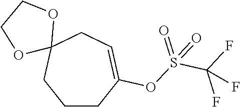 [SMILES: O=S(=O)(OC1=CCC2(CCC1)OCCO2)C(F)(F)F]

To a stirred solution of 1,4-dioxaspiro[4.6]undecan-8-one (1 g, 5.88 mmol) in tetrahydrofuran (50 mL) was added lithium bis(trimethylsilyl)azanide (1.00 mol/L, 12.9 mL, 12.9 mmol) at −78° C. and the resulting mixture was stirred for 30 minutes. After 30 minutes, was added 1,1,1-trifluoro-N-phenyl-N-(trifluoromethylsulfonyl)methanesulfonamide (2.10 g, 5.88 mmol) at −78° C., and the resulting reaction mixture was stirred at room temperature for 16 hours. After 16 hours, the reaction mixture was concentrated under reduced pressure and quenched with iced water (100 mL) and extracted with ethyl acetate (100 mL×2). The combined organic layers were dried over Na₂SO₄, filtered and concentrated under reduced pressure. The resulting residue was then purified on silica gel to give 1 g (56% yield) of 1,4-dioxaspiro[4.6]undec-7-en-8-yl trifluoromethanesulfonate as a yellow liquid.

### Step 4: 6-(1,4-dioxaspiro[4.6]undec-7-en-8-yl)-1-[1-[4-(trifluoromethoxy)benzoyl]-4-piperidyl]-3-(2-trimethylsilylethoxymethyl)imidazo[4,5-b]pyridin-2-one

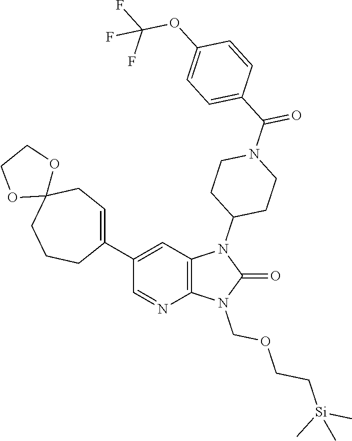 [SMILES: C[Si](C)(C)CCOCn1c(=O)n(C2CCN(C(=O)c3ccc(OC(F)(F)F)cc3)CC2)c2cc(C3=CCC4(CCC3)OCCO4)cnc21]

To a stirred solution of [2-oxo-1-[1-[4-(trifluoromethoxy)benzoyl]-4-piperidyl]-3-(2-trimethylsilylethoxymethyl)imidazo[4,5-b]pyridin-6-yl]boronic acid (800 mg, 1.38 mmol) and 1,4-dioxaspiro[4.6]undec-7-en-8-yl trifluoromethanesulfonate (500 mg, 1.65 mmol) in 1,4-dioxane (10 mL) was added dipotassium carbonate (381 mg, 2.76 mmol) in water (1 mL), and the resulting mixture was purged with argon for 10 minutes. Then, [1,1′-bis(diphenylphosphino)ferrocene]dichloropalladium (II) (56 mg, 0.07 mmol) was added and the resulting reaction mixture was stirred for 6 hours at 100° C. After 6 hours, the reaction mixture was poured onto ice water (20 mL) then extracted with EtOAc (150 mL). The organic layer was separated, washed with brine, dried over Na₂SO₄, filtered and concentrated under reduced pressure. The resulting residue was purified by flash chromatography, eluting with 30% EtOAc in hexane to give 500 mg (53% yield) of 6-(1,4-dioxaspiro[4.6]undec-7-en-8-yl)-1-[1-[4-(trifluoromethoxy)benzoyl]-4-piperidyl]-3-(2-trimethylsilylethoxymethyl)imidazo[4,5-b]pyridin-2-one. LC/MS (m/z, M+H): calc. 689.3, found 689.5.

### Step 5: 6-(1,4-dioxaspiro[4.6]undec-7-en-8-yl)-1-[1-[4-(trifluoromethoxy)benzoyl]-4-piperidyl]-3H-imidazo[4,5-b]pyridin-2-one

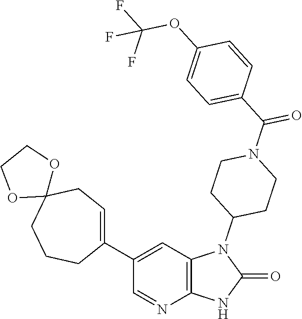 [SMILES: O=C(c1ccc(OC(F)(F)F)cc1)N1CCC(n2c(=O)[nH]c3ncc(C4=CCC5(CCC4)OCCO5)cc32)CC1]

To a stirred solution of 6-(1,4-dioxaspiro[4.6]undec-7-en-8-yl)-1-[1-[4-(trifluoromethoxy)benzoyl]-4-piperidyl]-3-(2-trimethylsilylethoxymethyl)imidazo[4,5-b]pyridin-2-one (949 mg, 1.38 mmol) in THF (5 mL) at 0° C., was added tetrabutylammonium;fluoride trihydrate (870 mg, 2.76 mmol) and the resulting reaction mixture was stirred for 2 hours at 80° C. After 2 hours, the reaction mixture was quenched with water (30 mL) and extracted with EtOAc (50 mL), and the separated organic layer was washed with brine, concentrated under reduced pressure to give 500 mg (65% yield) of crude 6-(1,4-dioxaspiro[4.6]undec-7-en-8-yl)-1-[1-[4-(trifluoromethoxy)benzoyl]-4-piperidyl]-3H-imidazo[4,5-b]pyridin-2-one as a yellow solid. LC/MS (m/z, M+H): calc. 559.2, found 559.4.

### Step 6: 6-(4-oxocyclohepten-1-yl)-1-[1-[4-(trifluoromethoxy)benzoyl]-4-piperidyl]-3H-imidazo[4,5-b]pyridin-2-one

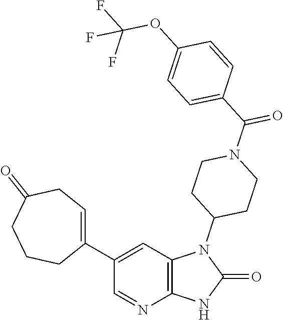 [SMILES: O=C1CC=C(c2cnc3[nH]c(=O)n(C4CCN(C(=O)c5ccc(OC(F)(F)F)cc5)CC4)c3c2)CCC1]

To a stirred solution of 6-(1,4-dioxaspiro[4.6]undec-7-en-8-yl)-1-[1-[4-(trifluoromethoxy)benzoyl]-4-piperidyl]-3H-imidazo[4,5-b]pyridin-2-one (300 mg, 0.54 mmol) in DCM (10 mL) at 0° C., was added a 4M solution of HCl in dioxane (4 mol/L, 1.34 mL, 5.4 mmol). The reaction mixture was then stirred for 2 hours at room temperature. After 2 hours, the reaction mixture was concentrated under reduced pressure. The resulting residue was triturated with diethyl ether (30 mL) to give 250 mg (90% yield) of 6-(4-oxocyclohepten-1-yl)-1-[1-[4-(trifluoromethoxy)benzoyl]-4-piperidyl]-3H-imidazo[4,5-b]pyridin-2-one as a yellow solid. LC/MS (m/z, M+H): calc. 515.2, found 515.1.

### Step 7 (Example 63): (rac)-6-(4-oxocycloheptyl)-1-[1-[4-(trifluoromethoxy)benzoyl]-4-piperidyl]-3H-imidazo[4,5-b]pyridine-2-one

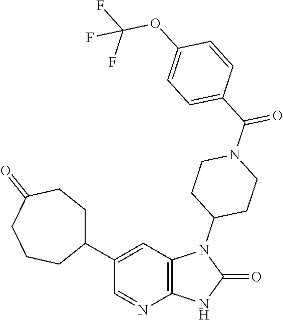 [SMILES: O=C1CCCC(c2cnc3[nH]c(=O)n(C4CCN(C(=O)c5ccc(OC(F)(F)F)cc5)CC4)c3c2)CC1]

To a stirred solution of 6-(4-oxocyclohepten-1-yl)-1-[1-[4-(trifluoromethoxy)benzoyl]-4-piperidyl]-3H-imidazo [4,5-b]pyridine-2-one (250 mg, 0.49 mmol) in MeOH (10 mL), was added 10% Pd/C (517 mg, 4.86 mmol) at room temperature. Then, the reaction mixture was stirred at room temperature for 48 hours under 1 atmosphere of H₂. After 48 hours, the reaction mixture was filtered through a celite bed and washed with ethanol. The resulting filtrate was concentrated under reduced pressure. The resulting residue was purified by flash chromatography, eluting with 5% MeOH in DCM, to give 250 mg (99% yield) of (rac)-6-(4-oxocycloheptyl)-1-[1-[4-(trifluoromethoxy)benzoyl]-4-piperidyl]-3H-imidazo[4,5-b]pyridine-2-one as a yellow solid. LC/MS (m/z, M+H): calc. 517.2, found 517.2.

### Step 8 (Examples 61 and 62): (rac)-trans-6-(4-hydroxycycloheptyl)-1-[1-[4-(trifluoromethoxy)benzoyl]-4-piperidyl]-3H-imidazo[4,5-b]pyridine-2-one & (rac)-cis-6-(4-hydroxycycloheptyl)-1-[1-[4-(trifluoromethoxy)benzoyl]-4-piperidyl]-3H-imidazo[4,5-b]pyridine-2-one

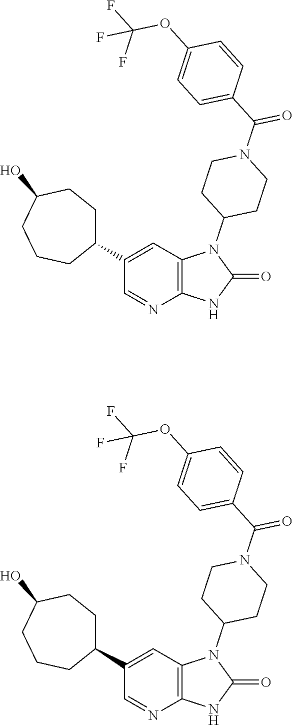 [SMILES: O=C(c1ccc(OC(F)(F)F)cc1)N1CCC(n2c(=O)[nH]c3ncc([C@@H]4CCC[C@@H](O)CC4)cc32)CC1 ; O=C(c1ccc(OC(F)(F)F)cc1)N1CCC(n2c(=O)[nH]c3ncc([C@H]4CCC[C@@H](O)CC4)cc32)CC1]

To a stirred solution of (rac)-6-(4-oxocycloheptyl)-1-[1-[4-(trifluoromethoxy)benzoyl]-4-piperidyl]-3H-imidazo[4,5-b]pyridin-2-one (100 mg, 0.19 mmol) in MeOH (5 mL), was added NaBH₄ (37 mg, 0.97 mmol) at room temperature and the resulting reaction mixture was stirred for 2 hours at room temperature. After 2 hours, the reaction mixture was diluted with water, extracted with EtOAc (30 mL). The organic layer was separated, dried over Na₂SO₄ and concentrated under reduced pressure. The resulting residue was purified by preparative HPLC (Conditions: MOBILE PHASE-A: 10 Mm ABC in water, MOBILE PHASE-B: acetonitrile, COLUMN CONDITION: XSELECT (19×150 mm) 10 μm, GRADIENT: time (minutes) % B, 0 min 25%, 2 min 35%, 8 min 55%, flow rate: 18 mL/min) to give 10 mg (10% yield) of (rac)-trans-6-[(4R)-4-hydroxycycloheptyl]-1-[1-[4-(trifluoromethoxy)benzoyl]-4-piperidyl]-3H-imidazo[4,5-b]pyridin-2-one. LC/MS (m/z, M+H): calc. 519.2, found 519.2. ¹H NMR (400 MHz, DMSO-d6) δ ppm 11.33-11.46 (m, 1H) 7.71-7.81 (m, 1H) 7.55-7.65 (m, 2H) 7.41-7.51 (m, 3H) 4.56-4.77 (m, 1H) 4.39-4.56 (m, 2H) 3.62-3.85 (m, 2H) 2.83-3.01 (m, 1H) 2.66-2.78 (m, 2H) 2.21-2.37 (m, 3H) 1.39-2.05 (m, 12H); and

9 mg (9% yield) of (rac)-cis-6-[(4R)-4-hydroxycycloheptyl]-1-[1-[4-(trifluoromethoxy)benzoyl]-4-piperidyl]-3H-imidazo[4,5-b]pyridin-2-one as an off white solid. LC/MS (m/z, M+H): calc. 519.2, found 519.2. ¹H NMR (400 MHz, DMSO-d6) δ ppm 11.24-11.51 (m, 1H) 4.57-4.72 (m, 1H) 4.39-4.52 (m, 2H) 3.78-3.91 (m, 1H) 3.61-3.72 (m, 1H) 2.88-2.99 (m, 1H) 1.90-2.04 (m, 3H) 1.66-1.87 (m, 7H) 1.28-1.66 (m, 6H).

### Examples 64 & 65: (rac)-6-[(4E/Z)-4-hydroxyiminocycloheptyl]-1-[1-[4-(trifluoromethoxy)benzoyl]-4-piperidyl]-3H-imidazo[4,5-b]pyridin-2-one, Isomers 1 & 2

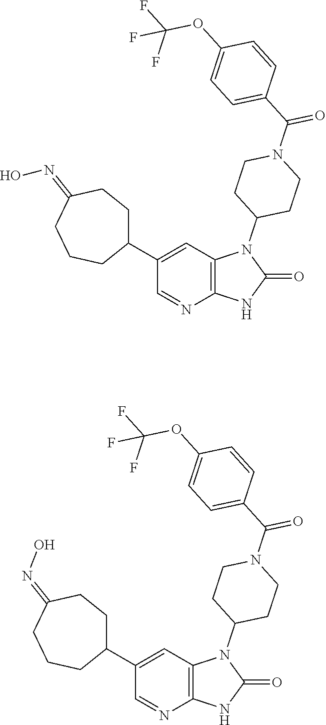 [SMILES: O=C(c1ccc(OC(F)(F)F)cc1)N1CCC(n2c(=O)[nH]c3ncc(C4CCC/C(=N\O)CC4)cc32)CC1 ; O=C(c1ccc(OC(F)(F)F)cc1)N1CCC(n2c(=O)[nH]c3ncc(C4CCC/C(=N/O)CC4)cc32)CC1]

To a stirred solution of (rac)-6-(4-oxocycloheptyl)-1-[1-[4-(trifluoromethoxy)benzoyl]-4-piperidyl]-3H-imidazo[4,5-b]pyridin-2-one (120 mg, 0.23 mmol) in EtOH (5 mL), were added NaHCO₃ (59 mg, 0.7 mmol) and NH₂OH·HCl (48 mg, 0.7 mmol) at room temperature and the resulting reaction mixture was stirred for 2 hours at room temperature. After 2 hours, the reaction mixture was diluted with water (10 mL), extracted with EtOAc (30 mL). The organic layer was separated, dried over Na₂SO₄, filtered and concentrated under reduced pressure. The resulting residue was purified by preparative HPLC (Conditions: A LINE: 10 mM Ammonium Bicarbonate in water; B LINE: ACN, COLUMN: X-Select (C18, 21.2 mm×250 mm), GRADIENT PROGRAMME: Flow rate 18 mL/min., Time (minutes) (0, 2, 6), % B (34, 45, 60)) to give 5 mg (4% yield) of (rac)-6-[(4E/Z)-4-hydroxyiminocycloheptyl]-1-[1-[4-(trifluoromethoxy)benzoyl]-4-piperidyl]-3H-imidazo[4,5-b]pyridin-2-one, Isomer 1: LC/MS (m/z, M+H): calc. 532.2, found 532.2. ¹H NMR (400 MHz, DMSO-d6) δ ppm 11.33-11.53 (m, 1H) 10.22 (s, 1H) 7.78 (s, 1H) 7.61 (d, J=8.51 Hz, 2H) 7.46-7.53 (m, 3H) 4.60-4.76 (m, 1H) 4.46 (br s, 1H) 3.60-3.78 (m, 1H) 3.18-3.28 (m, 1H) 2.84-3.05 (m, 2H) 2.63-2.78 (m, 3H) 1.91-2.06 (m, 3H) 1.76-1.89 (m, 2H) 1.56-1.73 (m, 2H) 1.33-1.51 (m, 4H); and 9 mg (7% yield) of (rac)-6-[(4E/Z)-4-hydroxyiminocycloheptyl]-1-[1-[4-(trifluoromethoxy)benzoyl]-4-piperidyl]-3H-imidazo[4,5-b]pyridin-2-one, Isomer 2 as an off white solid. LC/MS (m/z, M+H): calc. 532.2, found 532.2. ¹H NMR (400 MHz, DMSO-d6) δ ppm 11.30-11.52 (m, 1H) 10.24 (s, 1H) 7.73-7.81 (m, 1H) 7.57-7.66 (m, 2H) 7.44-7.57 (m, 3H) 4.58-4.78 (m, 1H) 4.38-4.53 (m, 1H) 3.60-3.75 (m, 1H) 3.18-3.28 (m, 1H) 2.84-2.96 (m, 1H) 2.63-2.78 (m, 3H) 2.21-2.33 (m, 2H) 1.80-2.01 (m, 4H) 1.60-1.76 (m, 3H) 1.48-1.60 (m, 1H)

### Examples 66 and 67: (rac)-trans-6-(3-hydroxycycloheptyl)-1-[1-[4-(trifluoromethoxy)benzoyl]-4-piperidyl]-3H-imidazo[4,5-b]pyridin-2-one and (rac)-cis-6-(3-hydroxycycloheptyl)-1-[1-[4-(trifluoromethoxy)benzoyl]-4-piperidyl]-3H-imidazo[4,5-b]pyridin-2-one

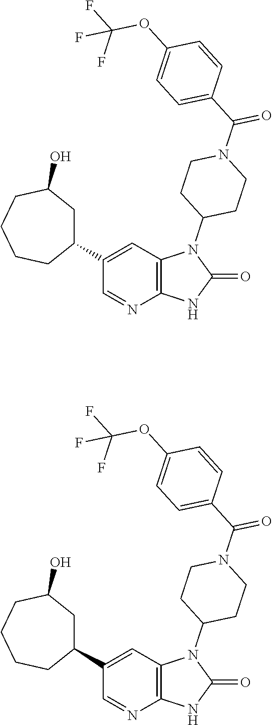 [SMILES: O=C(c1ccc(OC(F)(F)F)cc1)N1CCC(n2c(=O)[nH]c3ncc([C@@H]4CCCC[C@@H](O)C4)cc32)CC1 ; O=C(c1ccc(OC(F)(F)F)cc1)N1CCC(n2c(=O)[nH]c3ncc([C@H]4CCCC[C@@H](O)C4)cc32)CC1]

### Step 1: (3-oxocyclohepten-1-yl) trifluoromethanesulfonate

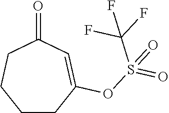 [SMILES: O=C1C=C(OS(=O)(=O)C(F)(F)F)CCCC1]

To a stirred solution of cycloheptane-1,3-dione (2 g, 15.9 mmol) in DCM (10 mL), was added Na₂CO₃ (1.68 g, 15.9 mmol) at 0° C., followed by a solution of trifluoromethylsulfonyl trifluoromethanesulfonate (5.37 g, 19 mmol) in DCM (40 mL) dropwise over 1 hour at room temperature under argon atmosphere. The resulting reaction mixture was stirred for 2 hours at room temperature. After 2 hours, the reaction mixture was diluted with ethyl acetate, filtered through a celite bed, and the resulting filtrate was concentrated under reduced pressure. The resulting residue was purified by flash chromatography eluting with 5% EtOAc in hexane to give 700 mg (12% yield) of (3-oxocyclohepten-1-yl) trifluoromethanesulfonate as a brown sticky solid.

### Step 2: 6-(3-oxocyclohepten-1-yl)-1-[1-[4-(trifluoromethoxy)benzoyl]-4-piperidyl]-3-(2-trimethylsilylethoxymethyl)imidazo[4,5-b]pyridin-2-one

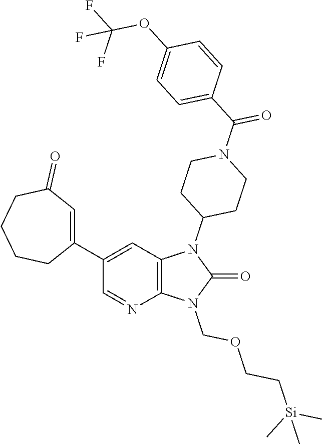 [SMILES: C[Si](C)(C)CCOCn1c(=O)n(C2CCN(C(=O)c3ccc(OC(F)(F)F)cc3)CC2)c2cc(C3=CC(=O)CCCC3)cnc21]

To a stirred solution of [2-oxo-1-[1-[4-(trifluoromethoxy)benzoyl]-4-piperidyl]-3-(2-trimethylsilylethoxymethyl)imidazo[4,5-b]pyridin-6-yl]boronic acid (800 mg, 1.38 mmol, prepared in step 2 of Examples 63, 61 & 62) and (3-oxocyclohepten-1-yl) trifluoro methanesulfonate (356 mg, 1.38 mmol) in 1,4-dioxane (10 mL) and water (2 mL) was added dipotassium carbonate (571 mg, 4.13 mmol) and the resulting reaction mixture was degassed with argon for 15 minutes. Then, PdCl₂(dppf). DCM complex (113 mg, 0.14 mmol) was added and the resulting reaction mixture was stirred at 100° C. for 16 hours. After 16 hours, the reaction mixture was cooled down to room temperature and then filtered through a celite bed. The filtrate was diluted with ethyl acetate (50 mL), and washed with water (50 mL). The organic layer was separated and dried over anhydrous Na₂SO₄, filtered and concentrated under reduced pressure. The resulting residue was purified by flash chromatography eluting with 50% EtOAc in hexane, to give 400 mg (40% yield) of 6-(3-oxocyclohepten-1-yl)-1-[1-[4-(trifluoromethoxy)benzoyl]-4-piperidyl]-3-(2-trimethylsilylethoxymethyl)imidazo[4,5-b]pyridin-2-one as a brown solid. LC/MS (m/z, M+H): calc. 645.3, found 645.1.

### Step 3: cis/trans mixture of (rac)-6-(3-hydroxycycloheptyl)-1-[1-[4-(trifluoromethoxy)benzoyl]-4-piperidyl]-3-(2-trimethylsilylethoxymethyl)imidazo[4,5-b]pyridin-2-one

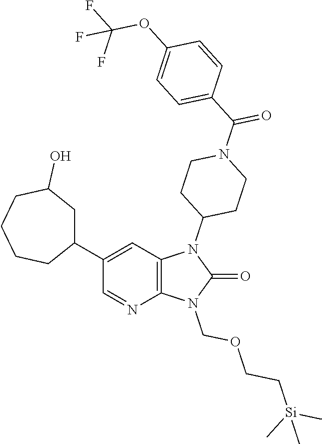 [SMILES: C[Si](C)(C)CCOCn1c(=O)n(C2CCN(C(=O)c3ccc(OC(F)(F)F)cc3)CC2)c2cc(C3CCCCC(O)C3)cnc21]

To a stirred solution of 6-(3-oxocyclohepten-1-yl)-1-[1-[4-(trifluoromethoxy)benzoyl]-4-piperidyl]-3-(2-trimethylsilylethoxymethyl)imidazo[4,5-b]pyridin-2-one (300 mg, 0.46 mmol) in methanol (10 mL), was added palladium (10% on Carbon) (149 mg, 1.40 mmol) followed by acetic acid (2.3 mg, 0.04 mmol) under an argon atmosphere and then the reaction mixture was submitted to hydrogenation under 4 atmospheres of H₂ at room temperature for 48 hours. After 48 hours, the reaction mixture was filtered through a celite bed and washed with ethanol. The resulting filtrate was concentrated under reduced pressure to give 290 mg (74% yield) of a cis/trans mixture of (rac)-6-(3-hydroxycycloheptyl)-1-[1-[4-(trifluoromethoxy)benzoyl]-4-piperidyl]-3-(2-trimethylsilylethoxymethyl)imidazo[4,5-b]pyridin-2-one as a brown solid. LC/MS (m/z, M+H): calc. 647.3, found 647.0.

### Step 4: (Examples 66 and 67): (rac)-trans-6-(3-hydroxycycloheptyl)-1-[1-[4-(trifluoromethoxy)benzoyl]-4-piperidyl]-3H-imidazo[4,5-b]pyridin-2-one and (rac)-cis-6-(3-hydroxycycloheptyl)-1-[1-[4-(trifluoromethoxy)benzoyl]-4-piperidyl]-3H-imidazo[4,5-b]pyridin-2-one

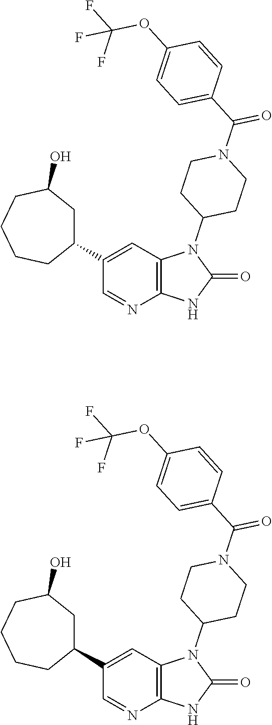 [SMILES: O=C(c1ccc(OC(F)(F)F)cc1)N1CCC(n2c(=O)[nH]c3ncc([C@@H]4CCCC[C@@H](O)C4)cc32)CC1 ; O=C(c1ccc(OC(F)(F)F)cc1)N1CCC(n2c(=O)[nH]c3ncc([C@H]4CCCC[C@@H](O)C4)cc32)CC1]

To a stirred solution of a cis/trans mixture of (rac)-6-(3-hydroxycycloheptyl)-1-[1-[4-(trifluoromethoxy)benzoyl]-4-piperidyl]-3-(2-trimethylsilylethoxymethyl)imidazo[4,5-b]pyridin-2-one (270 mg, 0.42 mmol) in tetrahydrofuran (5 mL), at 0° C., was added TBAF (263 mg, 0.84 mmol) and the resulting reaction mixture was stirred at 80° C. for 16 hours. After 16 hours, the reaction mixture was quenched with a saturated aqueous sodium bicarbonate solution (30 mL), extracted with ethyl acetate (2×50 mL). The combined organic layers were concentrated under reduced pressure. The resulting residue was purified by preparative HPLC (conditions: MOBILE PHASE-A: 0.1% HCOOH in water, MOBILE PHASE-B: ACN, flow rate: 16 mL/min.; COLUMN: X-SELECT-C18 (19×250 mm) 5 μm, GRADIENT: time (minutes) (0, 2, 8) % B (20, 25, 55) to give 2 mg (0.9% yield) of (rac)-trans-6-(3-hydroxycycloheptyl)-1-[1-[4-(trifluoromethoxy)benzoyl]-4-piperidyl]-3H-imidazo[4,5-b]pyridin-2-one as an off white solid. LC/MS (m/z, M−H): calc. 517.2, found 517.0. ¹H NMR (400 MHz, DMSO-d6) δ ppm 11.31 (s, 1H) 7.71-7.86 (m, 1H) 7.55-7.63 (m, 2H) 7.39-7.50 (m, 3H) 4.58-4.72 (m, 1H) 4.40-4.49 (m, 2H) 3.90-4.00 (m, 1H) 3.58-3.74 (m, 1H) 2.95-3.09 (m, 3H) 1.99-2.12 (m, 2H) 1.45-1.90 (m, 12H); and

10 mg (4% yield) of (rac)-cis-6-(3-hydroxycycloheptyl)-1-[1-[4-(trifluoromethoxy)benzoyl]-4-piperidyl]-3H-imidazo[4,5-b]pyridin-2-one as an off white solid. LC/MS (m/z, M+H): calc. 519.2, found 519.0. ¹H NMR (400 MHz, DMSO-d6) δ ppm 11.37-11.43 (m, 1H) 7.73-7.81 (m, 1H) 7.57-7.64 (m, 2H) 7.44-7.52 (m, 3H) 4.60-4.72 (m, 1H) 4.42-4.50 (m, 2H) 3.68-3.80 (m, 1H) 2.88-3.02 (m, 2H) 1.81-1.99 (m, 4H) 1.64-1.81 (m, 5H) 1.45-1.62 (m, 5H) 0.78-0.94 (m, 2H).

### Example 68—ERK5 Inhibitory Activity of Compounds

Two assays were performed to assess the ERK5 inhibitory activity of compounds of the Examples, a cell-based assay and a cell-free biochemical assay. Results of the assays are shown below in Table 2. Where repeat measurements were taken, the value reported is a mean average.

### Inhibition of Cellular ERK5 Activity

The renal cancer cell line SN12C was transduced by a lentivirus pGreenFire1 MEF2 EF1 Neo (ref TR030VA-N) from SBI using standard infection protocol. pGreenFire1 MEF2 EF1 Neo allows the expression of luciferase gene under the control of minimal promoter with MEF2 transcriptional response elements. Cells harboring the reporter construct were selected by geneticin treatment. The selected cells were then transposed by a piggyback based plasmid pCM4007 allowing the expression of constitutively activated MEKSDD under control of TREG3 promoter, a doxycycline regulated promoter. Transposed cells were selected by puromycin treatment. Upon doxycycline treatment (1p g/ml) MEKSDD was expressed. MEKSDD activates ERK5 that phosphorylates MEF2C protein. Activated MEF2C protein can bind to its transcriptional response elements. Then the luciferase is expressed. In a 96-well plate (96F nuncleon refl37101 thermofisher), 50,000 cells were inoculated in 142.5 μl of RPMI medium containing 10% fetal calf serum, 1% glutamine, and 1 μg/ml doxycycline. After 24 hours, compounds were added in 7.5 μl culture medium (with 2% DMSO to give a final concentration of 0.1%) in order to obtain the desired concentration (0.3-1000 nM). The luciferase activity was measured using the Kit Bright Glo Luminescent Cell Assay Cat E2610 (Promega) according to the manufacturer's protocol. Luminescence was determined using 0.2 second reading/well using a Tecan SPARK. IC₅₀ values were calculated using XLFIT5 for Microsoft Excel using method 205. The IC₅₀ values represent the concentration of compound which inhibits the measurable luminescence signal by 50% as compared to DMSO-treated control cells.

### Cell-Free Assay of ERK5 Inhibition

An assay was performed to measure the capacity of each compound to inhibit ERK5 enzymatic activity. Compound potency was evaluated by Time-Resolved Forster Resonance Energy Transfer (FRET system). The activated catalytic domain of protein ERK5 (CarnaBiosciences #04-146) was mixed at 4 nM with varying concentrations of compound and incubated for 30 minutes at room temperature. A mixture of 1 mM ATP and 1 μM biotinylated synthetic peptide was added (Biosyntan GmbH). This synthetic peptide represents amino acids 30-52 of Eukaryotic translation initiation factor 4E-binding protein 1 (see, e.g., the sequence under accession No. NP_004086.1) biotinylated at the N-terminus. After 30 minutes at 37° C., the peptide phosphorylation by ERK5 was measured by addition of FRET reagents consisting of 12.5 μg/ml Streptavidin-XL665, 1 nM Anti-P-4EBP1 antibody and 300 ng/ml Anti-rabbit-K antibody. Following 90 minutes at room temperature fluorescent signals were read on the Pherastar FSX multimod detector from BMG Labtech (Exc° 340 nm, Em1 620 nm, Em2 665 nm). The IC₅₀ values represent the concentration of compound which inhibits the measurable fluorescence signal by 50% as compared to the DMSO only control.

TABLE 2
Results of cell-based and cell-free assays

| Compound | Cell-based assay | Cell-free assay |
|---|---|---|
| (Example No.) | (IC₅₀ in nM) | (IC₅₀ in nM) |
| 1 | 48 | 29 |
| 2 | 68 | 48 |
| 3 | 72 | 77 |
| 4 | 132 | 135 |
| 5 | 206 | 150 |
| 6 | 150 | 77 |
| 7 | 248 | 63 |
| 8 | 279 | 505 |
| 9 | 413 | 287 |
| 10 | 870 | 103 |
| 11 | 1834 | 1051 |
| 12 | 1860 | 442 |
| 13 | 1332 | 225 |
| 14 | 118 | 122 |
| 15 | 282 | 203 |
| 16 | 138 | 85 |
| 17 | 178 | 198 |
| 18 | 90 | 54* |
| 19 | 199 | 257 |
| 20 | 486 | 1104 |
| 21 | 42 | 395 |
| 22 | 140 | 684 |
| 23 | 184 | 86 |
| 24 | 67 | 502 |
| 25 | 57 | 377 |
| 26 | 118 | 235 |
| 27 | 536 | 1179 |
| 28 | 4926 | 533 |
| 29 | 213 | 420 |
| 30 | 310 | 15288 |
| 31 | 170 | 288 |
| 32 | 347 | 1137 |
| 33 | 124 | 376 |
| 34 | 217 | 974 |
| 35 | 205 | 695 |
| 36 | 285 | 888 |
| 37 | 167 | 681 |
| 38 | 541 | 1475 |
| 39 | 75 | 117 |
| 40 | 168 | 291 |
| 41 | 152 | 557 |
| 42 | 136 | 354 |
| 43 | 74 | 150 |
| 44 | 169 | 310 |
| 45 | 29 | 107 |
| 46 | 199 | 643 |
| 47 | 273 | 239 |
| 48 | 117 | 78 |
| 49 | 278 | 587 |
| 50 | 361 | 1621 |
| 51 | 205 | 52 |
| 52 | 244 | 97 |
| 53 | 184 | 405 |
| 54 | 168 | 78 |
| 55 | 83 | 87 |
| 56 | 96 | 430 |
| 57 | 204 | 180 |
| 58 | 222 | 288 |
| 59 | 2148 | 30000 |
| 60 | 641 | 1084 |
| 61 | 202 | — |
| 62 | 257 | — |
| 63 | 254 | — |
| 64 | 358 | — |
| 65 | 575 | — |
| 66 | 547 | — |
| 67 | 358 | — |

*repeat measurement gave a mean average value of 82 nM
“—” denotes that the values were not measured

The data in Table 2 indicate that the compounds synthesised are active in the micromolar or nanomolar concentration range in cell-based and/or cell-free systems. All of the compounds synthesised have an IC₅₀ value below 10 μM in at least one of the cell-based and cell-free assays.

It is to be understood that while the disclosure has been described in conjunction with the above embodiments, that the foregoing description and examples are intended to illustrate and not limit the scope of the disclosure. Other aspects, advantages, and modifications within the scope of the disclosure will be apparent to those skilled in the art to which the disclosure pertains.

In addition, where features or aspects are described in terms of Markush groups, those skilled in the art will recognize that such features or aspects are also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A compound, being of Formula (N-I) 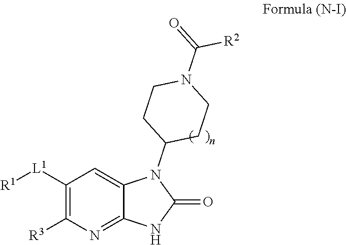
or a pharmaceutically acceptable salt thereof, wherein:
R¹ is selected from halo, —(C₁-C₆)alkyl, —(C₃-C₇)cycloalkyl, 4- to 10-membered heterocycloalkyl, and 5- to 10-membered heteroaryl, wherein R¹ is optionally substituted with one or more occurrences of R^{A},
wherein each R^{A} is independently selected from halo, —OH, oxo, ═N—OH, —(C₁-C₃)alkyl, and —O(C₁-C₃)alkyl, wherein each occurrence of —(C₁-C₃)alkyl is optionally substituted by one or more groups independently selected from halo and —OH,
and/or wherein two occurrences of R^{A} may be taken together with the atom(s) to which they are attached to form a (C₃-C₆)cycloalkyl group or a 3- to 6-membered heterocyclic group, wherein the cycloalkyl or heterocyclic group is optionally substituted by one or more halo;
L¹ is selected from a direct bond, —O—, and —CH₂—;
R² is —(C₆-C₁₀)aryl, wherein R² is substituted with one, or two occurrences of R^{B},
wherein each R^{B} is independently selected from —OH, —NH₂, —SF₅, —NHC(O)OC(CH₃)₃, and —O(C₁-C₃)alkyl, wherein each occurrence of —O(C₁-C₃)alkyl is optionally substituted by one or more halo;
R³ is selected from —H, —OH, and —(C₁-C₃)alkyl; and
n is 0, or 1.

2. The compound of claim 1, wherein R¹ is selected from: 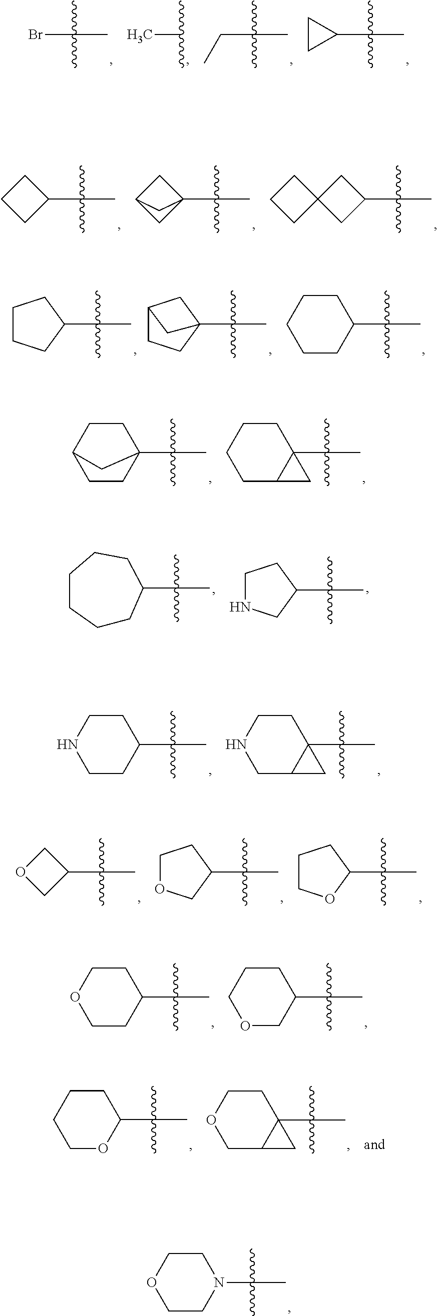
wherein R¹ is optionally substituted by one or two occurrences of R^{A} as defined in claim 1.

3. The compound of claim 2, wherein each R^{A} is independently selected from —F, —OH, oxo, ═N—OH, —OCH₃, and —(C₁-C₂)alkyl optionally substituted by one or more substituents selected from —F and —OH.

4. The compound of any one of claims 1-3, wherein L¹ is —O— or —CH₂—.

5. The compound of any one of claims 1-4, wherein R² is selected from:

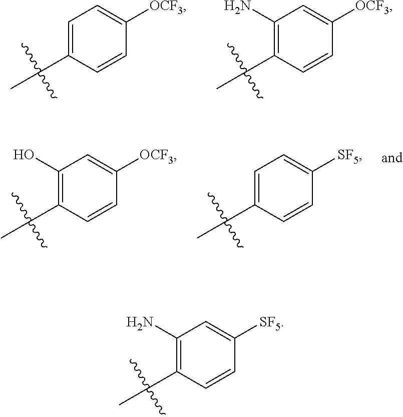

6. A compound, being of Formula (N-II) 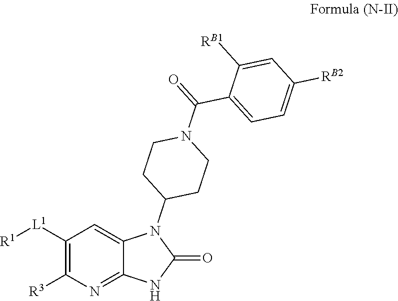
or a pharmaceutically acceptable salt thereof, wherein:
R¹ is selected from halo (e.g., —Br), —(C₁-C₆)alkyl, —(C₃-C₇)cycloalkyl, and 4- to 10-membered heterocycloalkyl, wherein R¹ is optionally substituted with one or more occurrences of R^{A},
wherein each R^{A} is independently selected from halo (e.g., —F), —OH, oxo, —O(C₁-C₃)alkyl, and —(C₁-C₃)alkyl optionally substituted by one or more groups independently selected from halo (e.g., —F) and —OH,
and/or wherein two occurrences of R^{A} may be taken together with the atom(s) to which they are attached to form a (C₃-C₆)cycloalkyl or 3- to 6-membered heterocycloalkyl group, wherein the cycloalkyl group is optionally substituted by —F;
L¹ is selected from a direct bond, —O—, and —CH₂—;
R^{B1} is selected from —H, —OH, —NH₂, and —NHC(O)OC(CH₃)₃;
R^{B2} is selected from —SF₅, and —OCF₃; and
R³ is selected from —H, and —(C₁-C₃)alkyl (e.g., —CH₃).

7. The compound of claim 6, wherein R¹ is selected from: 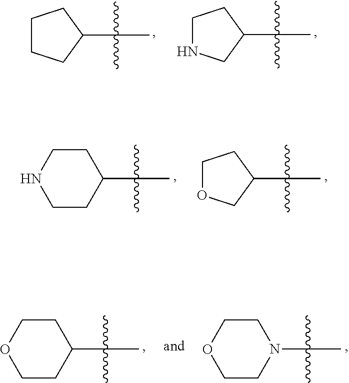
wherein R¹ is optionally substituted by one occurrence of R^{A} as defined in any one of claims 1 to 6.

8. A compound, being of Formula (III) or Formula (IV) 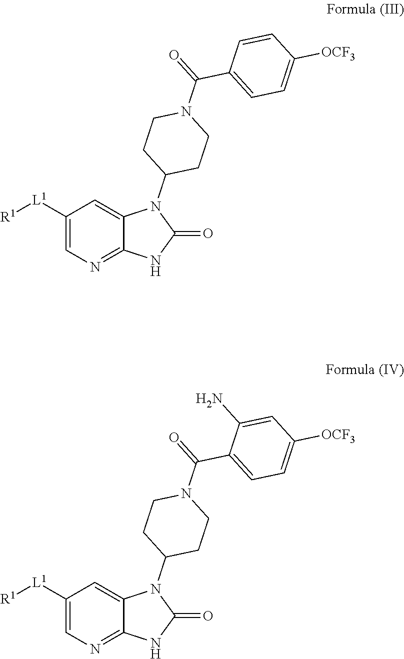
or a pharmaceutically acceptable salt thereof, wherein R¹ and L¹ are as defined in any one of claims 1 to 7.

9. A compound, being of Formula (V), Formula (VI), or Formula (VII) 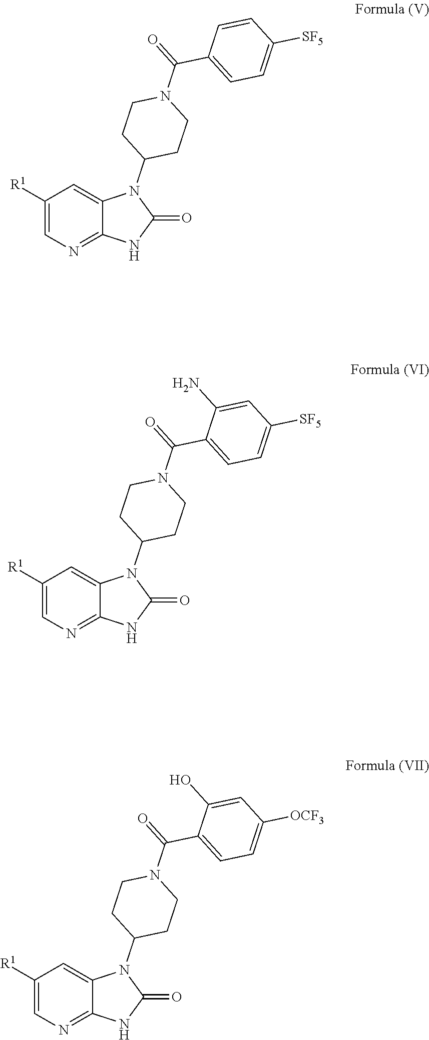
or a pharmaceutically acceptable salt thereof, wherein R¹ is selected from —(C₃-C₇)cycloalkyl, and 4- to 10-membered heterocycloalkyl, wherein R¹ is optionally substituted with one or more occurrences of R^{A}, and wherein each R^{A} is independently —(C₁-C₃)alkyl optionally substituted by —OH.

10. A compound selected from the group consisting of:
1-[1-[2-amino-4-(trifluoromethoxy)benzoyl]-4-piperidyl]-6-tetrahydropyran-4-yloxy-3H-imidazo[4,5-b]pyridin-2-one,
1-[1-[2-amino-4-(trifluoromethoxy)benzoyl]-4-piperidyl]-6-tetrahydropyran-4-yl-3H-imidazo[4,5-b]pyridin-2-one,
1-[1-[2-amino-4-(trifluoromethoxy)benzoyl]-4-piperidyl]-6-N-morpholino-3H-imidazo[4,5-b]pyridin-2-one,
6-tetrahydropyran-4-yloxy-1-[1-[4-(trifluoromethoxy)benzoyl]-4-piperidyl]-3H-imidazo[4,5-b]pyridin-2-one,
6-[(1-methyl-4-piperidyl)oxy]-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-(1-methylpyrrolidin-3-yl)oxy-3H-imidazo[4,5-b]pyridin-2-one,
1-[1-[2-amino-4-(trifluoromethoxy)benzoyl]-4-piperidyl]-6-[(1-methyl-4-piperidyl)oxy]-3H-imidazo[4,5-b]pyridin-2-one,
6-N-morpholino-1-[1-[4-(trifluoromethoxy)benzoyl]-4-piperidyl]-3H-imidazo[4,5-b]pyridin-2-one,
6-(1-methylpyrrolidin-3-yl)oxy-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[2-(hydroxymethyl)morpholin-4-yl]-3H-imidazo[4,5-b]pyridin-2-one,
6-[2-(hydroxymethyl)morpholin-4-yl]-1-[1-[4-(trifluoromethoxy)benzoyl]-4-piperidyl]-3H-imidazo[4,5-b]pyridin-2-one,
1-[1-[2-amino-4-(pentafluoro-λ⁶-sulfanyl)benzoyl]-4-piperidyl]-6-tetrahydropyran-4-yl-3H-imidazo[4,5-b]pyridin-2-one,
6-tetrahydrofuran-3-yl-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-tetrahydrofuran-3-yl-3H-imidazo[4,5-b]pyridin-2-one,
6-cyclopentyl-1-[1-[4-(trifluoromethoxy)benzoyl]-4-piperidyl]-3H-imidazo[4,5-b]pyridin-2-one,
1-[1-[2-hydroxy-4-(trifluoromethoxy)benzoyl]-4-piperidyl]-6-tetrahydropyran-4-yl-3H-imidazo[4,5-b]pyridin-2-one,
1-[1-[4-(pentafluoro-λ⁶-sulfanyl)benzoyl]-4-piperidyl]-6-tetrahydropyran-4-yl-3H-imidazo[4,5-b]pyridin-2-one,
1-[1-[2-amino-4-(trifluoromethoxy)benzoyl]-4-piperidyl]-5-methyl-6-tetrahydropyran-4-yl-3H-imidazo[4,5-b]pyridin-2-one,
1-[1-[2-amino-4-(trifluoromethoxy)benzoyl]-4-piperidyl]-6-(2-fluorospiro[3.3]heptan-6-yl)-3H-imidazo[4,5-b]pyridin-2-one,
1-[1-[2-amino-4-(trifluoromethoxy)benzoyl]-4-piperidyl]-6-(2-oxo-4-piperidyl)-3H-imidazo[4,5-b]pyridin-2-one,
1-[1-[2-amino-4-(trifluoromethoxy)benzoyl]-4-piperidyl]-6-(tetrahydrofuran-3-ylmethyl)-3H-imidazo[4,5-b]pyridin-2-one,
1-[1-[2-amino-4-(trifluoromethoxy)benzoyl]-4-piperidyl]-6-(2-methoxyethyl)-3H-imidazo[4,5-b]pyridin-2-one,
1-[1-[2-amino-4-(trifluoromethoxy)benzoyl]-4-piperidyl]-6-tetrahydrofuran-2-yl-3H-imidazo[4,5-b]pyridin-2-one,
trans-6-(4-methoxycyclohexyl)-1-[1-[4-(trifluoromethoxy)benzoyl]-4-piperidyl]-3H-imidazo[4,5-b]pyridin-2-one,
cis-6-(4-methoxycyclohexyl)-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-(3-azabicyclo[4.1.0]heptan-6-yl)-3H-imidazo[4,5-b]pyridin-2-one,
1-[1-[2-amino-4-(trifluoromethoxy)benzoyl]-4-piperidyl]-6-cyclobutyl-3H-imidazo[4,5-b]pyridin-2-one,
1-[1-[2-amino-4-(trifluoromethoxy)benzoyl]-4-piperidyl]-6-[3-(trifluoromethyl)-1-bicyclo[1.1.1]pentanyl]-3H-imidazo[4,5-b]pyridin-2-one,
1-[1-[2-amino-4-(trifluoromethoxy)benzoyl]-4-piperidyl]-6-(3-oxabicyclo[4.1.0]heptan-6-yl)-3H-imidazo[4,5-b]pyridin-2-one,
1-[1-[2-amino-4-(trifluoromethoxy)benzoyl]-4-piperidyl]-6-(1-bicyclo[2.1.1]hexanyl)-3H-imidazo[4,5-b]pyridin-2-one,
1-[1-[2-amino-4-(trifluoromethoxy)benzoyl]-4-piperidyl]-6-[3-(difluoromethyl)-1-bicyclo[1.1.1]pentanyl]-3H-imidazo[4,5-b]pyridin-2-one,
1-[1-[2-amino-4-(trifluoromethoxy)benzoyl]-4-piperidyl]-6-norcaran-1-yl-3H-imidazo[4,5-b]pyridin-2-one,
1-[1-[2-amino-4-(trifluoromethoxy)benzoyl]-4-piperidyl]-6-(4,4-difluorocyclohexyl)-3H-imidazo[4,5-b]pyridin-2-one,
1-[1-[2-amino-4-(trifluoromethoxy)benzoyl]-4-piperidyl]-6-cyclohexyl-3H-imidazo[4,5-b]pyridin-2-one,
1-[1-[2-amino-4-(trifluoromethoxy)benzoyl]-4-piperidyl]-6-norbornan-2-yl-3H-imidazo[4,5-b]pyridin-2-one,
1-[1-[2-amino-4-(trifluoromethoxy)benzoyl]-4-piperidyl]-6-tetrahydropyran-2-yl-3H-imidazo[4,5-b]pyridin-2-one,
1-[1-[2-amino-4-(trifluoromethoxy)benzoyl]-4-piperidyl]-6-(tetrahydropyran-4-ylmethyl)-3H-imidazo[4,5-b]pyridin-2-one,
1-[1-[2-amino-4-(trifluoromethoxy)benzoyl]-4-piperidyl]-6-tetrahydropyran-3-yl-3H-imidazo[4,5-b]pyridin-2-one,
6-tetrahydropyran-4-yl-1-[1-[4-(trifluoromethoxy)benzoyl]-4-piperidyl]-3H-imidazo[4,5-b]pyridin-2-one,
6-(oxetan-3-ylmethyl)-1-[1-[4-(trifluoromethoxy)benzoyl]-4-piperidyl]-3H-imidazo[4,5-b]pyridin-2-one,
1-[1-[2-amino-4-(trifluoromethoxy)benzoyl]-4-piperidyl]-6-[tetrahydrofuran-3-yl]oxy-3H-imidazo[4,5-b]pyridin-2-one,
trans-1-[1-[2-amino-4-(trifluoromethoxy)benzoyl]-4-piperidyl]-6-(4-methoxycyclohexyl)-3H-imidazo[4,5-b]pyridin-2-one,
cis-1-[1-[2-amino-4-(trifluoromethoxy)benzoyl]-4-piperidyl]-6-(4-methoxycyclohexyl)-3H-imidazo[4,5-b]pyridin-2-one,
1-[1-[2-amino-4-(trifluoromethoxy)benzoyl]-4-piperidyl]-6-[1-methylpyrrolidin-3-yl]oxy-3H-imidazo[4,5-b]pyridin-2-one,
1-[1-[2-amino-4-(pentafluoro-λ⁶-sulfanyl)benzoyl]pyrrolidin-3-yl]-6-tetrahydropyran-4-yl-3H-imidazo[4,5-b]pyridin-2-one,
1-[1-[4-(pentafluoro-λ⁶-sulfanyl)benzoyl]pyrrolidin-3-yl]-6-tetrahydropyran-4-yl-3H-imidazo[4,5-b]pyridin-2-one,
trans-1-[1-[2-amino-4-(trifluoromethoxy)benzoyl]-4-piperidyl]-6-(4-hydroxycyclohexyl)-3H-imidazo[4,5-b]pyridin-2-one,
cis-1-[1-[2-amino-4-(trifluoromethoxy)benzoyl]-4-piperidyl]-6-(4-hydroxycyclohexyl)-3H-imidazo[4,5-b]pyridin-2-one,
1-[1-[2-amino-4-(trifluoromethoxy)benzoyl]-4-piperidyl]-6-[1-(2,2,2-trifluoroethyl)-4-piperidyl]-3H-imidazo[4,5-b]pyridin-2-one,
1-[1-[2-amino-4-(trifluoromethoxy)benzoyl]-4-piperidyl]-6-(1-methyl-4-piperidyl)-3H-imidazo[4,5-b]pyridin-2-one,
1-[1-[2-amino-4-(trifluoromethoxy)benzoyl]-4-piperidyl]-6-tetrahydrofuran-3-yloxy-3H-imidazo[4,5-b]pyridin-2-one,
6-tetrahydrofuran-3-yloxy-1-[1-[4-(trifluoromethoxy)benzoyl]-4-piperidyl]-3H-imidazo[4,5-b]pyridin-2-one,
trans-6-(4-hydroxycyclohexyl)-1-[1-[4-(trifluoromethoxy)benzoyl]-4-piperidyl]-3H-imidazo[4,5-b]pyridin-2-one,
cis-6-(4-hydroxycyclohexyl)-1-[1-[4-(trifluoromethoxy)benzoyl]-4-piperidyl]-3H-imidazo[4,5-b]pyridin-2-one,
tert-butyl N-[2-[4-(6-bromo-2-oxo-3H-imidazo[4,5-b]pyridin-1-yl)piperidine-1-carbonyl]-5-(trifluoromethoxy)phenyl]carbamate,
6-bromo-1-[1-[4-(trifluoromethoxy)benzoyl]-4-piperidyl]-3H-imidazo[4,5-b]pyridin-2-one,
trans-6-(4-hydroxycycloheptyl)-1-[1-[4-(trifluoromethoxy)benzoyl]-4-piperidyl]-3H-imidazo[4,5-b]pyridin-2-one,
cis-6-(4-hydroxycycloheptyl)-1-[1-[4-(trifluoromethoxy)benzoyl]-4-piperidyl]-3H-imidazo[4,5-b]pyridin-2-one,
6-(4-oxocycloheptyl)-1-[1-[4-(trifluoromethoxy)benzoyl]-4-piperidyl]-3H-imidazo[4,5-b]pyridin-2-one,
6-[4-hydroxyiminocycloheptyl]-1-[1-[4-(trifluoromethoxy)benzoyl]-4-piperidyl]-3H-imidazo[4,5-b]pyridin-2-one,
trans-6-(3-hydroxycycloheptyl)-1-[1-[4-(trifluoromethoxy)benzoyl]-4-piperidyl]-3H-imidazo[4,5-b]pyridin-2-one,
cis-6-(3-hydroxycycloheptyl)-1-[1-[4-(trifluoromethoxy)benzoyl]-4-piperidyl]-3H-imidazo[4,5-b]pyridin-2-one,
and the pharmaceutically acceptable salts thereof.

11. A pharmaceutical composition comprising the compound of any one of claims 1-10 and at least one pharmaceutically acceptable excipient or carrier.

12. A compound according to any one of claims 1-10, or a pharmaceutical composition according to claim 11, for use in therapy.

13. A compound according to any one of claims 1-10, or a pharmaceutical composition according to claim 11, for use in the treatment or prevention of cancer.

14. The compound or pharmaceutical composition for use according to claim 13, wherein the cancer is characterized by increased MAPK7 expression and/or increased ERK5 activity.

15. The compound or pharmaceutical composition for use according to claim 13 or claim 14, wherein the cancer is selected from leukaemia (e.g., chronic myeloid leukaemia), breast cancer, multiple myeloma, colon cancer, colorectal cancer, lung cancer, pancreatic cancer, renal cell carcinoma, mesothelioma, adenocarcinoma, neuroblastoma, melanoma, and hepatocellular carcinoma.
